(19) United States
(12) Patent Application Publication
(10) Pub. No.: US 2025/0213664 A1
(43) Pub. Date: Jul. 3, 2025

(54) MRNAS ENCODING CHECKPOINT CANCER VACCINES AND USES THEREOF

(71) Applicant: ModernaTX, Inc., Cambridge, MA (US)

(72) Inventors: Joshua P. Frederick, Cambridge, MA (US); Praveen Aanur, Cambridge, MA (US); Brandon Coder, Cambridge, MA (US); Celine Robert-Tissot, Cambridge, MA (US); Robert Meehan, Cambridge, MA (US); Stephen Greene, Cambridge, MA (US); Maija Garnaas, Cambridge, MA (US)

(21) Appl. No.: 18/839,326

(22) PCT Filed: Feb. 17, 2023
(86) PCT No.: PCT/US2023/062844

§ 371 Date: Aug. 16, 2024

Related U.S. Application Data
(60) Provisional application No. 63/381,460, filed on Oct. 28, 2022.
(60) Provisional application No. 63/311,716, filed on Feb. 18, 2022.

(51) Int. Cl. A61K 39/00; A61K 9/00; A61K 9/1271; A61K 9/51; A61P 35/00; A61P 37/04; C07K 16/28

(52) U.S. Cl. CPC A61K 39/001111; A61K 9/0019; A61K 9/1271; A61K 9/5123; A61K 39/001154; A61P 35/00; A61P 37/04; C07K 16/2818; A61K 2039/53; A61K 2039/54; A61K 2039/545; A61K 2039/55555; A61K 2039/572; A61K 2039/812; A61K 2039/82; A61K 2039/876

(57) ABSTRACT

The disclosure features lipid nanoparticle (LNP) compositions comprising checkpoint cancer vaccines and uses thereof. The LNP compositions of the present disclosure comprise mRNA therapeutics encoding checkpoint cancer vaccine comprising IDO and PD-L1 antigenic peptides. The LNP compositions of the present disclosure can induce an immune response and stimulate T effector cells in vivo and, accordingly are useful in treating cancer.

### CROSS REFERENCE TO RELATED APPLICATIONS

This application is a U.S. National Phase Application under 35 U.S.C. § 371 of International Application No. PCT/US2023/062844, filed Feb. 17, 2023, which claims the benefit of U.S. Provisional Application No. 63/311,716, filed Feb. 18, 2022, and U.S. Provisional Application No. 63/381,460, filed Oct. 28, 2022. The contents of the aforementioned applications are hereby incorporated by reference in their entirety.

### SEQUENCE LISTING

This application contains a Sequence Listing which has been submitted electronically in XML format and is hereby incorporated by reference in its entirety. Said XML copy, created on Mar. 13, 2023, is named M2180-7017WO_SL.xml and is 671,603 bytes in size.

### BACKGROUND OF THE DISCLOSURE

Melanoma is the fifth most common cancer diagnosis in the U.S. It accounts for 5.3% of all new cancer diagnoses and 1.5% of all cancer-related deaths. Cutaneous melanoma is a cancer that starts in the melanocytes (pigment-producing cells) of the skin. If diagnosed at the local stage, the 5-year survival rate is approximately 95%. However, for regional or metastatic disease (stage IIIB+), 5-year survival rates decline to approximately 30 to 60%. Approximately 18,000 new patients are diagnosed with stage IIIB+cutaneous melanoma in the U.S. Advanced melanoma, a rare and serious type of skin cancer, is responsible for most skin cancer-related deaths, despite representing only 1% of skin cancer cases. Current standard of care pembrolizumab, nivolumab or the combination of nivolumab+ipilimumab.

NSCLC frequently goes undetected, remaining asymptomatic until it has progressed to later stages. Approximately, 115,000 people are diagnosed with metastatic NSCLC or progress to metastatic disease annually in the U.S. The current approach to treatment of metastatic NSCLC treatment is dependent on the presence of PD-L1 expression. If tumor PD-L1 expression is greater than 50% pembrolizumab or atezolizumab monotherapy are preferred, while a combination of chemotherapy and pembrolizumab is preferred for patients with PD-L1 expression less than 50%.

While there are several treatments for cancers, such as melanoma and NSCLC, there is an unmet need to develop new modalities and therapies that can improve therapeutic outcomes and prolong survival for patients with cancer.

### SUMMARY OF THE DISCLOSURE

The present disclosure provides, inter alia, polynucleotide constructs and lipid nanoparticle (LNP) compositions comprising such polynucleotides which encode checkpoint cancer vaccines (e.g., comprising one or more IDO antigenic peptides and one or more PD-L1 antigenic peptides) and uses thereof. The LNP compositions of the present disclosure comprise one or more mRNA molecules encoding (i) one or more IDO antigenic peptides; and (ii) one or more PD-L1 antigenic peptides and, optionally adjuvant amino acid sequences. In an aspect, the LNP compositions of the present disclosure can stimulate an immune response (e.g., stimulate effector T-cells to target and kill suppressive immune and tumor cells that express IDO or PD-L1); prime T cells to induce recognition of tumor-associated antigens, induce helper T cells, promote influx of T cells into tumor sites, and induce cytotoxic T cell-mediated killing of tumor cells. Also disclosed herein are methods of using LNP compositions comprising checkpoint cancer vaccines, for treating a cancer, or for stimulating an immune response in a subject.

Additional aspects of the disclosure are described in further detail below.

In an aspect, provided herein are polynucleotides (e.g., mRNA) which encode a checkpoint cancer vaccine comprising (i) one or more Indoleamine-pyrrole 2,3-dioxygenase (IDO) antigenic peptides and (ii) one or more programmed death-ligand 1 (PD-L1) antigenic peptides. Optionally, the polynucleotide can also comprise sequences which encode for adjuvant amino acid sequences. The invention also pertains to lipid nanoparticle (LNP) compositions comprising such polynucleotides.

In another aspect, the disclosure provides a lipid nanoparticle (LNP) composition for immunomodulation, e.g., for stimulating an immune response by IDO/PDL1 specific T cells or breaking immune tolerance (e.g., stimulating T effector cells by increasing their activation and/or attracting them to tumor cells which can express IDO and PDL1), the composition comprising an mRNA which (i) one or more Indoleamine-pyrrole 2,3-dioxygenase (IDO) antigenic peptides and (ii) one or more programmed death-ligand 1 (PD-L1) antigenic peptides. Optionally, the mRNA can also encode for adjuvant amino acid sequences. In one embodiment, the LNP composition promotes infiltration of tumor cells by CD4+ and/or CD8+ T cells and promotes killing of tumor cells expression IDO and/or PDL1.

In an aspect, provide herein is a lipid nanoparticle (LNP) composition, for stimulating T effector cells in a subject having melanoma or NSCLC, the composition comprising an mRNA which encodes a checkpoint cancer vaccine comprising (i) one or more Indoleamine-pyrrole 2,3-dioxygenase (IDO) antigenic peptides and (ii) one or more programmed death-ligand 1 (PD-L1) antigenic peptides. Optionally, the mRNA can also encode for adjuvant amino acid sequences.

In an embodiment administration of the LNP composition disclosed herein, results in amelioration or delay or progression of cancer, e.g., as described herein, in a subject, e.g., as measured by an assay described herein. In one embodiment, a checkpoint inhibitor, e.g., anti-PD1 antibody, anti-CTLA4 antibody, or combination thereof can also be administered to the subject.

In an embodiment of any of the LNP compositions disclosed herein comprise an mRNA encoding the checkpoint cancer vaccine comprises which mRNA comprises at least one chemical modification.

In an embodiment of any of the LNP compositions disclosed herein, the LNP composition comprises: (i) an ionizable lipid, e.g., an ionizable amino lipid; (ii) a sterol or other structural lipid; (iii) a non-cationic helper lipid or phospholipid; and (iv) a PEG-lipid.

In an aspect, provided herein is a pharmaceutical composition comprising an LNP composition disclosed herein.

In an aspect, provided herein is a method of modulating, e.g., inducing or promoting, an immune response in a subject, comprising administering to the subject in need thereof an effective amount of an LNP composition comprising an mRNA which encodes a checkpoint cancer vaccine comprising (i) one or more IDO antigenic peptides and (ii) one or more PD-L1 antigenic peptides. Optionally, the mRNA can also encode for adjuvant amino acid sequences.

In another aspect, the disclosure provides a method of stimulating T effector cells in a subject, comprising administering to the subject an effective amount of an LNP composition comprising an mRNA which encodes a checkpoint cancer vaccine comprising (i) one or more IDO antigenic peptides and (ii) one or more PD-L1 antigenic peptides. Optionally, the mRNA can also encode for adjuvant amino acid sequences.

In yet another aspect, provided herein is a method of treating, or preventing the spread of, or a symptom of, a cancer or a metastatic lesion thereof, e.g., a cutaneous melanoma (e.g., a 1 L cutaneous melanoma stage IIIB+) or an NSCLC (e.g., a 1 L NSCLC), comprising administering to the subject in need thereof an effective amount of an LNP composition comprising mRNA which encodes a checkpoint cancer vaccine comprising (i) one or more IDO peptides and (ii) one or more PD-L1 peptides. Optionally, the mRNA can also encode for adjuvant amino acid sequences.

In an embodiment of any of the methods disclosed herein, the checkpoint cancer vaccine comprises alternating antigenic peptides of IDO and PD-L1 (e.g., is a multimer).

In an embodiment of any of the methods disclosed herein, the LNP composition comprising an mRNA which encodes a checkpoint cancer vaccine comprising (i) one or more IDO antigenic peptides and (ii) one or more PD-L1 antigenic peptides, is administered in combination with an additional agent, e.g., an agent that further stimulates an immune response, e.g., a checkpoint inhibitor such as an anti-PD1 antibody or an anti-CTLA4 antibody.

In an embodiment, the additional agent is administered in the form of a therapeutic protein. In another embodiment, the additional agent is administered in the form of a polynucleotide encapsulated in an LNP. In one embodiment, the LNP composition and the additional agent are in the same composition or in separate compositions.

In an embodiment, the LNP composition and the additional agent are administered substantially simultaneously or sequentially. In an embodiment, for sequential administration the LNP composition is administered before the additional agent is administered. In an embodiment, the order of administration is reversed. In an embodiment of any of the methods disclosed herein, the cancer is a solid tumor, e.g., a locally advanced or metastatic solid tumor. In some embodiments, the cancer is chosen from: a cutaneous melanoma (e.g., a 1 L cutaneous melanoma stage IIIB+), an NSCLC (e.g., a 1 L NSCLC), a bladder cancer (e.g., a non-muscle invasive bladder cancer), a head and neck cancer (e.g., a head and neck squamous cell carcinoma), a colorectal cancer (e.g., a microsatellite stable colorectal cancer), a basal cell carcinoma, or a breast cancer (e.g., a triple negative breast cancer).

In an embodiment, the cancer is a cutaneous melanoma. In an embodiment, the cutaneous melanoma is a 1 L cutaneous melanoma stage IIIB+. In an embodiment, the melanoma is a refractory melanoma. In an embodiment, the cancer is a NSCLC. In an embodiment, the NSCLC is a 1 L NSCLC. In an embodiment, the NSCLC is a locally advanced or metastatic and/or checkpoint inhibitor refractory NSCLC.

In some embodiments of any of the methods disclosed herein, the LNP composition comprises: (i) an ionizable lipid, e.g., an amino lipid; (ii) a sterol or other structural lipid; (iii) a non-cationic helper lipid or phospholipid; and (iv) a PEG-lipid. In some embodiments, the ionizable lipid comprises Compound 25. In some embodiments of any of the methods disclosed herein, the LNP composition comprises an ionizable lipid comprising Compound 25 and a PEG-lipid comprising PEG DMG.

In yet another aspect, disclosed herein is a kit comprising a container comprising an LNP composition disclosed herein, or a pharmaceutical LNP composition disclosed herein.

In some embodiments, the kit comprises a package insert comprising instructions for administration of the LNP composition or pharmaceutical LNP composition for treating a cancer.

In some embodiments, the LNP composition comprises a pharmaceutically acceptable carrier.

Additional features of any of the LNP compositions, pharmaceutical composition comprising said LNPs, methods, or compositions for use disclosed herein include the following aspects or embodiments.

In an aspect, the disclosure provides an LNP composition comprising a polynucleotide, e.g., encoding a checkpoint cancer vaccine, e.g., comprising one or more IDO antigenic peptides and one or more PD-L1 antigenic peptides, e.g., as described herein. Optionally, the polynucleotide can also comprise sequences which encode for adjuvant amino acid sequences.

In an aspect, an LNP composition disclosed herein comprises a polynucleotide encoding a checkpoint cancer vaccine comprising one or more IDO antigenic peptides. In an embodiment, the IDO antigenic peptide comprises an amino acid sequence having at least 85%, 90%, 95%, 96%, 97%, 98%, 99% or 100% identity to an IDO antigenic peptide amino acid sequence provided in Table 1A, e.g., SEQ ID NO: 1, or an antigenic fragment thereof. In an embodiment, the IDO antigenic peptide comprises the amino acid sequence of an IDO amino acid sequence provided in Table 1A, e.g., SEQ ID NO: 1, or an antigenic fragment thereof. In an embodiment, the IDO antigenic peptide comprises the amino acid sequence of SEQ ID NO: 1, or an antigenic fragment thereof.

In an embodiment, the polynucleotide encoding the IDO antigenic peptide comprises a nucleotide sequence having at least 50%, 55%, 60%, 65%, 70%, 75%, 80%, 85%, 90%, 95%, 96%, 97%, 98%, 99% or 100% identity to the sequence of SEQ ID NO: 2, or an antigenic fragment thereof. In an embodiment, the polynucleotide (e.g., mRNA) encoding the IDO antigenic peptide comprises the nucleotide sequence of SEQ ID NO: 2, or an antigenic fragment thereof. In an embodiment, the polynucleotide encoding the IDO antigenic peptide comprises a codon-optimized nucleotide sequence.

In an aspect, an LNP composition disclosed herein comprises a polynucleotide encoding a checkpoint cancer vaccine comprising one or more PD-L1 antigenic peptides. In an embodiment, the PD-L1 antigenic peptide comprises an amino acid sequence having at least 85%, 90%, 95%, 96%, 97%, 98%, 99% or 100% identity to an PD-L1 antigenic peptide amino acid sequence provided in Table 1A, e.g., SEQ ID NO: 3, or an antigenic fragment thereof. In an embodiment, the PD-L1 molecule comprises the amino acid sequence of a PD-L1 amino acid sequence provided in Table 1A, e.g., SEQ ID NO: 3, or an antigenic fragment thereof. In an embodiment, the PD-L1 molecule comprises the amino acid sequence of SEQ ID NO: 3, or an antigenic fragment thereof.

In an embodiment, the polynucleotide encoding the PD-L1 antigenic peptide comprises a nucleotide sequence having at least 50%, 55%, 60%, 65%, 70%, 75%, 80%, 85%, 90%, 95%, 96%, 97%, 98%, 99% or 100% identity to the sequence of SEQ ID NO: 4, or an antigenic fragment thereof. In an embodiment, the polynucleotide (e.g., mRNA) encoding the PD-L1 antigenic peptide comprises the nucleotide sequence of SEQ ID NO: 4, or an antigenic fragment thereof. In an embodiment, the polynucleotide encoding the PD-L1 antigenic peptide comprises a codon-optimized nucleotide sequence.

In an aspect, an LNP composition disclosed herein comprises a polynucleotide encoding a checkpoint cancer vaccine, e.g., comprising one or more (e.g., 1, 2, 3, 4, or more) IDO antigenic peptides and one or more (e.g., 1, 2, 3, 4, or more) PD-L1 antigenic peptides. In some embodiments, the checkpoint cancer vaccine comprises alternating IDO and PD-L1 antigenic peptides. In an embodiment, the checkpoint cancer vaccine comprises one IDO antigenic peptide and one PD-L1 antigenic peptide. In an embodiment, the checkpoint cancer vaccine comprises two IDO antigenic peptides and two PD-L1 antigenic peptides. In an embodiment, the checkpoint cancer vaccine comprises three IDO antigenic peptides and three PD-L1 antigenic peptides. In an embodiment, the checkpoint cancer vaccine comprises four IDO antigenic peptides and four PD-L1 antigenic peptides. In some embodiments, the four IDO and four PD-L1 antigenic peptides are arranged in alternating manner. Accordingly, in an embodiment, the checkpoint cancer vaccine comprises an (i) IDO antigenic peptide, (ii) a PD-L1 antigenic peptide, (iii) an IDO antigenic peptide, (iv) a PD-L1 antigenic peptide, (v) an IDO antigenic peptide, (vi) a PD-L1 antigenic peptide, (vii) an IDO antigenic peptide, and (viii) a PD-L1 antigenic peptide).

In an embodiment, the alternating IDO and PD-L1 antigenic peptides comprise an amino acid sequence having at least 85%, 90%, 95%, 96%, 97%, 98%, 99% or 100% identity to an IDO amino acid sequence provided in Table 1A, e.g., SEQ ID NO: 5, or an antigenic fragment thereof. In an embodiment, the alternating IDO and PD-L1 antigenic peptides comprise the amino acid sequence of an amino acid sequence provided in Table 1A, e.g., SEQ ID NO: 5, or an antigenic fragment thereof. In an embodiment, the alternating IDO and PD-L1 antigenic peptides comprise the amino acid sequence of SEQ ID NO: 5, or an antigenic fragment thereof.

In an embodiment, the polynucleotide encoding the alternating IDO and PD-L1 antigenic peptides comprise a nucleotide sequence having at least 50%, 55%, 60%, 65%, 70%, 75%, 80%, 85%, 90%, 95%, 96%, 97%, 98%, 99%, or 100% identity to the sequence of SEQ ID NO: 6, 300, 301, or 302, or an antigenic fragment thereof. In an embodiment, the polynucleotide (e.g., mRNA) encoding the alternating IDO and PD-L1 antigenic peptides comprise the nucleotide sequence of SEQ ID NO: 6, 300, 301, or 302, or an antigenic fragment thereof. In an embodiment, the polynucleotide encoding the alternating IDO and PD-L1 antigenic peptides comprise a codon-optimized nucleotide sequence.

In an embodiment of any of the LNP compositions, methods or compositions for use disclosed herein, the polynucleotide comprises at least one chemical modification. In an embodiment, the chemical modification is selected from the group consisting of pseudouridine, N1-methylpseudouridine, 2-thiouridine, 4′-thiouridine, 5-methylcytosine, 2-thio-1-methyl-1-deaza-pseudouridine, 2-thio-1-methyl-pseudouridine, 2-thio-5-aza-uridine, 2-thio-dihydropseudouridine, 2-thio-dihydrouridine, 2-thio-pseudouridine, 4-methoxy-2-thio-pseudouridine, 4-methoxy-pseudouridine, 4-thio-1-methyl-pseudouridine, 4-thio-pseudouridine, 5-aza-uridine, dihydropseudouridine, 5-methyluridine, 5-methyluridine, 5-methoxyuridine, and 2′-O-methyl uridine. In an embodiment, the chemical modification is selected from the group consisting of pseudouridine, N1-methylpseudouridine, 5-methylcytosine, 5-methoxyuridine, and a combination thereof. In an embodiment, the chemical modification is N1-methylpseudouridine. In an embodiment, each mRNA in the lipid nanoparticle comprises fully modified N1-methylpseudouridine.

In an embodiment of any of the LNP compositions, methods or compositions for use disclosed herein, the LNP composition comprises: (i) an ionizable lipid, e.g., an amino lipid; (ii) a sterol or other structural lipid; (iii) a non-cationic helper lipid or phospholipid; and (iv) a PEG-lipid.

In an embodiment of any of the LNP compositions, methods or compositions for use disclosed herein, the LNP composition comprises an ionizable lipid comprising an amino lipid. In an embodiment, the ionizable lipid comprises a compound of any of Formulae (I), (I-a), (I-b), (I-c), (II), (II-a), (II-b), (II-c), (II-d), (II-e), (II-f), (II-g), (II-h), or (III). In an embodiment, the ionizable lipid comprises a compound of Formula (I). In an embodiment, the ionizable lipid comprises Compound 18. In an embodiment, the ionizable lipid comprises Compound 25.

In some embodiments, the lipid nanoparticle comprises a compound of Ionizable amino lipid Formula (I):

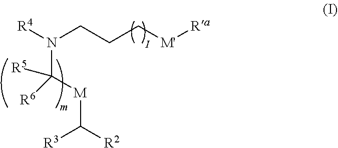

or its N-oxide, or a salt or isomer thereof,
wherein R′^{a} is R′^{branched}; wherein
   R′^{branched} is:

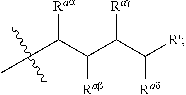

wherein

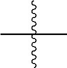

denotes a point of attachment;
wherein R^{aα}, R^{aβ}, R^{aγ}, and R^{aδ} are each independently selected from the group consisting of H, C₂₋₁₂ alkyl, and C₂₋₁₂ alkenyl;
R² and R³ are each independently selected from the group consisting of C₁₋₁₄ alkyl and C₂₋₁₄ alkenyl;
R⁴ is selected from the group consisting of —(CH2)ₙOH, wherein n is selected from the group consisting of 1, 2, 3, 4, and 5, and

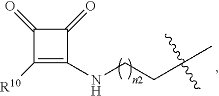

wherein

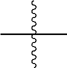

denotes a point of attachment; wherein
R¹⁰ is N(R)₂; each R is independently selected from the group consisting of C₁₋₆ alkyl, C₂₋₃ alkenyl, and H; and n2 is selected from the group consisting of 1, 2, 3, 4, 5, 6, 7, 8, 9, and 10;
each R⁵ is independently selected from the group consisting of C₁₋₃ alkyl, C₂₋₃ alkenyl, and H;
each R⁶ is independently selected from the group consisting of C₁₋₃ alkyl, C₂₋₃ alkenyl, and H;
M and M′ are each independently selected from the group consisting of —C(O)O— and —OC(O)—;
R′ is a C₁₋₁₂ alkyl or C₂₋₁₂ alkenyl;
l is selected from the group consisting of 1, 2, 3, 4, and 5; and
m is selected from the group consisting of 5, 6, 7, 8, 9, 10, 11, 12, and 13.

In some embodiments, the compound of ionizable amino lipid Formula (I) is selected from:

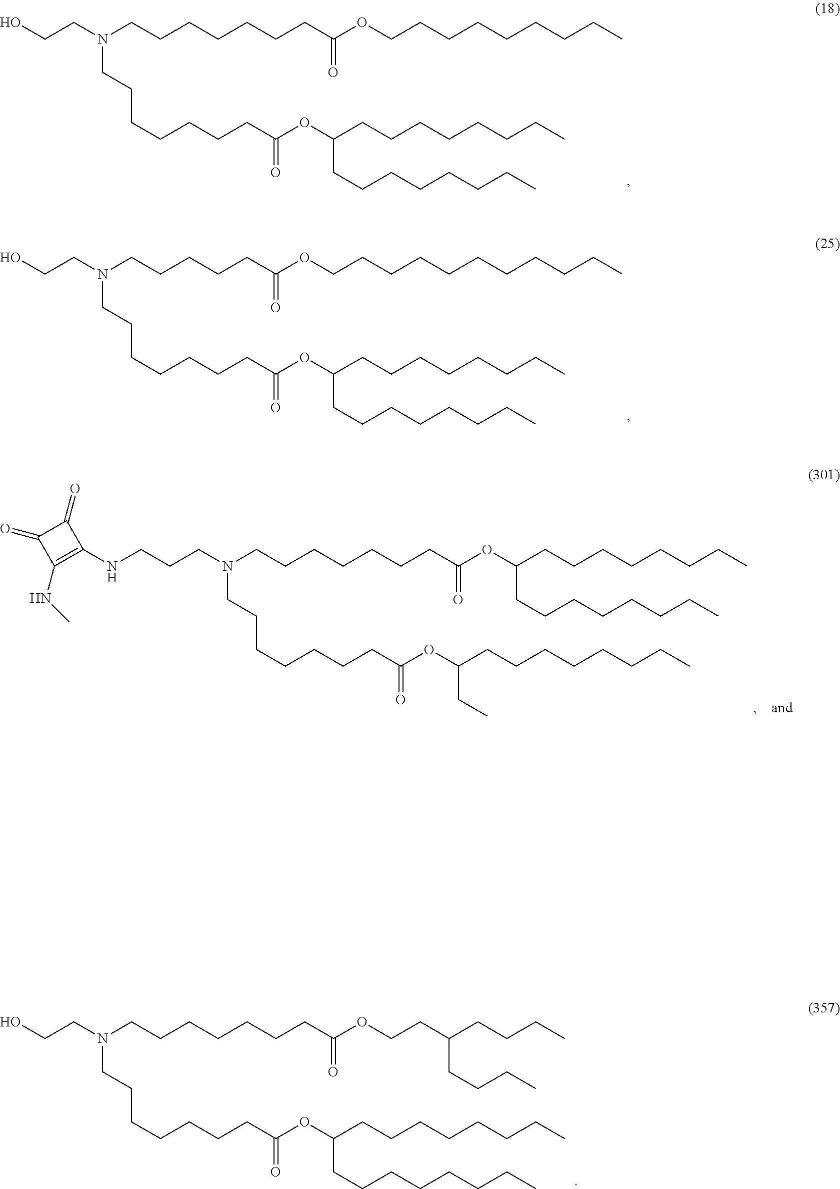 [SMILES: CCCCCCCCCOC(=O)CCCCCCCN(CCO)CCCCCCCC(=O)OC(CCCCCCCC)CCCCCCCC ; CCCCCCCCCCCOC(=O)CCCCCN(CCO)CCCCCCCC(=O)OC(CCCCCCCC)CCCCCCCC ; CCCCCCCCC(CC)OC(=O)CCCCCCCN(CCCCCCCC(=O)OC(CCCCCCCC)CCCCCCCC)CCCNc1c(NC)c(=O)c1=O ; CCCCCCCCC(CCCCCCCC)OC(=O)CCCCCCCN(CCO)CCCCCCCC(=O)OCCC(CCCC)CCCC]

In some embodiments, the lipid nanoparticle further comprises a phospholipid, a structural lipid, and a PEG-lipid.

In some embodiments, the PEG-lipid is PEG DMG.

In some embodiments, the lipid nanoparticle comprises:
(i) 40-50 mol % of the compound of Formula (I), 30-45 mol % of the structural lipid, 5-15 mol % of the phospholipid, and 1-5 mol % of the PEG-lipid; or
(ii) 45-50 mol % of the compound of Formula (I), 35-45 mol % of the structural lipid, 8-12 mol % of the phospholipid, and 1.5 to 3.5 mol % of the PEG-lipid.

In some embodiments, the lipid nanoparticle comprises Compound 25, DSPC, Cholesterol, and PEG DMG.

In an embodiment of any of the LNP compositions, methods or compositions for use disclosed herein, the LNP comprises about 20 mol % to about 60 mol % ionizable lipid, about 5 mol % to about 25 mol % non-cationic helper lipid or phospholipid, about 25 mol % to about 55 mol % sterol or other structural lipid, and about 0.5 mol % to about 15 mol % PEG lipid. In one embodiment of the LNPs or methods of the disclosure, the LNP comprises about 35 mol % to about 55 mol % ionizable lipid, about 5 mol % to about 25 mol % non-cationic helper lipid or phospholipid, about 30 mol % to about 40 mol % sterol or other structural lipid, and about 0 mol % to about 10 mol % PEG lipid. In one embodiment of the LNPs or methods of the disclosure, the LNP comprises about 50 mol % ionizable lipid, about 10 mol % non-cationic helper lipid or phospholipid, about 38.5 mol % sterol or other structural lipid, and about 1.5 mol % PEG lipid. In one embodiment of the LNPs or methods of the disclosure, the LNP comprises about 49.83 mol % ionizable lipid, about 9.83 mol % non-cationic helper lipid or phospholipid, about 30.33 mol % sterol or other structural lipid, and about 2.0 mol % PEG lipid. In an embodiment of any of the LNP compositions, methods or compositions for use disclosed herein, the LNP comprises about 45 mol % to about 50 mol % ionizable lipid. In one embodiment of the LNPs or methods of the disclosure, the LNP comprises about 45.5 mol % to about 49.5 mol % ionizable lipid. In one embodiment of the LNPs or methods of the disclosure, the LNP comprises about 46 mol % to about 49 mol % ionizable lipid. In one embodiment of the LNPs or methods of the disclosure, the LNP comprises about 46.5 mol % to about 48.5 mol % ionizable lipid. In one embodiment of the LNPs or methods of the disclosure, the LNP comprises about 47 mol % to about 48 mol % ionizable lipid.

In an embodiment of any of the LNP compositions, methods or compositions for use disclosed herein, the LNP comprises about 45 mol % to about 49.5 mol % ionizable lipid. In one embodiment of the LNPs or methods of the disclosure, the LNP comprises about 45 mol % to about 49 mol % ionizable lipid. In one embodiment of the LNPs or methods of the disclosure, the LNP comprises about 45 mol % to about 48.5 mol % ionizable lipid. In one embodiment of the LNPs or methods of the disclosure, the LNP comprises about 45 mol % to about 48 mol % ionizable lipid. In one embodiment of the LNPs or methods of the disclosure, the LNP comprises about 45 mol % to about 47.5 mol % ionizable lipid. In one embodiment of the LNPs or methods of the disclosure, the LNP comprises about 45 mol % to about 47 mol % ionizable lipid. In one embodiment of the LNPs or methods of the disclosure, the LNP comprises about 45 mol % to about 46.5 mol % ionizable lipid. In one embodiment of the LNPs or methods of the disclosure, the LNP comprises about 45 mol % to about 46 mol % ionizable lipid. In one embodiment of the LNPs or methods of the disclosure, the LNP comprises about 45 mol % to about 45.5 mol % ionizable lipid.

In an embodiment of any of the LNP compositions, methods or compositions for use disclosed herein, the LNP comprises about 45.5 mol % to about 50 mol % ionizable lipid. In one embodiment of the LNPs or methods of the disclosure, the LNP comprises about 46 mol % to about 50 mol % ionizable lipid. In one embodiment of the LNPs or methods of the disclosure, the LNP comprises about 46.5 mol % to about 50 mol % ionizable lipid. In one embodiment of the LNPs or methods of the disclosure, the LNP comprises about 47 mol % to about 50 mol % ionizable lipid. In one embodiment of the LNPs or methods of the disclosure, the LNP comprises about 47.5 mol % to about 50 mol % ionizable lipid. In one embodiment of the LNPs or methods of the disclosure, the LNP comprises about 48 mol % to about 50 mol % ionizable lipid. In one embodiment of the LNPs or methods of the disclosure, the LNP comprises about 48.5 mol % to about 50 mol % ionizable lipid. In one embodiment of the LNPs or methods of the disclosure, the LNP comprises about 49 mol % to about 50 mol % ionizable lipid. In one embodiment of the LNPs or methods of the disclosure, the LNP comprises about 49.5 mol % to about 50 mol % ionizable lipid.

In an embodiment of any of the LNP compositions, methods or compositions for use disclosed herein, the LNP comprises about 45 mol % to about 46 mol % ionizable lipid. In one embodiment of the LNPs or methods of the disclosure, the LNP comprises about 45.5 mol % to about 46.5 mol % ionizable lipid. In one embodiment of the LNPs or methods of the disclosure, the LNP comprises about 46 mol % to about 47 mol % ionizable lipid. In one embodiment of the LNPs or methods of the disclosure, the LNP comprises about 46.5 mol % to about 47.5 mol % ionizable lipid. In one embodiment of the LNPs or methods of the disclosure, the LNP comprises about 47 mol % to about 48 mol % ionizable lipid. In one embodiment of the LNPs or methods of the disclosure, the LNP comprises about 47.5 mol % to about 48.5 mol % ionizable lipid. In one embodiment of the LNPs or methods of the disclosure, the LNP comprises about 48 mol % to about 49 mol % ionizable lipid. In one embodiment of the LNPs or methods of the disclosure, the LNP comprises about 48.5 mol % to about 49.5 mol % ionizable lipid. In one embodiment of the LNPs or methods of the disclosure, the LNP comprises about 49 mol % to about 50 mol % ionizable lipid.

In an embodiment of any of the LNP compositions, methods or compositions for use disclosed herein, the LNP comprises about 45 mol % ionizable lipid. In one embodiment of the LNPs or methods of the disclosure, the LNP comprises about 45.5 mol % ionizable lipid. In one embodiment of the LNPs or methods of the disclosure, the LNP comprises about 46 mol % ionizable lipid. In one embodiment of the LNPs or methods of the disclosure, the LNP comprises about 46.5 mol % ionizable lipid. In one embodiment of the LNPs or methods of the disclosure, the LNP comprises about 47 mol % ionizable lipid. In one embodiment of the LNPs or methods of the disclosure, the LNP comprises about 47.5 mol % ionizable lipid. In one embodiment of the LNPs or methods of the disclosure, the LNP comprises about 48 mol % ionizable lipid. In one embodiment of the LNPs or methods of the disclosure, the LNP comprises about 48.5 mol % ionizable lipid. In one embodiment of the LNPs or methods of the disclosure, the LNP comprises about 49 mol % ionizable lipid. In one embodiment of the LNPs or methods of the disclosure, the LNP comprises about 49.5 mol % ionizable lipid. In one embodiment of the LNPs or methods of the disclosure, the LNP comprises about 50 mol % ionizable lipid.

In an embodiment of any of the LNP compositions, methods or compositions for use disclosed herein, the LNP comprises about 1 mol % to about 5 mol % PEG lipid. In one embodiment of the LNPs or methods of the disclosure, the LNP comprises about 1.5 mol % to about 4.5 mol % PEG lipid. In one embodiment of the LNPs or methods of the disclosure, the LNP comprises about 2 mol % to about 4 mol % PEG lipid. In one embodiment of the LNPs or methods of the disclosure, the LNP comprises about 2.5 mol % to about 3.5 mol % PEG lipid.

In an embodiment of any of the LNP compositions, methods or compositions for use disclosed herein, the LNP comprises about 1 mol % to about 4.5 mol % PEG lipid. In one embodiment of the LNPs or methods of the disclosure, the LNP comprises about 1 mol % to about 4 mol % PEG lipid. In one embodiment of the LNPs or methods of the disclosure, the LNP comprises about 1 mol % to about 3.5 mol % PEG lipid. In one embodiment of the LNPs or methods of the disclosure, the LNP comprises about 1 mol % to about 3 mol % PEG lipid. In one embodiment of the LNPs or methods of the disclosure, the LNP comprises about 1 mol % to about 2.5 mol % PEG lipid. In one embodiment of the LNPs or methods of the disclosure, the LNP comprises about 1 mol % to about 2 mol % PEG lipid. In one embodiment of the LNPs or methods of the disclosure, the LNP comprises about 1 mol % to about 1.5 mol % PEG lipid.

In an embodiment of any of the LNP compositions, methods or compositions for use disclosed herein, the LNP comprises about 1.5 mol % to about 5 mol % PEG lipid. In one embodiment of the LNPs or methods of the disclosure, the LNP comprises about 2 mol % to about 5 mol % PEG lipid. In one embodiment of the LNPs or methods of the disclosure, the LNP comprises about 2.5 mol % to about 5 mol % PEG lipid. In one embodiment of the LNPs or methods of the disclosure, the LNP comprises about 3 mol % to about 5 mol % PEG lipid. In one embodiment of the LNPs or methods of the disclosure, the LNP comprises about 3.5 mol % to about 5 mol % PEG lipid. In one embodiment of the LNPs or methods of the disclosure, the LNP comprises about 4 mol % to about 5 mol % PEG lipid. In one embodiment of the LNPs or methods of the disclosure, the LNP comprises about 4.5 mol % to about 5 mol % PEG lipid.

In one embodiment of the LNPs or methods of the disclosure, the LNP comprises about 1 mol % to about 2 mol % PEG lipid. In one embodiment of the LNPs or methods of the disclosure, the LNP comprises about 1.5 mol % to about 2.5 mol % PEG lipid. In one embodiment of the LNPs or methods of the disclosure, the LNP comprises about 2 mol % to about 3 mol % PEG lipid. In one embodiment of the LNPs or methods of the disclosure, the LNP comprises about 3.5 mol % to about 4.5 mol % PEG lipid. In one embodiment of the LNPs or methods of the disclosure, the LNP comprises about 4 mol % to about 5 mol % PEG lipid.

In an embodiment of any of the LNP compositions, methods or compositions for use disclosed herein, the LNP comprises about 1 mol % PEG lipid. In one embodiment of the LNPs or methods of the disclosure, the LNP comprises about 1.5 mol % PEG lipid. In one embodiment of the LNPs or methods of the disclosure, the LNP comprises about 2 mol % PEG lipid. In one embodiment of the LNPs or methods of the disclosure, the LNP comprises about 2.5 mol % PEG lipid. In one embodiment of the LNPs or methods of the disclosure, the LNP comprises about 3 mol % PEG lipid. In one embodiment of the LNPs or methods of the disclosure, the LNP comprises about 3.5 mol % PEG lipid. In one embodiment of the LNPs or methods of the disclosure, the LNP comprises about 4 mol % PEG lipid. In one embodiment of the LNPs or methods of the disclosure, the LNP comprises about 4.5 mol % PEG lipid. In one embodiment of the LNPs or methods of the disclosure, the LNP comprises about 5 mol % PEG lipid.

In one embodiment of the LNPs or methods of the disclosure, the LNP comprises about 50 mol % Compound 25 and about 10 mol % non-cationic helper lipid or phospholipid. In one embodiment of the LNPs or methods of the disclosure, the LNP comprises 50 mol % Compound 25 and about 10 mol % non-cationic helper lipid or phospholipid. In one embodiment of the LNPs or methods of the disclosure, the LNP comprises about 50 mol % Compound 25 and 10 mol % non-cationic helper lipid or phospholipid. In one embodiment of the LNPs or methods of the disclosure, the LNP comprises 50 mol % Compound 25 and 10 mol % non-cationic helper lipid or phospholipid. In one embodiment of the LNPs or methods of the disclosure, the LNP comprises about 49.83 mol % Compound 25, about 9.83 mol % non-cationic helper lipid or phospholipid, about 30.33 mol % sterol or other structural lipid, and about 2.0 mol % PEG lipid.

In one embodiment of the LNPs or methods of the disclosure, the LNP comprises about 48 mol % Compound 25, about 11 mol % non-cationic helper lipid or phospholipid, about 38.5 mol % sterol or other structural lipid, and about 2.5 mol % PEG lipid. In one embodiment of the LNPs or methods of the disclosure, the LNP comprises about 48 mol % Compound 25, about 11 mol % DSPC, about 38.5 mol % cholesterol, and about 2.5 mol % PEG-DMG.

In an embodiment of any of the LNP compositions, methods or compositions for use disclosed herein, the LNP is formulated for intravenous, subcutaneous, or intramuscular delivery. In an embodiment, the LNP is formulated for intramuscular delivery.

In one embodiment, an LNP composition as disclosed herein is administered to a subject having cancer. In some embodiments, the cancer is a solid tumor, e.g., is a locally advanced or metastatic solid tumor.

In an embodiment of any of the methods or compositions for use disclosed herein, the cancer is a melanoma. In some embodiments, the melanoma is a cutaneous melanoma. In an embodiment, the cutaneous melanoma is a 1 L cutaneous melanoma stage IIIB+. In an embodiment of any of the methods or compositions for use disclosed herein, the cancer is a NSCLC. In an embodiment, the NSCLC is a 1 L NSCLC. In an embodiment of any of the methods or compositions for use disclosed herein, the cancer is a bladder cancer. In some embodiments, the bladder cancer is a non-muscle invasive bladder cancer. In an embodiment of any of the methods or compositions for use disclosed herein, the cancer is a head and neck cancer. In some embodiments, the head and neck cancer is a head and neck squamous cell carcinoma. In an embodiment of any of the methods or compositions for use disclosed herein, the cancer is a colorectal cancer. In some embodiments, the colorectal cancer is a microsatellite stable colorectal cancer. In an embodiment of any of the methods or compositions for use disclosed herein, the cancer is a basal cell carcinoma. In an embodiment of any of the methods or compositions for use disclosed herein, the cancer is a breast cancer. In some embodiments, the breast cancer is a triple negative breast cancer.

In an embodiment of any of the methods or compositions for use disclosed herein, the LNP composition as administered to the subject according to a dosing interval. In some embodiments, the dosing interval comprises a cycle of three weeks. In some embodiments, the LNP composition is administered to the subject once every three weeks for one or more cycles. In some embodiments, the dosing regimen comprises two cycles, three cycles, four cycles, five cycles, six cycles, seven cycles, eight cycles, or nine cycles.

In some embodiments, the LNP composition is administered at a dose of about 50 μg to about 1 mg, e.g., about 100 μg to about 1 mg, about 200 μg to about 900 μg, about 300 μg to about 800 μg, about 400 μg to about 700 μg, about 500 μg to about 600 μg, about 200 μg to about 1 mg, about 300 μg to about 1 mg, about 400 μg to about 1 mg, about 500 μg to about 1 mg, about 600 μg to about 1 mg, about 700 μg to about 1 mg, about 800 μg to about 1 mg, about 900 μg to about 1 mg, about 100 μg to about 900 μg, about 100 μg to about 800 μg, about 100 μg to about 700 μg, about 100 μg to about 600 μg, about 100 μg to about 500 μg, about 100 μg to about 400 μg, about 100 μg to about 300 μg, or about 100 μg to about 200 μg. In some embodiments, the LNP composition is administered at a dose of about 100 μg to about 200 μg, about 200 μg to about 300 μg, about 300 μg to about 400 μg, about 400 μg to about 500 μg, about 500 μg to about 600 μg, about 600 μg to about 700 μg, about 700 μg to about 800 μg, about 800 μg to about 900 μg, or about 900 μg to about 1 mg.

In some embodiments, the LNP composition is administered at a dose of about 50 μg to about 75 μg (e.g., about 50 μg). In some embodiments, the LNP composition is administered at a dose of about 50 μg to about 150 μg (e.g., about 100 μg). In some embodiments, the LNP composition is administered at a dose of about 150 μg to about 250 μg (e.g., about 200 μg), In some embodiments, the LNP composition is administered at a dose of about 250 μg to about 350 μg (e.g., about 300 μg). In some embodiments, the LNP composition is administered at a dose of about 350 μg to about 450 μg (e.g., about 400 μg). In some embodiments, the LNP composition is administered at a dose of about 450 μg to about 550 μg (e.g., about 500 μg). In some embodiments, the LNP composition is administered at a dose of about 550 μg to about 650 μg (e.g., about 600 μg). In some embodiments, the LNP composition is administered at a dose of about 650 μg to about 750 μg (e.g., about 700 μg). In some embodiments, the LNP composition is administered at a dose of about 750 μg to about 850 μg (e.g., about 800 μg). In some embodiments, the LNP composition is administered at a dose of about 850 μg to about 950 μg (e.g., about 900 μg). In some embodiments, the LNP composition is administered at a dose of about 950 μg to about 1 mg (e.g., about 1 mg).

In some embodiments, the LNP composition is administered intramuscularly (IM).

In an embodiment of any of the methods or compositions for use disclosed herein, the subject is a mammal, e.g., a human.

Additional features of any of the aforesaid LNP compositions or methods of using said LNP compositions, include one or more of the following enumerated embodiments. Those skilled in the art will recognize or be able to ascertain using no more than routine experimentation, many equivalents to the specific embodiments of the invention described herein. Such equivalents are intended to be encompassed by the following enumerated embodiments.

### Other Embodiments of the Disclosure

E1. A lipid nanoparticle (LNP) composition comprising a polynucleotide comprising an mRNA which encodes a checkpoint cancer vaccine comprising (i) one or more (e.g., 1, 2, 3, 4, or more) Indoleamine-pyrrole 2,3-dioxygenase (IDO) antigenic peptides and (ii) one or more (e.g., 1, 2, 3, 4, or more) programmed death-ligand 1 (PD-L1) antigenic peptides.
E2. A lipid nanoparticle composition, for stimulating T effector cells, the composition comprising a polynucleotide comprising an mRNA which encodes a checkpoint cancer vaccine comprising (i) one or more (e.g., 1, 2, 3, 4, or more) IDO antigenic peptides and (ii) one or more (e.g., 1, 2, 3, 4, or more) PD-L1 antigenic peptides.
E3. A lipid nanoparticle composition, for stimulating T effector cells, the composition comprising a polynucleotide comprising an mRNA which encodes a checkpoint cancer vaccine comprising (i) one or more (e.g., 1, 2, 3, 4, or more) IDO antigenic peptides and (ii) one or more (e.g., 1, 2, 3, 4, or more) PD-L1 antigenic peptides.
E4. An mRNA construct comprising a polynucleotide which encodes a checkpoint cancer vaccine comprising (i) one or more (e.g., 1, 2, 3, 4, or more) IDO antigenic peptides and (ii) one or more (e.g., 1, 2, 3, 4, or more) PD-L1 antigenic peptides.
E5. The LNP composition or mRNA construct of any one of embodiments E1-E4, wherein the IDO antigenic peptide comprises a fragment of a naturally occurring IDO molecule, or a variant thereof.
E6. The LNP composition or mRNA construct of any one of embodiments E1-E5, wherein the IDO antigenic peptide is derived from IDO1 or IDO2.
E7. The LNP composition or mRNA construct of embodiment E6, wherein the IDO antigenic peptide is derived from IDO1.
E8. The LNP composition or mRNA construct of any one of embodiments E1-E7, wherein the IDO antigenic peptide comprises an amino acid sequence having at least 85%, 90%, 95%, 96%, 97%, 98%, 99% or 100% identity to the sequence of SEQ ID NO: 1.
E9. The LNP composition or mRNA construct of any one of embodiments E1-E8, wherein the polynucleotide encoding the IDO antigenic fragment comprises a nucleotide sequence having at least 60%, 65%, 70%, 75%, 80%, 85%, 90%, 95%, 96%, 97%, 98%, 99% or 100% identity to the sequence of SEQ ID NO: 2, or an antigenic fragment thereof.
E10. The LNP composition or mRNA construct of any one of embodiments E1-E9, wherein the PD-L1 antigenic peptide comprises a fragment of a naturally occurring PD-L1 molecule, or a variant thereof.
E11. The LNP composition or mRNA construct of any of the embodiments E1-E10, wherein the PD-L1 antigenic peptide comprises an amino acid sequence having at least 85%, 90%, 95%, 96%, 97%, 98%, 99% or 100% identity to the sequence of SEQ ID NO: 3.
E12. The LNP composition or mRNA construct of any of embodiments E1-E11, wherein the polynucleotide encoding the PD-L1 antigenic fragment comprises a nucleotide sequence having at least 60%, 65%, 70%, 75%, 80%, 85%, 90%, 95%, 96%, 97%, 98%, 99% or 100% identity to the sequence of SEQ ID NO: 4, or an antigenic fragment thereof.
E13. The LNP composition or mRNA construct of any of embodiments E1-E12, wherein the checkpoint cancer vaccine comprises two IDO antigenic peptides and two PD-L1 antigenic peptides.
E14. The LNP composition or mRNA construct of any one of embodiments E1-E12, wherein the checkpoint cancer vaccine comprises three IDO antigenic peptides and three PD-L1 antigenic peptides.
E15. The LNP composition or mRNA construct of any one of embodiments E1-E12, wherein the checkpoint cancer vaccine comprises four IDO antigenic peptides and four PD-L1 antigenic peptides.
E16. The LNP composition or mRNA construct of any of embodiments E1-E15, wherein the checkpoint cancer vaccine comprises alternating IDO and PD-L1 antigenic peptides.
E17. The LNP composition or mRNA construct of embodiment E15 or E16, wherein the checkpoint cancer vaccine comprises (i) an IDO antigenic peptide, (ii) a PD-L1 antigenic peptide, (iii) an IDO antigenic peptide, (iv) a PD-L1 antigenic peptide, (v) an IDO antigenic peptide, (vi) a PD-L1 antigenic peptide, (vii) an IDO antigenic peptide, and (viii) a PD-L1 antigenic peptide.
E18. The LNP composition or mRNA construct of any one of embodiments E15-E17, wherein the alternating IDO and PD-L1 antigenic peptides comprise an amino acid sequence having at least 85%, 90%, 95%, 96%, 97%, 98%, 99% or 100% identity to an IDO amino acid sequence provided in Table 1A, e.g., SEQ ID NO: 5, or an antigenic fragment thereof.
E19. The LNP composition or mRNA construct of any one of embodiments E16-E18, wherein the polynucleotide encoding the alternating IDO and PD-L1 antigenic peptides comprises a nucleotide sequence having at least 50%, 55%, 60%, 65%, 70%, 75%, 80%, 85%, 90%, 95%, 96%, 97%, 98%, 99% or 100% identity to the sequence of SEQ ID NO: 6, or an antigenic fragment thereof.
E20. The LNP composition or mRNA construct of any one of embodiments E16-E19, wherein the polynucleotide encoding the alternating IDO and PD-L1 antigenic peptides comprises a nucleotide sequence having at least 50%, 55%, 60%, 65%, 70%, 75%, 80%, 85%, 90%, 95%, 96%, 97%, 98%, 99% or 100% identity to the sequence of SEQ ID NO: 300, or an antigenic fragment thereof.
E21. The LNP composition or mRNA construct of any one of embodiments E16-E19, wherein the polynucleotide encoding the alternating IDO and PD-L1 antigenic peptides comprises a nucleotide sequence having at least 50%, 55%, 60%, 65%, 70%, 75%, 80%, 85%, 90%, 95%, 96%, 97%, 98%, 99% or 100% identity to the sequence of SEQ ID NO: 301, or an antigenic fragment thereof.
E22. The LNP composition or mRNA construct of any one of embodiments E16-E19, wherein the polynucleotide encoding the alternating IDO and PD-L1 antigenic peptides comprises a nucleotide sequence having at least 50%, 55%, 60%, 65%, 70%, 75%, 80%, 85%, 90%, 95%, 96%, 97%, 98%, 99% or 100% identity to the sequence of SEQ ID NO: 302, or an antigenic fragment thereof.
E23. The LNP composition or mRNA construct of any one of embodiments E16-E19, wherein the polynucleotide encoding the alternating IDO and PD-L1 antigenic peptides comprises the nucleotide sequence of SEQ ID NO: 300, which comprises from 5′ to 3′ end: 5′ UTR of SEQ ID NO: 56, ORF sequence of SEQ ID NO: 6, and 3′ UTR of SEQ ID NO: 108.
E24. The LNP composition or mRNA construct of any one of embodiments E16-E19, wherein the polynucleotide encoding the alternating IDO and PD-L1 antigenic peptides comprises the nucleotide sequence of SEQ ID NO: 301, which comprises from 5′ to 3′ end: 5′ UTR of SEQ ID NO: 272, ORF sequence of SEQ ID NO: 6, and 3′ UTR of SEQ ID NO: 108.
E25. The LNP composition or mRNA construct of any one of embodiments E16-E19, wherein the polynucleotide encoding the alternating IDO and PD-L1 antigenic peptides comprises the nucleotide sequence of SEQ ID NO: 302, which comprises from 5′ to 3′ end: 5′ UTR of SEQ ID NO: 273, ORF sequence of SEQ ID NO: 6, and 3′ UTR of SEQ ID NO: 108.
E26. The LNP composition or mRNA construct of any one of the preceding embodiments, further comprising a polynucleotide encoding for one or more adjuvant amino acid sequences.
E27. The LNP composition or mRNA construct of any of the preceding embodiments, which results in:
   (i) stimulation of T effector cells;
   (ii) cytotoxic T cell-mediated killing of suppressive immune and tumor cells that overexpress PD-L1 or IDO;
   (iii) induction of an anti-tumor immune response; and/or
   (iv) infiltration of tumor cells by CD4+ and/or CD8+ T cells and killing of tumor cells expressing IDO and/or PDL1.
E28. The LNP composition or mRNA construct of any of the preceding embodiments, which results in amelioration or delay of cancer progression, e.g., as described herein, in a subject.
E29. The LNP composition or mRNA construct of any one of the preceding embodiments, wherein the polynucleotide comprising an mRNA encoding the checkpoint cancer vaccine comprising one or more (e.g., 1, 2, 3, 4, or more) Indoleamine-pyrrole 2,3-dioxygenase (IDO) antigenic peptides and (ii) one or more (e.g., 1, 2, 3, 4, or more) programmed death-ligand 1 (PD-L1) antigenic peptides, comprises at least one chemical modification.
E30. The LNP composition or mRNA construct of embodiment E29, wherein the chemical modification is selected from the group consisting of pseudouridine, N1-methylpseudouridine, 2-thiouridine, 4′-thiouridine, 5-methylcytosine, 2-thio-1-methyl-1-deaza-pseudouridine, 2-thio-1-methyl-pseudouridine, 2-thio-5-aza-uridine, 2-thio-dihydropseudouridine, 2-thio-dihydrouridine, 2-thio-pseudouridine, 4-methoxy-2-thio-pseudouridine, 4-methoxy-pseudouridine, 4-thio-1-methyl-pseudouridine, 4-thio-pseudouridine, 5-aza-uridine, dihydropseudouridine, 5-methyluridine, 5-methyluridine, 5-methoxyuridine, and 2′-O-methyl uridine.
E31. The LNP composition or mRNA construct of embodiment E30, wherein the chemical modification is selected from the group consisting of pseudouridine, N1-methylpseudouridine, 5-methylcytosine, 5-methoxyuridine, and a combination thereof.
E32. The LNP composition or mRNA construct of embodiment E31, wherein the chemical modification is N1-methylpseudouridine.
E33. The LNP composition or mRNA construct of any one of the preceding embodiments, wherein the mRNA in the lipid nanoparticle comprises fully modified N1-methylpseudouridine.
E34. The LNP composition of any one of the preceding embodiments, wherein the LNP composition comprises: (i) an ionizable lipid, e.g., an amino lipid; (ii) a sterol or other structural lipid; (iii) a non-cationic helper lipid or phospholipid; and (iv) a PEG-lipid.
E35. The LNP composition of embodiment E34, wherein the ionizable lipid comprises an amino lipid.
E36. The LNP composition of embodiment E34 or E35, wherein the ionizable lipid comprises a compound of any of Formulae (I), (I-a), (I-b), (I-c), (II), (II-a), (II-b), (II-c), (II-d), (II-e), (II-f), (II-g), (II-h), or (III).
E37. The LNP composition of any one of embodiments E34-E36, wherein the ionizable lipid comprises Compound 18, Compound 25, Compound 301, or Compound 357.
E38. The LNP composition of embodiment E37, wherein the ionizable lipid comprises Compound 25.
E39. The LNP composition of any one of the embodiments E34-E38, wherein the sterol or other structural lipid comprises cholesterol.
E40. The LNP composition of any one of the embodiments E34-E39, wherein the non-cationic helper lipid or phospholipid comprises DSPC.
E41. The LNP composition of any one of the embodiments E34-E40, wherein the PEG lipid comprises PEG DMG.
E42. The LNP composition of any one of embodiments E34-E41, wherein the LNP comprises a molar ratio of about 20-60% ionizable lipid: 5-25% phospholipid: 25-55% cholesterol; and 0.5-15% PEG lipid.
E43. The LNP composition of embodiment E42, wherein the LNP comprises a molar ratio of about 48 mol % ionizable lipid: about 11 mol % phospholipid: about 38.5 mol % cholesterol; and about 2.5 mol % PEG lipid.
E44. The LNP composition of embodiment E43, wherein the LNP comprises a molar ratio of about 48 mol % Compound 25: about 11 mol % DSPC: about 38.5 mol % cholesterol; and about 2.5 mol % PEG DMG.
E45. The LNP composition or mRNA construct of any one of the preceding embodiments, which is formulated for, intramuscular, subcutaneous, intravenous intranasal, intraocular, rectal, or oral delivery.
E46. The LNP composition or mRNA construct of any one of the preceding embodiments, further comprising a pharmaceutically acceptable carrier or excipient.
E47. A pharmaceutical composition comprising the LNP composition or mRNA construct of any one of the preceding embodiments.
E48. A method of modulating, e.g., stimulating, an immune response in a subject, comprising administering to the subject in need thereof an effective amount of an LNP composition comprising a polynucleotide comprising an mRNA which encodes a checkpoint cancer vaccine comprising one or more (e.g., 1, 2, 3, 4, or more) Indoleamine-pyrrole 2,3-dioxygenase (IDO) antigenic peptides and (ii) one or more (e.g., 1, 2, 3, 4, or more) programmed death-ligand 1 (PD-L1) antigenic peptides.
E49. A method of stimulating T effector cells in a subject, comprising administering to the subject an effective amount of an LNP composition comprising a polynucleotide comprising an mRNA which encodes a checkpoint cancer vaccine comprising one or more (e.g., 1, 2, 3, 4, or more) Indoleamine-pyrrole 2,3-dioxygenase (IDO) antigenic peptides and (ii) one or more (e.g., 1, 2, 3, 4, or more) programmed death-ligand 1 (PD-L1) antigenic peptides.
E50. A method of treating, or preventing, a cancer, or a symptom thereof, comprising administering to the subject in need thereof an effective amount of an LNP composition comprising a polynucleotide comprising an mRNA which encodes a checkpoint cancer vaccine comprising one or more (e.g., 1, 2, 3, 4, or more) Indoleamine-pyrrole 2,3-dioxygenase (IDO) antigenic peptides and (ii) one or more (e.g., 1, 2, 3, 4, or more) programmed death-ligand 1 (PD-L1) antigenic peptides.
E51. The method of embodiment E50, wherein the cancer is a locally advanced or metastatic solid tumor. E52. The method of embodiment E50, wherein the cancer is a melanoma.
E53. The method of embodiment E52, wherein the melanoma is a cutaneous melanoma.
E54. The method of embodiment 34, wherein the cutaneous melanoma is a 1 L cutaneous melanoma stage IIIB+.
E55. The method of embodiment E54, wherein the cancer is a NSCLC.
E56. The method of embodiment E55, wherein the NSCLC is a 1 L NSCLC.
E57. The method of embodiment E50, wherein the cancer is a bladder cancer.
E58. The method of embodiment E57, wherein bladder cancer is a non-muscle invasive bladder cancer.
E59. The method of embodiment E50, wherein the cancer is a head and neck cancer.
E60. The method of embodiment E59, wherein the head and neck cancer is a head and neck squamous cell carcinoma.
E61. The method of embodiment E50, wherein the cancer is a colorectal cancer.
E62. The method of embodiment E61, wherein the colorectal cancer is a microsatellite stable colorectal cancer.
E63. The method of embodiment E50, wherein the cancer is a basal cell carcinoma.
E64. The method of embodiment E50, wherein the cancer is a breast cancer.
E65. The method of embodiment E64, wherein the breast cancer is a triple negative breast cancer.
E66. The method of any one of embodiments E48-E65, wherein the IDO antigenic peptide comprises a fragment of a naturally occurring IDO molecule, or a variant thereof.
E67. The method of any one of embodiments E48-E66, wherein the IDO antigenic peptide is derived from IDO1 or IDO2.
E68. The method of embodiment E67, wherein the IDO antigenic peptide is derived from IDO1.
E69. The method of any one of embodiments E48-E68, wherein the IDO antigenic peptide comprises an amino acid sequence having at least 85%, 90%, 95%, 96%, 97%, 98%, 99% or 100% identity to the sequence of SEQ ID NO: 1.
E70. The method of any one of embodiments E48-E69, wherein the polynucleotide encoding the IDO antigenic fragment comprises a nucleotide sequence having at least 60%, 65%, 70%, 75%, 80%, 85%, 90%, 95%, 96%, 97%, 98%, 99% or 100% identity to the sequence of SEQ ID NO: 2, or an antigenic fragment thereof.
E71. The method of any one of embodiments E48-E70, wherein the PD-L1 antigenic peptide comprises a fragment of a naturally occurring PD-L1 molecule, or a variant thereof.
E72. The method of any of the embodiments E48-E71, wherein the PD-L1 antigenic peptide comprises an amino acid sequence having at least 85%, 90%, 95%, 96%, 97%, 98%, 99% or 100% identity to the sequence of SEQ ID NO: 3.
E73. The method of any of embodiments E48-E72, wherein the polynucleotide encoding the PD-L1 antigenic fragment comprises a nucleotide sequence having at least 60%, 65%, 70%, 75%, 80%, 85%, 90%, 95%, 96%, 97%, 98%, 99% or 100% identity to the sequence of SEQ ID NO: 4, or an antigenic fragment thereof.
E74. The method of any of embodiments E48-E73, wherein the checkpoint cancer vaccine comprises two IDO antigenic peptides and two PD-L1 antigenic peptides.
E75. The method of any one of embodiments E48-E73, wherein the checkpoint cancer vaccine comprises three IDO antigenic peptides and three PD-L1 antigenic peptides.
E76. The method of any one of embodiments E48-E73, wherein the checkpoint cancer vaccine comprises four IDO antigenic peptides and four PD-L1 antigenic peptides.
E77. The method of any of embodiments E48-E76, wherein the checkpoint cancer vaccine comprises alternating IDO and PD-L1 antigenic peptides.
E78. The method of embodiment E76 or E77, wherein the checkpoint cancer vaccine comprises (i) an IDO antigenic peptide, (ii) a PD-L1 antigenic peptide, (iii) an IDO antigenic peptide, (iv) a PD-L1 antigenic peptide, (v) an IDO antigenic peptide, (vi) a PD-L1 antigenic peptide, (vii) an IDO antigenic peptide, and (viii) a PD-L1 antigenic peptide.
E79. The method of any one of embodiments E76-E78, wherein the alternating IDO and PD-L1 antigenic peptides comprise an amino acid sequence having at least 85%, 90%, 95%, 96%, 97%, 98%, 99% or 100% identity to an IDO amino acid sequence provided in Table 1A, e.g., SEQ ID NO: 5, or an antigenic fragment thereof.
E80. The method of any one of embodiments E77-E79, wherein the polynucleotide encoding the alternating IDO and PD-L1 antigenic peptides comprises a nucleotide sequence having at least 50%, 55%, 60%, 65%, 70%, 75%, 80%, 85%, 90%, 95%, 96%, 97%, 98%, 99% or 100% identity to the sequence of SEQ ID NO: 6, or an antigenic fragment thereof.
E81. The method of any one of embodiments E77-E80, wherein the polynucleotide encoding the alternating IDO and PD-L1 antigenic peptides comprises a nucleotide sequence having at least 50%, 55%, 60%, 65%, 70%, 75%, 80%, 85%, 90%, 95%, 96%, 97%, 98%, 99% or 100% identity to the sequence of SEQ ID NO: 300, or an antigenic fragment thereof.
E82. The method of any one of embodiments E77-E80, wherein the polynucleotide encoding the alternating IDO and PD-L1 antigenic peptides comprises a nucleotide sequence having at least 50%, 55%, 60%, 65%, 70%, 75%, 80%, 85%, 90%, 95%, 96%, 97%, 98%, 99% or 100% identity to the sequence of SEQ ID NO: 301, or an antigenic fragment thereof.
E83. The method of any one of embodiments E77-E80, wherein the polynucleotide encoding the alternating IDO and PD-L1 antigenic peptides comprises a nucleotide sequence having at least 50%, 55%, 60%, 65%, 70%, 75%, 80%, 85%, 90%, 95%, 96%, 97%, 98%, 99% or 100% identity to the sequence of SEQ ID NO: 302, or an antigenic fragment thereof.
E84. The method of any one of embodiments E77-E80, wherein the polynucleotide encoding the alternating IDO and PD-L1 antigenic peptides comprises the nucleotide sequence of SEQ ID NO: 300, which comprises from 5′ to 3′ end: 5′ UTR of SEQ ID NO: 56, ORF sequence of SEQ ID NO: 6, and 3′ UTR of SEQ ID NO: 108.
E85. The method of any one of embodiments E77-E80, wherein the polynucleotide encoding the alternating IDO and PD-L1 antigenic peptides comprises the nucleotide sequence of SEQ ID NO: 301, which comprises from 5′ to 3′ end: 5′ UTR of SEQ ID NO: 272, ORF sequence of SEQ ID NO: 6, and 3′ UTR of SEQ ID NO: 108.
E86. The method of any one of embodiments E77-E80, wherein the polynucleotide encoding the alternating IDO and PD-L1 antigenic peptides comprises the nucleotide sequence of SEQ ID NO: 302, which comprises from 5′ to 3′ end: 5′ UTR of SEQ ID NO: 273, ORF sequence of SEQ ID NO: 6, and 3′ UTR of SEQ ID NO: 108.
E87. The method of any one of the preceding embodiments, wherein the composition further comprises a polynucleotide encoding for one or more adjuvant amino acid sequences.
E88. The method of any of the preceding embodiments, which results in:
   (i) stimulation of T effector cells;
   (ii) cytotoxic T cell-mediated killing of suppressive immune and tumor cells that overexpress PD-L1 or IDO;
   (iii) induction of an anti-tumor immune response; and/or
   (iv) infiltration of tumor cells by CD4+ and/or CD8+ T cells and killing of tumor cells expressing IDO and/or PDL1.
E89. The method of any of the preceding embodiments, which results in amelioration or delay of cancer progression, e.g., as described herein, in a subject.
E90. The method of any one of the preceding embodiments, wherein the polynucleotide comprising an mRNA encoding the checkpoint cancer vaccine comprising one or more (e.g., 1, 2, 3, 4, or more) Indoleamine-pyrrole 2,3-dioxygenase (IDO) antigenic peptides and (ii) one or more (e.g., 1, 2, 3, 4, or more) programmed death-ligand 1 (PD-L1) antigenic peptides, comprises at least one chemical modification.
E91. The method of embodiment E90, wherein the chemical modification is selected from the group consisting of pseudouridine, N1-methylpseudouridine, 2-thiouridine, 4′-thiouridine, 5-methylcytosine, 2-thio-1-methyl-1-deaza-pseudouridine, 2-thio-1-methyl-pseudouridine, 2-thio-5-aza-uridine, 2-thio-dihydropseudouridine, 2-thio-dihydrouridine, 2-thio-pseudouridine, 4-methoxy-2-thio-pseudouridine, 4-methoxy-pseudouridine, 4-thio-1-methyl-pseudouridine, 4-thio-pseudouridine, 5-aza-uridine, dihydropseudouridine, 5-methyluridine, 5-methyluridine, 5-methoxyuridine, and 2′-O-methyl uridine.
E92. The method of embodiment E91, wherein the chemical modification is selected from the group consisting of pseudouridine, N1-methylpseudouridine, 5-methylcytosine, 5-methoxyuridine, and a combination thereof.
E93. The method of embodiment E92, wherein the chemical modification is N1-methylpseudouridine.
E94. The method of any one of the preceding embodiments, wherein the mRNA in the lipid nanoparticle comprises fully modified N1-methylpseudouridine.
E95. The method of any one of the preceding embodiments, wherein the LNP composition comprises: (i) an ionizable lipid, e.g., an amino lipid; (ii) a sterol or other structural lipid; (iii) a non-cationic helper lipid or phospholipid; and (iv) a PEG-lipid.
E96. The method of embodiment E95, wherein the ionizable lipid comprises an amino lipid.
E97. The method of embodiment E95 or E96, wherein the ionizable lipid comprises a compound of any of Formulae (I), (I-a), (I-b), (I-c), (II), (II-a), (II-b), (II-c), (II-d), (II-e), (II-f), (II-g), (II-h), or (III).
E98. The method of any one of embodiments E95-E97, wherein the ionizable lipid comprises a compound of Formula (I).
E99. The method of any one of embodiments E95-E98, wherein the ionizable lipid comprises Compound 18, Compound 25, Compound 301, or Compound 357.
E100. The method of embodiment E99, wherein the ionizable lipid comprises Compound 25.
E101. The method of any one of the embodiments E95-E100, wherein the sterol or other structural lipid comprises cholesterol.
E102. The method of any one of the embodiments E95-E101, wherein the non-cationic helper lipid or phospholipid comprises DSPC.
E103. The method of any one of the embodiments E95-E102, wherein the PEG lipid comprises PEG DMG.
E104. The method of any one of embodiments E95-E103, wherein the LNP comprises a molar ratio of about 20-60% ionizable lipid: 5-25% phospholipid: 25-55% cholesterol; and 0.5-15% PEG lipid.
E105. The method of embodiment E104, wherein the LNP comprises a molar ratio of about 48 mol % ionizable lipid: about 11 mol % phospholipid: about 38.5 mol % cholesterol; and about 2.5 mol % PEG lipid.
E106. The method of embodiment E105, wherein the LNP comprises a molar ratio of about 48 mol % Compound 25: about 11 mol % DSPC: about 38.5 mol % cholesterol; and about 2.5 mol % PEG DMG.
E107. The method of any one of embodiments E48-E106, wherein the LNP composition is administered at a dose of 100 μg to about 1 mg.
E108. The method of embodiment E107, wherein the LNP composition is administered at a dose of 50 μg to 150 μg, 150 μg to 250 μg, 250 μg to 350 μg, 350 μg to 450 μg, 450 μg to 550 μg, 550 μg to 650 μg, 650 μg to 750 μg, 750 μg to 850 μg, 850 μg to 950 μg, or 950 μg to 1 mg.
E109. The method of embodiment E108, wherein the LNP composition is administered at a dose of 50 μg, 100 μg, 200 μg, 300 μg, 400 μg, 500 μg, 600 μg, 700 μg, 800 μg, 900 μg, or 1 mg.
E110. The method of any one of embodiments E48-E109, wherein the LNP composition is administered intramuscularly.
E111. The method of any one of embodiments E48-E110, wherein the LNP composition is administered to the subject according to a dosing interval, e.g., as described herein.
E112. The method of embodiment E111, wherein the dosing interval is performed over at least 1 week, 2 weeks, 3 weeks, or 4 weeks.
E113. The method of embodiment E112, wherein the dosing interval comprises a cycle, e.g., a 21-day or 3-week cycle.
E114. The method of embodiment E112, wherein the LNP composition is administered to the subject on day 1 of a 3-week cycle.
E115. The method of embodiment E114, wherein the LNP composition is administered for at least 2, 3, 4, 5, 6, 7, 8, or 9 cycles.
E116. The method of any one of embodiments E48-E115, wherein the method further comprises administering an additional therapeutic agent to the subject.
E117. The method of embodiment E116, wherein the additional therapeutic agent is a checkpoint inhibitor.
E118. The method of embodiment E117, wherein the checkpoint inhibitor is selected from the group consisting of an anti-PD-1 agent and an anti-CTLA4 agent.
E119. The method of embodiment E118, wherein the checkpoint inhibitor comprises pembrolizumab.
E120. The method of E119, wherein pembrolizumab is administered to the subject according to a dosing interval, e.g., as described herein.
E121. The method of embodiment E121, wherein the dosing interval is performed over at least 1 week, 2 weeks, 3 weeks, 4 weeks, 5 weeks, or 6 weeks.
E122. The method of embodiment E121, wherein the dosing interval comprises a cycle, e.g., a 6-week cycle.
E123. The method of embodiment E122, wherein the LNP composition is administered to the subject on day 1 of a 6-week cycle.
E124. The method of embodiment E123, wherein the LNP composition is administered for at least 2, 3, 4, or 5 cycles.
E125. The method of any one of embodiments E119-E124, wherein pembrolizumab is administered at a dose of about 400 mg.

### BRIEF DESCRIPTION OF THE DRAWINGS

FIG. 1 is graph depicting a comparison of antigen-specific IFNγ ELISpot responses to mRNA-4359 with PD-L1 restimulation in HLA-A*02:01 transgenic and wild type mice.

FIG. 2 is graph depicting a comparison of antigen-specific IFNγ ELISpot responses to mRNA-4359 with IDO1 restimulation in HLA-A*02:01 transgenic and wild type mice.

FIG. 3 is a graph depicting flow cytometry analysis of antigen-specific CD8+IFNγ+ T cell responses to mRNA-4359 in HLA-A*02:01 transgenic and wild type mice.

FIG. 4A is a graph showing T-cell response following vaccination measured by the number of INFγ Spot Forming Units (SFU) normalized to 1 million PBMCs over time.

FIGS. 4B-4C are a pair of heat maps showing IDO1 (4B) and PD-L1 (4C) responses in peripheral blood before and after vaccination.

FIGS. 4D-4E are a pair of graphs showing change in IFNγ SFU/million PBMCs in IDO specific cells (4D) and PD-L1 specific cells (4E).

FIG. 5 is schematic representation of the IS/ID model.

FIGS. 6A-6D are a series of graphs showing IFNγ SFU/million PBMCs in IDO1 complete responders (6A), IDO1 partial responders (6B), PD-L1 complete responders (6C), or PD-L1 partial responders (6D).

FIGS. 7A-7D are a series of graphs showing the first order and total order sobol index for IDO1 complete responders (7A), IDO1 partial responders (7B), PD-L1 complete responders (7C), or PD-L1 partial responders (7D).

FIGS. 8A-8B are a pair of graphs showing predictive dose-parameter effect curves for the peptide vaccine (8A) and mRNA vaccine (8B)

FIGS. 9A-9D are a series of graphs showing expected steady-state peak and trough for different doses for IDO1 complete responders (9A), IDO1 partial responders (9B), PD-L1 complete responders (9C), or PD-L1 partial responders (9D).

FIGS. 10A-10C is a series of graphs showing dosing regimen simulations.

### DETAILED DESCRIPTION

Using the compositions and methods described herein, immune cells (e.g., T cells) can be primed to recognize tumor-associated antigens and/or to become activated, e.g., to have cytotoxic properties. For example, T effector cells can be primed to mediate an anti-cancer response in a subject, resulting in the killing suppressive immune cells and tumor cells that express IDO or PDL1. In one embodiment, the subject methods and compositions can be used to induce or promote an anti-tumor immune response in a subject having a cancer.

Exemplary methods of treating a cancer include administering to a subject a checkpoint cancer vaccine comprising a polynucleotide encoding comprising mRNA sequences encoding one or more (e.g., 1, 2, 3, 4, or more) IDO antigenic peptides and one or more (e.g., 1, 2, 3, 4, or more) PD-L1 antigenic peptides, e.g., as described herein. Optionally, the vaccine can also comprise mRNA sequences which encode for adjuvant amino acid sequences. Without wishing to be bound by theory, it is believed that in some embodiments, systemic PD-1/PD-L1 blockade may further amplify the effect, leading to further immune activation and superior disease control. Therefore, in one embodiment, a checkpoint inhibitor, e.g., and anti-PD1 antibody, anti-CTLA4 antibody or combination thereof can also be administered to the subject.

Accordingly, disclosed herein is a lipid nanoparticle (LNP) composition comprising an mRNA encoding a checkpoint cancer vaccine comprising one or more (e.g., 1, 2, 3, 4, or more) IDO antigenic peptides and one or more (e.g., 1, 2, 3, 4, or more) PD-L1 antigenic peptides, and optionally one or more adjuvant amino acid sequences, and uses thereof. The LNP compositions of the present disclosure comprise mRNA therapeutics encoding checkpoint cancer vaccines comprising (i) one or more IDO antigenic peptides and (ii) one or more PD-L1 antigenic peptides and optionally one or more adjuvant amino acid sequences. In an aspect, the LNP compositions of the present disclosure can prime T effector cells and/or stimulate an anti-tumor immune response in vivo. Also disclosed herein are methods of using an LNP composition comprising checkpoint cancer vaccines comprising (i) one or more IDO antigenic peptides and (ii) one or more PD-L1 antigenic peptides, for stimulating an immune response by IDO/PDL1 specific T cells to promote an immunostimulatory environment and/or promote killing of IDO/PDL1 expressing cancer cells, thereby treating a cancer, e.g., a melanoma or an NSCLC.

### Definitions

Administering: As used herein, “administering” refers to a method of delivering a composition to a subject or patient. A method of administration may be selected to target delivery (e.g., to specifically deliver) to a specific region or system of a body. For example, an administration may be parenteral (e.g., intramuscular).

Approximately, about: As used herein, the terms “approximately” or “about,” as applied to one or more values of interest, refers to a value that is similar to a stated reference value. In certain embodiments, the term “approximately” or “about” refers to a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value). For example, when used in the context of an amount of a given compound in a lipid component of an LNP, “about” may mean+/−5% of the recited value. For instance, an LNP including a lipid component having about 40% of a given compound may include 30-50% of the compound. As another example, an LNP including a lipid component having about 50% of a given compound may include 45-55% of the compound.

Chimeric molecule: As used herein, the term “chimeric molecule” refers to a molecule having at least two portions from different sources or origins, e.g., a IDO antigenic peptide and a PD-L1 antigenic peptide. For example, the two portions can be derived from two different polypeptides. Each portion can be a full-length polypeptide or a fragment (e.g., an antigenic fragment) thereof. In certain embodiments, the two polypeptides are from two different organisms. In other embodiments, the two polypeptides are from the same organism. The two different polypeptides can be both naturally occurring or synthetic, or one naturally occurring the other synthetic. In some embodiments, the two portions of the chimeric molecule have different properties. The property may be a biological property, such as a function or activity in vitro, ex vivo, or in vivo. The property can also be a physical or chemical property, such as a binding affinity or specificity. In some embodiments, the two portions are covalently linked together. For example, the two portions can be linked directly, e.g., by a single covalent bond (e.g., a peptide bond), or indirectly, e.g., through a linker (e.g., a peptide linker). In some embodiments, a chimeric molecule is produced through the joining of two or more polynucleotides that originally coded for separate polypeptides. In some embodiments, the two or more polynucleotides form a single open reading frame.

Conjugated: As used herein, the term “conjugated,” when used with respect to two or more moieties, means that the moieties are physically associated or connected with one another, either directly or via one or more additional moieties that serves as a linking agent, to form a structure that is sufficiently stable so that the moieties remain physically associated under the conditions in which the structure is used, e.g., physiological conditions. In some embodiments, two or more moieties may be conjugated by direct covalent chemical bonding. In other embodiments, two or more moieties may be conjugated by ionic bonding or hydrogen bonding.

Contacting: As used herein, the term “contacting” means establishing a physical connection between two or more entities. For example, contacting a cell with an mRNA or a lipid nanoparticle composition means that the cell and mRNA or lipid nanoparticle are made to share a physical connection. Methods of contacting cells with external entities both in vivo, in vitro, and ex vivo are well known in the biological arts. In exemplary embodiments of the disclosure, the step of contacting a mammalian cell with a composition (e.g., a nanoparticle, or pharmaceutical composition of the disclosure) is performed in vivo. For example, contacting a lipid nanoparticle composition and a cell (for example, a mammalian cell) which may be disposed within an organism (e.g., a mammal) may be performed by any suitable administration route (e.g., parenteral administration to the organism, including intravenous, intramuscular, intradermal, and subcutaneous administration). For a cell present in vitro, a composition (e.g., a lipid nanoparticle) and a cell may be contacted, for example, by adding the composition to the culture medium of the cell and may involve or result in transfection. Moreover, more than one cell may be contacted by a nanoparticle composition.

Delivering: As used herein, the term “delivering” means providing an entity to a destination. For example, delivering a therapeutic and/or prophylactic to a subject may involve administering an LNP including the therapeutic and/or prophylactic to the subject (e.g., by an intravenous, intramuscular, intradermal, or subcutaneous route). Administration of an LNP to a mammal or mammalian cell may involve contacting one or more cells with the lipid nanoparticle.

Encapsulate: As used herein, the term “encapsulate” means to enclose, surround, or encase. In some embodiments, a compound, polynucleotide (e.g., an mRNA), or other composition may be fully encapsulated, partially encapsulated, or substantially encapsulated. For example, in some embodiments, an mRNA of the disclosure may be encapsulated in a lipid nanoparticle, e.g., a liposome.

Encapsulation efficiency: As used herein, “encapsulation efficiency” refers to the amount of a therapeutic and/or prophylactic that becomes part of an LNP, relative to the initial total amount of therapeutic and/or prophylactic used in the preparation of an LNP. For example, if 97 mg of therapeutic and/or prophylactic are encapsulated in an LNP out of a total 100 mg of therapeutic and/or prophylactic initially provided to the composition, the encapsulation efficiency may be given as 97%. As used herein, “encapsulation” may refer to complete, substantial, or partial enclosure, confinement, surrounding, or encasement.

Effective amount: As used herein, the term “effective amount” of an agent is that amount sufficient to effect beneficial or desired results, for example, clinical results, and, as such, an “effective amount” depends upon the context in which it is being applied. For example, in the context of the amount of a target cell delivery potentiating lipid in a lipid composition (e.g., LNP) of the disclosure, an effective amount of a target cell delivery potentiating lipid is an amount sufficient to effect a beneficial or desired result as compared to a lipid composition (e.g., LNP) lacking the target cell delivery potentiating lipid. Non-limiting examples of beneficial or desired results effected by the lipid composition (e.g., LNP) include increasing the percentage of cells transfected and/or increasing the level of expression of a protein encoded by a nucleic acid associated with/encapsulated by the lipid composition (e.g., LNP). In the context of administering a target cell delivery potentiating lipid-containing lipid nanoparticle such that an effective amount of lipid nanoparticles is taken up by target cells in a subject, an effective amount of target cell delivery potentiating lipid-containing LNP is an amount sufficient to effect a beneficial or desired result as compared to an LNP lacking the target cell delivery potentiating lipid. Non-limiting examples of beneficial or desired results in the subject include increasing the percentage of cells transfected, increasing the level of expression of a protein encoded by a nucleic acid associated with/encapsulated by the target cell delivery potentiating lipid-containing LNP and/or increasing a prophylactic or therapeutic effect in vivo of a nucleic acid, or its encoded protein, associated with/encapsulated by the target cell delivery potentiating lipid-containing LNP, as compared to an LNP lacking the target cell delivery potentiating lipid. In some embodiments, a therapeutically effective amount of target cell delivery potentiating lipid-containing LNP is sufficient, when administered to a subject suffering from or susceptible to an infection, disease, disorder, and/or condition, to treat, improve symptoms of, diagnose, prevent, and/or delay the onset of the infection, disease, disorder, and/or condition. In another embodiment, an effective amount of a lipid nanoparticle is sufficient to result in expression of a desired protein in at least about 5%, 10%, 15%, 20%, 25% or more of target cells. For example, an effective amount of target cell delivery potentiating lipid-containing LNP can be an amount that results in transfection of at least 5%, 10%, 15%, 20%, 25%, 30%, or 35% of target cells after a single intravenous injection.

Expression: As used herein, “expression” of a nucleic acid sequence refers to one or more of the following events: (1) production of an RNA template from a DNA sequence (e.g., by transcription); (2) processing of an RNA transcript (e.g., by splicing, editing, 5′ cap formation, and/or 3′ end processing); (3) translation of an RNA into a polypeptide or protein; and (4) post-translational modification of a polypeptide or protein.

Ex vivo: As used herein, the term “ex vivo” refers to events that occur outside of an organism (e.g., animal, plant, or microbe or cell or tissue thereof). Ex vivo events may take place in an environment minimally altered from a natural (e.g., in vivo) environment.

Fragment: A “fragment,” as used herein, refers to a portion. For example, fragments of proteins may include polypeptides obtained by digesting full-length protein isolated from cultured cells or obtained through recombinant DNA techniques.

GC-rich: As used herein, the term “GC-rich” refers to the nucleobase composition of a polynucleotide (e.g., mRNA), or any portion thereof (e.g., an RNA element), comprising guanine (G) and/or cytosine (C) nucleobases, or derivatives or analogs thereof, wherein the GC-content is greater than about 50%. The term “GC-rich” refers to all, or to a portion, of a polynucleotide, including, but not limited to, a gene, a non-coding region, a 5′ UTR, a 3′ UTR, an open reading frame, an RNA element, a sequence motif, or any discrete sequence, fragment, or segment thereof which comprises about 50% GC-content. In some embodiments of the disclosure, GC-rich polynucleotides, or any portions thereof, are exclusively comprised of guanine (G) and/or cytosine (C) nucleobases.

GC-content: As used herein, the term “GC-content” refers to the percentage of nucleobases in a polynucleotide (e.g., mRNA), or a portion thereof (e.g., an RNA element), that are either guanine (G) and cytosine (C) nucleobases, or derivatives or analogs thereof, (from a total number of possible nucleobases, including adenine (A) and thymine (T) or uracil (U), and derivatives or analogs thereof, in DNA and in RNA). The term “GC-content” refers to all, or to a portion, of a polynucleotide, including, but not limited to, a gene, a non-coding region, a 5′ or 3′ UTR, an open reading frame, an RNA element, a sequence motif, or any discrete sequence, fragment, or segment thereof.

IDO antigenic peptide: As used herein, the term “IDO antigenic peptide” refers to a full length naturally-occurring IDO (e.g., a mammalian IDO, e.g., human IDO, e.g., associated with UniProt: P14902 and/or NCBI Gene ID: 3620; or associated with UniProt Q6ZQW0 and/or NCBI Gene ID 169355) a fragment (e.g., an antigenic fragment) of IDO, or a variant of IDO having at least 80%, 85%, 90%, 95%, 96%, 97%, 98%, or 99% sequence identity to: a naturally-occurring wild type IDO or a fragment (e.g., an antigenic fragment) thereof. In some embodiments, the IDO molecule is an IDO gene product, e.g., an IDO polypeptide.

PD-L1 antigenic peptide: As used herein, the term “PD-L1 antigenic peptide” refers to a full length naturally-occurring PD-L1 (e.g., a mammalian PD-L1, e.g., human PD-L1, e.g., associated with UniProt: Q9NZQ7; NCBI Gene ID: 29126) a fragment (e.g., an antigenic fragment) of PD-L1, or a variant of PD-L1 having at least 80%, 85%, 90%, 95%, 96%, 97%, 98%, or 99% sequence identity to: a naturally occurring wild type PD-L1 or a fragment (e.g., an antigenic fragment) thereof. In some embodiments, the PD-L1 molecule is a PD-L1 gene product, e.g., a PD-L1 polypeptide.

Heterologous: As used herein, “heterologous” indicates that a sequence (e.g., an amino acid sequence or the polynucleotide that encodes an amino acid sequence) is not normally present in a given polypeptide or polynucleotide. For example, an amino acid sequence that corresponds to a domain or motif of one protein may be heterologous to a second protein.

Isolated: As used herein, the term “isolated” refers to a substance or entity that has been separated from at least some of the components with which it was associated (whether in nature or in an experimental setting). Isolated substances may have varying levels of purity in reference to the substances from which they have been associated. Isolated substances and/or entities may be separated from at least about 10%, about 20%, about 30%, about 40%, about 50%, about 60%, about 70%, about 80%, about 90%, or more of the other components with which they were initially associated. In some embodiments, isolated agents are more than about 80%, about 85%, about 90%, about 91%, about 92%, about 93%, about 94%, about 95%, about 96%, about 97%, about 98%, about 99%, or more than about 99% pure. As used herein, a substance is “pure” if it is substantially free of other components.

Liposome: As used herein, by “liposome” is meant a structure including a lipid-containing membrane enclosing an aqueous interior. Liposomes may have one or more lipid membranes. Liposomes include single-layered liposomes (also known in the art as unilamellar liposomes) and multi-layered liposomes (also known in the art as multilamellar liposomes).

Modified: As used herein “modified” or “modification” refers to a changed state or a change in composition or structure of a polynucleotide (e.g., mRNA). Polynucleotides may be modified in various ways including chemically, structurally, and/or functionally. For example, polynucleotides may be structurally modified by the incorporation of one or more RNA elements, wherein the RNA element comprises a sequence and/or an RNA secondary structure(s) that provides one or more functions (e.g., translational regulatory activity). Accordingly, polynucleotides of the disclosure may be comprised of one or more modifications (e.g., may include one or more chemical, structural, or functional modifications, including any combination thereof).

Modified: As used herein “modified” refers to a changed state or structure of a molecule of the disclosure. Molecules may be modified in many ways including chemically, structurally, and functionally. In one embodiment, the mRNA molecules of the present disclosure are modified by the introduction of non-natural nucleosides and/or nucleotides, e.g., as it relates to the natural ribonucleotides A, U, G, and C. Noncanonical nucleotides such as the cap structures are not considered “modified” although they differ from the chemical structure of the A, C, G, U ribonucleotides. mRNA: As used herein, an “mRNA” refers to a messenger ribonucleic acid. An mRNA may be naturally or non-naturally occurring. For example, an mRNA may include modified and/or non-naturally occurring components such as one or more nucleobases, nucleosides, nucleotides, or linkers. An mRNA may include a cap structure, a chain terminating nucleoside, a stem loop, a polyA sequence, and/or a polyadenylation signal. An mRNA may have a nucleotide sequence encoding a polypeptide. Translation of an mRNA, for example, in vivo translation of an mRNA inside a mammalian cell, may produce a polypeptide. Traditionally, the basic components of an mRNA molecule include at least a coding region, a 5′-untranslated region (5′-UTR), a 3′UTR, a 5′ cap and a polyA sequence.

Nanoparticle: As used herein, “nanoparticle” refers to a particle having any one structural feature on a scale of less than about 1000 nm that exhibits novel properties as compared to a bulk sample of the same material. Routinely, nanoparticles have any one structural feature on a scale of less than about 500 nm, less than about 200 nm, or about 100 nm. Also routinely, nanoparticles have any one structural feature on a scale of from about 50 nm to about 500 nm, from about 50 nm to about 200 nm or from about 70 to about 120 nm. In exemplary embodiments, a nanoparticle is a particle having one or more dimensions of the order of about 1-1000 nm. In other exemplary embodiments, a nanoparticle is a particle having one or more dimensions of the order of about 10-500 nm. In other exemplary embodiments, a nanoparticle is a particle having one or more dimensions of the order of about 50-200 nm. A spherical nanoparticle would have a diameter, for example, of between about 50-100 or 70-120 nanometers. A nanoparticle most often behaves as a unit in terms of its transport and properties. It is noted that novel properties that differentiate nanoparticles from the corresponding bulk material typically develop at a size scale of under 1000 nm, or at a size of about 100 nm, but nanoparticles can be of a larger size, for example, for particles that are oblong, tubular, and the like. Although the size of most molecules would fit into the above outline, individual molecules are usually not referred to as nanoparticles.

Nucleic acid: As used herein, the term “nucleic acid” is used in its broadest sense and encompasses any compound and/or substance that includes a polymer of nucleotides. These polymers are often referred to as polynucleotides. Exemplary nucleic acids or polynucleotides of the disclosure include, but are not limited to, ribonucleic acids (RNAs), deoxyribonucleic acids (DNAs), DNA-RNA hybrids, RNAi-inducing agents, RNAi agents, siRNAs, shRNAs, miRNAs, antisense RNAs, ribozymes, catalytic DNA, RNAs that induce triple helix formation, threose nucleic acids (TNAs), glycol nucleic acids (GNAs), peptide nucleic acids (PNAs), locked nucleic acids (LNAs, including LNA having a β-D-ribo configuration, α-LNA having an α-L-ribo configuration (a diastereomer of LNA), 2′-amino-LNA having a 2′-amino functionalization, and 2′-amino-α-LNA having a 2′-amino functionalization) or hybrids thereof.

Nucleic Acid Structure: As used herein, the term “nucleic acid structure” (used interchangeably with “polynucleotide structure”) refers to the arrangement or organization of atoms, chemical constituents, elements, motifs, and/or sequence of linked nucleotides, or derivatives or analogs thereof that comprise a nucleic acid (e.g., an mRNA). The term also refers to the two-dimensional or three-dimensional state of a nucleic acid. Accordingly, the term “RNA structure” refers to the arrangement or organization of atoms, chemical constituents, elements, motifs, and/or sequence of linked nucleotides, or derivatives or analogs thereof, comprising an RNA molecule (e.g., an mRNA) and/or refers to a two-dimensional and/or three dimensional state of an RNA molecule. Nucleic acid structure can be further demarcated into four organizational categories referred to herein as “molecular structure”, “primary structure”, “secondary structure”, and “tertiary structure” based on increasing organizational complexity.

Nucleobase: As used herein, the term “nucleobase” (alternatively “nucleotide base” or “nitrogenous base”) refers to a purine or pyrimidine heterocyclic compound found in nucleic acids, including any derivatives or analogs of the naturally occurring purines and pyrimidines that confer improved properties (e.g., binding affinity, nuclease resistance, chemical stability) to a nucleic acid or a portion or segment thereof. Adenine, cytosine, guanine, thymine, and uracil are the nucleobases predominately found in natural nucleic acids. Other natural, non-natural, and/or synthetic nucleobases, as known in the art and/or described herein, can be incorporated into nucleic acids.

Nucleoside/Nucleotide: As used herein, the term “nucleoside” refers to a compound containing a sugar molecule (e.g., a ribose in RNA or a deoxyribose in DNA), or derivative or analog thereof, covalently linked to a nucleobase (e.g., a purine or pyrimidine), or a derivative or analog thereof (also referred to herein as “nucleobase”), but lacking an internucleoside linking group (e.g., a phosphate group). As used herein, the term “nucleotide” refers to a nucleoside covalently bonded to an internucleoside linking group (e.g., a phosphate group), or any derivative, analog, or modification thereof that confers improved chemical and/or functional properties (e.g., binding affinity, nuclease resistance, chemical stability) to a nucleic acid or a portion or segment thereof.

Open Reading Frame: As used herein, the term “open reading frame,” abbreviated as “ORF,” refers to a segment or region of an mRNA molecule that encodes a polypeptide. The ORF comprises a continuous stretch of non-overlapping, in-frame codons, beginning with the initiation codon and ending with a stop codon, and is translated by the ribosome.

Patient: As used herein, “patient” refers to a subject who may seek or be in need of treatment, requires treatment, is receiving treatment, will receive treatment, or a subject who is under care by a trained professional for a particular disease or condition. In particular embodiments, a patient is a human patient. In some embodiments, a patient is a patient suffering from ancourt of appeals autoimmune disease, e.g., as described herein.

Pharmaceutically acceptable: The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

Pharmaceutically acceptable excipient: The phrase “pharmaceutically acceptable excipient,” as used herein, refers any ingredient other than the compounds described herein (for example, a vehicle capable of suspending or dissolving the active compound) and having the properties of being substantially nontoxic and non-inflammatory in a patient. Excipients may include, for example: antiadherents, antioxidants, binders, coatings, compression aids, disintegrants, dyes (colors), emollients, emulsifiers, fillers (diluents), film formers or coatings, flavors, fragrances, glidants (flow enhancers), lubricants, preservatives, printing inks, sorbents, suspensing or dispersing agents, sweeteners, and waters of hydration. Exemplary excipients include, but are not limited to: butylated hydroxytoluene (BHT), calcium carbonate, calcium phosphate (dibasic), calcium stearate, croscarmellose, crosslinked polyvinyl pyrrolidone, citric acid, crospovidone, cysteine, ethylcellulose, gelatin, hydroxypropyl cellulose, hydroxypropyl methylcellulose, lactose, magnesium stearate, maltitol, mannitol, methionine, methylcellulose, methyl paraben, microcrystalline cellulose, polyethylene glycol, polyvinyl pyrrolidone, povidone, pregelatinized starch, propyl paraben, retinyl palmitate, shellac, silicon dioxide, sodium carboxymethyl cellulose, sodium citrate, sodium starch glycolate, sorbitol, starch (corn), stearic acid, sucrose, talc, titanium dioxide, vitamin A, vitamin E, vitamin C, and xylitol.

Pharmaceutically acceptable salts: As used herein, “pharmaceutically acceptable salts” refers to derivatives of the disclosed compounds wherein the parent compound is modified by converting an existing acid or base moiety to its salt form (e.g., by reacting the free base group with a suitable organic acid). Examples of pharmaceutically acceptable salts include, but are not limited to, mineral or organic acid salts of basic residues such as amines; alkali or organic salts of acidic residues such as carboxylic acids; and the like. Representative acid addition salts include acetate, acetic acid, adipate, alginate, ascorbate, aspartate, benzenesulfonate, benzene sulfonic acid, benzoate, bisulfate, borate, butyrate, camphorate, camphorsulfonate, citrate, cyclopentanepropionate, digluconate, dodecylsulfate, ethanesulfonate, fumarate, glucoheptonate, glycerophosphate, hemisulfate, heptonate, hexanoate, hydrobromide, hydrochloride, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, toluenesulfonate, undecanoate, valerate salts, and the like. Representative alkali or alkaline earth metal salts include sodium, lithium, potassium, calcium, magnesium, and the like, as well as nontoxic ammonium, quaternary ammonium, and amine cations, including, but not limited to ammonium, tetramethylammonium, tetraethylammonium, methylamine, dimethylamine, trimethylamine, triethylamine, ethylamine, and the like. The pharmaceutically acceptable salts of the present disclosure include the conventional non-toxic salts of the parent compound formed, for example, from non-toxic inorganic or organic acids. The pharmaceutically acceptable salts of the present disclosure can be synthesized from the parent compound which contains a basic or acidic moiety by conventional chemical methods. Generally, such salts can be prepared by reacting the free acid or base forms of these compounds with a stoichiometric amount of the appropriate base or acid in water or in an organic solvent, or in a mixture of the two; generally, nonaqueous media like ether, ethyl acetate, ethanol, isopropanol, or acetonitrile are preferred. Lists of suitable salts are found in Remington's Pharmaceutical Sciences, 17th ed., Mack Publishing Company, Easton, Pa., 1985, p. 1418, Pharmaceutical Salts: Properties, Selection, and Use, P. H. Stahl and C. G. Wermuth (eds.), Wiley-VCH, 2008, and Berge et al., Journal of Pharmaceutical Science, 66, 1-19 (1977), each of which is incorporated herein by reference in its entirety.

Polypeptide: As used herein, the term “polypeptide” or “polypeptide of interest” refers to a polymer of amino acid residues typically joined by peptide bonds that can be produced naturally (e.g., isolated or purified) or synthetically.

RNA: As used herein, an “RNA” refers to a ribonucleic acid that may be naturally or non-naturally occurring. For example, an RNA may include modified and/or non-naturally occurring components such as one or more nucleobases, nucleosides, nucleotides, or linkers. An RNA may include a cap structure, a chain terminating nucleoside, a stem loop, a polyA sequence, and/or a polyadenylation signal. An RNA may have a nucleotide sequence encoding a polypeptide of interest. For example, an RNA may be a messenger RNA (mRNA). Translation of an mRNA encoding a particular polypeptide, for example, in vivo translation of an mRNA inside a mammalian cell, may produce the encoded polypeptide. RNAs may be selected from the non-liming group consisting of small interfering RNA (siRNA), asymmetrical interfering RNA (aiRNA), microRNA (miRNA), Dicer-substrate RNA (dsRNA), small hairpin RNA (shRNA), mRNA, long non-coding RNA (lncRNA) and mixtures thereof.

RNA element: As used herein, the term “RNA element” refers to a portion, fragment, or segment of an RNA molecule that provides a biological function and/or has biological activity (e.g., translational regulatory activity). Modification of a polynucleotide by the incorporation of one or more RNA elements, such as those described herein, provides one or more desirable functional properties to the modified polynucleotide. RNA elements, as described herein, can be naturally-occurring, non-naturally occurring, synthetic, engineered, or any combination thereof. For example, naturally-occurring RNA elements that provide a regulatory activity include elements found throughout the transcriptomes of viruses, prokaryotic and eukaryotic organisms (e.g., humans). RNA elements in particular eukaryotic mRNAs and translated viral RNAs have been shown to be involved in mediating many functions in cells. Exemplary natural RNA elements include, but are not limited to, translation initiation elements (e.g., internal ribosome entry site (IRES), see Kieft et al., (2001) RNA 7(2):194-206), translation enhancer elements (e.g., the APP mRNA translation enhancer element, see Rogers et al., (1999) J Biol Chem 274(10):6421-6431), mRNA stability elements (e.g., AU-rich elements (AREs), see Garneau et al., (2007) Nat Rev Mol Cell Biol 8(2):113-126), translational repression element (see e.g., Blumer et al., (2002) Mech Dev 110(1-2):97-112), protein-binding RNA elements (e.g., iron-responsive element, see Selezneva et al., (2013) J Mol Biol 425(18):3301-3310), cytoplasmic polyadenylation elements (Villalba et al., (2011) Curr Opin Genet Dev 21(4):452-457), and catalytic RNA elements (e.g., ribozymes, see Scott et al., (2009) Biochim Biophys Acta 1789(9-10):634-641).

Substantially: As used herein, the term “substantially” refers to the qualitative condition of exhibiting total or near-total extent or degree of a characteristic or property of interest. One of ordinary skill in the biological arts will understand that biological and chemical phenomena rarely, if ever, go to completion and/or proceed to completeness or achieve or avoid an absolute result. The term “substantially” is therefore used herein to capture the potential lack of completeness inherent in many biological and chemical phenomena.

Suffering from: An individual who is “suffering from” a disease, disorder, and/or condition has been diagnosed with or displays one or more symptoms of a disease, disorder, and/or condition.

Therapeutic Agent: The term “therapeutic agent” refers to any agent that, when administered to a subject, has a therapeutic, diagnostic, and/or prophylactic effect and/or elicits a desired biological and/or pharmacological effect.

Transfection: As used herein, the term “transfection” refers to methods to introduce a species (e.g., a polynucleotide, such as a mRNA) into a cell.

Subject: As used herein, the term “subject” refers to any organism to which a composition in accordance with the disclosure may be administered, e.g., for experimental, diagnostic, prophylactic, and/or therapeutic purposes. Typical subjects include animals (e.g., mammals such as mice, rats, rabbits, non-human primates, and humans) and/or plants. In some embodiments, a subject may be a patient.

Treating: As used herein, the term “treating” refers to partially or completely alleviating, ameliorating, improving, relieving, delaying onset of, inhibiting progression of, reducing severity of, and/or reducing incidence of one or more symptoms or features of a particular infection, disease, disorder, and/or condition. For example, “treating” cancer may refer to inhibiting survival, growth, and/or spread of a tumor. Treatment may be administered to a subject who does not exhibit signs of a disease, disorder, and/or condition and/or to a subject who exhibits only early signs of a disease, disorder, and/or condition for the purpose of decreasing the risk of developing pathology associated with the disease, disorder, and/or condition.

Preventing: As used herein, the term “preventing” refers to partially or completely inhibiting the onset of one or more symptoms or features of a particular infection, disease, disorder, and/or condition.

Unmodified: As used herein, “unmodified” refers to any substance, compound or molecule prior to being changed in any way. Unmodified may, but does not always, refer to the wild type or native form of a biomolecule. Molecules may undergo a series of modifications whereby each modified molecule may serve as the “unmodified” starting molecule for a subsequent modification.

Variant: As used herein, the term “variant” refers to a molecule having at least 50%, 60%, 70%, 80%, 85%, 90%, 95%, 96%, 97%, 98%, 99% or 100% activity of the wild type molecule, e.g., as measured by an art-recognized assay.

### IDO Antigenic Peptides

Indoleamine-pyrrole 2,3-dioxygenase (IDO), is an intracellular monomeric, heme-containing enzyme that controls the breakdown of Tryptophan in the Kynurenine pathway (Cemil B and Sarisozen C (2017) Journal of Oncological Sciences 3:2 pp. 52-56). There are two isoforms of IDO, IDO1 and IDO2, which both convert Tryptophan to Kynurenine at different enzymatic rates. IDO2 is narrowly expressed and IDO1 is more broadly expressed, e.g., in endothelial cells, antigen presenting cells, fibroblasts, macrophages and dendritic cells.

In an aspect, the disclosure provides an LNP composition comprising a polynucleotide, e.g., encoding checkpoint cancer vaccine comprising one or more (e.g., 1, 2, 3, 4, or more) IDO antigenic peptides, e.g., derived from IDO1 or IDO2, e.g., as described herein.

In an embodiment, the IDO antigenic peptide is derived from IDO1. In an embodiment the one or more IDO antigenic peptides comprise a naturally occurring IDO1 molecule, a fragment (e.g., an antigenic fragment) of a naturally occurring IDO1 molecule, or a variant thereof. In an embodiment, the IDO antigenic peptides comprise a variant of a naturally occurring IDO1 molecule (e.g., an IDO1 variant), or a fragment thereof. In an embodiment, the LNP composition comprising a polynucleotide encoding a checkpoint cancer vaccine comprising one or more (e.g., 1, 2, 3, 4, or more) IDO1 antigenic peptides and one or more (e.g., 1, 2, 3, 4, or more) PD-L1 antigenic peptides can be administered alone or in combination with an additional agent.

In an aspect, an LNP composition disclosed herein comprises a polynucleotide encoding checkpoint cancer vaccine comprising one or more (e.g., 1, 2, 3, 4, or more) IDO1 antigenic peptides. In an embodiment, the IDO antigenic peptides comprise an amino acid sequence having at least 85%, 90%, 95%, 96%, 97%, 98%, 99% or 100% identity to an IDO amino acid sequence provided in Table 1A, e.g., SEQ ID NO: 1, or an antigenic fragment thereof. In an embodiment, the IDO antigenic peptide comprises the amino acid sequence of an IDO amino acid sequence provided in Table 1A, e.g., SEQ ID NO: 1, or an antigenic fragment thereof. In an embodiment, the IDO antigenic peptide comprises the amino acid sequence of SEQ ID NO: 1, or an antigenic fragment thereof. In an embodiment, the IDO antigenic peptide comprises an amino acid sequence having at least 85%, 90%, 95%, 96%, 97%, 98%, 99% or 100% identity to SEQ ID NO: 1, or an antigenic fragment thereof. In an embodiment, the IDO antigenic peptide comprises SEQ ID NO: 1, or an antigenic fragment thereof.

In an embodiment, the polynucleotide encoding the checkpoint cancer vaccine comprising one or more (e.g., 1, 2, 3, 4, or more) IDO1 antigenic peptides comprises a nucleotide sequence (e.g., a codon-optimized nucleotide sequence) having at least 50%, 55%, 60%, 65%, 70%, 75%, 80%, 85%, 90%, 95%, 96%, 97%, 98%, 99% or 100% identity to the sequence of SEQ ID NO: 2, or an antigenic fragment thereof. In an embodiment, the polynucleotide encoding the checkpoint cancer vaccine comprising one or more (e.g., 1, 2, 3, 4, or more) IDO1 antigenic peptides comprises a nucleotide sequence (e.g., a codon-optimized nucleotide sequence) of SEQ ID NO: 2, or an antigenic fragment thereof.

In an embodiment, the polynucleotide (e.g., mRNA) encoding the checkpoint cancer vaccine comprising one or more (e.g., 1, 2, 3, 4, or more) IDO1 antigenic peptides further comprises one or more elements, e.g., a 5′ UTR and/or a 3′ UTR. In an embodiment, the 5′ UTR and/or 3′UTR comprise one or more micro RNA (mIR) binding sites, e.g., as disclosed herein. Exemplary 5′ UTRs and 3′ UTRs are disclosed in the section entitled “5′ UTR and 3′UTR” herein.

### PD-L1 Molecule

PD-L1 (also known as programmed death ligand 1, CD274, B7-H1) is a membrane-anchored protein that is expressed on hematopoietic cells including antigen-presenting cells such as dendritic cells and macrophages. PD-L1 is also expressed on activated T cells, B cells, and monocytes as well as peripheral nonhematopoietic tissues including liver, heart, skeletal muscle, placenta, lung, and kidney (Dai S et al. (2014) Cell Immunol 290, 72-79). PD-L1 binds to its cognate receptor PD-1, which is a co-inhibitory transmembrane receptor expressed on T cells, B cells, natural killer cells, and thymocytes. Engagement of PD-1 to PD-L1 can inhibit T cell Receptor (TCR) signal transduction through recruitment of regulatory phosphatases which result in decreased IL2 production and glucose metabolism. Continued interaction of PD-1 with PD-L1 can lead to induction of T cell anergy or conversion of naïve cells into induced Regulatory T cells (iTregs). The PD-L1/PD-1 pathway has an important function in immune regulation (e.g., inhibition of T cell proliferation, cytotoxic activity, and cytokine production) and promotes development and function of Tregs.

In an aspect, the disclosure provides an LNP composition comprising a polynucleotide, e.g., encoding checkpoint cancer vaccine comprising one or more (e.g., 1, 2, 3, 4, or more) PD-L1 antigenic peptides, e.g., as described herein.

In an embodiment the one or more PD-L1 antigenic peptides comprise a naturally occurring PD-L1 molecule, a fragment (e.g., an antigenic fragment) of a naturally occurring PD-L1 molecule, or a variant thereof. In an embodiment, the PD-L1 antigenic peptides comprise a variant of a naturally occurring PD-L1 molecule (e.g., a PD-L1 variant), or a fragment thereof. In an embodiment, the LNP composition comprising a polynucleotide encoding checkpoint cancer vaccine comprising one or more (e.g., 1, 2, 3, 4, or more) IDO antigenic peptides and one or more (e.g., 1, 2, 3, 4, or more) PD-L1 antigenic peptides can be administered alone or in combination with an additional agent.

In an aspect, an LNP composition disclosed herein comprises a polynucleotide encoding checkpoint cancer vaccine comprising one or more (e.g., 1, 2, 3, 4, or more) PD-L1 antigenic peptides. In an embodiment, the PD-L1 antigenic peptides comprise an amino acid sequence having at least 85%, 90%, 95%, 96%, 97%, 98%, 99% or 100% identity to an IDO amino acid sequence provided in Table 1A, e.g., SEQ ID NO: 3, or an antigenic fragment thereof. In an embodiment, the PD-L1 antigenic peptide comprises the amino acid sequence of a PD-L1 amino acid sequence provided in Table 1A, e.g., SEQ ID NO: 3, or an antigenic fragment thereof. In an embodiment, the PD-L1 antigenic peptide comprises the amino acid sequence of SEQ ID NO: 3, or an antigenic fragment thereof. In an embodiment, the PD-L1 antigenic peptide comprises an amino acid sequence having at least 85%, 90%, 95%, 96%, 97%, 98%, 99% or 100% identity to SEQ ID NO: 3, or an antigenic fragment thereof. In an embodiment, the PD-L1 antigenic peptide comprises SEQ ID NO: 3, or an antigenic fragment thereof.

In an embodiment, the PD-L1 antigenic peptide comprises an amino acid sequence for a leader sequence and/or an affinity tag (e.g., a leader sequence described herein and/or an affinity tag described herein). In an embodiment, the PD-L1 antigenic peptide does not comprise an amino acid sequence for a leader sequence and/or an affinity tag (e.g., a leader sequence described herein and/or an affinity tag described herein).

In an embodiment, the polynucleotide encoding the checkpoint cancer vaccine comprising one or more (e.g., 1, 2, 3, 4, or more) PD-L1 antigenic peptides comprises a nucleotide sequence (e.g., a codon-optimized nucleotide sequence) having at least 50%, 55%, 60%, 65%, 70%, 75%, 80%, 85%, 90%, 95%, 96%, 97%, 98%, 99% or 100% identity to the sequence of SEQ ID NO: 4, or an antigenic fragment thereof. In an embodiment, the polynucleotide encoding the checkpoint cancer vaccine comprising one or more (e.g., 1, 2, 3, 4, or more) PD-L1 antigenic peptides comprises a nucleotide sequence (e.g., a codon-optimized nucleotide sequence) of SEQ ID NO: 4, or an antigenic fragment thereof.

In an embodiment, the polynucleotide (e.g., mRNA) encoding the checkpoint cancer vaccine comprising one or more (e.g., 1, 2, 3, 4, or more) PD-L1 antigenic peptides comprises a nucleotide sequence that encodes for a leader sequence and/or an affinity tag (e.g., a leader sequence described herein and/or an affinity tag described herein). In an embodiment, the polynucleotide (e.g., mRNA) encoding the checkpoint cancer vaccine comprising one or more (e.g., 1, 2, 3, 4, or more) PD-L1 antigenic peptides does not comprise a nucleotide sequence that encodes for a leader sequence and/or an affinity tag (e.g., a leader sequence described herein and/or an affinity tag described herein).

In an embodiment, the polynucleotide (e.g., mRNA) encoding the checkpoint cancer vaccine comprising one or more (e.g., 1, 2, 3, 4, or more) PD-L1 antigenic peptides further comprises one or more elements, e.g., a 5′ UTR and/or a 3′ UTR. In an embodiment, the 5′ UTR and/or 3′UTR comprise one or more micro RNA (mIR) binding sites, e.g., as disclosed herein. Exemplary 5′ UTRs and 3′ UTRs are disclosed in the section entitled “5′ UTR and 3′UTR” herein.

### Exemplary Checkpoint Cancer Vaccines

Exemplary checkpoint cancer vaccines include, but are not limited to, those containing one or more (e.g., 1, 2, 3, 4, or more) IDO antigenic peptides and one or more (e.g., 1, 2, 3, 4, or more) PD-L1 antigenic peptides. In an embodiment, the checkpoint cancer vaccine comprises alternating IDO and PD-L1 antigenic peptides.

In an embodiment, the checkpoint cancer vaccine comprises one IDO antigenic peptide and one PD-L1 antigenic peptide. In an embodiment, the checkpoint cancer vaccine comprises two IDO antigenic peptides and two PD-L1 antigenic peptides. In an embodiment, the checkpoint cancer vaccine comprises three IDO antigenic peptides and three PD-L1 antigenic peptides. In an embodiment, the checkpoint cancer vaccine comprises four IDO antigenic peptides and four PD-L1 antigenic peptides. In some embodiments, the four IDO and four PD-L1 antigenic peptides are arranged in alternating manner. Accordingly, in an embodiment, the checkpoint cancer vaccine comprises (i) an IDO antigenic peptide, (ii) a PD-L1 antigenic peptide, (iii) an IDO antigenic peptide, (iv) a PD-L1 antigenic peptide, (v) an IDO antigenic peptide, (vi) a PD-L1 antigenic peptide, (vii) an IDO antigenic peptide, and (viii) a PD-L1 antigenic peptide).

In an embodiment, the alternating IDO and PD-L1 antigenic peptides comprise an amino acid sequence having at least 85%, 90%, 95%, 96%, 97%, 98%, 99% or 100% identity to an IDO amino acid sequence provided in Table 1A, e.g., SEQ ID NO: 5, or an antigenic fragment thereof. In an embodiment, the alternating IDO and PD-L1 antigenic peptides comprise the amino acid sequence of an amino acid sequence provided in Table 1A, e.g., SEQ ID NO: 5, or an antigenic fragment thereof. In an embodiment, the alternating IDO and PD-L1 antigenic peptides comprise the amino acid sequence of SEQ ID NO: 5, or an antigenic fragment thereof.

In an embodiment, the alternating IDO and PD-L1 antigenic peptides comprise an amino acid sequence for a leader sequence and/or an affinity tag. In an embodiment, the alternating IDO and PD-L1 antigenic peptides comprise does not comprise an amino acid sequence for a leader sequence and/or an affinity tag.

In an embodiment, the polynucleotide encoding checkpoint cancer vaccine comprising the alternating IDO and PD-L1 antigenic peptides comprise a nucleotide sequence having at least 50%, 55%, 60%, 65%, 70%, 75%, 80%, 85%, 90%, 95%, 96%, 97%, 98%, 99% or 100% identity to the sequence of SEQ ID NO: 6, 300, 301, or 302, or an antigenic fragment thereof. In an embodiment, the polynucleotide (e.g., mRNA) encoding the checkpoint cancer vaccine comprising alternating IDO and PD-L1 antigenic peptides comprise the nucleotide sequence of SEQ ID NO: 6, 300, 301, or 302, or an antigenic fragment thereof. In an embodiment, the polynucleotide encoding the checkpoint cancer vaccine comprising alternating IDO and PD-L1 antigenic peptides comprise a codon-optimized nucleotide sequence.

In some embodiments, the polynucleotide comprising an mRNA nucleotide sequence encoding the checkpoint cancer vaccine comprising one or more (e.g., 1, 2, 3, 4, or more) IDO antigenic peptides and one or more (e.g., 1, 2, 3, 4, or more) PD-L1 antigenic peptides comprises the nucleotide sequence of SEQ ID NO: 300, which consists of from 5′ to 3′ end: 5′ UTR of SEQ ID NO: 56, ORF sequence of SEQ ID NO: 6, and 3′ UTR of SEQ ID NO: 108.

In some embodiments, the polynucleotide encoding the checkpoint cancer vaccine comprising one or more (e.g., 1, 2, 3, 4, or more) IDO antigenic peptides and one or more (e.g., 1, 2, 3, 4, or more) PD-L1 antigenic peptides comprises from 5′ to 3′ end
(i) a 5′ cap such as provided herein, e.g., Cap C1;
(ii) a 5′ UTR, such as the sequences provided herein, for example, SEQ ID NO:56;
(iii) an open reading frame encoding an alternating IDO and PD-L1 antigenic peptides, e.g., a sequence optimized nucleic acid sequence encoding alternating IDO and PD-L1 antigenic peptides set forth as SEQ ID NO: 6;
(iv) at least one stop codon (if not present at 5′ terminus of 3′UTR);
(v) a 3′ UTR, such as the sequences provided herein, for example, SEQ ID NO: 108; and
(vi) a poly-A tail provided herein (e.g., SEQ ID NO:502).

In some embodiments, the polynucleotide comprising an mRNA nucleotide sequence encoding the checkpoint cancer vaccine comprising one or more (e.g., 1, 2, 3, 4, or more) IDO antigenic peptides and one or more (e.g., 1, 2, 3, 4, or more) PD-L1 antigenic peptides comprises the nucleotide sequence of SEQ ID NO: 301, which consists of from 5′ to 3′ end: 5′ UTR of SEQ ID NO: 272, ORF sequence of SEQ ID NO: 6, and 3′ UTR of SEQ ID NO: 108.

In some embodiments, the polynucleotide encoding the checkpoint cancer vaccine comprising one or more (e.g., 1, 2, 3, 4, or more) IDO antigenic peptides and one or more (e.g., 1, 2, 3, 4, or more) PD-L1 antigenic peptides comprises from 5′ to 3′ end
(i) a 5′ cap such as provided herein, e.g., Cap C1;
(ii) a 5′ UTR, such as the sequences provided herein, for example, SEQ ID NO:272;
(iii) an open reading frame encoding an alternating IDO and PD-L1 antigenic peptides, e.g., a sequence optimized nucleic acid sequence encoding alternating IDO and PD-L1 antigenic peptides set forth as SEQ ID NO: 6;
(iv) at least one stop codon (if not present at 5′ terminus of 3′UTR);
(v) a 3′ UTR, such as the sequences provided herein, for example, SEQ ID NO: 108; and
(vi) a poly-A tail provided herein (e.g., SEQ ID NO:502).

In some embodiments, the polynucleotide comprising an mRNA nucleotide sequence encoding the checkpoint cancer vaccine comprising one or more (e.g., 1, 2, 3, 4, or more) IDO antigenic peptides and one or more (e.g., 1, 2, 3, 4, or more) PD-L1 antigenic peptides comprises the nucleotide sequence of SEQ ID NO: 302, which consists of from 5′ to 3′ end: 5′ UTR of SEQ ID NO: 273, ORF sequence of SEQ ID NO: 6, and 3′ UTR of SEQ ID NO: 108.

In some embodiments, the polynucleotide encoding the checkpoint cancer vaccine comprising one or more (e.g., 1, 2, 3, 4, or more) IDO antigenic peptides and one or more (e.g., 1, 2, 3, 4, or more) PD-L1 antigenic peptides comprises from 5′ to 3′ end
(i) a 5′ cap such as provided herein, e.g., Cap C1;
(ii) a 5′ UTR, such as the sequences provided herein, for example, SEQ ID NO:273;
(iii) an open reading frame encoding an alternating IDO and PD-L1 antigenic peptides, e.g., a sequence optimized nucleic acid sequence encoding alternating IDO and PD-L1 antigenic peptides set forth as SEQ ID NO: 6;
(iv) at least one stop codon (if not present at 5′ terminus of 3′ UTR);
(v) a 3′ UTR, such as the sequences provided herein, for example, SEQ ID NO:108; and
(vi) a poly-A tail provided herein (e.g., SEQ ID NO:502).

In an embodiment, the polynucleotide encoding the checkpoint cancer vaccine comprises the nucleotide sequence of Variant 1, Variant 2, or Variant 3, as described in Table 2A.

In an aspect, an LNP composition disclosed herein comprises a polynucleotide encoding a checkpoint cancer vaccine comprising one or more IDO antigenic peptides and one or more PD-L1 antigenic peptides, e.g., as described herein. In an embodiment, the checkpoint cancer vaccine comprises a half-life extender, e.g., a protein (or fragment thereof) that binds to a serum protein such as albumin, IgG, FcRn or transferrin. In an embodiment, the half-life extender is an immunoglobulin Fc region or a variant thereof, e.g., an IgG1 Fc.

In an embodiment, the checkpoint cancer vaccine further comprises a targeting moiety. In an embodiment, the targeting moiety comprises an antibody molecule (e.g., Fab or scFv), a receptor molecule (e.g., a receptor, a receptor fragment or functional variant thereof), a ligand molecule (e.g., a ligand, a ligand fragment or functional variant thereof), or a combination thereof.

TABLE 1A
Exemplary IDO and PD-L1 antigenic peptide sequences

| SEQ | Sequence | |
|---|---|---|
| ID NO | information | Sequence |
| 1 | IDO antigenic | ERDTLLKALLEIASCLEKALQVFHQ |
| | peptide amino acid, | |
| | monomer | |
| 2 | IDO antigenic | GAGCGGGAUACCCUCCUGAAGGCUCUGCUGGAGAUCGC |
| | peptide nucleotide, | AAGCUGCCUGGAGAAAGCCCUCCAGGUGUUCCACCAA |
| | monomer | |
| 3 | PD-L1 antigenic | VFIFMTYWHLLNAFTVTVPKDLYVV |
| | peptide amino acid, | |
| | monomer | |
| 4 | PD-L1 antigenic | GUGUUCAUCUUCAUGACAUACUGGCAUCUGCUGAACGC |
| | peptide nucleic acid, | CUUUACCGUGACCGUACCCAAGGACCUGUACGUGGUG |
| | monomer | |
| 5 | Alternating IDO PD- | ERDTLLKALLEIASCLEKALQVFHQVFIFMTYWHLLNAFTV |
| | L1 antigenic peptide | TVPKDLYVVERDTLLKALLEIASCLEKALQVFHQVFIFMTY |
| | amino acid, multimer | WHLLNAFTVTVPKDLYVVERDTLLKALLEIASCLEKALQV |
| | | FHQVFIFMTYWHLLNAFTVTVPKDLYVVERDTLLKALLEIA |
| | | SCLEKALQVFHQVFIFMTYWHLLNAFTVTVPKDLYVV |
| 6 | Alternating IDO PD- | AUGGAGCGGGAUACCCUCCUGAAGGCUCUGCUGGAGA |
| | L1 antigenic peptide | UCGCAAGCUGCCUGGAGAAAGCCCUCCAGGUGUUCCAC |
| | nucleic acid, | CAAGUGUUCAUCUUCAUGACAUACUGGCAUCUGCUGA |
| | multimer | ACGCCUUUACCGUGACCGUACCCAAGGACCUGUACGUG |
| | | GUGGAGAGAGAUACCCUGCUGAAAGCCCUGCUGGAAA |
| | | UCGCCUCGUGCCUGGAGAAGGCCCUGCAGGUGUUUCAC |
| | | CAGGUGUUCAUUUUCAUGACCUAUUGGCACCUGUUGA |
| | | ACGCUUUUACUGUGACCGUGCCAAAGGAUUUAUACGU |
| | | GGUGGAGCGGGACACACUGCUGAAGGCCCUGCUUGAG |
| | | AUCGCCAGCUGUCUCGAGAAAGCCCUGCAAGUGUUCCA |
| | | CCAGGUAUUCAUCUUUAUGACUUACUGGCACCUGCUCA |
| | | ACGCCUUCACCGUGACAGUGCCCAAGGACUUGUACGUG |
| | | GUCGAGCGGGACACCCUGUUAAAGGCCCUGCUGGAGAU |
| | | UGCCAGCUGUCUGGAGAAGGCACUGCAGGUGUUCCAUC |
| | | AGGUGUUUAUCUUUAUGACCUACUGGCACCUCCUGAAC |
| | | GCCUUCACAGUGACCGUGCCCAAGGAUCUCUACGUGGU |
| | | G |

In some embodiments, a polynucleotide of the present disclosure, for example a polynucleotide comprising an mRNA nucleotide sequence encoding a checkpoint cancer vaccine comprising one or more IDO antigenic peptides and one or more PD-L1 antigenic peptides, comprises (1) a 5′ cap, e.g., as disclosed herein, e.g., as provided in Table 2A or described herein, (2) a 5′ UTR, e.g., as provided in Table 2A, (3) a nucleotide sequence ORF provided in Table 2A, e.g., SEQ ID NO: 2 or 5, (4) a stop codon, (5) a 3′UTR, e.g., as provided in Table 2A, and (6) a tail (e.g., poly-A tail), e.g., as disclosed herein, e.g., a poly-A tail of about 100 residues (e.g., SEQ ID NO: 502).

In some embodiments, the polynucleotide comprises an mRNA nucleotide sequence encoding a checkpoint cancer vaccine comprising one or more IDO antigenic peptides and one or more PD-L1 antigenic peptides comprises the nucleotide sequence of SEQ ID NO: 300, which comprises from 5′ to 3′ end: 5′ UTR of SEQ ID NO: 56, ORF sequence of SEQ ID NO: 6, and 3′ UTR of SEQ ID NO: 108.

In some embodiments, the polynucleotide comprises an mRNA nucleotide sequence encoding a checkpoint cancer vaccine comprising one or more IDO antigenic peptides and one or more PD-L1 antigenic peptides comprises the nucleotide sequence of SEQ ID NO: 301, which comprises from 5′ to 3′ end: 5′ UTR of SEQ ID NO: 272, ORF sequence of SEQ ID NO: 6, and 3′ UTR of SEQ ID NO: 108.

In some embodiments, the polynucleotide comprises an mRNA nucleotide sequence encoding a checkpoint cancer vaccine comprising one or more IDO antigenic peptides and one or more PD-L1 antigenic peptides comprises the nucleotide sequence of SEQ ID NO: 302, which comprises from 5′ to 3′ end: 5′ UTR of SEQ ID NO: 273, ORF sequence of SEQ ID NO: 6, and 3′ UTR of SEQ ID NO: 108.

In some embodiments, all of the 5′ UTR, ORF, and/or 3′ UTR sequences include the modification(s) described in Table 2A. In some embodiments, one, two, or all of the 5′ UTR, ORF, and/or 3′ UTR sequences do not include the modification(s) described in Table 2A. In some embodiments, the 5′ UTRs described in Table 2A additionally comprise a first nucleotide that is an “A” or a “G.”

TABLE 2A
Exemplary checkpoint cancer vaccine construct sequences
Notes: “G5” indicates that all uracils (U) in the mRNA are replaced by N1-methylpseudouracils.

| mRNA | Additional | ORF Sequence | | 5′ UTR | 3′ UTR | Construct |
|---|---|---|---|---|---|---|
| Name | Information | (Amino Acid) | ORF Sequence (Nucleotide) | Sequence | Sequence | Sequence |
| SEQ ID NO: | | 716 | 6 | 56 | 108 | 300 |
| IDO/PD-L1 | G5 | MERDTLLKALL | AUGGAGCGGGAUACCCUCCUGAAGGCUC | GGAAAUAAG | UGAUAA | SEQ ID NO: |
| Checkpoint | Cap C1 | EIASCLEKALQ | UGCUGGAGAUCGCAAGCUGCCUGGAGA | AGAGAAAAG | UAGGCU | 300 comprises |
| cancer | Poly A | VFHQVFIFMTY | AAGCCCUCCAGGUGUUCCACCAAGUGUU | AAGAGUAAG | GGAGCC | 5′ to 3′ end: |
| vaccine | tail: 100 nt | WHLLNAFTVTV | CAUCUUCAUGACAUACUGGCAUCUGCUG | AAGAAAUAU | UCGGUG | 5′ UTR of |
| Variant 1 | (SEQ ID NO: | PKDLYVVERDT | AACGCCUUUACCGUGACCGUACCCAAGG | AAGACCCCG | GCCUAG | SEQ ID NO: |
| | 502) | LLKALLEIASCL | ACCUGUACGUGGUGGAGAGAGAUACCC | GCGCCGCCA | CUUCUU | 56, ORF |
| | | EKALQVFHQVF | UGCUGAAAGCCCUGCUGGAAAUCGCCUC | CC | GCCCCU | sequence of |
| | | IFMTYWHLLNA | GUGCCUGGAGAAGGCCCUGCAGGUGUU | | UGGGCC | SEQ ID NO: |
| | | FTVTVPKDLYV | UCACCAGGUGUUCAUUUUCAUGACCUAU | | UCCCCC | 6, and 3′ UTR |
| | | VERDTLLKALL | UGGCACCUGUUGAACGCUUUUACUGUG | | CAGCCC | sequence of |
| | | EIASCLEKALQ | ACCGUGCCAAAGGAUUUAUACGUGGUG | | CUCCUC | SEQ ID NO: |
| | | VFHQVFIFMTY | GAGCGGGACACACUGCUGAAGGCCCUGC | | CCCUUC | 108. |
| | | WHLLNAFTVTV | UUGAGAUCGCCAGCUGUCUCGAGAAAGC | | CUGCAC | |
| | | PKDLYVVERDT | CCUGCAAGUGUUCCACCAGGUAUUCAUC | | CCGUAC | |
| | | LLKALLEIASCL | UUUAUGACUUACUGGCACCUGCUCAACG | | CCCCGU | |
| | | EKALQVFHQVF | CCUUCACCGUGACAGUGCCCAAGGACUU | | GGUCUU | |
| | | IFMTYWHLLNA | GUACGUGGUCGAGCGGGACACCCUGUUA | | UGAAUA | |
| | | FTVTVPKDLYV | AAGGCCCUGCUGGAGAUUGCCAGCUGUC | | AAGUCU | |
| | | V | UGGAGAAGGCACUGCAGGUGUUCCAUC | | GAGUGG | |
| | | | AGGUGUUUAUCUUUAUGACCUACUGGC | | GCGGC | |
| | | | ACCUCCUGAACGCCUUCACAGUGACCGU | | | |
| | | | GCCCAAGGAUCUCUACGUGGUG | | | |
| SEQ ID NO: | | 716 | 6 | 272 | 108 | 301 |
| IDO/PD-L1 | G5 | MERDTLLKALL | AUGGAGCGGGAUACCCUCCUGAAGGCUC | GGGAAAUAA | UGAUAA | SEQ ID NO: |
| Checkpoint | Cap C1 | EIASCLEKALQ | UGCUGGAGAUCGCAAGCUGCCUGGAGA | GAGAGAAAA | UAGGCU | 301 comprises |
| cancer | Poly A | VFHQVFIFMTY | AAGCCCUCCAGGUGUUCCACCAAGUGUU | GAAGAGUAA | GGAGCC | from 5′ to 3′ |
| vaccine | tail: 100 nt | WHLLNAFTVTV | CAUCUUCAUGACAUACUGGCAUCUGCUG | GAAGAAAUA | UCGGUG | end: 5′ UTR |
| Variant 2 | (SEQ ID NO: | PKDLYVVERDT | AACGCCUUUACCGUGACCGUACCCAAGG | UAAGACCCC | GCCUAG | of SEQ ID |
| | 502) | LLKALLEIASCL | ACCUGUACGUGGUGGAGAGAGAUACCC | GGCGCCGCC | CUUCUU | NO: 272, |
| | | EKALQVFHQVF | UGCUGAAAGCCCUGCUGGAAAUCGCCUC | ACC | GCCCCU | ORF |
| | | IFMTYWHLLNA | GUGCCUGGAGAAGGCCCUGCAGGUGUU | | UGGGCC | sequence of |
| | | FTVTVPKDLYV | UCACCAGGUGUUCAUUUUCAUGACCUAU | | UCCCCC | SEQ ID NO: |
| | | VERDTLLKALL | UGGCACCUGUUGAACGCUUUUACUGUG | | CAGCCC | 6, and 3′ UTR |
| | | EIASCLEKALQ | ACCGUGCCAAAGGAUUUAUACGUGGUG | | CUCCUC | sequence of |
| | | VFHQVFIFMTY | GAGCGGGACACACUGCUGAAGGCCCUGC | | CCCUUC | SEQ ID NO: |
| | | WHLLNAFTVTV | UUGAGAUCGCCAGCUGUCUCGAGAAAGC | | CUGCAC | 108. |
| | | PKDLYVVERDT | CCUGCAAGUGUUCCACCAGGUAUUCAUC | | CCGUAC | |
| | | LLKALLEIASCL | UUUAUGACUUACUGGCACCUGCUCAACG | | CCCCGU | |
| | | EKALQVFHQVF | CCUUCACCGUGACAGUGCCCAAGGACUU | | GGUCUU | |
| | | IFMTYWHLLNA | GUACGUGGUCGAGCGGGACACCCUGUUA | | UGAAUA | |
| | | FTVTVPKDLYV | AAGGCCCUGCUGGAGAUUGCCAGCUGUC | | AAGUCU | |
| | | V | UGGAGAAGGCACUGCAGGUGUUCCAUC | | GAGUGG | |
| | | | AGGUGUUUAUCUUUAUGACCUACUGGC | | GCGGC | |
| | | | ACCUCCUGAACGCCUUCACAGUGACCGU | | | |
| | | | GCCCAAGGAUCUCUACGUGGUG | | | |
| SEQ ID NO: | | 716 | 6 | 273 | 108 | 302 |
| IDO/PD-L1 | G5 | MERDTLLKALL | AUGGAGCGGGAUACCCUCCUGAAGGCUC | AGGAAAUAA | UGAUAA | SEQ ID NO: |
| Checkpoint | Cap C1 | EIASCLEKALQ | UGCUGGAGAUCGCAAGCUGCCUGGAGA | GAGAGAAAA | UAGGCU | 302 comprises |
| cancer | Poly A | VFHQVFIFMTY | AAGCCCUCCAGGUGUUCCACCAAGUGUU | GAAGAGUAA | GGAGCC | or consists |
| vaccine | tail: 100 nt | WHLLNAFTVTV | CAUCUUCAUGACAUACUGGCAUCUGCUG | GAAGAAAUA | UCGGUG | from 5′ to 3′ |
| Variant 3 | (SEQ ID NO: | PKDLYVVERDT | AACGCCUUUACCGUGACCGUACCCAAGG | UAAGACCCC | GCCUAG | end: 5′ UTR |
| | 502) | LLKALLEIASCL | ACCUGUACGUGGUGGAGAGAGAUACCC | GGCGCCGCC | CUUCUU | of SEQ ID |
| | | EKALQVFHQVF | UGCUGAAAGCCCUGCUGGAAAUCGCCUC | ACC | GCCCCU | NO: 273, |
| | | IFMTYWHLLNA | GUGCCUGGAGAAGGCCCUGCAGGUGUU | | UGGGCC | ORF |
| | | FTVTVPKDLYV | UCACCAGGUGUUCAUUUUCAUGACCUAU | | UCCCCC | sequence of |
| | | VERDTLLKALL | UGGCACCUGUUGAACGCUUUUACUGUG | | CAGCCC | SEQ ID NO: |
| | | EIASCLEKALQ | ACCGUGCCAAAGGAUUUAUACGUGGUG | | CUCCUC | 6, and 3′ UTR |
| | | VFHQVFIFMTY | GAGCGGGACACACUGCUGAAGGCCCUGC | | CCCUUC | sequence of |
| | | WHLLNAFTVTV | UUGAGAUCGCCAGCUGUCUCGAGAAAGC | | CUGCAC | SEQ ID NO: |
| | | PKDLYVVERDT | CCUGCAAGUGUUCCACCAGGUAUUCAUC | | CCGUAC | 108. |
| | | LLKALLEIASCL | UUUAUGACUUACUGGCACCUGCUCAACG | | CCCCGU | |
| | | EKALQVFHQVF | CCUUCACCGUGACAGUGCCCAAGGACUU | | GGUCUU | |
| | | IFMTYWHLLNA | GUACGUGGUCGAGCGGGACACCCUGUUA | | UGAAUA | |
| | | FTVTVPKDLYV | AAGGCCCUGCUGGAGAUUGCCAGCUGUC | | AAGUCU | |
| | | V | UGGAGAAGGCACUGCAGGUGUUCCAUC | | GAGUGG | |
| | | | AGGUGUUUAUCUUUAUGACCUACUGGC | | GCGGC | |
| | | | ACCUCCUGAACGCCUUCACAGUGACCGU | | | |
| | | | GCCCAAGGAUCUCUACGUGGUG | | | |

### LNPs for Therapy

Disclosed herein is, inter alia, an LNP composition comprising a polynucleotide encoding a checkpoint cancer vaccine comprising one or more IDO antigenic peptides and one or more PD-L1 antigenic peptides that can be administered alone or in combination with an additional agent, e.g., a standard of care therapy. In an embodiment, the additional agent is a polypeptide, e.g., a protein, a fusion protein, a soluble protein, or an antibody (e.g., an antibody fragment, a Fab, an scFv, a single domain Ab, a humanized antibody, a bispecific antibody and/or a multispecific antibody). In an embodiment, the LNP composition and the additional agent are in the same composition or in separate compositions. In an embodiment, the LNP composition and the additional agent are administered substantially simultaneously or sequentially.

### Lipid Content of LNPs

As set forth above, with respect to lipids, LNPs disclosed herein comprise an (i) ionizable lipid; (ii) sterol or other structural lipid; (iii) a non-cationic helper lipid or phospholipid; a (iv) PEG lipid. These categories of lipids are set forth in more detail below.

### Ionizable Lipids

The lipid nanoparticles of the present disclosure include one or more ionizable lipids. In certain embodiments, the ionizable lipids of the disclosure comprise a central amine moiety and at least one biodegradable group. The ionizable lipids described herein may be advantageously used in lipid nanoparticles of the disclosure for the delivery of nucleic acid molecules to mammalian cells or organs. The structures of ionizable lipids set forth below include the prefix I to distinguish them from other lipids of the invention.

In some aspects, the disclosure relates to a compound of Ionizable amino lipid Formula (I):

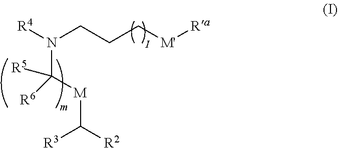

or its N-oxide, or a salt or isomer thereof,
wherein R′^{a} is R′^{branched}; wherein
   R′^{branched} is:

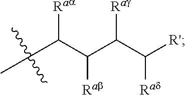

wherein

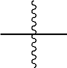

denotes a point of attachment;
wherein R^{aα}, R^{aβ}, R^{aγ}, and R^{aδ} are each independently selected from the group consisting of H, C₂₋₁₂ alkyl, and C₂₋₁₂ alkenyl;
R² and R³ are each independently selected from the group consisting of C₁₋₁₄ alkyl and C₂₋₁₄ alkenyl;
R⁴ is selected from the group consisting of —(CH2)ₙOH, wherein n is selected from the group consisting of 1, 2, 3, 4, and 5, and

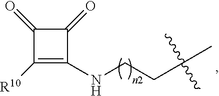

wherein

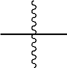

denotes a point of attachment; wherein
R¹⁰ is N(R)₂; each R is independently selected from the group consisting of C₁₋₆ alkyl, C₂₋₃ alkenyl, and H;
and n2 is selected from the group consisting of 1, 2, 3, 4, 5, 6, 7, 8, 9, and 10;
each R⁵ is independently selected from the group consisting of C₁₋₃ alkyl,
C₂₋₃ alkenyl, and H;
each R⁶ is independently selected from the group consisting of C₁₋₃ alkyl,
C₂₋₃ alkenyl, and H;
M and M′ are each independently selected from the group consisting of —C(O)O— and
—OC(O)—;
R′ is a C₁₋₁₂ alkyl or C₂₋₁₂ alkenyl;
l is selected from the group consisting of 1, 2, 3, 4, and 5; and
m is selected from the group consisting of 5, 6, 7, 8, 9, 10, 11, 12, and 13.

In some embodiments of the compounds of Formula (I), R′^{a} is R′^{branched}. R′^{branched} is

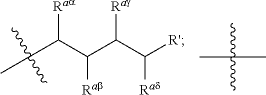

denotes a point of attachment; R^{aα}, R^{aβ}, R^{aγ}, and R^{aδ} are each H; R² and R³ are each C₁₋₁₄ alkyl; R⁴ is —(CH₂)ₙOH; n is 2; each R⁵ is H; each R⁶ is H; M and M′ are each —C(O)O—; R′ is a C₁₋₁₂ alkyl; 1 is 5; and m is 7.

In some embodiments of the compounds of Formula (I), R′^{a} is R′^{branched}. R′^{branched} is

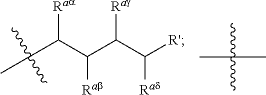

denotes a point of attachment; R^{aα}, R^{aβ}, R^{aγ}, and R^{aδ} are each H; R² and R³ are each C₁₋₁₄ alkyl; R⁴ is —(CH₂)ₙOH; n is 2; each R⁵ is H; each R⁶ is H; M and M′ are each —C(O)O—; R′ is a C₁₋₁₂ alkyl; 1 is 3; and m is 7.

In some embodiments of the compounds of Formula (I), R′^{a} is R′^{branched}.

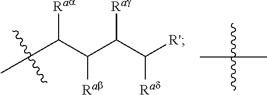

denotes a point of attachment; R^{aα} is C₂₋₁₂ alkyl; R^{aβ}, R^{aγ}, and R^{aδ} are each H; R² and R³ are each C₁₋₁₄ alkyl; R⁴ is

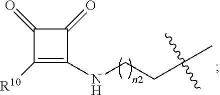

R¹⁰ is NH(C₁₋₆ alkyl); n2 is 2; R⁵ is H; each R⁶ is H; M and M′ are each —C(O)O—; R′ is a C₁₋₁₂ alkyl; 1 is 5; and m is 7.

In some embodiments of the compounds of Formula (I), R′^{a} is R′^{branched}. R′^{branched} is

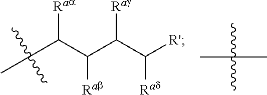

denotes a point of attachment; R^{aα}, R^{aβ}, and R^{aδ} are each H; R^{aγ} is C₂₋₁₂ alkyl; R² and R³ are each C₁₋₁₄ alkyl; R⁴ is —(CH₂)ₙOH; n is 2; each R⁵ is H; each R⁶ is H; M and M′ are each —C(O)O—; R′ is a C₁₋₁₂ alkyl; 1 is 5; and m is 7.

In some embodiments, the compound of Formula (I) is selected from:

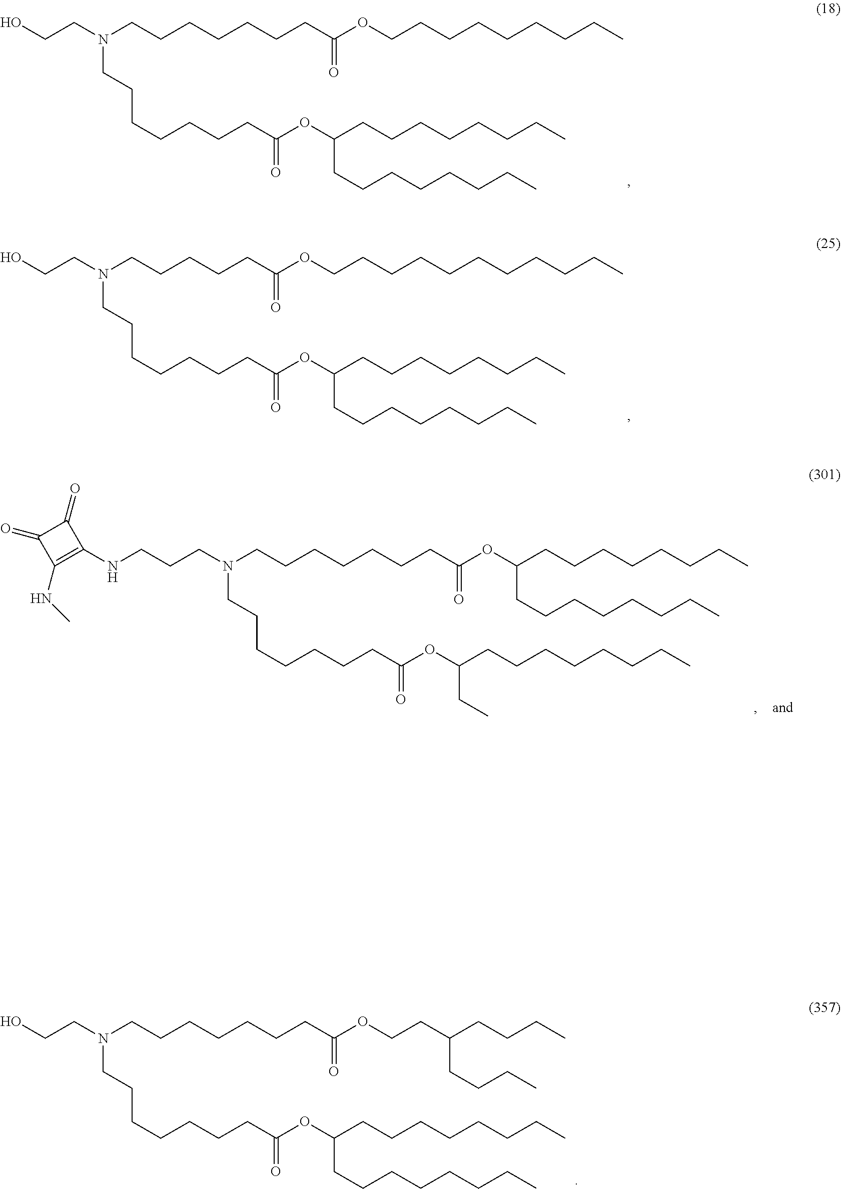 [SMILES: CCCCCCCCCOC(=O)CCCCCCCN(CCO)CCCCCCCC(=O)OC(CCCCCCCC)CCCCCCCC ; CCCCCCCCCCCOC(=O)CCCCCN(CCO)CCCCCCCC(=O)OC(CCCCCCCC)CCCCCCCC ; CCCCCCCCC(CC)OC(=O)CCCCCCCN(CCCCCCCC(=O)OC(CCCCCCCC)CCCCCCCC)CCCNc1c(NC)c(=O)c1=O ; CCCCCCCCC(CCCCCCCC)OC(=O)CCCCCCCN(CCO)CCCCCCCC(=O)OCCC(CCCC)CCCC]

In some embodiments, the compound of Formula (I) is:

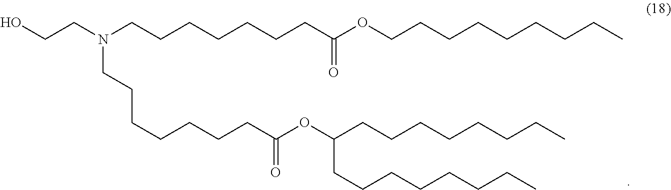 [SMILES: CCCCCCCCCOC(=O)CCCCCCCN(CCO)CCCCCCCC(=O)OC(CCCCCCCC)CCCCCCCC]

In some embodiments, the compound of Formula (I) is:

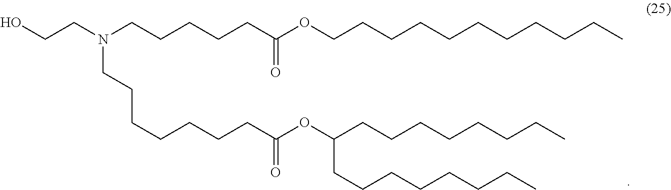 [SMILES: CCCCCCCCCCCOC(=O)CCCCCN(CCO)CCCCCCCC(=O)OC(CCCCCCCC)CCCCCCCC]

In some embodiments, the compound of Formula (I) is:

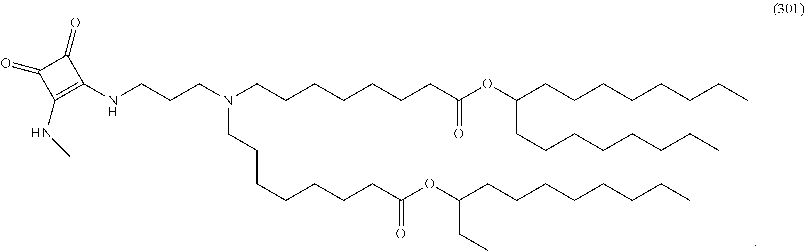 [SMILES: CCCCCCCCC(CC)OC(=O)CCCCCCCN(CCCCCCCC(=O)OC(CCCCCCCC)CCCCCCCC)CCCNc1c(NC)c(=O)c1=O]

In some embodiments, the compound of Formula (I) is:

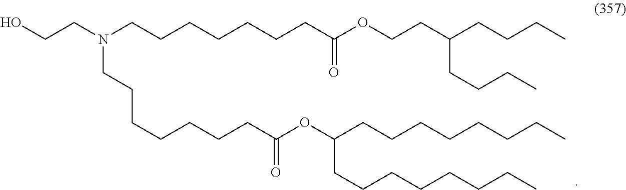 [SMILES: CCCCCCCCC(CCCCCCCC)OC(=O)CCCCCCCN(CCO)CCCCCCCC(=O)OCCC(CCCC)CCCC]

In some aspects, the disclosure relates to a compound of Formula (I-a):

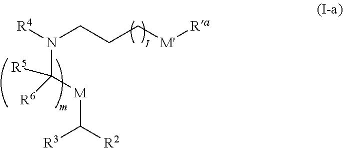

or its N-oxide, or a salt or isomer thereof,
wherein R′^{a} is R′^{branched}, wherein
   R′^{branched} is:

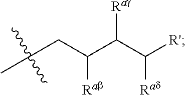

wherein

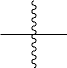

denotes a point of attachment;
wherein R^{aβ}, R^{aγ}, and R^{aδ} are each independently selected from the group consisting of H, C₂₋₁₂ alkyl, and C₂₋₁₂ alkenyl;
R² and R³ are each independently selected from the group consisting of C₁₋₁₄ alkyl and C₂₋₁₄ alkenyl;
R⁴ is selected from the group consisting of —(CH₂)ₙOH, wherein n is selected from the group consisting of 1, 2, 3, 4, and 5, and

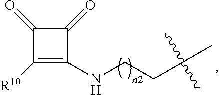

wherein

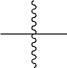

denotes a point of attachment; wherein
R¹⁰ is N(R)₂; each R is independently selected from the group consisting of C₁₋₆ alkyl, C₂₋₃ alkenyl, and H; and n2 is selected from the group consisting of 1, 2, 3, 4, 5, 6, 7, 8, 9, and 10;
each R⁵ is independently selected from the group consisting of C₁₋₃ alkyl,
C₂₋₃ alkenyl, and H;
each R⁶ is independently selected from the group consisting of C₁₋₃ alkyl,
C₂₋₃ alkenyl, and H;
M and M′ are each independently selected from the group consisting of —C(O)O— and
—OC(O)—;
R′ is a C₁₋₁₂ alkyl or C₂₋₁₂ alkenyl;
l is selected from the group consisting of 1, 2, 3, 4, and 5; and
m is selected from the group consisting of 5, 6, 7, 8, 9, 10, 11, 12, and 13.

In some aspects, the disclosure relates to a compound of Formula (I-b):

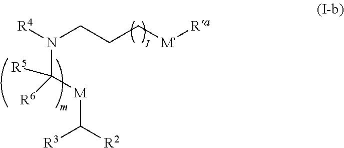

or its N-oxide, or a salt or isomer thereof,
wherein R′^{a} is R′^{branched}; wherein
   R′^{branched} is:

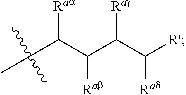

wherein

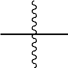

denotes a point of attachment;
   wherein R^{aα}, R^{aβ}, R^{aγ}, and R^{aδ} are each independently selected from the group consisting of H, C₂₋₁₂ alkyl, and C₂₋₁₂ alkenyl;
   R² and R³ are each independently selected from the group consisting of C₁₋₁₄ alkyl and C₂₋₁₄ alkenyl;
   R⁴ is —(CH₂)ₙOH, wherein n is selected from the group consisting of 1, 2, 3, 4, and 5;
   each R⁵ is independently selected from the group consisting of C₁₋₃ alkyl,
   C₂₋₃ alkenyl, and H;
   each R⁶ is independently selected from the group consisting of C₁₋₃ alkyl,
   C₂₋₃ alkenyl, and H;
   M and M′ are each independently selected from the group consisting of —C(O)O— and

### —OC(O)—;

R′ is a C₁₋₁₂ alkyl or C₂₋₁₂ alkenyl;
l is selected from the group consisting of 1, 2, 3, 4, and 5; and
m is selected from the group consisting of 5, 6, 7, 8, 9, 10, 11, 12, and 13.

In some embodiments of Formula (I) or (I-b), R′^{a} is R′^{branched}. R′^{branched} is

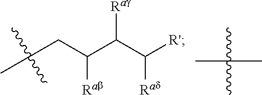

denotes a point of attachment; R^{aβ}, R^{aγ}, and R^{aδ} are each H; R² and R³ are each C₁₋₁₄ alkyl; R⁴ is —(CH₂)ₙOH; n is 2; each R⁵ is H; each R⁶ is H; M and M′ are each —C(O)O—; R′ is a C₁₋₁₂ alkyl; 1 is 5; and m is 7.

In some embodiments of Formula (I) or (I-b), R′^{a} is R′^{branched}. R′^{branched} is

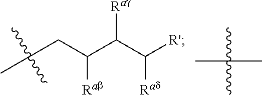

denotes a point of attachment; R^{aβ}, R^{aγ}, and R^{aδ} are each H; R² and R³ are each C₁₋₁₄ alkyl; R⁴ is —(CH₂)ₙOH; n is 2; each R⁵ is H; each R⁶ is H; M and M′ are each —C(O)O—; R′ is a C₁₋₁₂ alkyl; 1 is 3; and m is 7.

In some embodiments of Formula (I) or (I-b), R′^{a} is R′^{branched}. R′^{branched} is

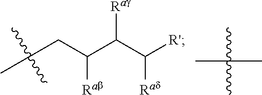

denotes a point of attachment; R^{aβ} and R^{aδ} are each H; R^{aγ} is C₂₋₁₂ alkyl; R² and R³ are each C₁₋₁₄ alkyl; R⁴ is —(CH₂)ₙOH; n is 2; each R⁵ is H; each R⁶ is H; M and M′ are each —C(O)O—; R′ is a C₁₋₁₂ alkyl; 1 is 5; and m is 7.

In some aspects, the disclosure relates to a compound of Formula (I-c):

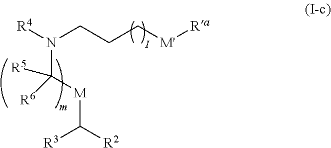

or its N-oxide, or a salt or isomer thereof,
wherein R′^{a} is R′^{branched}, wherein
   R′^{branched} is:

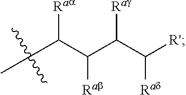

wherein

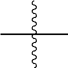

denotes a point of attachment;
wherein R^{aα}, R^{aβ}, R^{aγ}, and R^{aδ} are each independently selected from the group consisting of H, C₂₋₁₂ alkyl, and C₂₋₁₂ alkenyl;
R² and R³ are each independently selected from the group consisting of C₁₋₁₄ alkyl and
C₂₋₁₄ alkenyl;
R⁴ is

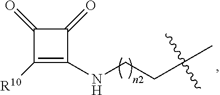

wherein

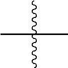

denotes a point of attachment;
wherein
R¹⁰ is N(R)₂; each R is independently selected from the group consisting of C₁₋₆ alkyl, C₂₋₃ alkenyl, and H;
n2 is selected from the group consisting of 1, 2, 3, 4, 5, 6, 7, 8, 9, and 10;
each R⁵ is independently selected from the group consisting of C₁₋₃ alkyl,
C₂₋₃ alkenyl, and H;
each R⁶ is independently selected from the group consisting of C₁₋₃ alkyl,
C₂₋₃ alkenyl, and H;
M and M′ are each independently selected from the group consisting of —C(O)O— and
—OC(O)—;
R′ is a C₁₋₁₂ alkyl or C₂₋₁₂ alkenyl;
l is selected from the group consisting of 1, 2, 3, 4, and 5; and
m is selected from the group consisting of 5, 6, 7, 8, 9, 10, 11, 12, and 13.

In some embodiments, R′^{a} is R′^{branched}. R′^{branched} is

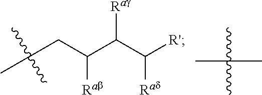

denotes a point of attachment; R^{aβ}, R^{aγ}, and R^{aδ} are each H; R^{aα} is C₂₋₁₂ alkyl; R² and R³ are each C₁₋₁₄ alkyl; R⁴ is

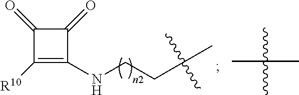

denotes a point of attachment; R¹⁰ is NH(C₁₋₆ alkyl); n2 is 2; each R⁵ is H; each R⁶ is H; M and M′ are each —C(O)O—; R′ is a C₁₋₁₂ alkyl; 1 is 5; and m is 7.

In some embodiments, the compound of Formula (I-c) is:

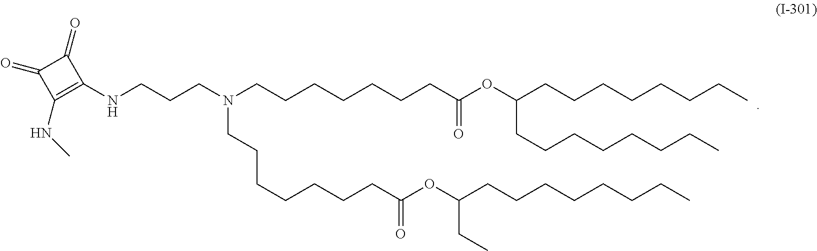 [SMILES: CCCCCCCCC(CC)OC(=O)CCCCCCCN(CCCCCCCC(=O)OC(CCCCCCCC)CCCCCCCC)CCCNc1c(NC)c(=O)c1=O]

In some aspects, the disclosure relates to a compound of Formula (II):

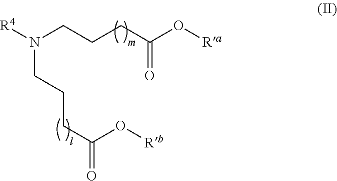

or its N-oxide, or a salt or isomer thereof,
wherein R′^{a} is R′^{branched} or R′^{cyclic}; wherein
R′^{branched} is:

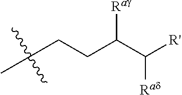

and R′^{cyclic} is:

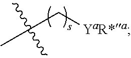

and
R′^{b} is:

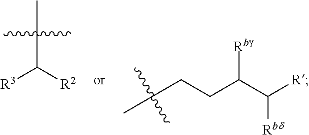

wherein

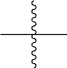

denotes a point of attachment;
R^{aγ} and R^{aδ} are each independently selected from the group consisting of H, C₁₋₁₂ alkyl, and C₂₋₁₂ alkenyl, wherein at least one of R^{aγ} and R^{aδ} is selected from the group consisting of C₁₋₁₂ alkyl and C₂₋₁₂ alkenyl;
R^{bγ} and R^{bδ} are each independently selected from the group consisting of H, C₁₋₁₂ alkyl, and C₂₋₁₂ alkenyl, wherein at least one of R^{bγ} and R^{bδ} is selected from the group consisting of C₁₋₁₂ alkyl and C₂₋₁₂ alkenyl;
R² and R³ are each independently selected from the group consisting of C₁₋₁₄ alkyl and
C₂₋₁₄ alkenyl;
R⁴ is selected from the group consisting of —(CH₂)ₙOH wherein n is selected from the group consisting of 1, 2, 3, 4, and 5, and

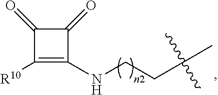

wherein

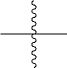

denotes a point of attachment; wherein
R¹⁰ is N(R)₂; each R is independently selected from the group consisting of C₁₋₆ alkyl, C₂₋₃ alkenyl, and H; and n2 is selected from the group consisting of 1, 2, 3, 4, 5, 6, 7, 8, 9, and 10;
each R′ independently is a C₁₋₁₂ alkyl or C₂₋₁₂ alkenyl;
Y^{a} is a C₃₋₆ carbocycle;
R*″^{a} is selected from the group consisting of C₁₋₁₅ alkyl and C₂₋₁₅ alkenyl; and
s is 2 or 3;
m is selected from 1, 2, 3, 4, 5, 6, 7, 8, and 9;
l is selected from 1, 2, 3, 4, 5, 6, 7, 8, and 9.

In some aspects, the disclosure relates to a compound of Formula (II-a):

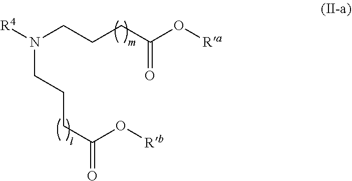

or its N-oxide, or a salt or isomer thereof,
wherein R′^{a} is R′^{branched} or R′^{cyclic}; wherein
R′^{branched} is:

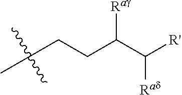

and R′^{b} is:

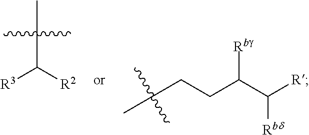

wherein

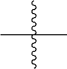

denotes a point of attachment;
R^{aγ} and R^{aδ} are each independently selected from the group consisting of H, C₁₋₁₂ alkyl, and C₂₋₁₂ alkenyl, wherein at least one of R^{aγ} and R^{aδ} is selected from the group consisting of C₁₋₁₂ alkyl and C₂₋₁₂ alkenyl;
R^{bγ} and R^{bδ} are each independently selected from the group consisting of H, C₁₋₁₂ alkyl, and C₂₋₁₂ alkenyl, wherein at least one of R^{bγ} and R^{bδ} is selected from the group consisting of C₁₋₁₂ alkyl and C₂₋₁₂ alkenyl;
R² and R³ are each independently selected from the group consisting of C₁₋₁₄ alkyl and C₂₋₁₄ alkenyl;
R⁴ is selected from the group consisting of —(CH₂)ₙOH wherein n is selected from the group consisting of 1, 2, 3, 4, and 5, and

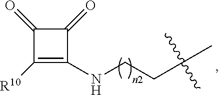

wherein

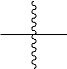

denotes a point of attachment; wherein
R¹⁰ is N(R)₂; each R is independently selected from the group consisting of C₁₋₆ alkyl, C₂₋₃ alkenyl, and H;
and n2 is selected from the group consisting of 1, 2, 3, 4, 5, 6, 7, 8, 9, and 10;
each R′ independently is a C₁₋₁₂ alkyl or C₂₋₁₂ alkenyl;
m is selected from 1, 2, 3, 4, 5, 6, 7, 8, and 9;
l is selected from 1, 2, 3, 4, 5, 6, 7, 8, and 9.

In some aspects, the disclosure relates to a compound of Formula (II-b):

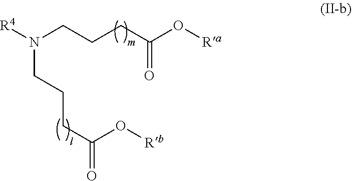

or its N-oxide, or a salt or isomer thereof,
wherein R′^{a} is R′^{branched} or R′^{cyclic}; wherein
R′^{branched} is:

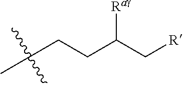

and R′^{b} is:

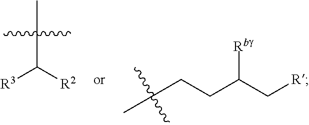

wherein

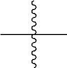

denotes a point of attachment;
R^{aγ} and R^{bγ} are each independently selected from the group consisting of C₁₋₁₂ alkyl and C₂₋₁₂ alkenyl;
R² and R³ are each independently selected from the group consisting of C₁₋₁₄ alkyl and
C₂₋₁₄ alkenyl;
R⁴ is selected from the group consisting of —(CH₂)ₙOH wherein n is selected from the group consisting of 1, 2, 3, 4, and 5, and

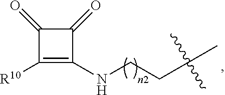

wherein

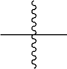

denotes a point of attachment; wherein
R¹⁰ is N(R)₂; each R is independently selected from the group consisting of C₁₋₆ alkyl, C₂₋₃ alkenyl, and H;
and n2 is selected from the group consisting of 1, 2, 3, 4, 5, 6, 7, 8, 9, and 10;
each R′ independently is a C₁₋₁₂ alkyl or C₂₋₁₂ alkenyl;
m is selected from 1, 2, 3, 4, 5, 6, 7, 8, and 9;
l is selected from 1, 2, 3, 4, 5, 6, 7, 8, and 9.

In some aspects, the disclosure relates to a compound of Formula (II-c):

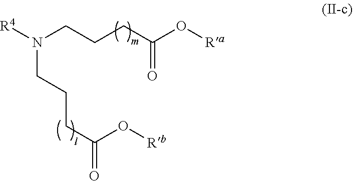

or its N-oxide, or a salt or isomer thereof,
wherein R′^{a} is R′^{branched} or R′^{cyclic}; wherein
R′^{branched} is:

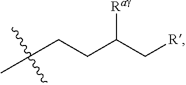

and R′^{b} is:

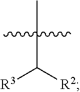

wherein

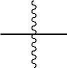

denotes a point of attachment;
wherein R^{aγ} is selected from the group consisting of C₁₋₁₂ alkyl and C₂₋₁₂ alkenyl;
R² and R³ are each independently selected from the group consisting of C₁₋₁₄ alkyl and
C₂₋₁₄ alkenyl;
R⁴ is selected from the group consisting of —(CH₂)ₙOH wherein n is selected from the group consisting of 1, 2, 3, 4, and 5, and

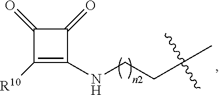

wherein

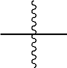

denotes a point of attachment; wherein
R¹⁰ is N(R)₂; each R is independently selected from the group consisting of C₁₋₆ alkyl, C₂₋₃ alkenyl, and H;
and n2 is selected from the group consisting of 1, 2, 3, 4, 5, 6, 7, 8, 9, and 10;
R′ is a C₁₋₁₂ alkyl or C₂₋₁₂ alkenyl;
m is selected from 1, 2, 3, 4, 5, 6, 7, 8, and 9;
l is selected from 1, 2, 3, 4, 5, 6, 7, 8, and 9.

In some aspects, the disclosure relates to a compound of Formula (II-d):

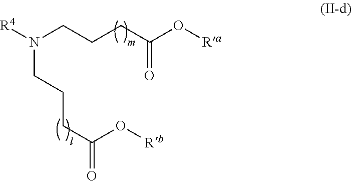

or its N-oxide, or a salt or isomer thereof,
wherein R′^{a} is R′^{branched} or R′^{cyclic}; wherein
R′^{branched} is:

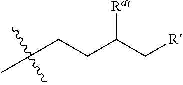

and R′^{b} is:

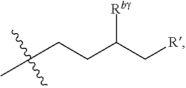

wherein

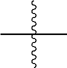

denotes a point of attachment;
wherein R^{aγ} and R^{bγ} are each independently selected from the group consisting of C₁₋₁₂ alkyl and C₂₋₁₂ alkenyl;
R⁴ is selected from the group consisting of —(CH₂)ₙOH wherein n is selected from the group consisting of 1, 2, 3, 4, and 5, and

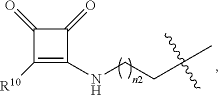

wherein

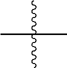

denotes a point of attachment; wherein
R¹⁰ is N(R)₂; each R is independently selected from the group consisting of C₁₋₆ alkyl, C₂₋₃ alkenyl, and H;
and n2 is selected from the group consisting of 1, 2, 3, 4, 5, 6, 7, 8, 9, and 10;
each R′ independently is a C₁₋₁₂ alkyl or C₂₋₁₂ alkenyl;
m is selected from 1, 2, 3, 4, 5, 6, 7, 8, and 9;
l is selected from 1, 2, 3, 4, 5, 6, 7, 8, and 9.

In some aspects, the disclosure relates to a compound of Formula (II-e):

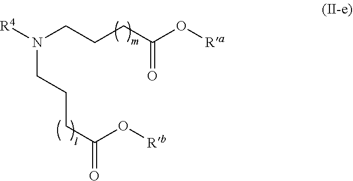

or its N-oxide, or a salt or isomer thereof,
wherein R′^{a} is R′^{branched} or R′^{cyclic}; wherein
R′^{branched} is:

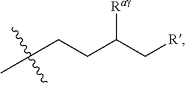

and R′^{b} is:

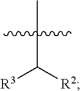

wherein

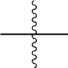

denotes a point of attachment;
wherein R^{aγ} is selected from the group consisting of C₁₋₁₂ alkyl and C₂₋₁₂ alkenyl;
R² and R³ are each independently selected from the group consisting of C₁₋₁₄ alkyl and
C₂₋₁₄ alkenyl;
R⁴ is —(CH₂)ₙOH wherein n is selected from the group consisting of 1, 2, 3, 4, and 5;
R′ is a C₁₋₁₂ alkyl or C₂₋₁₂ alkenyl;
m is selected from 1, 2, 3, 4, 5, 6, 7, 8, and 9;
l is selected from 1, 2, 3, 4, 5, 6, 7, 8, and 9.

In some embodiments of the compound of Formula (II), (II-a), (II-b), (II-c), (II-d), or (II-e), m and l are each independently selected from 4, 5, and 6. In some embodiments of the compound of Formula (II), (II-a), (II-b), (II-c), (II-d), or (II-e), m and l are each 5.

In some embodiments of the compound of Formula (II), (II-a), (II-b), (II-c), (II-d), or (II-e), each R′ independently is a C₁₋₁₂ alkyl. In some embodiments of the compound of Formula (II), (II-a), (II-b), (II-c), (II-d), or (II-e), each R′ independently is a C₂₋₅ alkyl.

In some embodiments of the compound of Formula (II), (II-a), (II-b), (II-c), (II-d), or (II-e), R′^{b} is:

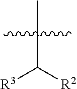

and R² and R³ are each independently a C₁₋₁₄ alkyl. In some embodiments of the compound of Formula (II), (II-a), (II-b), (II-c), (II-d), or (II-e), R′^{b} is:

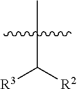

and R² and R³ are each independently a C₆₋₁₀ alkyl. In some embodiments of the compound of Formula (II), (II-a), (II-b), (II-c), (II-d), or (II-e), R′^{b} is:

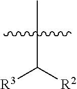

and R² and R³ are each a C₈ alkyl.

In some embodiments of the compound of Formula (II), (II-a), (II-b), (II-c), (II-d), or (II-e), R′^{branched} is:

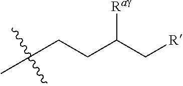

### and R′^{b} is:

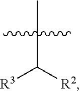

R^{aγ} is a C₁₋₁₂ alkyl and R² and R³ are each independently a C₆₋₁₀ alkyl. In some embodiments of the compound of Formula (II), (II-a), (II-b), (II-c), (II-d), or (II-e), R′^{branched} is:

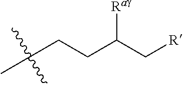

### and R′^{b} is:

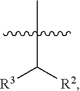

R^{aγ} is a C₂₋₆ alkyl and R² and R³ are each independently a C₆₋₁₀ alkyl. In some embodiments of the compound of Formula (II), (II-a), (II-b), (II-c), (II-d), or (II-e), R′^{branched} is:

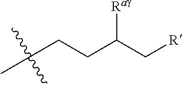

### and R′^{b} is:

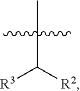

R^{aγ} is a C₂₋₆ alkyl, and R² and R³ are each a C₈ alkyl.

In some embodiments of the compound of Formula (II), (II-a), (II-b), (II-c), (II-d), or (II-e), R′^{branched} is:

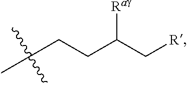

### R′^{b} is:

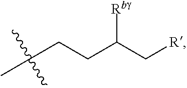

and R^{aγ} and R^{bγ} are each a C¹⁻¹² alkyl. In some embodiments of the compound of Formula (II), (II-a), (II-b), (II-c), (II-d), or (II-e), R′^{branched} is:

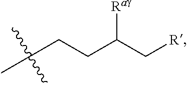

### R′^{b} is:

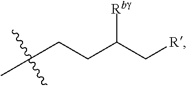

and R^{aγ} and R^{bγ} are each a C₂₋₆ alkyl.

In some embodiments of the compound of Formula (II), (II-a), (II-b), (II-c), (II-d), or (II-e), m and 1 are each independently selected from 4, 5, and 6 and each R′ independently is a C₁₋₁₂ alkyl. In some embodiments of the compound of Formula (II), (II-a), (II-b), (II-c), (II-d), or (II-e), m and l are each 5 and each R′ independently is a C₂₋₅ alkyl.

In some embodiments of the compound of Formula (II), (II-a), (II-b), (II-c), (II-d), or (II-e), R′^{branched} is:

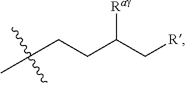

### R′^{b} is:

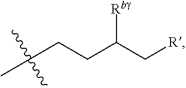

m and l are each independently selected from 4, 5, and 6, each R′ independently is a C₁₋₁₂ alkyl, and R^{aγ} and R^{bγ} are each a C₁₋₁₂ alkyl. In some embodiments of the compound of Formula (II), (II-a), (II-b), (II-c), (II-d), or (II-e), R′^{branched} is:

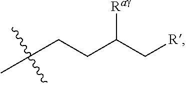

### R′^{b} is:

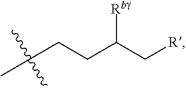

m and l are each 5, each R′ independently is a C₂₋₅ alkyl, and R^{aγ} and R^{bγ} are each a C₂₋₆ alkyl.

In some embodiments of the compound of Formula (II), (II-a), (II-b), (II-c), (II-d), or (II-e), R′^{branched} is:

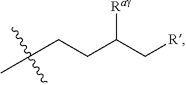

### and R′^{b} is:

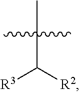

m and l are each independently selected from 4, 5, and 6, R′ is a C₁₋₁₂ alkyl, R^{aγ} is a C₁₋₁₂ alkyl and R² and R³ are each independently a C₆₋₁₀ alkyl. In some embodiments of the compound of Formula (II), (II-a), (II-b), (II-c), (II-d), or (II-e), R′^{branched} is:

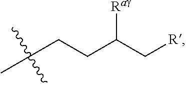

### and R′^{b} is:

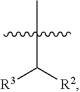

m and l are each 5, R′ is a C₂₋₅ alkyl, R^{aγ} is a C₂₋₆ alkyl, and R² and R³ are each a C₈ alkyl.

In some embodiments of the compound of Formula (II), (II-a), (II-b), (II-c), (II-d), or (II-e), R⁴ is

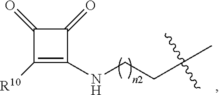

wherein R¹⁰ is NH(C₁₋₆ alkyl) and n2 is 2. In some embodiments of the compound of Formula (II), (II-a), (II-b), (II-c), (II-d), or (II-e), R⁴ is

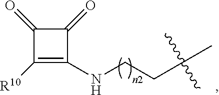

wherein R¹⁰ is NH(CH₃) and n2 is 2.

In some embodiments of the compound of Formula (II), (II-a), (II-b), (II-c), (II-d), or (II-e), R′^{branched} is:

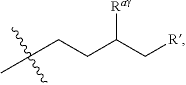

### R′^{b} is:

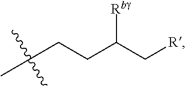

m and l are each independently selected from 4, 5, and 6, each R′ independently is a C₁₋₁₂ alkyl, R^{aγ} and R^{bγ} are each a C₁₋₁₂ alkyl, and R⁴ is

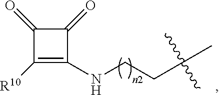

wherein R¹⁰ is NH(C₁₋₆ alkyl), and n2 is 2. In some embodiments of the compound of Formula (II), (II-a), (II-b), (II-c), (II-d), or (II-e), R′^{branched} is:

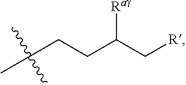

### R′^{b} is:

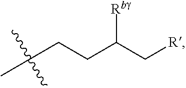

m and l are each 5, each R′ independently is a C₂₋₅ alkyl, R^{aγ} and R^{bγ} are each a C₂₋₆ alkyl, and R⁴ is

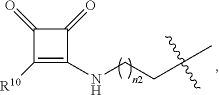

wherein R¹⁰ is NH(CH₃) and n2 is 2.

In some embodiments of the compound of Formula (II), (II-a), (II-b), (II-c), (II-d), or (II-e), R′^{branched} is:

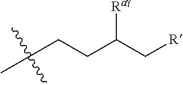

### and R′^{b} is:

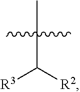

m and l are each independently selected from 4, 5, and 6, R′ is a C₁₋₁₂ alkyl, R² and R³ are each independently a C₆₋₁₀ alkyl, R^{aγ} is a C₁₋₁₂ alkyl, and R⁴ is

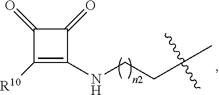

wherein R¹⁰ is NH(C₁₋₆ alkyl) and n2 is 2. In some embodiments of the compound of Formula (II), (II-a), (II-b), (II-c), (II-d), or (II-e), R′^{branched} is:

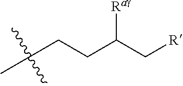

### and R′b is:

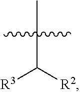

m and l are each 5, R′ is a C₂₋₅ alkyl, R^{aγ} is a C₂₋₆ alkyl, R² and R³ are each a C₈ alkyl, and R⁴ is

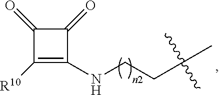

wherein R¹⁰ is NH(CH₃) and n2 is 2.

In some embodiments of the compound of Formula (II), (II-a), (II-b), (II-c), (II-d), or (II-e), R⁴ is —(CH₂)ₙOH and n is 2, 3, or 4. In some embodiments of the compound of Formula (II), (II-a), (II-b), (II-c), (II-d), or (II-e), R⁴ is —(CH₂)ₙOH and n is 2.

In some embodiments of the compound of Formula (II), (II-a), (II-b), (II-c), (II-d), or (II-e), R′^{branched} is:

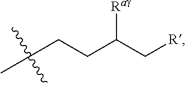

### R′^{b} is:

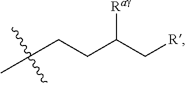

m and l are each independently selected from 4, 5, and 6, each R′ independently is a C₁₋₁₂ alkyl, R^{aγ} and R^{bγ} are each a C₁₋₁₂ alkyl, R⁴ is —(CH₂)ₙOH, and n is 2, 3, or 4. In some embodiments of the compound of Formula (II), (II-a), (II-b), (II-c), (II-d), or (II-e), R′^{branched} is:

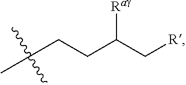

### R′^{b} is:

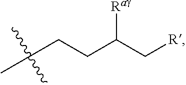

m and l are each 5, each R′ independently is a C₂₋₅ alkyl, R^{aγ} and R^{bγ} are each a C₂₋₆ alkyl, R⁴ is —(CH₂)ₙOH, and n is 2.

In some aspects, the disclosure relates to a compound of Formula (II-f):

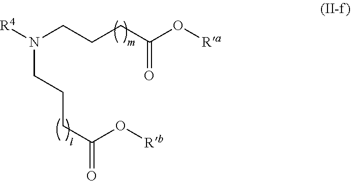

or its N-oxide, or a salt or isomer thereof,
wherein R′^{a} is R′^{branched} or R′^{cyclic}; wherein
R′^{branched} is:

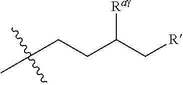

and R′^{b} is:

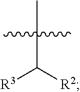

wherein

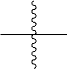

denotes a point of attachment;
R^{aγ} is a C₁₋₁₂ alkyl;
R² and R³ are each independently a C₁₋₁₄ alkyl;
R⁴ is —(CH₂)ₙOH wherein n is selected from the group consisting of 1, 2, 3, 4, and 5;
R′ is a C₁₋₁₂ alkyl;
m is selected from 4, 5, and 6; and
l is selected from 4, 5, and 6.

In some embodiments of the compound of Formula (II-f), m and l are each 5, and n is 2, 3, or 4.

In some embodiments of the compound of Formula (II-f) R′ is a C₂₋₅ alkyl, R^{aγ} is a C₂₋₆ alkyl, and R² and R³ are each a C₆₋₁₀ alkyl.

In some embodiments of the compound of Formula (II-f), m and l are each 5, n is 2, 3, or 4, R′ is a C₂₋₅ alkyl, R^{aγ} is a C₂₋₆ alkyl, and R² and R³ are each a C₆₋₁₀ alkyl.

In some aspects, the disclosure relates to a compound of Formula (II-g):

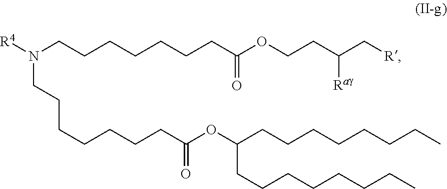

wherein
R^{aγ} is a C₂₋₆ alkyl;
R′ is a C₂₋₅ alkyl; and
R⁴ is selected from the group consisting of —(CH₂)ₙOH wherein n is selected from the group consisting of 3, 4, and 5, and

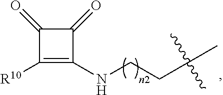

wherein

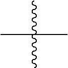

denotes a point of attachment, R¹⁰ is NH(C₁₋₆ alkyl), and n2 is selected from the group consisting of 1, 2, and 3.

In some aspects, the disclosure relates to a compound of Formula (II-h):

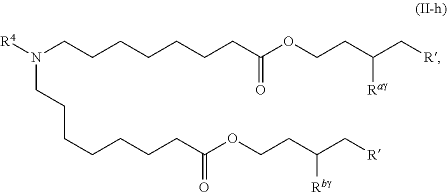

wherein
R^{aγ} and R^{bγ} are each independently a C₂₋₆ alkyl;
each R′ independently is a C₂₋₅ alkyl; and
R⁴ is selected from the group consisting of —(CH₂)ₙOH wherein n is selected from the group consisting of 3, 4, and 5, and

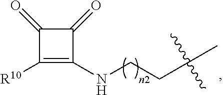

wherein

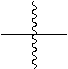

denotes a point of attachment, R¹⁰ is NH(C₁₋₆ alkyl), and n2 is selected from the group consisting of 1, 2, and 3.

In some embodiments of the compound of Formula (II-g) or (II-h), R⁴ is

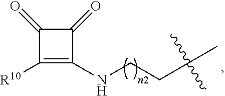

wherein
R¹⁰ is NH(CH₃) and n2 is 2.

In some embodiments of the compound of Formula (II-g) or (II-h), R⁴ is —(CH₂)₂OH.

In some aspects, the disclosure relates to a compound having the Formula (III):

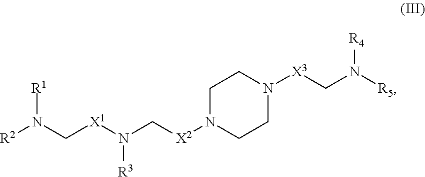

or a salt or isomer thereof, wherein
R¹, R², R³, R⁴, and R₅ are independently selected from the group consisting of C₅₋₂₀ alkyl, C₅₋₂₀ alkenyl, —R″MR′, —R*YR″, —YR″, and —R*OR″;
each M is independently selected from the group consisting
of —C(O)O—, —OC(O)—, —OC(O)O—, —C(O)N(R′)—, —N(R′) C(O)—, —C(O)—, —C(S)—, —C(S)S—, —SC(S)—, —CH(OH)—, —P(O)(OR′)O—, —S(O)₂—, an aryl group, and a heteroaryl group;
X¹, X², and X³ are independently selected from the group consisting of a bond, —CH₂—, (CH₂)₂—, —CHR—, —CHY—, —C(O)—, —C(O)O—, —OC(O)—, —C(O)—CH₂—, —CH₂—C(O)—, —C(O)O—CH₂—, —OC(O)—CH₂—, —CH₂—C(O)O—, —CH₂—OC(O)—, —CH(OH)—, —C(S)—, and —CH(SH)—; each Y is independently a C₃₋₆ carbocycle;
each R* is independently selected from the group consisting of C₁₋₁₂ alkyl and C₂₋₁₂ alkenyl;
each R is independently selected from the group consisting of C₁₋₃ alkyl and a C₃₋₆ carbocycle;
each R′ is independently selected from the group consisting of C₁₋₁₂ alkyl, C₂₋₁₂ alkenyl, and H; and
each R″ is independently selected from the group consisting of C₃₋₁₂ alkyl and C₃₋₁₂ alkenyl, and wherein:
i) at least one of X¹, X², and X³ is not-CH₂—; and/or
ii) at least one of R¹, R², R³, R⁴, and R⁵ is —R″MR′.

In some embodiments, R¹, R², R³, R⁴, and R₅ are each C₅₋₂₀ alkyl; X¹ is —CH₂—; and X² and X³ are each —C(O)—.

In some embodiments, the compound of Formula (III) is:

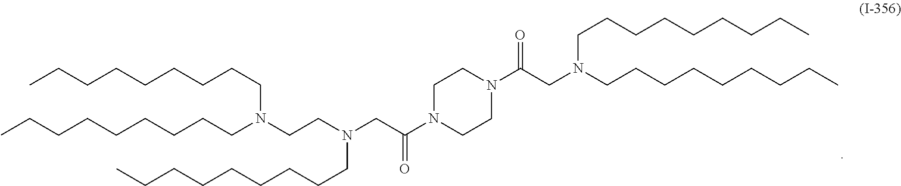 [SMILES: CCCCCCCCCN(CCCCCCCCC)CCN(CCCCCCCCC)CC(=O)N1CCN(C(=O)CN(CCCCCCCCC)CCCCCCCCC)CC1]

The central amine moiety of a lipid according to any of the Formulae herein, e.g. a compound having any of Formula (I), (I-a), (I-b), (I-c), (II), (II-a), (II-b), (II-c), (II-d), (II-e), (II-f), (II-g), (II-h), or (III) (each of these preceded by the letter I for clarity) may be protonated at a physiological pH. Thus, a lipid may have a positive or partial positive charge at physiological pH. Such lipids may be referred to as cationic or ionizable (amino) lipids. Lipids may also be zwitterionic, i.e., neutral molecules having both a positive and a negative charge.

In some embodiments, the amount the ionizable amino lipid of the invention, e.g. a compound having any of Formula (I), (I-a), (I-b), (I-c), (II), (II-a), (II-b), (II-c), (II-d), (II-e), (II-f), (II-g), (II-h), or (III) (each of these preceded by the letter I for clarity) ranges from about 1 mol % to 99 mol % in the lipid composition.

In one embodiment, the amount of the ionizable amino lipid of the invention, e.g. a compound having any of Formula (I), (I-a), (I-b), (I-c), (II), (II-a), (II-b), (II-c), (II-d), (II-e), (II-f), (II-g), (II-h), or (III) (each of these preceded by the letter I for clarity) is at least about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, or 99 mol % in the lipid composition.

In one embodiment, the amount of the ionizable amino lipid of the invention, e.g. a compound having any of Formula (I), (I-a), (I-b), (I-c), (II), (II-a), (II-b), (II-c), (II-d), (II-e), (II-f), (II-g), (II-h), or (III) (each of these preceded by the letter I for clarity) ranges from about 30 mol % to about 70 mol %, from about 35 mol % to about 65 mol %, from about 40 mol % to about 60 mol %, and from about 45 mol % to about 55 mol % in the lipid composition.

In one specific embodiment, the amount of the ionizable amino lipid of the invention, e.g. a compound having any of Formula (I), (I-a), (I-b), (I-c), (II), (II-a), (II-b), (II-c), (II-d), (II-e), (II-f), (II-g), (II-h), or (III) (each of these preceded by the letter I for clarity) is about 45 mol % in the lipid composition.

In one specific embodiment, the amount of the ionizable amino lipid of the invention, e.g. a compound having any of Formula (I), (I-a), (I-b), (I-c), (II), (II-a), (II-b), (II-c), (II-d), (II-e), (II-f), (II-g), (II-h), or (III) (each of these preceded by the letter I for clarity) is about 40 mol % in the lipid composition.

In one specific embodiment, the amount of the ionizable amino lipid of the invention, e.g. a compound having any of Formula (I), (I-a), (I-b), (I-c), (II), (II-a), (II-b), (II-c), (II-d), (II-e), (II-f), (II-g), (II-h), or (III) (each of these preceded by the letter I for clarity) is about 50 mol % in the lipid composition.

In addition to the ionizable amino lipid disclosed herein, e.g. a compound having any of Formula (I), (I-a), (I-b), (I-c), (II), (II-a), (II-b), (II-c), (II-d), (II-e), (II-f), (II-g), (II-h), or (III), (each of these preceded by the letter I for clarity) the lipid-based composition (e.g., lipid nanoparticle) disclosed herein can comprise additional components such as cholesterol and/or cholesterol analogs, non-cationic helper lipids, structural lipids, PEG-lipids, and any combination thereof.

Additional ionizable lipids of the invention can be selected from the non-limiting group consisting of 3-(didodecylamino)-N1,N1,4-tridodecyl-1-piperazineethanamine (KL10),
N1-[2-(didodecylamino)ethyl]-N1,N4,N4-tridodecyl-1,4-piperazinediethanamine (KL22),
14,25-ditridecyl-15,18,21,24-tetraaza-octatriacontane (KL25),
1,2-dilinoleyloxy-N,N-dimethylaminopropane (DLin-DMA),
2,2-dilinoleyl-4-dimethylaminomethyl-[1,3]-dioxolane (DLin-K-DMA), heptatriaconta-6,9,28,31-tetraen-19-yl 4-(dimethylamino) butanoate (DLin-MC3-DMA),
2,2-dilinoleyl-4-(2-dimethylaminoethyl)-[1,3]-dioxolane (DLin-KC2-DMA),
1,2-dioleyloxy-N,N-dimethylaminopropane (DODMA), (13Z,165Z)—N,N-dimethyl-3-nonydocosa-13-16-dien-1-amine (L608),
2-({8-[(3B)-cholest-5-en-3-yloxy]octyl}oxy)-N,N-dimethyl-3-[(9Z,12Z)-octadeca-9,12-dien-1-yloxy]propan-1-amine (Octyl-CLinDMA),
(2R)-2-({8-[(3B)-cholest-5-en-3-yloxy]octyl}oxy)-N,N-dimethyl-3-[(9Z,12Z)-octadeca-9,12-dien-1-yloxy]propan-1-amine (Octyl-CLinDMA (2R)), and
(2S)-2-({8-[(3B)-cholest-5-en-3-yloxy]octyl}oxy)-N,N-dimethyl-3-[(9Z,12Z)-octadeca-9,12-dien-1-yloxy]propan-1-amine (Octyl-CLinDMA (2S)). In addition to these, an ionizable amino lipid can also be a lipid including a cyclic amine group.

Ionizable lipids of the invention can also be the compounds disclosed in International Publication No. WO 2017/075531 A1, hereby incorporated by reference in its entirety.

Ionizable lipids of the invention can also be the compounds disclosed in International Publication No. WO 2015/199952 A1, hereby incorporated by reference in its entirety.

In any of the foregoing or related aspects, the ionizable lipid of the LNP of the disclosure comprises a compound included in any e.g. a compound having any of Formula (I), (I-a), (I-b), (I-c), (II), (II-a), (II-b), (II-c), (II-d), (II-e), (II-f), (II-g), (II-h), or (III) (each of these preceded by the letter I for clarity).

In any of the foregoing or related aspects, the ionizable lipid of the LNP of the disclosure comprises a compound comprising any of Compound Nos. 18, 25, 301, and 357.

In any of the foregoing or related aspects, the ionizable lipid of the LNP of the disclosure comprises at least one compound selected from the group consisting of: Compound Nos. 18, 25, 301, and 357. In another embodiment, the ionizable lipid of the LNP of the disclosure comprises a compound selected from the group consisting of: Compound Nos. 18, 25, 301, and 357. In another embodiment, the ionizable lipid of the LNP of the disclosure comprises Compound 18. In another embodiment, the ionizable lipid of the LNP of the disclosure comprises Compound 25.

In any of the foregoing or related aspects, the synthesis of compounds of the invention, e.g. compounds comprising any of Compound Nos. 18, 25, 301, and 357, follows the synthetic descriptions in U.S. Provisional Patent Application No. 62/733,315, filed Sep. 19, 2018.

### Representative Synthetic Routes:

### Compound I-182: Heptadecan-9-yl 8-((3-((2-(methylamino)-3,4-dioxocyclobut-1-en-1-yl)amino)propyl)(8-(nonyloxy)-8-oxooctyl)amino)octanoate 3-Methoxy-4-(methylamino)cyclobut-3-ene-1,2-dione

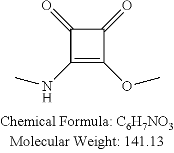

To a solution of 3,4-dimethoxy-3-cyclobutene-1,2-dione (1 g, 7 mmol) in 100 mL diethyl ether was added a 2M methylamine solution in THF (3.8 mL, 7.6 mmol) and a precipitate formed. The mixture was stirred at room temperature for 24 hours, then filtered to collect the solid. The solid was washed with diethyl ether and air-dried, then dissolved in hot EtOAc and filtered. The filtrate was allowed to cool to room temperature, then cooled to 0° C. to afford a precipitate that was isolated via filtration, washed with cold EtOAc, air-dried, then dried under vacuum to yield 3-methoxy-4-(methylamino)cyclobut-3-ene-1,2-dione (0.70 g, 5 mmol, 73%) as a solid. ¹H NMR (300 MHz, DMSO-d₆) δ: ppm 8.50 (br. d, 1H, J=69 Hz); 4.27 (s, 3H); 3.02 (sdd, 3H, J=42 Hz, 4.5 Hz).

### Heptadecan-9-yl 8-((3-((2-(methylamino)-3,4-dioxocyclobut-1-en-1-yl)amino)propyl)(8-(nonyloxy)-8-oxooctyl)amino)octanoate

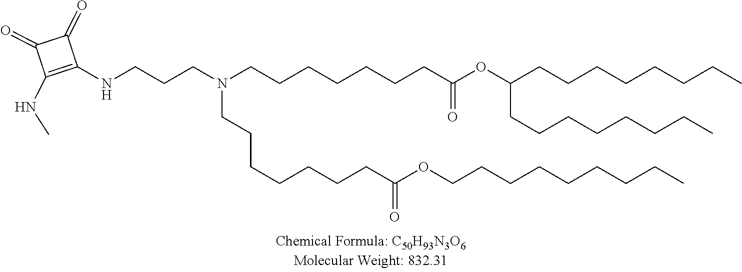 [SMILES: CCCCCCCCCOC(=O)CCCCCCCN(CCCCCCCC(=O)OC(CCCCCCCC)CCCCCCCC)CCCNc1c(NC)c(=O)c1=O]

To a solution of heptadecan-9-yl 8-((3-aminopropyl)(8-(nonyloxy)-8-oxooctyl)amino)octanoate (200 mg, 0.28 mmol) in 10 mL ethanol was added 3-methoxy-4-(methylamino)cyclobut-3-ene-1,2-dione (39 mg, 0.28 mmol). The reaction mixture stirred at room temperature for 20 hours, then concentrated in vacuo to yield a residue. The residue was purified by silica gel chromatography (0-100% (mixture of 1% NH₄OH, 20% MeOH in dichloromethane) in dichloromethane) to give heptadecan-9-yl 8-((3-((2-(methylamino)-3,4-dioxocyclobut-1-en-1-yl)amino)propyl)(8-(nonyloxy)-8-oxooctyl)amino)octanoate (138 mg, 0.17 mmol, 60%) as a solid. UPLC/ELSD: RT=3. min. MS(ES): m/z (MH⁺) 833.4 for C₅₁H₉₅N₃O₆. ¹H NMR (300 MHz, CDCl₃) δ: ppm 7.86 (br. s., 1H); 4.86 (quint., 1H, J=6 Hz); 4.05 (t, 2H, J=6 Hz); 3.92 (d, 2H, J=3 Hz); 3.20 (s, 6H); 2.63 (br. s, 2H); 2.42 (br. s, 3H); 2.28 (m, 4H); 1.74 (br. s, 2H); 1.61 (m, 8H); 1.50 (m, 5H); 1.41 (m, 3H); 1.25 (br. m, 47H); 0.88 (t, 9H, J=7.5 Hz).

### Compound I-301: Heptadecan-9-yl 8-((3-((2-(methylamino)-3,4-dioxocyclobut-1-en-1-yl)amino)propyl)(8-oxo-8-(undecan-3-yloxy)octyl)amino)octanoate

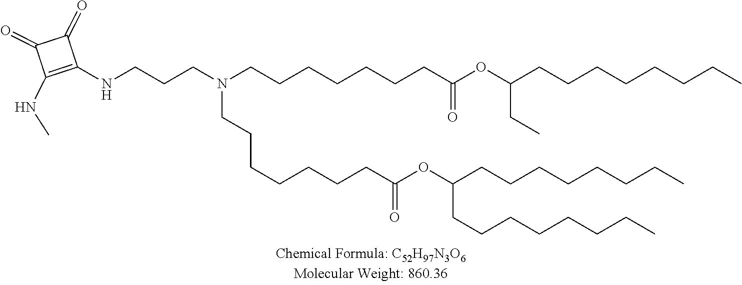 [SMILES: CCCCCCCCC(CC)OC(=O)CCCCCCCN(CCCCCCCC(=O)OC(CCCCCCCC)CCCCCCCC)CCCNc1c(NC)c(=O)c1=O]

Compound I-301 was prepared analogously to compound 182 except that heptadecan-9-yl 8-((3-aminopropyl)(8-oxo-8-(undecan-3-yloxy)octyl)amino)octanoate (500 mg, 0.66 mmol) was used instead of heptadecan-9-yl 8-((3-aminopropyl)(8-(nonyloxy)-8-oxooctyl)amino)octanoate. Following an aqueous workup, the residue was purified by silica gel chromatography (0-50% (mixture of 1% NH₄OH, 20% MeOH in dichloromethane) in dichloromethane) to give heptadecan-9-yl 8-((3-((2-(methylamino)-3,4-dioxocyclobut-1-en-1-yl)amino)propyl)(8-oxo-8-(undecan-3-yloxy)octyl)amino)octanoate (180 mg, 32%) as a solid. HPLC/UV (254 nm): RT=6.77 min. MS (CI): m/z (MH⁺) 860.7 for C₅₂H₉₇N₃O₆. 1H NMR (300 MHz, CDCl₃): δ ppm 4.86-4.79 (m, 2H); 3.66 (bs, 2H); 3.25 (d, 3H, J=4.9 Hz); 2.56-2.52 (m, 2H); 2.42-2.37 (m, 4H); 2.28 (dd, 4H, J=2.7 Hz, 7.4 Hz); 1.78-1.68 (m, 3H); 1.64-1.50 (m, 16H); 1.48-1.38 (m, 6H); 1.32-1.18 (m, 43H); 0.88-0.84 (m, 12H).

### Cholesterol/Structural Lipids

The LNP described herein comprises one or more structural lipids.

As used herein, the term “structural lipid” refers to sterols and also to lipids containing sterol moieties. Incorporation of structural lipids in the lipid nanoparticle may help mitigate aggregation of other lipids in the particle. Structural lipids can include, but are not limited to, cholesterol, fecosterol, ergosterol, bassicasterol, tomatidine, tomatine, ursolic, alpha-tocopherol, and mixtures thereof. In certain embodiments, the structural lipid is cholesterol. In certain embodiments, the structural lipid includes cholesterol and a corticosteroid (such as, for example, prednisolone, dexamethasone, prednisone, and hydrocortisone), or a combination thereof.

In some embodiments, the structural lipid is a sterol. As defined herein, “sterols” are a subgroup of steroids consisting of steroid alcohols. In certain embodiments, the structural lipid is a steroid. In certain embodiments, the structural lipid is cholesterol. In certain embodiments, the structural lipid is an analog of cholesterol. In certain embodiments, the structural lipid is alpha-tocopherol. Examples of structural lipids include, but are not limited to, the following:

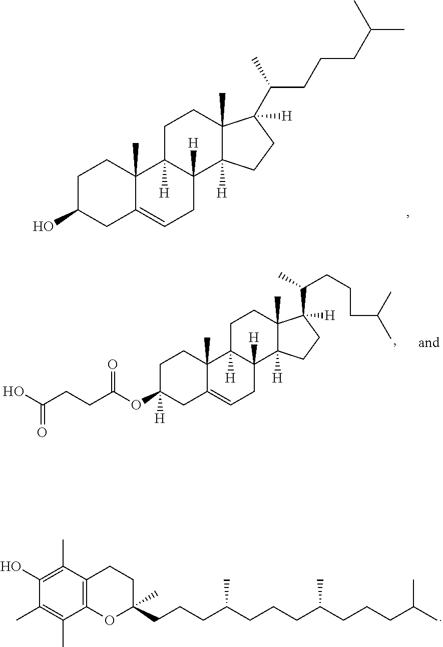

The target cell target cell delivery LNPs described herein comprises one or more structural lipids.

As used herein, the term “structural lipid” refers to sterols and also to lipids containing sterol moieties. Incorporation of structural lipids in the lipid nanoparticle may help mitigate aggregation of other lipids in the particle. In certain embodiments, the structural lipid includes cholesterol and a corticosteroid (such as, for example, prednisolone, dexamethasone, prednisone, and hydrocortisone), or a combination thereof.

In some embodiments, the structural lipid is a sterol. As defined herein, “sterols” are a subgroup of steroids consisting of steroid alcohols. Structural lipids can include, but are not limited to, sterols (e.g., phytosterols or zoosterols).

In certain embodiments, the structural lipid is a steroid. For example, sterols can include, but are not limited to, cholesterol, β-sitosterol, fecosterol, ergosterol, sitosterol, campesterol, stigmasterol, brassicasterol, ergosterol, tomatidine, tomatine, ursolic acid, alpha-tocopherol, or the like.

In certain embodiments, the structural lipid is cholesterol. In certain embodiments, the structural lipid is an analog of cholesterol.

### Ratio of Compounds

A lipid nanoparticle of the invention can include a structural component as described herein. The structural component of the lipid nanoparticle can be an individual compound or a mixture of one or more structural compounds of the invention.

### Non-Cationic Helper Lipids/Phospholipids

In some embodiments, the lipid-based composition (e.g., LNP) described herein comprises one or more non-cationic helper lipids. In some embodiments, the non-cationic helper lipid is a phospholipid. In some embodiments, the non-cationic helper lipid is a phospholipid substitute or replacement.

As used herein, the term “non-cationic helper lipid” refers to a lipid comprising at least one fatty acid chain of at least 8 carbons in length and at least one polar head group moiety. In one embodiment, the helper lipid is not a phosphatidyl choline (PC). In one embodiment the non-cationic helper lipid is a phospholipid or a phospholipid substitute. In some embodiments, the phospholipid or phospholipid substitute can be, for example, one or more saturated or (poly) unsaturated phospholipids, or phospholipid substitutes, or a combination thereof. In general, phospholipids comprise a phospholipid moiety and one or more fatty acid moieties.

A phospholipid moiety can be selected, for example, from the non-limiting group consisting of phosphatidyl choline, phosphatidyl ethanolamine, phosphatidyl glycerol, phosphatidyl serine, phosphatidic acid, 2-lysophosphatidyl choline, and a sphingomyelin.

A fatty acid moiety can be selected, for example, from the non-limiting group consisting of lauric acid, myristic acid, myristoleic acid, palmitic acid, palmitoleic acid, stearic acid, oleic acid, linoleic acid, alpha-linolenic acid, erucic acid, phytanoic acid, arachidic acid, arachidonic acid, eicosapentaenoic acid, behenic acid, docosapentaenoic acid, and docosahexaenoic acid.

Phospholipids include, but are not limited to, glycerophospholipids such as phosphatidylcholines, phosphatidylethanolamines, phosphatidylserines, phosphatidylinositols, phosphatidyl glycerols, and phosphatidic acids. Phospholipids also include phosphosphingolipid, such as sphingomyelin.

In some embodiments, the non-cationic helper lipid is a DSPC analog, a DSPC substitute, oleic acid, or an oleic acid analog.

In some embodiments, a non-cationic helper lipid is a non-phosphatidyl choline (PC) zwitterionic lipid, a DSPC analog, oleic acid, an oleic acid analog, or a 1,2-distearoyl-i77-glycero-3-phosphocholine (DSPC) substitute.

### Phospholipids

### Phospholipids

The lipid composition of the lipid nanoparticle composition disclosed herein can comprise one or more phospholipids, for example, one or more saturated or (poly) unsaturated phospholipids or a combination thereof. In general, phospholipids comprise a phospholipid moiety and one or more fatty acid moieties.

A phospholipid moiety can be selected, for example, from the non-limiting group consisting of phosphatidyl choline, phosphatidyl ethanolamine, phosphatidyl glycerol, phosphatidyl serine, phosphatidic acid, 2-lysophosphatidyl choline, and a sphingomyelin.

A fatty acid moiety can be selected, for example, from the non-limiting group consisting of lauric acid, myristic acid, myristoleic acid, palmitic acid, palmitoleic acid, stearic acid, oleic acid, linoleic acid, alpha-linolenic acid, erucic acid, phytanoic acid, arachidic acid, arachidonic acid, eicosapentaenoic acid, behenic acid, docosapentaenoic acid, and docosahexaenoic acid.

Particular phospholipids can facilitate fusion to a membrane. For example, a cationic phospholipid can interact with one or more negatively charged phospholipids of a membrane (e.g., a cellular or intracellular membrane). Fusion of a phospholipid to a membrane can allow one or more elements (e.g., a therapeutic agent) of a lipid-containing composition (e.g., LNPs) to pass through the membrane permitting, e.g., delivery of the one or more elements to a target tissue.

Non-natural phospholipid species including natural species with modifications and substitutions including branching, oxidation, cyclization, and alkynes are also contemplated. For example, a phospholipid can be functionalized with or cross-linked to one or more alkynes (e.g., an alkenyl group in which one or more double bonds is replaced with a triple bond). Under appropriate reaction conditions, an alkyne group can undergo a copper-catalyzed cycloaddition upon exposure to an azide. Such reactions can be useful in functionalizing a lipid bilayer of a nanoparticle composition to facilitate membrane permeation or cellular recognition or in conjugating a nanoparticle composition to a useful component such as a targeting or imaging moiety (e.g., a dye).

Phospholipids include, but are not limited to, glycerophospholipids such as phosphatidylcholines, phosphatidylethanolamines, phosphatidylserines, phosphatidylinositols, phosphatidy glycerols, and phosphatidic acids. Phospholipids also include phosphosphingolipid, such as sphingomyelin.

In some embodiments, a phospholipid of the invention comprises 1,2-distearoyl-sn-glycero-3-phosphocholine (DSPC), 1,2-dioleoyl-sn-glycero-3-phosphoethanolamine (DOPE), 1,2-dilinoleoyl-sn-glycero-3-phosphocholine (DLPC), 1,2-dimyristoyl-sn-gly cero-phosphocholine (DMPC), 1,2-dioleoyl-sn-glycero-3-phosphocholine (DOPC), 1,2-dipalmitoyl-sn-glycero-3-phosphocholine (DPPC), 1,2-diundecanoyl-sn-glycero-phosphocholine (DUPC), 1-palmitoyl-2-oleoyl-sn-glycero-3-phosphocholine (POPC), 1,2-di-O-octadecenyl-sn-glycero-3-phosphocholine (18:0 Diether PC), 1-oleoyl-2 cholesterylhemisuccinoyl-sn-glycero-3-phosphocholine (OChemsPC), 1-hexadecyl-sn-glycero-3-phosphocholine (C16 Lyso PC), 1,2-dilinolenoyl-sn-glycero-3-phosphocholine, 1,2-diarachidonoyl-sn-glycero-3-phosphocholine, 1,2-didocosahexaenoyl-sn-glycero-3-phosphocholine, 1,2-diphytanoyl-sn-glycero-3-phosphoethanolamine (ME 16.0 PE), 1,2-distearoyl-sn-glycero-3-phosphoethanolamine, 1,2-dilinoleoyl-sn-glycero-3-phosphoethanolamine, 1,2-dilinolenoyl-sn-glycero-3-phosphoethanolamine, 1,2-diarachidonoyl-sn-glycero-3-phosphoethanolamine, 1,2-didocosahexaenoyl-sn-glycero-3-phosphoethanolamine, 1,2-dioleoyl-sn-glycero-3-phospho-rac-(1-glycerol) sodium salt (DOPG), sphingomyelin, and mixtures thereof.

In certain embodiments, a phospholipid useful or potentially useful in the present invention is an analog or variant of DSPC. In certain embodiments, a phospholipid useful or potentially useful in the present invention is a compound of Formula (IV):

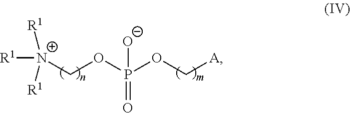

or a salt thereof, wherein:
each R¹ is independently optionally substituted alkyl; or optionally two R¹ are joined together with the intervening atoms to form optionally substituted monocyclic carbocyclyl or optionally substituted monocyclic heterocyclyl; or optionally three R¹ are joined together with the intervening atoms to form optionally substituted bicyclic carbocyclyl or optionally substitute bicyclic heterocyclyl;
n is 1, 2, 3, 4, 5, 6, 7, 8, 9, or 10;
m is 0, 1, 2, 3, 4, 5, 6, 7, 8, 9, or 10;

A is of the Formula:

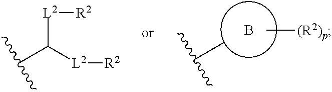

each instance of L2 is independently a bond or optionally substituted C₁₋₆ alkylene, wherein one methylene unit of the optionally substituted C₁₋₆ alkylene is optionally replaced with O, N(R^{N}), S, C(O), C(O)N(R^{N}), NR^{N}C(O), C(O)O, OC(O), OC(O)O, OC(O)N(R^{N}), NR^{N}C(O)O, or NR^{N}C(O)N(R^{N});

each instance of R² is independently optionally substituted C₁₋₃₀ alkyl, optionally substituted C₁₋₃₀ alkenyl, or optionally substituted C₁₋₃₀ alkynyl; optionally wherein one or more methylene units of R² are independently replaced with optionally substituted carbocyclylene, optionally substituted heterocyclylene, optionally substituted arylene, optionally substituted heteroarylene, N(R^{N}), O, S, C(O), C(O)N(R^{N}), —NR^{N}C(O), NR^{N}C(O)N(R^{N}), C(O)O, OC(O), OC(O)O, OC(O)N(R^{N}), NR^{N}C(O)O, C(O) S, SC(O), —C(═NR^{N}), C(═NR^{N})N(R^{N}), NR^{N}C(═NR^{N}), NR^{N}C(═NR^{N})N(R^{N}), C(S), C(S)N(R^{N}), NR^{N}C(S), —NR^{N}C(S)N(R^{N}), S(O), OS(O), S(O)O, OS(O)O, OS(O)₂, S(O)₂O, OS(O)₂O, N(R^{N})S(O), S(O)N(R^{N}), —N(R^{N})S(O)N(R^{N}), OS(O)N(R^{N}), N(R^{N})S(O)O, S(O)₂, N(R^{N})S(O)₂, S(O)₂N(R^{N}), N(R^{N})S(O)₂N(R^{N}), —OS(O)₂N(R^{N}), or N(R^{N})S(O)₂O;
each instance of R^{N} is independently hydrogen, optionally substituted alkyl, or a nitrogen protecting group;
Ring B is optionally substituted carbocyclyl, optionally substituted heterocyclyl, optionally substituted aryl, or optionally substituted heteroaryl; and
p is 1 or 2;
provided that the compound is not of the Formula:

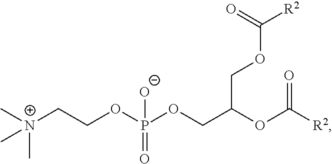

wherein each instance of R² is independently unsubstituted alkyl, unsubstituted alkenyl, or unsubstituted alkynyl.

In some embodiments, the phospholipids may be one or more of the phospholipids described in U.S. Application No. 62/520,530.

### Phospholipid Head Modifications

In certain embodiments, a phospholipid useful or potentially useful in the present invention comprises a modified phospholipid head (e.g., a modified choline group). In certain embodiments, a phospholipid with a modified head is DSPC, or analog thereof, with a modified quaternary amine. For example, in embodiments of Formula (IV), at least one of R¹ is not methyl. In certain embodiments, at least one of R¹ is not hydrogen or methyl. In certain embodiments, the compound of Formula (IV) is of one of the following Formulae:

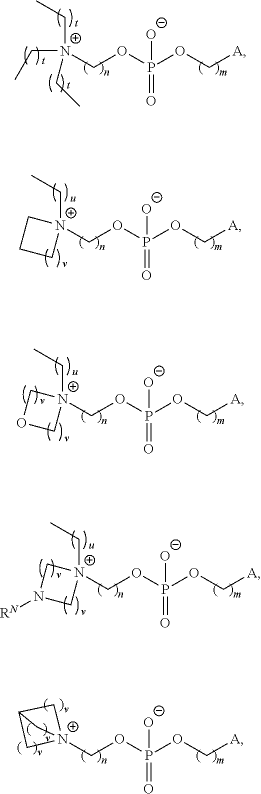

or a salt thereof, wherein:
each t is independently 1, 2, 3, 4, 5, 6, 7, 8, 9, or 10;
each u is independently 0, 1, 2, 3, 4, 5, 6, 7, 8, 9, or 10; and
each v is independently 1, 2, or 3.

In certain embodiments, a compound of Formula (IV) is of Formula (IV-a):

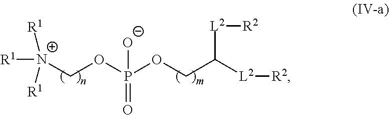

or a salt thereof.

In certain embodiments, a phospholipid useful or potentially useful in the present invention comprises a cyclic moiety in place of the glyceride moiety. In certain embodiments, a phospholipid useful in the present invention is DSPC, or analog thereof, with a cyclic moiety in place of the glyceride moiety. In certain embodiments, the compound of Formula (IV) is of Formula (IV-b):

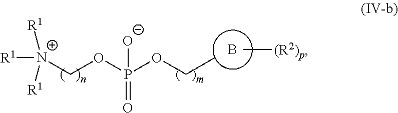

or a salt thereof.

### Phospholipid Tail Modifications

In certain embodiments, a phospholipid useful or potentially useful in the present invention comprises a modified tail. In certain embodiments, a phospholipid useful or potentially useful in the present invention is DSPC, or analog thereof, with a modified tail. As described herein, a “modified tail” may be a tail with shorter or longer aliphatic chains, aliphatic chains with branching introduced, aliphatic chains with substituents introduced, aliphatic chains wherein one or more methylenes are replaced by cyclic or heteroatom groups, or any combination thereof. For example, in certain embodiments, the compound of (IV) is of Formula (IV-a), or a salt thereof, wherein at least one instance of R² is each instance of R² is optionally substituted C₁₋₃₀ alkyl, wherein one or more methylene units of R² are independently replaced with optionally substituted carbocyclylene, optionally substituted heterocyclylene, optionally substituted arylene, optionally substituted heteroarylene, N(R^{N}), O, S, C(O), C(O)N(R^{N}), —NR^{N}C(O), NR^{N}C(O)N(R^{N}), C(O)O, OC(O), OC(O)O, OC(O)N(R^{N}), NR^{N}C(O)O, C(O) S, SC(O), —C(═NR^{N}), C(═NR^{N})N(R^{N}), NR^{N}C(═NR^{N}), NR^{N}C(═NR^{N})N(R^{N}), C(S), C(S)N(R^{N}), NR^{N}C(S), —NR^{N}C(S)N(R^{N}), S(O), OS(O), S(O)O, OS(O)O, OS(O)₂, S(O)₂O, OS(O)₂O, N(R^{N})S(O), S(O)N(R^{N}), —N(R^{N})S(O)N(R^{N}), OS(O)N(R^{N}), N(R^{N})S(O)O, S(O)₂, N(R^{N})S(O)₂, S(O)₂N(R^{N}), N(R^{N})S(O)₂N(R^{N}), —OS(O)₂N(R^{N}), or N(R^{N})S(O)₂O.

In certain embodiments, the compound of Formula (IV) is of Formula (IV-c):

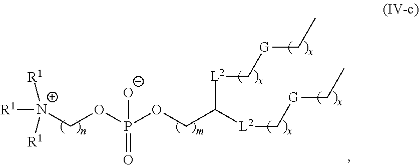

or a salt thereof, wherein:
each x is independently an integer between 0-30, inclusive; and
each instance is G is independently selected from the group consisting of optionally substituted carbocyclylene, optionally substituted heterocyclylene, optionally substituted arylene, optionally substituted heteroarylene, N(R^{N}), O, S, C(O), C(O)N(R^{N}), NR^{N}C(O), NR^{N}C(O)N(R^{N}), C(O)O, OC(O), OC(O)O, OC(O)N(R^{N}), NR^{N}C(O)O, C(O) S, SC(O), C(═NR^{N}), C(═NR^{N})N(R^{N}), NR^{N}C(═NR^{N}), —NR^{N}C(═NR^{N})N(R^{N}), C(S), C(S)N(R^{N}), NR^{N}C(S), NR^{N}C(S)N(R^{N}), S(O), OS(O), S(O)O, OS(O)O, —OS(O)₂, S(O)₂O, OS(O)₂O, N(R^{N})S(O), S(O)N(R^{N}), N(R^{N})S(O)N(R^{N}), OS(O)N(R^{N}), N(R^{N})S(O)O, S(O)₂, N(R^{N})S(O)₂, S(O)₂N(R^{N}), N(R^{N})S(O)₂N(R^{N}), OS(O)₂N(R^{N}), or N(R^{N})S(O)₂O. Each possibility represents a separate embodiment of the present invention.

In certain embodiments, a phospholipid useful or potentially useful in the present invention comprises a modified phosphocholine moiety, wherein the alkyl chain linking the quaternary amine to the phosphoryl group is not ethylene (e.g., n is not 2). Therefore, in certain embodiments, a phospholipid useful or potentially useful in the present invention is a compound of Formula (IV), wherein n is 1, 3, 4, 5, 6, 7, 8, 9, or 10. For example, in certain embodiments, a compound of Formula (IV) is of one of the following Formulae:

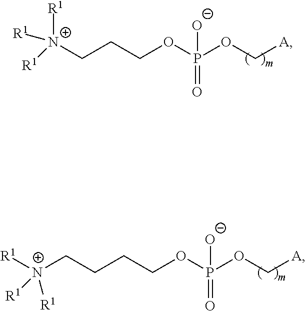

or a salt thereof.

### Alternative Lipids

In certain embodiments, a phospholipid useful or potentially useful in the present invention comprises a modified phosphocholine moiety, wherein the alkyl chain linking the quaternary amine to the phosphoryl group is not ethylene (e.g., n is not 2). Therefore, in certain embodiments, a phospholipid useful.

In certain embodiments, an alternative lipid is used in place of a phospholipid of the present disclosure.

In certain embodiments, an alternative lipid of the invention is oleic acid.

In certain embodiments, the alternative lipid is one of the following:

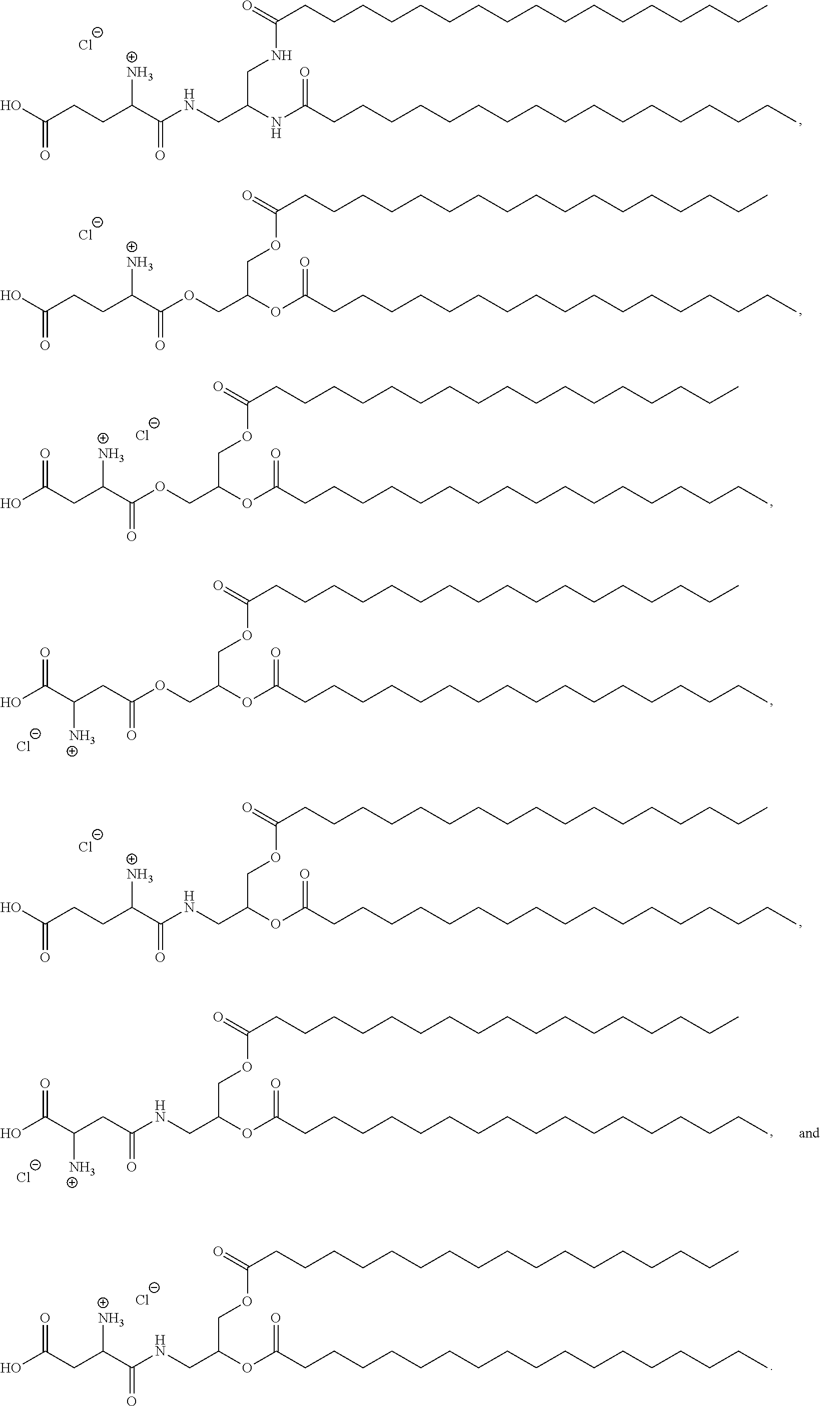

### PEG Lipids

The lipid composition of a pharmaceutical composition disclosed herein can comprise one or more a polyethylene glycol (PEG) lipid.

As used herein, the term “PEG-lipid” refers to polyethylene glycol (PEG)-modified lipids. Non-limiting examples of PEG-lipids include PEG-modified phosphatidylethanolamine and phosphatidic acid, PEG-ceramide conjugates (e.g., PEG-CerC14 or PEG-CerC20), PEG-modified dialkylamines and PEG-modified 1,2-diacyloxypropan-3-amines. Such lipids are also referred to as PEGylated lipids. For example, a PEG lipid can be PEG-c-DOMG, PEG-DMG, PEG-DLPE, PEG-DMPE, PEG-DPPC, or a PEG-DSPE lipid.

In some embodiments, the PEG-lipid includes, but not limited to 1,2-dimyristoyl-sn-glycerol methoxypolyethylene glycol (PEG-DMG), 1,2-distearoyl-sn-glycero-3-phosphoethanolamine-N-[amino(polyethylene glycol)] (PEG-DSPE), PEG-disteryl glycerol (PEG-DSG), PEG-dipalmetoleyl, PEG-dioleyl, PEG-distearyl, PEG-diacylglycamide (PEG-DAG), PEG-dipalmitoyl phosphatidylethanolamine (PEG-DPPE), or PEG-1,2-dimyristyloxlpropyl-3-amine (PEG-c-DMA).

In one embodiment, the PEG-lipid is selected from the group consisting of a PEG-modified phosphatidylethanolamine, a PEG-modified phosphatidic acid, a PEG-modified ceramide, a PEG-modified dialkylamine, a PEG-modified diacylglycerol, a PEG-modified dialkylglycerol, and mixtures thereof.

In some embodiments, the lipid moiety of the PEG-lipids includes those having lengths of from about C₁₄ to about C₂₂, preferably from about C₁₄ to about C₁₆. In some embodiments, a PEG moiety, for example an mPEG-NH₂, has a size of about 1000, 2000, 5000, 10,000, 15,000 or 20,000 daltons. In one embodiment, the PEG-lipid is PEG2k-DMG.

In one embodiment, the lipid nanoparticles described herein can comprise a PEG lipid which is a non-diffusible PEG. Non-limiting examples of non-diffusible PEGs include PEG-DSG and PEG-DSPE.

PEG-lipids are known in the art, such as those described in U.S. Pat. No. 8,158,601 and International Publ. No. WO 2015/130584 A2, which are incorporated herein by reference in their entirety.

In general, some of the other lipid components (e.g., PEG lipids) of various Formulae, described herein may be synthesized as described International Patent Application No. PCT/US2016/000129, filed Dec. 10, 2016, entitled “Compositions and Methods for Delivery of Therapeutic Agents,” which is incorporated by reference in its entirety.

The lipid component of a lipid nanoparticle composition may include one or more molecules comprising polyethylene glycol, such as PEG or PEG-modified lipids. Such species may be alternately referred to as PEGylated lipids. A PEG lipid is a lipid modified with polyethylene glycol. A PEG lipid may be selected from the non-limiting group including PEG-modified phosphatidylethanolamines, PEG-modified phosphatidic acids, PEG-modified ceramides, PEG-modified dialkylamines, PEG-modified diacylglycerols, PEG-modified dialkylglycerols, and mixtures thereof. For example, a PEG lipid may be PEG-c-DOMG, PEG-DMG, PEG-DLPE, PEG-DMPE, PEG-DPPC, or a PEG-DSPE lipid.

In some embodiments the PEG-modified lipids are a modified form of PEG DMG. PEG-DMG has the following structure:

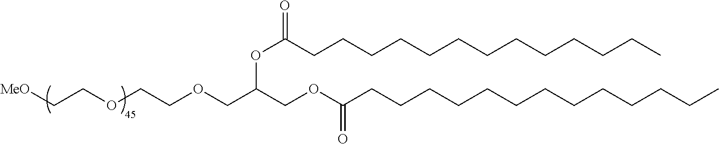

In one embodiment, PEG lipids useful in the present invention can be PEGylated lipids described in International Publication No. WO2012099755, the contents of which is herein incorporated by reference in its entirety. Any of these exemplary PEG lipids described herein may be modified to comprise a hydroxyl group on the PEG chain. In certain embodiments, the PEG lipid is a PEG-OH lipid. As generally defined herein, a “PEG-OH lipid” (also referred to herein as “hydroxy-PEGylated lipid”) is a PEGylated lipid having one or more hydroxyl (—OH) groups on the lipid. In certain embodiments, the PEG-OH lipid includes one or more hydroxyl groups on the PEG chain. In certain embodiments, a PEG-OH or hydroxy-PEGylated lipid comprises an —OH group at the terminus of the PEG chain. Each possibility represents a separate embodiment of the present invention.

In certain embodiments, a PEG lipid useful in the present invention is a compound of Formula (V). Provided herein are compounds of Formula (V):

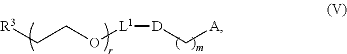

or salts thereof, wherein:
R³ is —OR^{◯};
R^{◯} is hydrogen, optionally substituted alkyl, or an oxygen protecting group;
r is an integer between 1 and 100, inclusive;
L¹ is optionally substituted C₁₋₁₀ alkylene, wherein at least one methylene of the optionally substituted C₁₋₁₀ alkylene is independently replaced with optionally substituted carbocyclylene, optionally substituted heterocyclylene, optionally substituted arylene, optionally substituted heteroarylene, O, —N(R^{N}), S, C(O), C(O)N(R^{N}), NR^{N}C(O), C(O)O, OC(O), OC(O)O, OC(O)N(R^{N}), NR^{N}C(O)O, or —NR^{N}C(O)N(R^{N});
D is a moiety obtained by click chemistry or a moiety cleavable under physiological conditions;
m is 0, 1, 2, 3, 4, 5, 6, 7, 8, 9, or 10;

A is of the Formula:

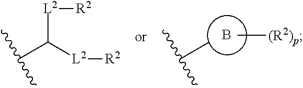

each instance of L2 is independently a bond or optionally substituted C₁₋₆ alkylene, wherein one methylene unit of the optionally substituted C₁₋₆ alkylene is optionally replaced with O, N(R^{N}), S, C(O), C(O)N(R^{N}), NR^{N}C(O), C(O)O, OC(O), OC(O)O, OC(O)N(R^{N}), NR^{N}C(O)O, or NR^{N}C(O)N(R^{N});
each instance of R² is independently optionally substituted C₁₋₃₀ alkyl, optionally substituted C₁₋₃₀ alkenyl, or optionally substituted C₁₋₃₀ alkynyl; optionally wherein one or more methylene units of R² are independently replaced with optionally substituted carbocyclylene, optionally substituted heterocyclylene, optionally substituted arylene, optionally substituted heteroarylene, N(R^{N}), O, S, C(O), C(O)N(R^{N}), —NR^{N}C(O), NR^{N}C(O)N(R^{N}), C(O)O, OC(O), OC(O)O, OC(O)N(R^{N}), NR^{N}C(O)O, C(O) S, SC(O), —C(═NR^{N}), C(═NR^{N})N(R^{N}), NR^{N}C(═NR^{N}), NR^{N}C(═NR^{N})N(R^{N}), C(S), C(S)N(R^{N}), NR^{N}C(S), —NR^{N}C(S)N(R^{N}), S(O), OS(O), S(O)O, OS(O)O, OS(O)₂, S(O)₂O, OS(O)₂O, N(R^{N})S(O), S(O)N(R^{N}), —N(R^{N})S(O)N(R^{N}), OS(O)N(R^{N}), N(R^{N})S(O)O, S(O)₂, N(R^{N})S(O)₂, S(O)₂N(R^{N}), N(R^{N})S(O)₂N(R^{N}), —OS(O)₂N(R^{N}), or N(R^{N})S(O)₂O;
each instance of R^{N} is independently hydrogen, optionally substituted alkyl, or a nitrogen protecting group;
Ring B is optionally substituted carbocyclyl, optionally substituted heterocyclyl, optionally substituted aryl, or optionally substituted heteroaryl; and
p is 1 or 2.

In certain embodiments, the compound of Formula (V) is a PEG-OH lipid (i.e., R³ is —OR^{◯}, and R^{◯} is hydrogen). In certain embodiments, the compound of Formula (V) is of Formula (V—OH):

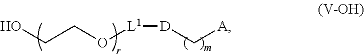

or a salt thereof.

In certain embodiments, a PEG lipid useful in the present invention is a PEGylated fatty acid. In certain embodiments, a PEG lipid useful in the present invention is a compound of Formula (VI). Provided herein are compounds of Formula (VI):

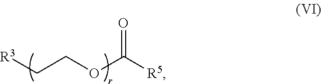

or a salts thereof, wherein:
R³ is —OR^{◯};
R^{◯} is hydrogen, optionally substituted alkyl or an oxygen protecting group;
r is an integer between 1 and 100, inclusive;
R⁵ is optionally substituted C₁₀₋₄₀ alkyl, optionally substituted C₁₀₋₄₀ alkenyl, or optionally substituted C₁₀₋₄₀ alkynyl; and optionally one or more methylene groups of R⁵ are replaced with optionally substituted carbocyclylene, optionally substituted heterocyclylene, optionally substituted arylene, optionally substituted heteroarylene, N(R^{N}), O, S, C(O), C(O)N(R^{N}), NR^{N}C(O), NR^{N}C(O)N(R^{N}), C(O)O, OC(O), OC(O)O, OC(O)N(R^{N}), NR^{N}C(O)O, C(O) S, SC(O), C(═NR^{N}), C(═NR^{N})N(R^{N}), NR^{N}C(═NR^{N}), NR^{N}C(═NR^{N})N(R^{N}), C(S), C(S)N(R^{N}), NR^{N}C(S), NR^{N}C(S)N(R^{N}), S(O), OS(O), S(O)O, OS(O)O, —OS(O)₂, S(O)₂O, OS(O)₂O, N(R^{N})S(O), S(O)N(R^{N}), N(R^{N})S(O)N(R^{N}), OS(O)N(R^{N}), N(R^{N})S(O)O, S(O)₂, N(R^{N})S(O)₂, S(O)₂N(R^{N}), N(R^{N})S(O)₂N(R^{N}), OS(O)₂N(R^{N}), or N(R^{N})S(O)₂O; and each instance of R^{N} is independently hydrogen, optionally substituted alkyl, or a nitrogen protecting group.

In certain embodiments, the compound of Formula (VI) is of Formula (VI—OH):

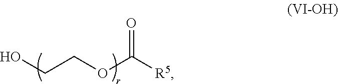

or a salt thereof. In some embodiments, r is 45. In another of the foregoing or related aspects, a PEG lipid of the invention is featured wherein r is 40-50.

In yet other embodiments the compound of Formula (VI) is:

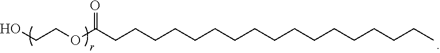

or a salt thereof.

In one embodiment, the compound of Formula (VI) is

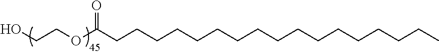

### (PEG Compound I).

In some aspects, the lipid composition of the pharmaceutical compositions disclosed herein does not comprise a PEG-lipid.

In some embodiments, the PEG-lipids may be one or more of the PEG lipids described in U.S. Application No. 62/520,530.

In some embodiments, a PEG lipid of the invention comprises a PEG-modified phosphatidylethanolamine, a PEG-modified phosphatidic acid, a PEG-modified ceramide, a PEG-modified dialkylamine, a PEG-modified diacylglycerol, a PEG-modified dialkylglycerol, and mixtures thereof. In some embodiments, the PEG-modified lipid is PEG-DMG, PEG-c-DOMG (also referred to as PEG-DOMG), PEG-DSG and/or PEG-DPG.

The LNPs provided herein, in certain embodiments, exhibit increased PEG shedding compared to existing LNP formulations comprising PEG lipids. “PEG shedding,” as used herein, refers to the cleavage of a PEG group from a PEG lipid. In many instances, cleavage of a PEG group from a PEG lipid occurs through serum-driven esterase-cleavage or hydrolysis. The PEG lipids provided herein, in certain embodiments, have been designed to control the rate of PEG shedding. In certain embodiments, an LNP provided herein exhibits greater than 5%, 10%, 15%, 20%, 25%, 30%, 35%, 40%, 45%, 50%, 55%, 60%, 65%, 70%, 75%, 80%, 85%, 90%, 95%, or 98% PEG shedding after about 6 hours in human serum In certain embodiments, an LNP provided herein exhibits greater than 50% PEG shedding after about 6 hours in human serum. In certain embodiments, an LNP provided herein exhibits greater than 60% PEG shedding after about 6 hours in human serum. In certain embodiments, an LNP provided herein exhibits greater than 70% PEG shedding after about 6 hours in human serum. In certain embodiments, the LNP exhibits greater than 80% PEG shedding after about 6 hours in human serum. In certain embodiments, the LNP exhibits greater than 90% PEG shedding after about 6 hours in human serum. In certain embodiments, an LNP provided herein exhibits greater than 90% PEG shedding after about 6 hours in human serum.

In other embodiments, an LNP provided herein exhibits less than 5%, 10%, 15%, 20%, 25%, 30%, 35%, 40%, 45%, 50%, 55%, 60%, 65%, 70%, 75%, 80%, 85%, 90%, 95%, or 98% PEG shedding after about 6 hours in human serum In certain embodiments, an LNP provided herein exhibits less than 60% PEG shedding after about 6 hours in human serum. In certain embodiments, an LNP provided herein exhibits less than 70% PEG shedding after about 6 hours in human serum. In certain embodiments, an LNP provided herein exhibits less than 80% PEG shedding after about 6 hours in human serum.

In addition to the PEG lipids provided herein, the LNP may comprise one or more additional lipid components. In certain embodiments, the PEG lipids are present in the LNP in a molar ratio of 0.15-15% with respect to other lipids. In certain embodiments, the PEG lipids are present in a molar ratio of 0.15-5% with respect to other lipids. In certain embodiments, the PEG lipids are present in a molar ratio of 1-5% with respect to other lipids. In certain embodiments, the PEG lipids are present in a molar ratio of 0.15-2% with respect to other lipids. In certain embodiments, the PEG lipids are present in a molar ratio of 1-2% with respect to other lipids. In certain embodiments, the PEG lipids are present in a molar ratio of approximately 1%, 1.1%, 1.2%, 1.3%, 1.4%, 1.5%, 1.6%, 1.7%, 1.8%, 1.9%, or 2% with respect to other lipids. In certain embodiments, the PEG lipids are present in a molar ratio of approximately 1.5% with respect to other lipids.

In one embodiment, the amount of PEG-lipid in the lipid composition of a pharmaceutical composition disclosed herein ranges from about 0.1 mol % to about 5 mol %, from about 0.5 mol % to about 5 mol %, from about 1 mol % to about 5 mol %, from about 1.5 mol % to about 5 mol %, from about 2 mol % to about 5 mol %, from about 0.1 mol % to about 4 mol %, from about 0.5 mol % to about 4 mol %, from about 1 mol % to about 4 mol %, from about 1.5 mol % to about 4 mol %, from about 2 mol % to about 4 mol %, from about 0.1 mol % to about 3 mol %, from about 0.5 mol % to about 3 mol %, from about 1 mol % to about 3 mol %, from about 1.5 mol % to about 3 mol %, from about 2 mol % to about 3 mol %, from about 0.1 mol % to about 2 mol %, from about 0.5 mol % to about 2 mol %, from about 1 mol % to about 2 mol %, from about 1.5 mol % to about 2 mol %, from about 0.1 mol % to about 1.5 mol %, from about 0.5 mol % to about 1.5 mol %, or from about 1 mol % to about 1.5 mol %.

In one embodiment, the amount of PEG-lipid in the lipid composition disclosed herein is about 2 mol %. In one embodiment, the amount of PEG-lipid in the lipid composition disclosed herein is about 1.5 mol %.

In one embodiment, the amount of PEG-lipid in the lipid composition disclosed herein is at least about 0.1, 0.2, 0.3, 0.4, 0.5, 0.6, 0.7, 0.8, 0.9, 1, 1.1, 1.2, 1.3, 1.4, 1.5, 1.6, 1.7, 1.8, 1.9, 2, 2.1, 2.2, 2.3, 2.4, 2.5, 2.6, 2.7, 2.8, 2.9, 3, 3.1, 3.2, 3.3, 3.4, 3.5, 3.6, 3.7, 3.8, 3.9, 4, 4.1, 4.2, 4.3, 4.4, 4.5, 4.6, 4.7, 4.8, 4.9, or 5 mol %.

### Exemplary Synthesis

Compound: HO-PEG₂₀₀₀-ester-C18

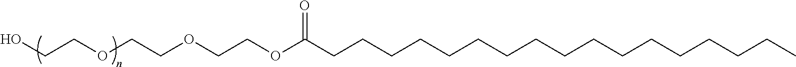

To a nitrogen filled flask containing palladium on carbon (10 wt. %, 74 mg, 0.070 mmol) was added Benzyl-PEG2000-ester-C18 (822 mg, 0.35 mmol) and MeOH (20 mL). The flask was evacuated and backfilled with H₂ three times, and allowed to stir at RT and 1 atm H2 for 12 hours. The mixture was filtered through celite, rinsing with DCM, and the filtrate was concentrated in vacuo to provide the desired product (692 mg, 88%). Using this methodology n=40-50. In one embodiment, n of the resulting polydispersed mixture is referred to by the average, 45.

For example, the value of r can be determined on the basis of a molecular weight of the PEG moiety within the PEG lipid. For example, a molecular weight of 2,000 (e.g., PEG2000) corresponds to a value of n of approximately 45. For a given composition, the value for n can connote a distribution of values within an art-accepted range, since polymers are often found as a distribution of different polymer chain lengths. For example, a skilled artisan understanding the polydispersity of such polymeric compositions would appreciate that an n value of 45 (e.g., in a structural formula) can represent a distribution of values between 40-50 in an actual PEG-containing composition, e.g., a DMG PEG200 peg lipid composition.

In some aspects, a target cell delivery lipid of the pharmaceutical compositions disclosed herein does not comprise a PEG-lipid.

In one embodiment, a target cell target cell delivery LNP of the disclosure comprises a PEG-lipid. In one embodiment, the PEG lipid is not PEG DMG. In some aspects, the PEG-lipid is selected from the group consisting of a PEG-modified phosphatidylethanolamine, a PEG-modified phosphatidic acid, a PEG-modified ceramide, a PEG-modified dialkylamine, a PEG-modified diacylglycerol, a PEG-modified dialkylglycerol, and mixtures thereof. In some aspects, the PEG lipid is selected from the group consisting of PEG-c-DOMG, PEG-DMG, PEG-DLPE, PEG-DMPE, PEG-DPPC and PEG-DSPE lipid. In other aspects, the PEG-lipid is PEG-DMG.

In one embodiment, a target cell target cell delivery LNP of the disclosure comprises a PEG-lipid which has a chain length longer than about 14 or than about 10, if branched.

As used herein, the term “alkyl”, “alkyl group”, or “alkylene” means a linear or branched, saturated hydrocarbon including one or more carbon atoms (e.g., one, two, three, four, five, six, seven, eight, nine, ten, eleven, twelve, thirteen, fourteen, fifteen, sixteen, seventeen, eighteen, nineteen, twenty, or more carbon atoms), which is optionally substituted. The notation “C₁₋₁₄ alkyl” means an optionally substituted linear or branched, saturated hydrocarbon including 1-14 carbon atoms. Unless otherwise specified, an alkyl group described herein refers to both unsubstituted and substituted alkyl groups.

As used herein, the term “alkenyl”, “alkenyl group”, or “alkenylene” means a linear or branched hydrocarbon including two or more carbon atoms (e.g., two, three, four, five, six, seven, eight, nine, ten, eleven, twelve, thirteen, fourteen, fifteen, sixteen, seventeen, eighteen, nineteen, twenty, or more carbon atoms) and at least one double bond, which is optionally substituted. The notation “C₂₋₁₄ alkenyl” means an optionally substituted linear or branched hydrocarbon including 2-14 carbon atoms and at least one carbon-carbon double bond. An alkenyl group may include one, two, three, four, or more carbon-carbon double bonds. For example, C₁₈ alkenyl may include one or more double bonds. A C₁₈ alkenyl group including two double bonds may be a linoleyl group. Unless otherwise specified, an alkenyl group described herein refers to both unsubstituted and substituted alkenyl groups.

As used herein, the term “alkynyl”, “alkynyl group”, or “alkynylene” means a linear or branched hydrocarbon including two or more carbon atoms (e.g., two, three, four, five, six, seven, eight, nine, ten, eleven, twelve, thirteen, fourteen, fifteen, sixteen, seventeen, eighteen, nineteen, twenty, or more carbon atoms) and at least one carbon-carbon triple bond, which is optionally substituted. The notation “C₂₋₁₄ alkynyl” means an optionally substituted linear or branched hydrocarbon including 2-14 carbon atoms and at least one carbon-carbon triple bond. An alkynyl group may include one, two, three, four, or more carbon-carbon triple bonds. For example, C₁₈ alkynyl may include one or more carbon-carbon triple bonds. Unless otherwise specified, an alkynyl group described herein refers to both unsubstituted and substituted alkynyl groups.

As used herein, the term “carbocycle” or “carbocyclic group” means an optionally substituted mono- or multi-cyclic system including one or more rings of carbon atoms. Rings may be three, four, five, six, seven, eight, nine, ten, eleven, twelve, thirteen, fourteen, fifteen, sixteen, seventeen, eighteen, nineteen, or twenty membered rings. The notation “C₃₋₆ carbocycle” means a carbocycle including a single ring having 3-6 carbon atoms. Carbocycles may include one or more carbon-carbon double or triple bonds and may be non-aromatic or aromatic (e.g., cycloalkyl or aryl groups). Examples of carbocycles include cyclopropyl, cyclopentyl, cyclohexyl, phenyl, naphthyl, and 1,2 dihydronaphthyl groups. The term “cycloalkyl” as used herein means a non-aromatic carbocycle and may or may not include any double or triple bond. Unless otherwise specified, carbocycles described herein refers to both unsubstituted and substituted carbocycle groups, i.e., optionally substituted carbocycles.

As used herein, the term “heterocycle” or “heterocyclic group” means an optionally substituted mono- or multi-cyclic system including one or more rings, where at least one ring includes at least one heteroatom. Heteroatoms may be, for example, nitrogen, oxygen, or sulfur atoms. Rings may be three, four, five, six, seven, eight, nine, ten, eleven, twelve, thirteen, or fourteen membered rings. Heterocycles may include one or more double or triple bonds and may be non-aromatic or aromatic (e.g., heterocycloalkyl or heteroaryl groups). Examples of heterocycles include imidazolyl, imidazolidinyl, oxazolyl, oxazolidinyl, thiazolyl, thiazolidinyl, pyrazolidinyl, pyrazolyl, isoxazolidinyl, isoxazolyl, isothiazolidinyl, isothiazolyl, morpholinyl, pyrrolyl, pyrrolidinyl, furyl, tetrahydrofuryl, thiophenyl, pyridinyl, piperidinyl, quinolyl, and isoquinolyl groups. The term “heterocycloalkyl” as used herein means a non-aromatic heterocycle and may or may not include any double or triple bond. Unless otherwise specified, heterocycles described herein refers to both unsubstituted and substituted heterocycle groups, i.e., optionally substituted heterocycles.

As used herein, the term “heteroalkyl”, “heteroalkenyl”, or “heteroalkynyl”, refers respectively to an alkyl, alkenyl, alkynyl group, as defined herein, which further comprises one or more (e.g., 1, 2, 3, or 4) heteroatoms (e.g., oxygen, sulfur, nitrogen, boron, silicon, phosphorus) wherein the one or more heteroatoms is inserted between adjacent carbon atoms within the parent carbon chain and/or one or more heteroatoms is inserted between a carbon atom and the parent molecule, i.e., between the point of attachment. Unless otherwise specified, heteroalkyls, heteroalkenyls, or heteroalkynyls described herein refers to both unsubstituted and substituted heteroalkyls, heteroalkenyls, or heteroalkynyls, i.e., optionally substituted heteroalkyls, heteroalkenyls, or heteroalkynyls.

As used herein, a “biodegradable group” is a group that may facilitate faster metabolism of a lipid in a mammalian entity. A biodegradable group may be selected from the group consisting of, but is not limited to, —C(O)O—, —OC(O)—, —C(O)N(R′)—, —N(R′) C(O)—, —C(O)—, —C(S)—, —C(S)S—, —SC(S)—, —CH(OH)—, —P(O)(OR′)O—, —S(O)₂—, an aryl group, and a heteroaryl group. As used herein, an “aryl group” is an optionally substituted carbocyclic group including one or more aromatic rings. Examples of aryl groups include phenyl and naphthyl groups. As used herein, a “heteroaryl group” is an optionally substituted heterocyclic group including one or more aromatic rings. Examples of heteroaryl groups include pyrrolyl, furyl, thiophenyl, imidazolyl, oxazolyl, and thiazolyl. Both aryl and heteroaryl groups may be optionally substituted. For example, M and M′ can be selected from the non-limiting group consisting of optionally substituted phenyl, oxazole, and thiazole. In the Formulas herein, M and M′ can be independently selected from the list of biodegradable groups above. Unless otherwise specified, aryl or heteroaryl groups described herein refers to both unsubstituted and substituted groups, i.e., optionally substituted aryl or heteroaryl groups.

Alkyl, alkenyl, and cyclyl (e.g., carbocyclyl and heterocyclyl) groups may be optionally substituted unless otherwise specified. Optional substituents may be selected from the group consisting of, but are not limited to, a halogen atom (e.g., a chloride, bromide, fluoride, or iodide group), a carboxylic acid (e.g., C(O)OH), an alcohol (e.g., a hydroxyl, OH), an ester (e.g., C(O)OR OC(O) R), an aldehyde (e.g., C(O) H), a carbonyl (e.g., C(O)R, alternatively represented by C═O), an acyl halide (e.g., C(O) X, in which X is a halide selected from bromide, fluoride, chloride, and iodide), a carbonate (e.g., OC(O)OR), an alkoxy (e.g., OR), an acetal (e.g., C(OR)2R″″, in which each OR are alkoxy groups that can be the same or different and R″″ is an alkyl or alkenyl group), a phosphate (e.g., P(O)₄³⁻), a thiol (e.g., SH), a sulfoxide (e.g., S(O)R), a sulfinic acid (e.g., S(O)OH), a sulfonic acid (e.g., S(O)₂OH), a thial (e.g., C(S)H), a sulfate (e.g., S(O)₄²⁻), a sulfonyl (e.g., S(O)₂), an amide (e.g., C(O)NR₂, or N(R)C(O)R), an azido (e.g., N3), a nitro (e.g., NO₂), a cyano (e.g., CN), an isocyano (e.g., NC), an acyloxy (e.g., OC(O) R), an amino(e.g., NR₂, NRH, or NH₂), a carbamoyl (e.g., OC(O)NR₂, OC(O)NRH, or OC(O)NH₂), a sulfonamide (e.g., S(O)₂NR₂, S(O)₂NRH, S(O)₂NH₂, N(R)S(O)₂R, N(H)S(O)₂R, N(R)S(O)₂H, or N(H)S(O)₂H), an alkyl group, an alkenyl group, and a cyclyl (e.g., carbocyclyl or heterocyclyl) group. In any of the preceding, R is an alkyl or alkenyl group, as defined herein. In some embodiments, the substituent groups themselves may be further substituted with, for example, one, two, three, four, five, or six substituents as defined herein. For example, a C₁₋₆ alkyl group may be further substituted with one, two, three, four, five, or six substituents as described herein.

Compounds of the disclosure that contain nitrogens can be converted to N-oxides by treatment with an oxidizing agent (e.g., 3-chloroperoxybenzoic acid (mCPBA) and/or hydrogen peroxides) to afford other compounds of the disclosure. Thus, all shown and claimed nitrogen-containing compounds are considered, when allowed by valency and structure, to include both the compound as shown and its N-oxide derivative (which can be designated as N→O or N+—O—). Furthermore, in other instances, the nitrogens in the compounds of the disclosure can be converted to N-hydroxy or N-alkoxy compounds. For example, N-hydroxy compounds can be prepared by oxidation of the parent amine by an oxidizing agent such as m CPBA. All shown and claimed nitrogen-containing compounds are also considered, when allowed by valency and structure, to cover both the compound as shown and its N-hydroxy (i.e., N—OH) and N-alkoxy (i.e., N—OR, wherein R is substituted or unsubstituted C₁-C₆ alkyl, C₁-C₆ alkenyl, C₁-C₆ alkynyl, 3-14-membered carbocycle or 3-14-membered heterocycle) derivatives.

### Exemplary Additional LNP Components

The lipid composition of a pharmaceutical composition disclosed herein can include one or more components in addition to those described above. For example, the lipid composition can include one or more permeability enhancer molecules, carbohydrates, polymers, surface altering agents (e.g., surfactants), or other components. For example, a permeability enhancer molecule can be a molecule described by U.S. Patent Application Publication No. 2005/0222064. Carbohydrates can include simple sugars (e.g., glucose) and polysaccharides (e.g., glycogen and derivatives and analogs thereof).

A polymer can be included in and/or used to encapsulate or partially encapsulate a pharmaceutical composition disclosed herein (e.g., a pharmaceutical composition in lipid nanoparticle form). A polymer can be biodegradable and/or biocompatible. A polymer can be selected from, but is not limited to, polyamines, polyethers, polyamides, polyesters, polycarbamates, polyureas, polycarbonates, polystyrenes, polyimides, polysulfones, polyurethanes, polyacetylenes, polyethylenes, polyethyleneimines, polyisocyanates, polyacrylates, polymethacrylates, polyacrylonitriles, and polyarylates.

### LNPs Comprising Checkpoint Cancer Vaccines

Disclosed herein are, inter alia, LNP compositions comprising polynucleotides encoding checkpoint cancer vaccines comprising one or more IDO antigenic peptides and one or more PD-L1 antigenic peptides for use in stimulating T cells (e.g., T effector cells), for treating a cancer in a subject. In another embodiment, the invention pertains to LNPs comprising a polynucleotide comprising an mRNA encoding a checkpoint cancer vaccine comprising (i) one or more IDO antigenic peptides and (ii) one or more PD-L1 antigenic peptides. The LNP compositions of the present disclosure can be used to prime T cells, stimulate and activate T effector cells and/or induce killing of immunosuppressive (regulatory) immune cells and cancer cells that overexpress IDO and PD-L1 in vivo or ex vivo.

In an aspect, an LNP composition comprising a polynucleotide encoding a checkpoint cancer vaccine, comprises: (i) an ionizable lipid, e.g., an amino lipid; (ii) a sterol or other structural lipid; (iii) a non-cationic helper lipid or phospholipid; and (iv) a PEG-lipid.

In an aspect, an LNP composition comprising a polynucleotide encoding IDO (e.g., IDO1 or IDO2), comprises: (i) an ionizable lipid, e.g., an amino lipid; (ii) a sterol or other structural lipid; (iii) a non-cationic helper lipid or phospholipid; and (iv) a PEG-lipid.

In an aspect, an LNP composition comprising a polynucleotide a checkpoint cancer vaccine comprising (i) one or more IDO antigenic peptides and (ii) one or more PD-L1 antigenic peptides, comprises: (i) an ionizable lipid, e.g., an amino lipid; (ii) a sterol or other structural lipid; (iii) a non-cationic helper lipid or phospholipid; and (iv) a PEG-lipid.

In another aspect, the LNP compositions of the disclosure are used in a method of treating a cancer in a subject or a method of stimulating an immune response in a subject.

In an aspect, an LNP composition comprising a polynucleotide encoding a checkpoint cancer vaccine comprising (i) one or more IDO antigenic peptides and (ii) one or more PD-L1 antigenic peptides, can be administered with an additional agent, e.g., as described herein.

Additional features of LNP compositions for use in combination therapy are provided in the section titled “LNPs for therapy.”

In one embodiment, the ratio between the lipid composition and the polynucleotide range can be from about 10:1 to about 60:1 (wt/wt).

### Nanoparticle Compositions

In some embodiments, the pharmaceutical compositions disclosed herein are Formulated as lipid nanoparticles (LNP). Accordingly, the present disclosure also provides nanoparticle compositions comprising (i) a lipid composition comprising a delivery agent such as compound as described herein, and (ii) a polynucleotide encoding a polypeptide of the invention. In such nanoparticle composition, the lipid composition disclosed herein can encapsulate the polynucleotide encoding a polypeptide of the invention.

Nanoparticle compositions are typically sized on the order of micrometers or smaller and can include a lipid bilayer. Nanoparticle compositions encompass lipid nanoparticles (LNPs), liposomes (e.g., lipid vesicles), and lipoplexes. For example, a nanoparticle composition can be a liposome having a lipid bilayer with a diameter of 500 nm or less.

Nanoparticle compositions include, for example, lipid nanoparticles (LNPs), liposomes, and lipoplexes. In some embodiments, nanoparticle compositions are vesicles including one or more lipid bilayers. In certain embodiments, a nanoparticle composition includes two or more concentric bilayers separated by aqueous compartments. Lipid bilayers can be functionalized and/or crosslinked to one another. Lipid bilayers can include one or more ligands, proteins, or channels.

In one embodiment, a lipid nanoparticle comprises an ionizable amino lipid, a structural lipid, a phospholipid, and mRNA. In some embodiments, the LNP comprises an ionizable amino lipid, a PEG-modified lipid, a sterol and a structural lipid. In some embodiments, the LNP has a molar ratio of about 40-50% ionizable amino lipid; about 5-15% structural lipid; about 30-45% sterol; and about 1-5% PEG-modified lipid.

In some embodiments, the LNP has a polydispersity value of less than 0.4. In some embodiments, the LNP has a net neutral charge at a neutral pH. In some embodiments, the LNP has a mean diameter of 50-150 nm. In some embodiments, the LNP has a mean diameter of 80-100 nm.

As generally defined herein, the term “lipid” refers to a small molecule that has hydrophobic or amphiphilic properties. Lipids may be naturally occurring or synthetic. Examples of classes of lipids include, but are not limited to, fats, waxes, sterol-containing metabolites, vitamins, fatty acids, glycerolipids, glycerophospholipids, sphingolipids, saccharolipids, and polyketides, and prenol lipids. In some instances, the amphiphilic properties of some lipids leads them to form liposomes, vesicles, or membranes in aqueous media.

In some embodiments, a lipid nanoparticle (LNP) may comprise an ionizable amino lipid. As used herein, the term “ionizable amino lipid” has its ordinary meaning in the art and may refer to a lipid comprising one or more charged moieties. In some embodiments, an ionizable amino lipid may be positively charged or negatively charged. An ionizable amino lipid may be positively charged, in which case it can be referred to as “cationic lipid”. In certain embodiments, an ionizable amino lipid molecule may comprise an amine group, and can be referred to as an ionizable amino lipid. As used herein, a “charged moiety” is a chemical moiety that carries a formal electronic charge, e.g., monovalent (+1, or −1), divalent (+2, or −2), trivalent (+3, or −3), etc. The charged moiety may be anionic (i.e., negatively charged) or cationic (i.e., positively charged). Examples of positively-charged moieties include amine groups (e.g., primary, secondary, and/or tertiary amines), ammonium groups, pyridinium group, guanidine groups, and imidizolium groups. In a particular embodiment, the charged moieties comprise amine groups. Examples of negatively-charged groups or precursors thereof, include carboxylate groups, sulfonate groups, sulfate groups, phosphonate groups, phosphate groups, hydroxyl groups, and the like. The charge of the charged moiety may vary, in some cases, with the environmental conditions, for example, changes in pH may alter the charge of the moiety, and/or cause the moiety to become charged or uncharged. In general, the charge density of the molecule may be selected as desired.

It should be understood that the terms “charged” or “charged moiety” does not refer to a “partial negative charge” or “partial positive charge” on a molecule. The terms “partial negative charge” and “partial positive charge” are given its ordinary meaning in the art. A “partial negative charge” may result when a functional group comprises a bond that becomes polarized such that electron density is pulled toward one atom of the bond, creating a partial negative charge on the atom. Those of ordinary skill in the art will, in general, recognize bonds that can become polarized in this way.

The ionizable amino lipid is sometimes referred to in the art as an “ionizable cationic lipid”. In one embodiment, the ionizable amino lipid may have a positively charged hydrophilic head and a hydrophobic tail that are connected via a linker structure.

In addition to these, an ionizable amino lipid may also be a lipid including a cyclic amine group.

In one embodiment, the ionizable amino lipid may be selected from, but not limited to, an ionizable amino lipid described in International Publication Nos. WO2013086354 and WO2013116126; the contents of each of which are herein incorporated by reference in their entirety.

In yet another embodiment, the ionizable amino lipid may be selected from, but not limited to, Formula CLI-CLXXXXII of U.S. Pat. No. 7,404,969; each of which is herein incorporated by reference in their entirety.

In one embodiment, the lipid may be a cleavable lipid such as those described in International Publication No. WO2012170889, herein incorporated by reference in its entirety. In one embodiment, the lipid may be synthesized by methods known in the art and/or as described in International Publication Nos. WO2013086354; the contents of each of which are herein incorporated by reference in their entirety.

Nanoparticle compositions can be characterized by a variety of methods. For example, microscopy (e.g., transmission electron microscopy or scanning electron microscopy) can be used to examine the morphology and size distribution of a nanoparticle composition. Dynamic light scattering or potentiometry (e.g., potentiometric titrations) can be used to measure zeta potentials. Dynamic light scattering can also be utilized to determine particle sizes. Instruments such as the Zetasizer Nano ZS (Malvern Instruments Ltd, Malvern, Worcestershire, UK) can also be used to measure multiple characteristics of a nanoparticle composition, such as particle size, polydispersity index, and zeta potential.

The size of the nanoparticles can help counter biological reactions such as, but not limited to, inflammation, or can increase the biological effect of the polynucleotide.

As used herein, “size” or “mean size” in the context of nanoparticle compositions refers to the mean diameter of a nanoparticle composition.

In one embodiment, the polynucleotide encoding a polypeptide are Formulated in lipid nanoparticles having a diameter from about 10 to about 100 nm such as, but not limited to, about 10 to about 20 nm, about 10 to about 30 nm, about 10 to about 40 nm, about 10 to about 50 nm, about 10 to about 60 nm, about 10 to about 70 nm, about 10 to about 80 nm, about 10 to about 90 nm, about 20 to about 30 nm, about 20 to about 40 nm, about 20 to about 50 nm, about 20 to about 60 nm, about 20 to about 70 nm, about 20 to about 80 nm, about 20 to about 90 nm, about 20 to about 100 nm, about 30 to about 40 nm, about 30 to about 50 nm, about 30 to about 60 nm, about 30 to about 70 nm, about 30 to about 80 nm, about 30 to about 90 nm, about 30 to about 100 nm, about 40 to about 50 nm, about 40 to about 60 nm, about 40 to about 70 nm, about 40 to about 80 nm, about 40 to about 90 nm, about 40 to about 100 nm, about 50 to about 60 nm, about 50 to about 70 nm, about 50 to about 80 nm, about 50 to about 90 nm, about 50 to about 100 nm, about 60 to about 70 nm, about 60 to about 80 nm, about 60 to about 90 nm, about 60 to about 100 nm, about 70 to about 80 nm, about 70 to about 90 nm, about 70 to about 100 nm, about 80 to about 90 nm, about 80 to about 100 nm and/or about 90 to about 100 nm.

In one embodiment, the nanoparticles have a diameter from about 10 to 500 nm. In one embodiment, the nanoparticle has a diameter greater than 100 nm, greater than 150 nm, greater than 200 nm, greater than 250 nm, greater than 300 nm, greater than 350 nm, greater than 400 nm, greater than 450 nm, greater than 500 nm, greater than 550 nm, greater than 600 nm, greater than 650 nm, greater than 700 nm, greater than 750 nm, greater than 800 nm, greater than 850 nm, greater than 900 nm, greater than 950 nm or greater than 1000 nm.

In some embodiments, the largest dimension of a nanoparticle composition is 1 μm or shorter (e.g., 1 μm, 900 nm, 800 nm, 700 nm, 600 nm, 500 nm, 400 nm, 300 nm, 200 nm, 175 nm, 150 nm, 125 nm, 100 nm, 75 nm, 50 nm, or shorter).

A nanoparticle composition can be relatively homogenous. A polydispersity index can be used to indicate the homogeneity of a nanoparticle composition, e.g., the particle size distribution of the nanoparticle composition. A small (e.g., less than 0.3) polydispersity index generally indicates a narrow particle size distribution. A nanoparticle composition can have a polydispersity index from about 0 to about 0.25, such as 0.01, 0.02, 0.03, 0.04, 0.05, 0.06, 0.07, 0.08, 0.09, 0.10, 0.11, 0.12, 0.13, 0.14, 0.15, 0.16, 0.17, 0.18, 0.19, 0.20, 0.21, 0.22, 0.23, 0.24, or 0.25. In some embodiments, the polydispersity index of a nanoparticle composition disclosed herein can be from about 0.10 to about 0.20.

The zeta potential of a nanoparticle composition can be used to indicate the electro kinetic potential of the composition. For example, the zeta potential can describe the surface charge of a nanoparticle composition. Nanoparticle compositions with relatively low charges, positive or negative, are generally desirable, as more highly charged species can interact undesirably with cells, tissues, and other elements in the body. In some embodiments, the zeta potential of a nanoparticle composition disclosed herein can be from about −10 mV to about +20 mV, from about −10 mV to about +15 mV, from about 10 mV to about +10 mV, from about −10 mV to about +5 mV, from about −10 mV to about 0 mV, from about −10 mV to about −5 mV, from about −5 mV to about +20 mV, from about −5 mV to about +15 mV, from about −5 mV to about +10 mV, from about −5 mV to about +5 mV, from about −5 mV to about 0 mV, from about 0 mV to about +20 mV, from about 0 mV to about +15 mV, from about 0 mV to about +10 mV, from about 0 mV to about +5 mV, from about +5 mV to about +20 mV, from about +5 mV to about +15 mV, or from about +5 mV to about +10 mV.

In some embodiments, the zeta potential of the lipid nanoparticles can be from about 0 mV to about 100 mV, from about 0 mV to about 90 mV, from about 0 mV to about 80 mV, from about 0 mV to about 70 mV, from about 0 mV to about 60 mV, from about 0 mV to about 50 mV, from about 0 mV to about 40 mV, from about 0 mV to about 30 mV, from about 0 mV to about 20 mV, from about 0 mV to about 10 mV, from about 10 mV to about 100 mV, from about 10 mV to about 90 mV, from about 10 mV to about 80 mV, from about 10 mV to about 70 mV, from about 10 mV to about 60 mV, from about 10 mV to about 50 mV, from about 10 mV to about 40 mV, from about 10 mV to about 30 mV, from about 10 mV to about 20 mV, from about 20 mV to about 100 mV, from about 20 mV to about 90 mV, from about 20 mV to about 80 mV, from about 20 mV to about 70 mV, from about 20 mV to about 60 mV, from about 20 mV to about 50 mV, from about 20 mV to about 40 mV, from about 20 mV to about 30 mV, from about 30 mV to about 100 mV, from about 30 mV to about 90 mV, from about 30 mV to about 80 mV, from about 30 mV to about 70 mV, from about 30 mV to about 60 mV, from about 30 mV to about 50 mV, from about 30 mV to about 40 mV, from about 40 mV to about 100 mV, from about 40 mV to about 90 mV, from about 40 mV to about 80 mV, from about 40 mV to about 70 mV, from about 40 mV to about 60 mV, and from about 40 mV to about 50 mV. In some embodiments, the zeta potential of the lipid nanoparticles can be from about 10 mV to about 50 mV, from about 15 mV to about 45 mV, from about 20 mV to about 40 mV, and from about 25 mV to about 35 mV. In some embodiments, the zeta potential of the lipid nanoparticles can be about 10 mV, about 20 mV, about 30 mV, about 40 mV, about 50 mV, about 60 mV, about 70 mV, about 80 mV, about 90 mV, and about 100 mV.

The term “encapsulation efficiency” of a polynucleotide describes the amount of the polynucleotide that is encapsulated by or otherwise associated with a nanoparticle composition after preparation, relative to the initial amount provided. As used herein, “encapsulation” can refer to complete, substantial, or partial enclosure, confinement, surrounding, or encasement.

Encapsulation efficiency is desirably high (e.g., close to 100%). The encapsulation efficiency can be measured, for example, by comparing the amount of the polynucleotide in a solution containing the nanoparticle composition before and after breaking up the nanoparticle composition with one or more organic solvents or detergents.

Fluorescence can be used to measure the amount of free polynucleotide in a solution. For the nanoparticle compositions described herein, the encapsulation efficiency of a polynucleotide can be at least 50%, for example 50%, 55%, 60%, 65%, 70%, 75%, 80%, 85%, 90%, 91%, 92%, 93%, 94%, 95%, 96%, 97%, 98%, 99%, or 100%. In some embodiments, the encapsulation efficiency can be at least 80%. In certain embodiments, the encapsulation efficiency can be at least 90%.

The amount of a polynucleotide present in a pharmaceutical composition disclosed herein can depend on multiple factors such as the size of the polynucleotide, desired target and/or application, or other properties of the nanoparticle composition as well as on the properties of the polynucleotide.

For example, the amount of an mRNA useful in a nanoparticle composition can depend on the size (expressed as length, or molecular mass), sequence, and other characteristics of the mRNA. The relative amounts of a polynucleotide in a nanoparticle composition can also vary.

The relative amounts of the lipid composition and the polynucleotide present in a lipid nanoparticle composition of the present disclosure can be optimized according to considerations of efficacy and tolerability. For compositions including an mRNA as a polynucleotide, the N:P ratio can serve as a useful metric.

As the N:P ratio of a nanoparticle composition controls both expression and tolerability, nanoparticle compositions with low N:P ratios and strong expression are desirable. N:P ratios vary according to the ratio of lipids to RNA in a nanoparticle composition.

In general, a lower N:P ratio is preferred. The one or more RNA, lipids, and amounts thereof can be selected to provide an N:P ratio from about 2:1 to about 30:1, such as 2:1, 3:1, 4:1, 5:1, 6:1, 7:1, 8:1, 9:1, 10:1, 12:1, 14:1, 16:1, 18:1, 20:1, 22:1, 24:1, 26:1, 28:1, or 30:1. In certain embodiments, the N:P ratio can be from about 2:1 to about 8:1. In other embodiments, the N:P ratio is from about 5:1 to about 8:1. In certain embodiments, the N:P ratio is between 5:1 and 6:1. In one specific aspect, the N:P ratio is about is about 5.67:1.

In addition to providing nanoparticle compositions, the present disclosure also provides methods of producing lipid nanoparticles comprising encapsulating a polynucleotide. Such method comprises using any of the pharmaceutical compositions disclosed herein and producing lipid nanoparticles in accordance with methods of production of lipid nanoparticles known in the art. See, e.g., Wang et al. (2015) “Delivery of oligonucleotides with lipid nanoparticles” Adv. Drug Deliv. Rev. 87:68-80; Silva et al. (2015) “Delivery Systems for Biopharmaceuticals. Part I: Nanoparticles and Microparticles” Curr. Pharm. Technol. 16:940-954; Naseri et al. (2015) “Solid Lipid Nanoparticles and Nanostructured Lipid Carriers: Structure, Preparation and Application” Adv. Pharm. Bull. 5:305-13; Silva et al. (2015) “Lipid nanoparticles for the delivery of biopharmaceuticals” Curr. Pharm. Biotechnol. 16:291-302, and references cited therein.

In some embodiments, the ratio between the lipid composition and the polynucleotide can be about 10:1, 11:1, 12:1, 13:1, 14:1, 15:1, 16:1, 17:1, 18:1, 19:1, 20:1, 21:1, 22:1, 23:1, 24:1, 25:1, 26:1, 27:1, 28:1, 29:1, 30:1, 31:1, 32:1, 33:1, 34:1, 35:1, 36:1, 37:1, 38:1, 39:1, 40:1, 41:1, 42:1, 43:1, 44:1, 45:1, 46:1, 47:1, 48:1, 49:1, 50:1, 51:1, 52:1, 53:1, 54:1, 55:1, 56:1, 57:1, 58:1, 59:1 or 60:1 (wt/wt). In some embodiments, the wt/wt ratio of the lipid composition to the polynucleotide encoding a therapeutic agent is about 20:1 or about 15:1.

In some embodiments, the pharmaceutical composition disclosed herein can contain more than one polypeptide. For example, a pharmaceutical composition disclosed herein can contain two or more polynucleotides (e.g., RNA, e.g., mRNA).

In one embodiment, the lipid nanoparticles described herein can comprise polynucleotides (e.g., mRNA) in a lipid: polynucleotide weight ratio of 5:1, 10:1, 15:1, 20:1, 25:1, 30:1, 35:1, 40:1, 45:1, 50:1, 55:1, 60:1 or 70:1, or a range or any of these ratios such as, but not limited to, 5:1 to about 10:1, from about 5:1 to about 15:1, from about 5:1 to about 20:1, from about 5:1 to about 25:1, from about 5:1 to about 30:1, from about 5:1 to about 35:1, from about 5:1 to about 40:1, from about 5:1 to about 45:1, from about 5:1 to about 50:1, from about 5:1 to about 55:1, from about 5:1 to about 60:1, from about 5:1 to about 70:1, from about 10:1 to about 15:1, from about 10:1 to about 20:1, from about 10:1 to about 25:1, from about 10:1 to about 30:1, from about 10:1 to about 35:1, from about 10:1 to about 40:1, from about 10:1 to about 45:1, from about 10:1 to about 50:1, from about 10:1 to about 55:1, from about 10:1 to about 60:1, from about 10:1 to about 70:1, from about 15:1 to about 20:1, from about 15:1 to about 25:1,from about 15:1 to about 30:1, from about 15:1 to about 35:1, from about 15:1 to about 40:1, from about 15:1 to about 45:1, from about 15:1 to about 50:1, from about 15:1 to about 55:1, from about 15:1 to about 60:1 or from about 15:1 to about 70:1.

In one embodiment, the lipid nanoparticles described herein can comprise the polynucleotide in a concentration from approximately 0.1 mg/ml to 2 mg/ml such as, but not limited to, 0.1 mg/ml, 0.2 mg/ml, 0.3 mg/ml, 0.4 mg/ml, 0.5 mg/ml, 0.6 mg/ml, 0.7 mg/ml, 0.8 mg/ml, 0.9 mg/ml, 1.0 mg/ml, 1.1 mg/ml, 1.2 mg/ml, 1.3 mg/ml, 1.4 mg/ml, 1.5 mg/ml, 1.6 mg/ml, 1.7 mg/ml, 1.8 mg/ml, 1.9 mg/ml, 2.0 mg/ml or greater than 2.0 mg/ml.

### Methods of Using LNP Compositions Comprising a Checkpoint Cancer Vaccine

In an aspect, the disclosure provides a composition comprising a lipid nanoparticle (LNP) (e.g., an LNP composition described herein) comprising a polynucleotide comprising an mRNA which encodes a checkpoint cancer vaccine comprising (i) one or more IDO antigenic peptides and (ii) one or more PD-L1 antigenic peptides, and optionally one or more adjuvant amino acid sequences in the treatment of a cancer in a subject, e.g., in accordance with a method described herein.

In another aspect, the disclosure provides a composition comprising a lipid nanoparticle (LNP) (e.g., an LNP composition described herein) comprising a polynucleotide comprising an mRNA which encodes a a checkpoint cancer vaccine comprising (i) one or more IDO antigenic peptides and (ii) one or more PD-L1 antigenic peptides and optionally one or more adjuvant amino acid sequences, for stimulating an immune response in a subject, e.g., in accordance with a method described herein.

In another aspect, the disclosure provides a composition comprising a lipid nanoparticle (LNP) (e.g., an LNP composition described herein) comprising a polynucleotide comprising an mRNA which encodes a checkpoint cancer vaccine comprising (i) one or more IDO antigenic peptides and (ii) one or more PD-L1 antigenic peptides, for stimulating effector T-cells to target and kill tumor cells that express IDO or PD-L1, e.g., in a subject, e.g., in accordance with a method described herein.

In another aspect, the disclosure provides a composition comprising a lipid nanoparticle (LNP) (e.g., an LNP composition described herein) comprising a polynucleotide comprising an mRNA which encodes a checkpoint cancer vaccine comprising (i) one or more IDO antigenic peptides and (ii) one or more PD-L1 antigenic peptides and optionally one or more adjuvant amino acid sequences, or a combination thereof, for stimulating T cells, e.g., T effector cells, e.g., in accordance with a method described herein.

In another aspect, the disclosure provides a composition comprising a lipid nanoparticle (LNP) (e.g., an LNP composition described herein) comprising a polynucleotide comprising an mRNA which encodes a checkpoint cancer vaccine comprising (i) one or more IDO antigenic peptides and (ii) one or more PD-L1 antigenic peptides and optionally one or more adjuvant amino acid sequences, for inducing T-cell mediated killing of tumor cells by vaccine-activated T cells, e.g., in accordance with a method described herein.

In another aspect, the disclosure provides a composition comprising a lipid nanoparticle (LNP) (e.g., an LNP composition described herein) comprising a polynucleotide comprising an mRNA which encodes a checkpoint cancer vaccine comprising (i) one or more IDO antigenic peptides and (ii) one or more PD-L1 antigenic peptides and optionally one or more adjuvant amino acid sequences, for use, in the treatment of a cancer in a subject.

In a related aspect, provided herein is a method of treating a cancer in a subject, comprising administering to the subject an effective amount of a lipid nanoparticle (LNP) (e.g., an LNP composition described herein) comprising a polynucleotide comprising an mRNA which encodes a checkpoint cancer vaccine comprising (i) one or more IDO antigenic peptides and (ii) one or more PD-L1 antigenic peptides and optionally one or more adjuvant amino acid sequences.

In embodiments of any of the methods disclosed herein, administration of the LNP results in amelioration or delay of progression of cancer, e.g., as described herein, in a subject, e.g., as measured by an assay described herein. In embodiments, the amelioration or delay of disease progression is compared to disease progression in an otherwise similar subject, e.g., a subject who has not been contacted with the LNP composition comprising a checkpoint cancer vaccine comprising (i) one or more IDO antigenic peptides and (ii) one or more PD-L1 antigenic peptides and optionally one or more adjuvant amino acid sequences.

In embodiments, the delay in progression of cancer is a delay of at least 1 week, 2 weeks, 3 weeks, 1 month, 2 months, 3 months, 4 months, 5 months, 6 months, 7 months, 8 months, 9 months, 10 months, 11 months, 12 months, 1.5 years, 2 years, 3 years, 4 years, or 5 years or greater.

In some embodiments, the checkpoint cancer vaccine stimulates effector T cells that target and kill suppressive immune and tumor cells that express the target antigens. Accordingly, in some embodiments, IDO- and PD-L1-specific T cells kill immunosuppressive (regulatory) immune cells and cancer cells that overexpress IDO and PD-L1. In some embodiments, treatment results in additional tumor killing by vaccine-activated T cells. Additionally, in some embodiments, administration of the checkpoint cancer vaccine results in T cell priming, leading to recognition of additional tumor-associated antigens and to increased tumor killing by tumor-specific cytotoxic T cells. Without wishing to be bound by theory it is thought that systemic PD-1/PD-L1 blockade may further amplify the effect, leading to further immune activation and superior disease control.

In an embodiment, the cancer is a solid tumor, e.g., is a locally advanced or metastatic solid tumor. In an embodiment, the cancer is a melanoma. In an embodiment, the melanoma is a cutaneous melanoma. In an embodiment, the cutaneous melanoma is a 1 L cutaneous melanoma stage IIIB+. In an embodiment, the cancer is a NSCLC. In an embodiment, the NSCLC is a 1 L NSCLC. In an embodiment, the cancer is a bladder cancer. In some embodiments, the bladder cancer is a non-muscle invasive bladder cancer. In an embodiment, the cancer is a head and neck cancer. In some embodiments, the head and neck cancer is a head and neck squamous cell carcinoma. In an embodiment, the cancer is a colorectal cancer. In some embodiments, the colorectal cancer is a microsatellite stable colorectal cancer. In an embodiment, the cancer is a basal cell carcinoma. In an embodiment, the cancer is a breast cancer. In some embodiments, the breast cancer is a triple negative breast cancer.

### LNP Dosing and Dosing Regimen

In some embodiments, any of the LNP disclosed herein can be administered according to a dosing interval, e.g., as described herein. In some embodiments, the dosing interval comprises an initial dose of the LNP composition and one or more subsequent doses (e.g., 1-50 doses, 5-50 doses, 10-50 doses, 15-50 doses, 20-50 doses, 25-50 doses, 30-50 doses, 35-50 doses, 40-50 doses, 45-50 doses, 1-45 doses, 1-40 doses, 1-35 doses, 1-30 doses, 1-25 doses, 1-20 doses, 1-15 doses, 1-10 doses, 1-5 doses) of the same LNP composition.

In some embodiments, the dosing interval comprises one or more doses of the LNP composition and one or more doses of an additional agent.

In some embodiments, the dosing interval is performed over at least 1 week, 2 weeks, 3 weeks, or 4 weeks.

In some embodiments, the dosing interval comprises a cycle, e.g., a seven-day cycle. In some embodiments, the cycle comprises 3 weeks (e.g., 21 days).

In some embodiments, the LNP composition is administered once every three weeks for one or more cycles. In some embodiments, the dosing regimen comprises two cycles, three cycles, four cycles, five cycles, six cycles, seven cycles, eight cycles, or nine cycles.

In some embodiments, the LNP composition is administered to the subject at a dose of about 50 μg to about 1 mg, e.g., about 100 μg to about 1 mg, about 200 μg to about 900 μg, about 300 μg to about 800 μg, about 400 μg to about 700 μg, about 500 μg to about 600 μg, about 200 μg to about 1 mg, about 300 μg to about 1 mg, about 400 μg to about 1 mg, about 500 μg to about 1 mg, about 600 μg to about 1 mg, about 700 μg to about 1 mg, about 800 μg to about 1 mg, about 900 μg to about 1 mg, about 100 μg to about 900 μg, about 100 μg to about 800 μg, about 100 μg to about 700 μg, about 100 μg to about 600 μg, about 100 μg to about 500 μg, about 100 μg to about 400 μg, about 100 μg to about 300 μg, about 100 μg to about 200 μg, about 200 μg to about 400 μg, about 300 μg to about 500 μg, about 400 μg to about 600 μg, about 500 μg to about 700 μg, about 600 μg to about 800 μg, or about 700 μg to about 900 μg.

In some embodiments, the LNP composition is administered at a dose of about 100 μg to about 200 μg, about 200 μg to about 300 μg, about 300 μg to about 400 μg, about 400 μg to about 500 μg, about 500 μg to about 600 μg, about 600 μg to about 700 μg, about 700 μg to about 800 μg, about 800 μg to about 900 μg, or about 900 μg to about 1 mg. In some embodiments, the LNP composition is administered at a dose of about 50 μg to about 150 μg, about 150 μg to about 250 μg, about 250 μg to about 350 μg. about 350 μg to about 450 μg, about 450 μg to about 550 μg, about 550 μg to about 650 μg, about 650 μg to about 750 μg, about 750 μg to about 850 μg, about 850 μg to about 950 μg, or about 950 μg to about 1 mg. In some embodiments, the LNP composition is administered at a dose of about 100 μg, about 200 μg, about 300 μg, about 400 μg, about 500 μg, about 600 μg, about 700 μg, about 800 μg, about 900 μg, or about 1 mg.

In some embodiments of any of the LNP compositions disclosed herein, the LNP composition is administered at a dose, e.g., total dose, of about 0.1-10 mg per kg, about 0.1-9.5 mg per kg, about 0.1-9 mg per kg, about 0.1-8.5 mg per kg, about 0.1-8 mg per kg, about 0.1-7.5 mg per kg, about 0.1-7 mg per kg, about 0.1-6.5 mg per kg, about 0.1-6 mg per kg, about 0.1-5.5 mg per kg, about 0.1-5 mg per kg, about 0.1-4.5 mg per kg, about 0.1-4 mg per kg, about 0.1-3.5 mg per kg, about 0.1-3 mg per kg, about 0.1-2.5 mg per kg, about 0.1-2 mg per kg, about 0.1-1.5 mg per kg, about 0.1-1 mg per kg, about 0.1-0.9 mg per kg, about 0.1-0.8 mg per kg, about 0.1-0.7 mg per kg, about 0.1-0.6 mg per kg, or about 0.1-0.5 mg per kg.

In some embodiments of any of the LNP compositions disclosed herein, the LNP composition is administered at a dose, e.g., total dose, of about 0.2-10 mg per kg, about, 0.3-10 mg per kg, about 0.4-10 mg per kg, about 0.5-10 mg per kg, about 0.6-10 mg per kg, about 0.7-10 mg per kg, about 0.8-10 mg per kg, about 0.9-10 mg per kg, about 1-10 mg per kg, about 1.5-10 mg per kg, about 2-10 mg per kg, about 2.5-10 mg per kg, about 3-10 mg per kg, about 3.5-10 mg per kg, about 4-10 mg per kg, about 4.5-10 mg per kg, about 5-10 mg per kg, about 5.5-10 mg per kg, about 6-10 mg per kg, about 6.5-10 mg per kg, about 7-10 mg per kg, about 7.5-10 mg per kg, about 8-10 mg per kg, about 8.5-10 mg per kg, about 9-10 mg per kg, or about 9.5-10 mg per kg.

In some embodiments, any of the LNP disclosed herein is administered intramuscularly (IM).

### Diseases and Disorders

In an embodiment of any of the methods of treatment or compositions for use disclosed herein, the subject has, or is identified as having, a cancer. In an embodiment, an LNP disclosed herein is administered to the subject to treat or ameliorate a symptom of the cancer. In an embodiment, an LNP disclosed herein is administered to a subject to stimulate an immune response in the subject.

In an embodiment, the cancer is a solid tumor, e.g., a locally advanced or metastatic solid tumor. In an embodiment, the cancer is a melanoma. In some embodiments, the melanoma is a cutaneous melanoma. In some embodiments, the cutaneous melanoma is a 1 L cutaneous melanoma stage IIIB+. In an embodiment, the cancer is a non-small cell lung cancer (NSCLC). In some embodiments, the NSCLC is a 1 L NSCLC. In an embodiment, the cancer is a bladder cancer. In some embodiments, the bladder cancer is a non-muscle invasive bladder cancer. In an embodiment, the cancer is a head and neck cancer. In some embodiments, the head and neck cancer is a head and neck squamous cell carcinoma. In an embodiment, the cancer is a colorectal cancer. In some embodiments, the colorectal cancer is a microsatellite stable colorectal cancer. In an embodiment, the cancer is a basal cell carcinoma. In an embodiment, the cancer is a breast cancer. In some embodiments, the breast cancer is a triple negative breast cancer.

In an embodiment the subject is a mammal, e.g., a human.

### Further Combination Therapies

In some embodiments, the methods of treatment or compositions for use disclosed herein, comprise administering an LNP disclosed herein in combination with an additional agent. In an embodiment, the additional agent is a standard of care for the disease or disorder, e.g., autoimmune disease. In an embodiment, the additional agent is an mRNA

In some aspects, the subject for the present methods or compositions has been treated with one or more standard of care therapies. In other aspects, the subject for the present methods or compositions has not been responsive to one or more standard of care therapies.

For example, a checkpoint inhibitor, such as anti-PD1 antibody or anti-CTLA4 antibody, is additionally administered to the subject. In some embodiments, the anti-PD-1 antibody is pembrolizumab.

Dosing of combination therapies can be determined by one of skill in the art. In some embodiments, an anti-PD-1 antibody, e.g., pembrolizumab is administered (e.g., by intravenous administration) to a subject at a dose of 200 mg once every three weeks or 400 mg once every six weeks, e.g., for a total of twenty-four weeks. In some embodiments, an anti-PD-1 antibody, e.g., pembrolizumab is administered (e.g., by intravenous administration) to a subject at a dose of 400 mg once every 6 weeks (e.g., on day 1 of every other 3-week cycle, e.g., day 1 of cycle 1, cycle 3, cycle 5, cycle 7, and cycle 9).

In some embodiments, the additional therapy is administered to the patient concurrently with (e.g., on the same day as) the LNP composition. In some embodiments, the additional therapy is administered separately from the LNP composition (e.g., administered on a separate day).

### Sequence Optimization and Methods Thereof

In some embodiments, a polynucleotide of the disclosure comprises a sequence-optimized nucleotide sequence encoding a checkpoint cancer vaccine comprising (i) one or more IDO antigenic peptides and (ii) one or more PD-L1 antigenic peptides. In some embodiments, the polynucleotide of the disclosure comprises an open reading frame (ORF) encoding one or more IDO antigenic peptides and one or more PD-L1 antigenic peptides, wherein the ORF has been sequence optimized.

The sequence-optimized nucleotide sequences disclosed herein are distinct from the corresponding wild type nucleotide acid sequences and from other known sequence-optimized nucleotide sequences, e.g., these sequence-optimized nucleic acids have unique compositional characteristics.

In some embodiments, the percentage of uracil or thymine nucleobases in a sequence-optimized nucleotide sequence (e.g., encoding a checkpoint cancer vaccine) is modified (e.g., reduced) with respect to the percentage of uracil or thymine nucleobases in the reference wild-type nucleotide sequence. Such a sequence is referred to as a uracil-modified or thymine-modified sequence. The percentage of uracil or thymine content in a nucleotide sequence can be determined by dividing the number of uracils or thymines in a sequence by the total number of nucleotides and multiplying by 100. In some embodiments, the sequence-optimized nucleotide sequence has a lower uracil or thymine content than the uracil or thymine content in the reference wild-type sequence. In some embodiments, the uracil or thymine content in a sequence-optimized nucleotide sequence of the disclosure is greater than the uracil or thymine content in the reference wild-type sequence and still maintain beneficial effects, e.g., increased expression and/or signaling response when compared to the reference wild-type sequence.

In some embodiments, the optimized sequences of the present disclosure contain unique ranges of uracils or thymine (if DNA) in the sequence. The uracil or thymine content of the optimized sequences can be expressed in various ways, e.g., uracil or thymine content of optimized sequences relative to the theoretical minimum (% UTM or % TTM), relative to the wild-type (% UWT or % TWT), and relative to the total nucleotide content (% UTL or % TTL). For DNA it is recognized that thymine is present instead of uracil, and one would substitute T where U appears. Thus, all the disclosures related to, e.g., % UTM, % UWT, or % UTL, with respect to RNA are equally applicable to % TTM, % TWT, or % TTL with respect to DNA.

Uracil- or thymine-content relative to the uracil or thymine theoretical minimum, refers to a parameter determined by dividing the number of uracils or thymines in a sequence-optimized nucleotide sequence by the total number of uracils or thymines in a hypothetical nucleotide sequence in which all the codons in the hypothetical sequence are replaced with synonymous codons having the lowest possible uracil or thymine content and multiplying by 100. This parameter is abbreviated herein as % UTM or % TTM.

In some embodiments, a uracil-modified sequence encoding a checkpoint cancer vaccine of the disclosure has a reduced number of consecutive uracils with respect to the corresponding wild-type nucleic acid sequence. For example, two consecutive leucines can be encoded by the sequence CUUUUG, which includes a four uracil cluster. Such a subsequence can be substituted, e.g., with CUGCUC, which removes the uracil cluster. Phenylalanine can be encoded by UUC or UUU. Thus, even if phenylalanines encoded by UUU are replaced by UUC, the synonymous codon still contains a uracil pair (UU). Accordingly, the number of phenylalanines in a sequence establishes a minimum number of uracil pairs (UU) that cannot be eliminated without altering the number of phenylalanines in the encoded polypeptide.

In some embodiments, a uracil-modified sequence encoding a checkpoint cancer vaccine of the disclosure has a reduced number of uracil triplets (UUU) with respect to the wild-type nucleic acid sequence. In some embodiments, a uracil-modified sequence encoding a checkpoint cancer vaccine has a reduced number of uracil pairs (UU) with respect to the number of uracil pairs (UU) in the wild-type nucleic acid sequence. In some embodiments, a uracil-modified sequence encoding a checkpoint cancer vaccine of the disclosure has a number of uracil pairs (UU) corresponding to the minimum possible number of uracil pairs (UU) in the wild-type nucleic acid sequence.

The phrase “uracil pairs (UU) relative to the uracil pairs (UU) in the wild type nucleic acid sequence,” refers to a parameter determined by dividing the number of uracil pairs (UU) in a sequence-optimized nucleotide sequence by the total number of uracil pairs (UU) in the corresponding wild-type nucleotide sequence and multiplying by 100. This parameter is abbreviated herein as % UUwt. In some embodiments, a uracil-modified sequence encoding checkpoint cancer vaccine has a % UUwt between below 100%.

In some embodiments, the polynucleotide of the disclosure comprises a uracil-modified sequence encoding a checkpoint cancer vaccine disclosed herein. In some embodiments, the uracil-modified sequence encoding a checkpoint cancer vaccine comprises at least one chemically modified nucleobase, e.g., 5-methoxyuracil. In some embodiments, at least 95% of a nucleobase (e.g., uracil) in a uracil-modified sequence encoding a checkpoint cancer vaccine of the disclosure are modified nucleobases. In some embodiments, at least 95% of uracil in a uracil-modified sequence encoding a checkpoint cancer vaccine is 5-methoxyuracil. In some embodiments, the polynucleotide comprising a uracil-modified sequence further comprises a miRNA binding site, e.g., a miRNA binding site that binds to miR-122. In some embodiments, the polynucleotide comprising a uracil-modified sequence is formulated with a delivery agent, e.g., a compound having Formula (I), e.g., any of Compound Nos. 18, 25, 301, or 357.

In some embodiments, a polynucleotide of the disclosure (e.g., a polynucleotide comprising a nucleotide sequence encoding a checkpoint cancer vaccine (e.g., the wild-type sequence, functional fragment, or variant thereof) is sequence optimized.

A sequence optimized nucleotide sequence (nucleotide sequence is also referred to as “nucleic acid” herein) comprises at least one codon modification with respect to a reference sequence (e.g., a wild-type sequence encoding a checkpoint cancer vaccine). Thus, in a sequence optimized nucleic acid, at least one codon is different from a corresponding codon in a reference sequence (e.g., a wild-type sequence).

In general, sequence optimized nucleic acids are generated by at least a step comprising substituting codons in a reference sequence with synonymous codons (i.e., codons that encode the same amino acid). Such substitutions can be effected, for example, by applying a codon substitution map (i.e., a table providing the codons that will encode each amino acid in the codon optimized sequence), or by applying a set of rules (e.g., if glycine is next to neutral amino acid, glycine would be encoded by a certain codon, but if it is next to a polar amino acid, it would be encoded by another codon). In addition to codon substitutions (i.e., “codon optimization”) the sequence optimization methods disclosed herein comprise additional optimization steps which are not strictly directed to codon optimization such as the removal of deleterious motifs (destabilizing motif substitution).

Additional and exemplary methods of sequence optimization are disclosed in International PCT application WO 2017/201325, filed on 18 May 2017, the entire contents of which are hereby incorporated by reference.

MicroRNA (miRNA) Binding Sites

Polynucleotides of the invention can include regulatory elements, for example, microRNA (miRNA) binding sites, transcription factor binding sites, structured mRNA sequences and/or motifs, artificial binding sites engineered to act as pseudo-receptors for endogenous nucleic acid binding molecules, and combinations thereof. In some embodiments, polynucleotides including such regulatory elements are referred to as including “sensor sequences”.

In some embodiments, a polynucleotide (e.g., a ribonucleic acid (RNA), e.g., a messenger RNA (mRNA)) of the invention comprises an open reading frame (ORF) encoding a polypeptide of interest and further comprises one or more miRNA binding site(s). Inclusion or incorporation of miRNA binding site(s) provides for regulation of polynucleotides of the invention, and in turn, of the polypeptides encoded therefrom, based on tissue-specific and/or cell-type specific expression of naturally occurring miRNAs.

The present invention also provides pharmaceutical compositions and formulations that comprise any of the polynucleotides described above. In some embodiments, the composition or formulation further comprises a delivery agent.

In some embodiments, the composition or formulation can contain a polynucleotide comprising a sequence optimized nucleic acid sequence disclosed herein which encodes a polypeptide. In some embodiments, the composition or formulation can contain a polynucleotide (e.g., a RNA, e.g., an mRNA) comprising a polynucleotide (e.g., an ORF) having significant sequence identity to a sequence optimized nucleic acid sequence disclosed herein which encodes a polypeptide. In some embodiments, the polynucleotide further comprises a miRNA binding site, e.g., a miRNA binding site that binds

A miRNA, e.g., a natural-occurring miRNA, is a 19-25 nucleotide long noncoding RNA that binds to a polynucleotide and down-regulates gene expression either by reducing stability or by inhibiting translation of the polynucleotide. A miRNA sequence comprises a “seed” region, i.e., a sequence in the region of positions 2-8 of the mature miRNA. A miRNA seed can comprise positions 2-8 or 2-7 of the mature miRNA.

MicroRNAs derive enzymatically from regions of RNA transcripts that fold back on themselves to form short hairpin structures often termed a pre-miRNA (precursor-miRNA). A pre-miRNA typically has a two-nucleotide overhang at its 3′ end, and has 3′ hydroxyl and 5′ phosphate groups. This precursor-mRNA is processed in the nucleus and subsequently transported to the cytoplasm where it is further processed by DICER (a RNase III enzyme), to form a mature microRNA of approximately 22 nucleotides. The mature microRNA is then incorporated into a ribonuclear particle to form the RNA-induced silencing complex, RISC, which mediates gene silencing. Art-recognized nomenclature for mature miRNAs typically designates the arm of the pre-miRNA from which the mature miRNA derives; “5p” means the microRNA is from the 5-prime arm of the pre-miRNA hairpin and “3p” means the microRNA is from the 3-prime end of the pre-miRNA hairpin. A miR referred to by number herein can refer to either of the two mature microRNAs originating from opposite arms of the same pre-miRNA (e.g., either the 3p or 5p microRNA). All miRs referred to herein are intended to include both the 3p and 5p arms/sequences, unless particularly specified by the 3p or 5p designation.

As used herein, the term “microRNA (miRNA or miR) binding site” refers to a sequence within a polynucleotide, e.g., within a DNA or within an RNA transcript, including in the 5′UTR and/or 3′UTR, that has sufficient complementarity to all or a region of a miRNA to interact with, associate with or bind to the miRNA. In some embodiments, a polynucleotide of the invention comprising an ORF encoding a polypeptide of interest and further comprises one or more miRNA binding site(s). In exemplary embodiments, a 5′ UTR and/or 3′ UTR of the polynucleotide (e.g., a ribonucleic acid (RNA), e.g., a messenger RNA (mRNA)) comprises the one or more miRNA binding site(s).

A miRNA binding site having sufficient complementarity to a miRNA refers to a degree of complementarity sufficient to facilitate miRNA-mediated regulation of a polynucleotide, e.g., miRNA-mediated translational repression or degradation of the polynucleotide. In exemplary aspects of the invention, a miRNA binding site having sufficient complementarity to the miRNA refers to a degree of complementarity sufficient to facilitate miRNA-mediated degradation of the polynucleotide, e.g., miRNA-guided RNA-induced silencing complex (RISC)-mediated cleavage of mRNA. The miRNA binding site can have complementarity to, for example, a 19-25 nucleotide long miRNA sequence, to a 19-23 nucleotide long miRNA sequence, or to a 22-nucleotide long miRNA sequence. A miRNA binding site can be complementary to only a portion of a miRNA, e.g., to a portion less than 1, 2, 3, or 4 nucleotides of the full length of a naturally occurring miRNA sequence, or to a portion less than 1, 2, 3, or 4 nucleotides shorter than a naturally occurring miRNA sequence. Full or complete complementarity (e.g., full complementarity or complete complementarity over all or a significant portion of the length of a naturally occurring miRNA) is preferred when the desired regulation is mRNA degradation. In some embodiments, a miRNA binding site includes a sequence that has complementarity (e.g., partial or complete complementarity) with a miRNA seed sequence. In some embodiments, the miRNA binding site includes a sequence that has complete complementarity with a miRNA seed sequence. In some embodiments, a miRNA binding site includes a sequence that has complementarity (e.g., partial or complete complementarity) with a miRNA sequence. In some embodiments, the miRNA binding site includes a sequence that has complete complementarity with a miRNA sequence. In some embodiments, a miRNA binding site has complete complementarity with a miRNA sequence but for 1, 2, or 3 nucleotide substitutions, terminal additions, and/or truncations.

In some embodiments, the miRNA binding site is the same length as the corresponding miRNA. In other embodiments, the miRNA binding site is one, two, three, four, five, six, seven, eight, nine, ten, eleven or twelve nucleotide(s) shorter than the corresponding miRNA at the 5′ terminus, the 3′ terminus, or both. In still other embodiments, the microRNA binding site is two nucleotides shorter than the corresponding microRNA at the 5′ terminus, the 3′ terminus, or both. The miRNA binding sites that are shorter than the corresponding miRNAs are still capable of degrading the mRNA incorporating one or more of the miRNA binding sites or preventing the mRNA from translation.

In some embodiments, the miRNA binding site binds the corresponding mature miRNA that is part of an active RISC containing Dicer. In another embodiment, binding of the miRNA binding site to the corresponding miRNA in RISC degrades the mRNA containing the miRNA binding site or prevents the mRNA from being translated. In some embodiments, the miRNA binding site has sufficient complementarity to miRNA so that a RISC complex comprising the miRNA cleaves the polynucleotide comprising the miRNA binding site. In other embodiments, the miRNA binding site has imperfect complementarity so that a RISC complex comprising the miRNA induces instability in the polynucleotide comprising the miRNA binding site. In another embodiment, the miRNA binding site has imperfect complementarity so that a RISC complex comprising the miRNA represses transcription of the polynucleotide comprising the miRNA binding site.

In some embodiments, the miRNA binding site has one, two, three, four, five, six, seven, eight, nine, ten, eleven, or twelve mismatch(es) from the corresponding miRNA.

In some embodiments, the miRNA binding site has at least about ten, at least about eleven, at least about twelve, at least about thirteen, at least about fourteen, at least about fifteen, at least about sixteen, at least about seventeen, at least about eighteen, at least about nineteen, at least about twenty, or at least about twenty-one contiguous nucleotides complementary to at least about ten, at least about eleven, at least about twelve, at least about thirteen, at least about fourteen, at least about fifteen, at least about sixteen, at least about seventeen, at least about eighteen, at least about nineteen, at least about twenty, or at least about twenty-one, respectively, contiguous nucleotides of the corresponding miRNA.

By engineering one or more miRNA binding sites into a polynucleotide of the invention, the polynucleotide can be targeted for degradation or reduced translation, provided the miRNA in question is available. This can reduce off-target effects upon delivery of the polynucleotide. For example, if a polynucleotide of the invention is not intended to be delivered to a tissue or cell but ends up is said tissue or cell, then a miRNA abundant in the tissue or cell can inhibit the expression of the gene of interest if one or multiple binding sites of the miRNA are engineered into the 5′ UTR and/or 3′ UTR of the polynucleotide. Thus, in some embodiments, incorporation of one or more miRNA binding sites into an mRNA of the disclosure may reduce the hazard of off-target effects upon nucleic acid molecule delivery and/or enable tissue-specific regulation of expression of a polypeptide encoded by the mRNA. In yet other embodiments, incorporation of one or more miRNA binding sites into an mRNA of the disclosure can modulate immune responses upon nucleic acid delivery in vivo. In further embodiments, incorporation of one or more miRNA binding sites into an mRNA of the disclosure can modulate accelerated blood clearance (ABC) of lipid-comprising compounds and compositions described herein.

Conversely, miRNA binding sites can be removed from polynucleotide sequences in which they naturally occur to increase protein expression in specific tissues. For example, a binding site for a specific miRNA can be removed from a polynucleotide to improve protein expression in tissues or cells containing the miRNA.

Regulation of expression in multiple tissues can be accomplished through introduction or removal of one or more miRNA binding sites, e.g., one or more distinct miRNA binding sites. The decision whether to remove or insert a miRNA binding site can be made based on miRNA expression patterns and/or their profiling in tissues and/or cells in development and/or disease. Identification of miRNAs, miRNA binding sites, and their expression patterns and role in biology have been reported (e.g., Bonauer et al., Curr Drug Targets 2010 11:943-949; Anand and Cheresh Curr Opin Hematol 2011 18:171-176; Contreras and Rao Leukemia 2012 26:404-413 (2011 Dec. 20. doi: 10.1038/leu.2011.356); Bartel Cell 2009 136:215-233; Landgraf et al, Cell, 2007 129:1401-1414; Gentner and Naldini, Tissue Antigens. 2012 80:393-403 and all references therein; each of which is incorporated herein by reference in its entirety).

Examples of tissues where miRNA are known to regulate mRNA, and thereby protein expression, include, but are not limited to, liver (miR-122), muscle (miR-133, miR-206, miR-208), endothelial cells (miR-17-92, miR-126), myeloid cells (miR-142-3p, miR-142-5p, miR-16, miR-21, miR-223, miR-24, miR-27), adipose tissue (let-7, miR-30c), heart (miR-1d, miR-149), kidney (miR-192, miR-194, miR-204), and lung epithelial cells (let-7, miR-133, miR-126).

Specifically, miRNAs are known to be differentially expressed in immune cells (also called hematopoietic cells), such as antigen presenting cells (APCs) (e.g., dendritic cells and macrophages), macrophages, monocytes, B lymphocytes, T lymphocytes, granulocytes, natural killer cells, etc. Immune cell specific miRNAs are involved in immunogenicity, autoimmunity, the immune response to infection, inflammation, as well as unwanted immune response after gene therapy and tissue/organ transplantation. Immune cells specific miRNAs also regulate many aspects of development, proliferation, differentiation, and apoptosis of hematopoietic cells (immune cells). For example, miR-142 and miR-146 are exclusively expressed in immune cells, particularly abundant in myeloid dendritic cells. It has been demonstrated that the immune response to a polynucleotide can be shut-off by adding miR-142 binding sites to the 3′-UTR of the polynucleotide, enabling more stable gene transfer in tissues and cells. miR-142 efficiently degrades exogenous polynucleotides in antigen presenting cells and suppresses cytotoxic elimination of transduced cells (e.g., Annoni A et al., blood, 2009, 114, 5152-5161; Brown B D, et al., Nat med. 2006, 12(5), 585-591; Brown B D, et al., blood, 2007, 110(13): 4144-4152, each of which is incorporated herein by reference in its entirety).

In some embodiments, a polynucleotide of the invention comprises a miRNA binding site, wherein the miRNA binding site comprises one or more nucleotide sequences selected from Table 3C or Table 4A, including one or more copies of any one or more of the miRNA binding site sequences. In some embodiments, a polynucleotide of the invention further comprises at least one, two, three, four, five, six, seven, eight, nine, ten, or more of the same or different miRNA binding sites selected from Table 3C or Table 4A, including any combination thereof.

In some embodiments, the miRNA binding site binds to miR-142 or is complementary to miR-142. In some embodiments, the miR-142 comprises SEQ ID NO: 200. In some embodiments, the miRNA binding site binds to miR-142-3p or miR-142-5p. In some embodiments, the miR-142-3p binding site comprises SEQ ID NO:202. In some embodiments, the miR-142-5p binding site comprises SEQ ID NO: 204. In some embodiments, the miRNA binding site comprises a nucleotide sequence at least 80%, at least 85%, at least 90%, at least 95%, or 100% identical to SEQ ID NO:202 or SEQ ID NO:204.

In some embodiments, the miRNA binding site binds to miR-126 or is complementary to miR-126. In some embodiments, the miR-126 comprises SEQ ID NO: 205. In some embodiments, the miRNA binding site binds to miR-126-3p or miR-126-5p. In some embodiments, the miR-126-3p binding site comprises SEQ ID NO: 207. In some embodiments, the miR-126-5p binding site comprises SEQ ID NO: 710. In some embodiments, the miRNA binding site comprises a nucleotide sequence at least 80%, at least 85%, at least 90%, at least 95%, or 100% identical to SEQ ID NO: 121 or SEQ ID NO: 123.

In one embodiment, the 3′ UTR comprises two miRNA binding sites, wherein a first miRNA binding site binds to miR-142 and a second miRNA binding site binds to miR-126. In a specific embodiment, the 3′ UTR binding to miR-142 and miR-126 comprises, consists, or consists essentially of the sequence of SEQ ID NO: 249.

TABLE 3C
miR-142, miR-126, and miR-142 and miR-126
binding sites

| SEQ | | |
|---|---|---|
| ID | | |
| NO. | Description | Sequence |
| 200 | miR-142 | GACAGUGCAGUCACCCAUAAAGUAGAAAG |
| | | CACUACUAACAGCACUGGAGGGUGUAGUG |
| | | UUUCCUACUUUAUGGAUGAGUGUACUGUG |
| 201 | miR-142-3p | UGUAGUGUUUCCUACUUUAUGGA |
| 202 | miR-142-3p | UCCAUAAAGUAGGAAACACUACA |
| | binding site | |
| 203 | miR-142-5p | CAUAAAGUAGAAAGCACUACU |
| 204 | miR-142-5p | AGUAGUGCUUUCUACUUUAUG |
| | binding site | |
| 205 | miR-126 | CGCUGGCGACGGGACAUUAUUACUUUUGG |
| | | UACGCGCUGUGACACUUCAAACUCGUACC |
| | | GUGAGUAAUAAUGCGCCGUCCACGGCA |
| 206 | miR-126-3p | UCGUACCGUGAGUAAUAAUGCG |
| 207 | miR-126-3p | CGCAUUAUUACUCACGGUACGA |
| | binding site | |
| 208 | miR-126-5p | CAUUAUUACUUUUGGUACGCG |
| 710 | miR-126-5p | CGCGUACCAAAAGUAAUAAUG |
| | binding site | |

In some embodiments, a miRNA binding site is inserted in the polynucleotide of the invention in any position of the polynucleotide (e.g., the 5′ UTR and/or 3′ UTR). In some embodiments, the 5′ UTR comprises a miRNA binding site. In some embodiments, the 3′ UTR comprises a miRNA binding site. In some embodiments, the 5′ UTR and the 3′ UTR comprise a miRNA binding site. The insertion site in the polynucleotide can be anywhere in the polynucleotide as long as the insertion of the miRNA binding site in the polynucleotide does not interfere with the translation of a functional polypeptide in the absence of the corresponding miRNA; and in the presence of the miRNA, the insertion of the miRNA binding site in the polynucleotide and the binding of the miRNA binding site to the corresponding miRNA are capable of degrading the polynucleotide or preventing the translation of the polynucleotide.

In some embodiments, a miRNA binding site is inserted in at least about 30 nucleotides downstream from the stop codon of an ORF in a polynucleotide of the invention comprising the ORF. In some embodiments, a miRNA binding site is inserted in at least about 10 nucleotides, at least about 15 nucleotides, at least about 20 nucleotides, at least about 25 nucleotides, at least about 30 nucleotides, at least about 35 nucleotides, at least about 40 nucleotides, at least about 45 nucleotides, at least about 50 nucleotides, at least about 55 nucleotides, at least about 60 nucleotides, at least about 65 nucleotides, at least about 70 nucleotides, at least about 75 nucleotides, at least about 80 nucleotides, at least about 85 nucleotides, at least about 90 nucleotides, at least about 95 nucleotides, or at least about 100 nucleotides downstream from the stop codon of an ORF in a polynucleotide of the invention. In some embodiments, a miRNA binding site is inserted in about 10 nucleotides to about 100 nucleotides, about 20 nucleotides to about 90 nucleotides, about 30 nucleotides to about 80 nucleotides, about 40 nucleotides to about 70 nucleotides, about 50 nucleotides to about 60 nucleotides, about 45 nucleotides to about 65 nucleotides downstream from the stop codon of an ORF in a polynucleotide of the invention.

In some embodiments, a miRNA binding site is inserted within the 3′ UTR immediately following the stop codon of the coding region within the polynucleotide of the invention, e.g., mRNA. In some embodiments, if there are multiple copies of a stop codon in the construct, a miRNA binding site is inserted immediately following the final stop codon. In some embodiments, a miRNA binding site is inserted further downstream of the stop codon, in which case there are 3′ UTR bases between the stop codon and the miR binding site(s). In some embodiments, three non-limiting examples of possible insertion sites for a miR in a 3′ UTR are shown in SEQ ID NOs: 248, 249, and 250, which show a 3′ UTR sequence with a miR-142-3p site inserted in one of three different possible insertion sites, respectively, within the 3′ UTR.

In some embodiments, one or more miRNA binding sites can be positioned within the 5′ UTR at one or more possible insertion sites. For example, three non-limiting examples of possible insertion sites for a miR in a 5′ UTR are shown in SEQ ID NOs: 251, 252, or 253, which show a 5′ UTR sequence with a miR-142-3p site inserted into one of three different possible insertion sites, respectively, within the 5′ UTR.

In one embodiment, a codon optimized open reading frame encoding a polypeptide of interest comprises a stop codon and the at least one microRNA binding site is located within the 3′ UTR 1-100 nucleotides after the stop codon. In one embodiment, the codon optimized open reading frame encoding the polypeptide of interest comprises a stop codon and the at least one microRNA binding site for a miR expressed in immune cells is located within the 3′ UTR 30-50 nucleotides after the stop codon. In another embodiment, the codon optimized open reading frame encoding the polypeptide of interest comprises a stop codon and the at least one microRNA binding site for a miR expressed in immune cells is located within the 3′ UTR at least 50 nucleotides after the stop codon. In other embodiments, the codon optimized open reading frame encoding the polypeptide of interest comprises a stop codon and the at least one microRNA binding site for a miR expressed in immune cells is located within the 3′ UTR immediately after the stop codon, or within the 3′ UTR 15-20 nucleotides after the stop codon or within the 3′ UTR 70-80 nucleotides after the stop codon. In other embodiments, the 3′ UTR comprises more than one miRNA binding site (e.g., 2-4 miRNA binding sites), wherein there can be a spacer region (e.g., of 10-100, 20-70 or 30-50 nucleotides in length) between each miRNA binding site. In another embodiment, the 3′ UTR comprises a spacer region between the end of the miRNA binding site(s) and the poly A tail nucleotides. For example, a spacer region of 10-100, 20-70 or 30-50 nucleotides in length can be situated between the end of the miRNA binding site(s) and the beginning of the poly A tail.

In one embodiment, a codon optimized open reading frame encoding a polypeptide of interest comprises a start codon and the at least one microRNA binding site is located within the 5′ UTR 1-100 nucleotides before (upstream of) the start codon. In one embodiment, the codon optimized open reading frame encoding the polypeptide of interest comprises a start codon and the at least one microRNA binding site for a miR expressed in immune cells is located within the 5′ UTR 10-50 nucleotides before (upstream of) the start codon. In another embodiment, the codon optimized open reading frame encoding the polypeptide of interest comprises a start codon and the at least one microRNA binding site for a miR expressed in immune cells is located within the 5′ UTR at least 25 nucleotides before (upstream of) the start codon. In other embodiments, the codon optimized open reading frame encoding the polypeptide of interest comprises a start codon and the at least one microRNA binding site for a miR expressed in immune cells is located within the 5′ UTR immediately before the start codon, or within the 5′ UTR 15-20 nucleotides before the start codon or within the 5′ UTR 70-80 nucleotides before the start codon. In other embodiments, the 5′ UTR comprises more than one miRNA binding site (e.g., 2-4 miRNA binding sites), wherein there can be a spacer region (e.g., of 10-100, 20-70 or 30-50 nucleotides in length) between each miRNA binding site.

In one embodiment, the 3′ UTR comprises more than one stop codon, wherein at least one miRNA binding site is positioned downstream of the stop codons. For example, a 3′ UTR can comprise 1, 2 or 3 stop codons. Non-limiting examples of triple stop codons that can be used include: UGAUAAUAG, UGAUAGUAA, UAAUGAUAG, UGAUAAUAA, UGAUAGUAG, UAAUGAUGA, UAAUAGUAG, UGAUGAUGA, UAAUAAUAA, and UAGUAGUAG. Within a 3′ UTR, for example, 1, 2, 3 or 4 miRNA binding sites, e.g., miR-142-3p binding sites, can be positioned immediately adjacent to the stop codon(s) or at any number of nucleotides downstream of the final stop codon. When the 3′ UTR comprises multiple miRNA binding sites, these binding sites can be positioned directly next to each other in the construct (i.e., one after the other) or, alternatively, spacer nucleotides can be positioned between each binding site.

In one embodiment, the 3′ UTR comprises three stop codons with a single miR-142-3p binding site located downstream of the 3rd stop codon. Non-limiting examples of sequences of 3′ UTR having three stop codons and a single miR-142-3p binding site located at different positions downstream of the final stop codon are shown in SEQ ID NOs: 237, 248, 249, and 250.

TABLE 4A
5′ UTRs, 3′UTRs, miR sequences, and miR binding sites

| SEQ ID NO: | Sequence |
|---|---|
| 220 | GCUGGAGCCUCGGUGGCCAUGCUUCUUGCCCCUUGGGCCUCCCCCCAGC |
| | CCCUCCUCCCCUUCCUGCACCCGUACCCCCUCCAUAAAGUAGGAAACAC |
| | UACAGUGGUCUUUGAAUAAAGUCUGAGUGGGCGGC |
| | (3′ UTR with miR 142-3p binding site) |
| 202 | UCCAUAAAGUAGGAAACACUACA |
| | (miR 142-3p binding site) |
| 201 | UGUAGUGUUUCCUACUUUAUGGA |
| | (miR 142-3p sequence) |
| 203 | CAUAAAGUAGAAAGCACUACU |
| | (miR 142-5p sequence) |
| 221 | CCUCUGAAAUUCAGUUCUUCAG |
| | (miR 146-3p sequence) |
| 222 | UGAGAACUGAAUUCCAUGGGUU |
| | (miR 146-5p sequence) |
| 223 | CUCCUACAUAUUAGCAUUAACA |
| | (miR 155-3p sequence) |
| 224 | UUAAUGCUAAUCGUGAUAGGGGU |
| | (miR 155-5p sequence) |
| 206 | UCGUACCGUGAGUAAUAAUGCG |
| | (miR 126-3p sequence) |
| 208 | CAUUAUUACUUUUGGUACGCG |
| | (miR 126-5p sequence) |
| 225 | CCAGUAUUAACUGUGCUGCUGA |
| | (miR 16-3p sequence) |
| 226 | UAGCAGCACGUAAAUAUUGGCG |
| | (miR 16-5p sequence) |
| 227 | CAACACCAGUCGAUGGGCUGU |
| | (miR 21-3p sequence) |
| 228 | UAGCUUAUCAGACUGAUGUUGA |
| | (miR 21-5p sequence) |
| 143 | UGUCAGUUUGUCAAAUACCCCA |
| | (miR 223-3p sequence) |
| 230 | CGUGUAUUUGACAAGCUGAGUU |
| | (miR 223-5p sequence) |
| 231 | UGGCUCAGUUCAGCAGGAACAG |
| | (miR 24-3p sequence) |
| 232 | UGCCUACUGAGCUGAUAUCAGU |
| | (miR 24-5p sequence) |
| 233 | UUCACAGUGGCUAAGUUCCGC |
| | (miR 27-3p sequence) |
| 234 | AGGGCUUAGCUGCUUGUGAGCA |
| | (miR 27-5p sequence) |
| 207 | CGCAUUAUUACUCACGGUACGA |
| | (miR 126-3p binding site) |
| 235 | UGAUAAUAGGCUGGAGCCUCGGUGGCCAUGCUUCUUGCCCCUUGGGCC |
| | UCCCCCCAGCCCCUCCUCCCCUUCCUGCACCCGUACCCCC [symbol] |
| | [symbol] GUGGUCUUUGAAUAAAGUCUGAGUGGGCGGC |
| | (3′ UTR with miR 126-3p binding site) |
| 236 | UGAUAAUAGGCUGGAGCCUCGGUGGCCAUGCUUCUUGCCCCUUGGGCC |
| | UCCCCCCAGCCCCUCCUCCCCUUCCUGCACCCGUACCCCCGUGGUCUUU |
| | GAAUAAAGUCUGAGUGGGCGGC |
| | (3′ UTR, no miR binding sites) |
| 237 | UGAUAAUAGGCUGGAGCCUCGGUGGCCAUGCUUCUUGCCCCUUGGGCC |
| | UCCCCCCAGCCCCUCCUCCCCUUCCUGCACCCGUACCCCCUCCAUAAAG |
| | UAGGAAACACUACAGUGGUCUUUGAAUAAAGUCUGAGUGGGCGGC |
| | (3′ UTR with miR 142-3p binding site) |
| 199 | UGAUAAUAGUCCAUAAAGUAGGAAACACUACAGCUGGAGCCUCGGUGG |
| | CCAUGCUUCUUGCCCCUUGGGCCUCCCCCCAGCCCCUCCUCCCCUUCCU |
| | GCACCCGUACCCCC [symbol] GUGGUCUUUGAA |
| | UAAAGUCUGAGUGGGCGGC |
| | (3′ UTR with miR 142-3p and miR 126-3p binding sites variant 1) |
| 239 | UUAAUGCUAAUUGUGAUAGGGGU |
| | (miR 155-5p sequence) |
| 240 | ACCCCUAUCACAAUUAGCAUUAA |
| | (miR 155-5p binding site) |
| 241 | UGAUAAUAGUCCAUAAAGUAGGAAACACUACAGCUGGAGCCUCGGUGG |
| | CCAUGCUUCUUGCCCCUUGGGCCUCCAUAAAGUAGGAAACACUACAUC |
| | CCCCCAGCCCCUCCUCCCCUUCCUGCACCCGUACCCCCUCCAUAAAGUA |
| | GGAAACACUACAGUGGUCUUUGAAUAAAGUCUGAGUGGGCGGC |
| | (3′ UTR with 3 miR 142-3p binding sites) |
| 242 | UGAUAAUAGGCUGGAGCCUCGGUGGCCAUGCUUCUUGCCCCUUGGGCC |
| | UCCCCCCAGCCCCUCCUCCCCUUCCUGCACCCGUACCCCC [symbol] |
| | [symbol] GUGGUCUUUGAAUAAAGUCUGAGUGGGCGGC |
| | (3′ UTR with miR 142-5p binding site) |
| 243 | UGAUAAUAG [symbol] GCUGGAGCCUCGGUGGCC |
| | AUGCUUCUUGCCCCUUGGGCC [symbol] UCCCCCC |
| | AGCCCCUCCUCCCCUUCCUGCACCCGUACCCCC [symbol] |
| | [symbol] GUGGUCUUUGAAUAAAGUCUGAGUGGGCGGC |
| | (3′ UTR with 3 miR 142-5p binding sites) |
| 244 | UGAUAAUAG [symbol] GCUGGAGCCUCGGUGGCC |
| | AUGCUUCUUGCCCCUUGGGCCUCCAUAAAGUAGGAAACACUACAUCCC |
| | CCCAGCCCCUCCUCCCCUUCCUGCACCCGUACCCCC [symbol] |
| | [symbol] GUGGUCUUUGAAUAAAGUCUGAGUGGGCGGC |
| | (3′ UTR with 2 miR 142-5p binding sites and 1 miR 142-3p binding site) |
| 245 | UGAUAAUAGGCUGGAGCCUCGGUGGCCAUGCUUCUUGCCCCUUGGGCC |
| | UCCCCCCAGCCCCUCCUCCCCUUCCUGCACCCGUACCCCCACCCCUAUCA |
| | CAAUUAGCAUUAAGUGGUCUUUGAAUAAAGUCUGAGUGGGCGGC |
| | (3′ UTR with miR 155-5p binding site) |
| 246 | UGAUAAUAGACCCCUAUCACAAUUAGCAUUAAGCUGGAGCCUCGGUGGC |
| | CAUGCUUCUUGCCCCUUGGGCCACCCCUAUCACAAUUAGCAUUAAUCCCC |
| | CCAGCCCCUCCUCCCCUUCCUGCACCCGUACCCCCACCCCUAUCACAAUU |
| | AGCAUUAAGUGGUCUUUGAAUAAAGUCUGAGUGGGCGGC |
| | (3′ UTR with 3 miR 155-5p binding sites) |
| 247 | UGAUAAUAGACCCCUAUCACAAUUAGCAUUAAGCUGGAGCCUCGGUGGC |
| | CAUGCUUCUUGCCCCUUGGGCCUCCAUAAAGUAGGAAACACUACAUCC |
| | CCCCAGCCCCUCCUCCCCUUCCUGCACCCGUACCCCCACCCCUAUCACAA |
| | UUAGCAUUAAGUGGUCUUUGAAUAAAGUCUGAGUGGGCGGC |
| | (3′ UTR with 2 miR 155-5p binding sites and 1 miR 142-3p binding site) |
| 248 | UGAUAAUAGUCCAUAAAGUAGGAAACACUACAGCUGGAGCCUCGGUGG |
| | CCAUGCUUCUUGCCCCUUGGGCCUCCCCCCAGCCCCUCCUCCCCUUCCU |
| | GCACCCGUACCCCCGUGGUCUUUGAAUAAAGUCUGAGUGGGCGGC |
| | (3′ UTR with miR 142-3p binding site, P1 insertion) |
| 249 | UGAUAAUAGGCUGGAGCCUCGGUGGCUCCAUAAAGUAGGAAACACUAC |
| | ACAUGCUUCUUGCCCCUUGGGCCUCCCCCCAGCCCCUCCUCCCCUUCCU |
| | GCACCCGUACCCCCGUGGUCUUUGAAUAAAGUCUGAGUGGGCGGC |
| | (3′ UTR with miR 142-3p binding site, P2 insertion) |
| 250 | UGAUAAUAGGCUGGAGCCUCGGUGGCCAUGCUUCUUGCCCCUUGGGCC |
| | UCCAUAAAGUAGGAAACACUACAUCCCCCCAGCCCCUCCUCCCCUUCCU |
| | GCACCCGUACCCCCGUGGUCUUUGAAUAAAGUCUGAGUGGGCGGC |
| | (3′ UTR with miR 142-3p binding site, P3 insertion) |
| 204 | AGUAGUGCUUUCUACUUUAUG |
| | (miR-142-5p binding site) |
| 200 | GACAGUGCAGUCACCCAUAAAGUAGAAAGCACUACUAACAGCACUGGA |
| | GGGUGUAGUGUUUCCUACUUUAUGGAUGAGUGUACUGUG |
| | (miR-142) |
| 708 | GGGAAAUAAGAGAGAAAAGAAGAGUAAGAAGAAAUAUAAGAGCCACC |
| | (5′ UTR) |
| 251 | GGGAAAUAAGAGUCCAUAAAGUAGGAAACACUACAAGAAAAGAAGAGU |
| | AAGAAGAAAUAUAAGAGCCACC |
| | (5′ UTR with miR142-3p binding site at position p1) |
| 252 | GGGAAAUAAGAGAGAAAAGAAGAGUAAUCCAUAAAGUAGGAAACACU |
| | ACAGAAGAAAUAUAAGAGCCACC |
| | (5′ UTR with miR142-3p binding site at position p2) |
| 253 | GGGAAAUAAGAGAGAAAAGAAGAGUAAGAAGAAAUAUAAUCCAUAAA |
| | GUAGGAAACACUACAGAGCCACC |
| | (5′ UTR with miR142-3p binding site at position p3) |
| 254 | ACCCCUAUCACAAUUAGCAUUAA |
| | (miR 155-5p binding site) |
| 255 | UGAUAAUAG [symbol] GCUGGAGCCUCGGUGGCC |
| | AUGCUUCUUGCCCCUUGGGCC [symbol] UCCCCCC |
| | AGCCCCUCUCCCCUUCCUGCACCCGUACCCCC [symbol] |
| | [symbol] GUGGUCUUUGAAUAAAGUCUGAGUGGGCGGC |
| | (3′ UTR with 3 miR 142-5p binding sites) |
| 256 | UGAUAAUAGGCUGGAGCCUCGGUGGCCAUGCUUCUUGCCCCUUCCAUA |
| | AAGUAGGAAACACUACAUGGGCCUCCCCCCAGCCCCUCCUCCCCUUCCU |
| | GCACCCGUACCCCCGUGGUCUUUGAAUAAAGUCUGAGUGGGCGGC |
| | (3′UTR including miR142-3p binding site) |
| 257 | UGAUAAUAGGCUGGAGCCUCGGUGGCCAUGCUUCUUGCCCCUUGGGCC |
| | UCCCCCCAGUCCAUAAAGUAGGAAACACUACACCCCUCCUCCCCUUCCU |
| | GCACCCGUACCCCCGUGGUCUUUGAAUAAAGUCUGAGUGGGCGGC |
| | (3′UTR including miR142-3p binding site) |
| 258 | UGAUAAUAGGCUGGAGCCUCGGUGGCCAUGCUUCUUGCCCCUUGGGCC |
| | UCCCCCCAGCCCCUCCUCCCCUUCUCCAUAAAGUAGGAAACACUACACU |
| | GCACCCGUACCCCCGUGGUCUUUGAAUAAAGUCUGAGUGGGCGGC |
| | (3′UTR including miR142-3p binding site) |
| 259 | UGAUAAUAGGCUGGAGCCUCGGUGGCCAUGCUUCUUGCCCCUUGGGCC |
| | UCCCCCCAGCCCCUCCUCCCCUUCCUGCACCCGUACCCCCGUGGUCUUU |
| | GAAUAAAGUUCCAUAAAGUAGGAAACACUACACUGAGUGGGCGGC |
| | (3′UTR including miR142-3p binding site) |
| 260 | UGAUAAUAGUCCAUAAAGUAGGAAACACUACAGCUGGAGCCUCGGUGG |
| | CCUAGCUUCUUGCCCCUUGGGCCUCCCCCCAGCCCCUCCUCCCCUUCCU |
| | GCACCCGUACCCCC [symbol] GUGGUCUUUGAA |
| | UAAAGUCUGAGUGGGCGGC |
| | (3′ UTR with miR 142-3p and miR 126-3p binding sites variant 2) |
| 261 | UGAUAAUAGGCUGGAGCCUCGGUGGCCUAGCUUCUUGCCCCUUGGGCC |
| | UCCCCCCAGCCCCUCCUCCCCUUCCUGCACCCGUACCCCCGUGGUCUUU |
| | GAAUAAAGUCUGAGUGGGCGGC |
| | (3′ UTR, no miR binding sites variant 2) |
| 111 | UGAUAAUAGGCUGGAGCCUCGGUGGCCUAGCUUCUUGCCCCUUGGGCC |
| | UCCCCCCAGCCCCUCCUCCCCUUCCUGCACCCGUACCCCCUCCAUAAAG |
| | UAGGAAACACUACAGUGGUCUUUGAAUAAAGUCUGAGUGGGCGGC |
| | (3′ UTR with miR 142-3p binding site variant 3) |
| 262 | UGAUAAUAGGCUGGAGCCUCGGUGGCCUAGCUUCUUGCCCCUUGGGCC |
| | UCCCCCCAGCCCCUCCUCCCCUUCCUGCACCCGUACCCCC [symbol] |
| | [symbol] GUGGUCUUUGAAUAAAGUCUGAGUGGGCGGC |
| | (3′ UTR with miR 126-3p binding site variant 3) |
| 263 | UGAUAAUAGUCCAUAAAGUAGGAAACACUACAGCUGGAGCCUCGGUGG |
| | CCUAGCUUCUUGCCCCUUGGGCCUCCAUAAAGUAGGAAACACUACAUC |
| | CCCCCAGCCCCUCCUCCCCUUCCUGCACCCGUACCCCCUCCAUAAAGUA |
| | GGAAACACUACAGUGGUCUUUGAAUAAAGUCUGAGUGGGGGC |
| | (3′ UTR with 3 miR 142-3p binding sites variant 2) |
| 264 | UGAUAAUAGUCCAUAAAGUAGGAAACACUACAGCUGGAGCCUCGGUGG |
| | CCUAGCUUCUUGCCCCUUGGGCCUCCCCCCAGCCCCUCCUCCCCUUCCU |
| | GCACCCGUACCCCCGUGGUCUUUGAAUAAAGUCUGAGUGGGGGC |
| | (3′UTR with miR 142-3p binding site, P1 insertion variant 2) |
| 265 | UGAUAAUAGGCUGGAGCCUCGGUGGCUCCAUAAAGUAGGAAACACUAC |
| | ACUAGCUUCUUGCCCCUUGGGCCUCCCCCCAGCCCCUCCUCCCCUUCCU |
| | GCACCCGUACCCCCGUGGUCUUUGAAUAAAGUCUGAGUGGGCGGC |
| | (3′UTR with miR 142-3p binding site, P2 insertion variant 2) |
| 266 | UGAUAAUAGGCUGGAGCCUCGGUGGCCUAGCUUCUUGCCCCUUGGGCC |
| | UCCAUAAAGUAGGAAACACUACAUCCCCCCAGCCCCUCCUCCCCUUCCU |
| | GCACCCGUACCCCCGUGGUCUUUGAAUAAAGUCUGAGUGGGCGGC |
| | (3′UTR with miR 142-3p binding site, P3 insertion variant 2) |
| 267 | UGAUAAUAGGCUGGAGCCUCGGUGGCCUAGCUUCUUGCCCCUUGGGCC |
| | UCCCCCCAGCCCCUCCUCCCCUUCCUGCACCCGUACCCCCACCCCUAUCA |
| | CAAUUAGCAUUAAGUGGUCUUUGAAUAAAGUCUGAGUGGGCGGC |
| | (3′UTR with miR 155-5p binding site variant 2) |
| 268 | UGAUAAUAGACCCCUAUCACAAUUAGCAUUAAGCUGGAGCCUCGGUGGC |
| | CUAGCUUCUUGCCCCUUGGGCCACCCCUAUCACAAUUAGCAUUAAUCCCC |
| | CCAGCCCCUCCUCCCCUUCCUGCACCCGUACCCCCACCCCUAUCACAAUU |
| | AGCAUUAAGUGGUCUUUGAAUAAAGUCUGAGUGGGCGGC |
| | (3′ UTR with 3 miR 155-5p binding sites variant 2) |
| 269 | UGAUAAUAGACCCCUAUCACAAUUAGCAUUAAGCUGGAGCCUCGGUGGC |
| | CUAGCUUCUUGCCCCUUGGGCCUCCAUAAAGUAGGAAACACUACAUCC |
| | CCCCAGCCCCUCCUCCCCUUCCUGCACCCGUACCCCCACCCCUAUCACAA |
| | UUAGCAUUAAGUGGUCUUUGAAUAAAGUCUGAGUGGGCGGC |
| | (3′UTR with 2 miR 155-5p binding sites and 1 miR 142-3p binding site |
| | variant 2) |
| 271 | AGGAAAUAAGAGAGAAAAGAAGAGUAAGAAGAAAUAUAAGAGCCACC |
| | 5′-UTR (v1 plus A-start) |
| 272 | GGGAAAUAAGAGAGAAAAGAAGAGUAAGAAGAAAUAUAAGACCCCGGC |
| | GCCGCCACC |
| | (5′ UTR v1.1 plus G-start) |
| 273 | AGGAAAUAAGAGAGAAAAGAAGAGUAAGAAGAAAUAUAAGACCCCGGC |
| | GCCGCCACC |
| | (5′UTR v1.1 plus A-start) |
| 274 | GGGAGAUCAGAGAGAAAAGAAGAGUAAGAAGAAAUAUAAGAGCCACC |
| | 5′ UTR 002 (upstream UTR plus G-start) |
| 80 | GGGAGACAAGCUUGGCAUUCCGGUACUGUUGGUAAAGCCACC |
| | 5′ UTR-004 (Upstream UTR plus G-start) |
| 81 | GGGAAUUAACAGAGAAAAGAAGAGUAAGAAGAAAUAUAAGAGCCACC |
| | 5′ UTR-008 (Upstream UTR plus G-start) |
| 82 | GGGAAAUUAGACAGAAAAGAAGAGUAAGAAGAAAUAUAAGAGCCACC |
| | 5′ UTR-009 (Upstream UTR plus G-start) |
| 83 | GGGAAAUAAGAGAGUAAAGAACAGUAAGAAGAAAUAUAAGAGCCACC |
| | 5′ UTR-010, (Upstream UTR plus G-start) |
| 84 | GGGAAAAAAGAGAGAAAAGAAGACUAAGAAGAAAUAUAAGAGCCACC |
| | 5′ UTR-011 (Upstream UTR plus G-start) |
| 85 | GGGAAAUAAGAGAGAAAAGAAGAGUAAGAAGAUAUAUAAGAGCCACC |
| | 5′ UTR-012 (Upstream UTR plus G-start) |
| 86 | GGGAAAUAAGAGACAAAACAAGAGUAAGAAGAAAUAUAAGAGCCACC |
| | 5′ UTR-013 (Upstream UTR plus G-start) |
| 87 | GGGAAAUUAGAGAGUAAAGAACAGUAAGUAGAAUUAAAAGAGCCACC |
| | 5′ UTR-014 (Upstream UTR plus G-start) |
| 88 | GGGAAAUAAGAGAGAAUAGAAGAGUAAGAAGAAAUAUAAGAGCCACC |
| | 5′ UTR-015 (Upstream UTR plus G-start) |
| 89 | GGGAAAUAAGAGAGAAAAGAAGAGUAAGAAGAAAAUUAAGAGCCACC |
| | 5′ UTR-016 (Upstream UTR plus G-start) |
| 90 | GGGAAAUAAGAGAGAAAAGAAGAGUAAGAAGAAAUUUAAGAGCCACC |
| | 5′ UTR-017 (Upstream UTR plus G-start) |
| 91 | UCAAGCUUUUGGACCCUCGUACAGAAGCUAAUACGACUCACUAUAGGG |
| | AAAUAAGAGAGAAAAGAAGAGUAAGAAGAAAUAUAAGAGCCACC |
| | 5′ UTR-018 (Upstream UTR) |

Stop codon = bold
miR 142-3p binding site = underline
miR 126-3p binding site = bold underline
miR 155-5p binding site = italicized
miR 142-5p binding site = italicized and bold underline

In one embodiment, the polynucleotide of the invention comprises a 5′ UTR, a codon optimized open reading frame encoding a polypeptide of interest, a 3′ UTR comprising the at least one miRNA binding site for a miR expressed in immune cells, and a 3′ tailing region of linked nucleosides. In various embodiments, the 3′ UTR comprises 1-4, at least two, one, two, three, or four miRNA binding sites for miRs expressed in immune cells, preferably abundantly or preferentially expressed in immune cells.

In one embodiment, the at least one miRNA expressed in immune cells is a miR-142-3p microRNA binding site. In one embodiment, the miR-142-3p microRNA binding site comprises the sequence shown in SEQ ID NO: 202. In one embodiment, the 3′ UTR of the mRNA comprising the miR-142-3p microRNA binding site comprises the sequence shown in SEQ ID NO: 220.

In one embodiment, the at least one miRNA expressed in immune cells is a miR-126 microRNA binding site. In one embodiment, the miR-126 binding site is a miR-126-3p binding site. In one embodiment, the miR-126-3p microRNA binding site comprises the sequence shown in SEQ ID NO: 207. In one embodiment, the 3′ UTR of the mRNA of the invention comprising the miR-126-3p microRNA binding site comprises the sequence shown in SEQ ID NO: 235.

Non-limiting exemplary sequences for miRs to which a microRNA binding site(s) of the disclosure can bind include the following: miR-142-3p (SEQ ID NO: 201), miR-142-5p (SEQ ID NO: 203), miR-146-3p (SEQ ID NO: 221), miR-146-5p (SEQ ID NO: 222), miR-155-3p (SEQ ID NO: 223), miR-155-5p (SEQ ID NO: 224), miR-126-3p (SEQ ID NO: 206), miR-126-5p (SEQ ID NO: 208), miR-16-3p (SEQ ID NO: 225), miR-16-5p (SEQ ID NO: 226), miR-21-3p (SEQ ID NO: 227), miR-21-5p (SEQ ID NO: 228), miR-223-3p (SEQ ID NO: 143), miR-223-5p (SEQ ID NO: 230), miR-24-3p (SEQ ID NO: 231), miR-24-5p (SEQ ID NO: 232), miR-27-3p (SEQ ID NO: 233) and miR-27-5p (SEQ ID NO: 234). Other suitable miR sequences expressed in immune cells (e.g., abundantly or preferentially expressed in immune cells) are known and available in the art, for example at the University of Manchester's microRNA database, miRBase. Sites that bind any of the aforementioned miRs can be designed based on Watson-Crick complementarity to the miR, typically 100% complementarity to the miR, and inserted into an mRNA construct of the disclosure as described herein.

In another embodiment, a polynucleotide of the present invention (e.g., and mRNA, e.g., the 3′ UTR thereof) can comprise at least one miRNA binding site to thereby reduce or inhibit accelerated blood clearance, for example by reducing or inhibiting production of IgMs, e.g., against PEG, by B cells and/or reducing or inhibiting proliferation and/or activation of pDCs, and can comprise at least one miRNA binding site for modulating tissue expression of an encoded protein of interest.

miRNA gene regulation can be influenced by the sequence surrounding the miRNA such as, but not limited to, the species of the surrounding sequence, the type of sequence (e.g., heterologous, homologous, exogenous, endogenous, or artificial), regulatory elements in the surrounding sequence and/or structural elements in the surrounding sequence. The miRNA can be influenced by the 5′UTR and/or 3′UTR. As a non-limiting example, a non-human 3′UTR can increase the regulatory effect of the miRNA sequence on the expression of a polypeptide of interest compared to a human 3′ UTR of the same sequence type.

In one embodiment, other regulatory elements and/or structural elements of the 5′ UTR can influence miRNA mediated gene regulation. One example of a regulatory element and/or structural element is a structured IRES (Internal Ribosome Entry Site) in the 5′ UTR, which is necessary for the binding of translational elongation factors to initiate protein translation. EIF4A2 binding to this secondarily structured element in the 5′-UTR is necessary for miRNA mediated gene expression (Meijer H A et al., Science, 2013, 340, 82-85, herein incorporated by reference in its entirety). The polynucleotides of the invention can further include this structured 5′ UTR to enhance microRNA mediated gene regulation.

At least one miRNA binding site can be engineered into the 3′ UTR of a polynucleotide of the invention. In this context, at least two, at least three, at least four, at least five, at least six, at least seven, at least eight, at least nine, at least ten, or more miRNA binding sites can be engineered into a 3′ UTR of a polynucleotide of the invention. For example, 1 to 10, 1 to 9, 1 to 8, 1 to 7, 1 to 6, 1 to 5, 1 to 4, 1 to 3, 2, or 1 miRNA binding sites can be engineered into the 3′UTR of a polynucleotide of the invention. In one embodiment, miRNA binding sites incorporated into a polynucleotide of the invention can be the same or can be different miRNA sites. A combination of different miRNA binding sites incorporated into a polynucleotide of the invention can include combinations in which more than one copy of any of the different miRNA sites are incorporated. In another embodiment, miRNA binding sites incorporated into a polynucleotide of the invention can target the same or different tissues in the body. As a non-limiting example, through the introduction of tissue-, cell-type-, or disease-specific miRNA binding sites in the 3′-UTR of a polynucleotide of the invention, the degree of expression in specific cell types (e.g., myeloid cells, endothelial cells, etc.) can be reduced.

In one embodiment, a miRNA binding site can be engineered near the 5′ terminus of the 3′UTR, about halfway between the 5′ terminus and 3′ terminus of the 3′UTR and/or near the 3′ terminus of the 3′ UTR in a polynucleotide of the invention. As a non-limiting example, a miRNA binding site can be engineered near the 5′ terminus of the 3′UTR and about halfway between the 5′ terminus and 3′ terminus of the 3′UTR. As another non-limiting example, a miRNA binding site can be engineered near the 3′ terminus of the 3′UTR and about halfway between the 5′ terminus and 3′ terminus of the 3′ UTR. In another non-limiting example, a miRNA binding site can be engineered near the 5′ terminus of the 3′ UTR and near the 3′ terminus of the 3′ UTR.

In another embodiment, a 3′UTR can comprise 1, 2, 3, 4, 5, 6, 7, 8, 9, or 10 miRNA binding sites. The miRNA binding sites can be complementary to a miRNA, miRNA seed sequence, and/or miRNA sequences flanking the seed sequence.

In some embodiments, the expression of a polynucleotide of the invention can be controlled by incorporating at least one sensor sequence in the polynucleotide and formulating the polynucleotide for administration. As a non-limiting example, a polynucleotide of the invention can be targeted to a tissue or cell by incorporating a miRNA binding site and formulating the polynucleotide in a lipid nanoparticle comprising an ionizable lipid, including any of the lipids described herein.

A polynucleotide of the invention can be engineered for more targeted expression in specific tissues, cell types, or biological conditions based on the expression patterns of miRNAs in the different tissues, cell types, or biological conditions. Through introduction of tissue-specific miRNA binding sites, a polynucleotide of the invention can be designed for optimal protein expression in a tissue or cell, or in the context of a biological condition.

In some embodiments, a polynucleotide of the invention can be designed to incorporate miRNA binding sites that either have 100% identity to known miRNA seed sequences or have less than 100% identity to miRNA seed sequences. In some embodiments, a polynucleotide of the invention can be designed to incorporate miRNA binding sites that have at least: 60%, 65%, 70%, 75%, 80%, 85%, 90%, 95%, 96%, 97%, 98%, or 99% identity to known miRNA seed sequences. The miRNA seed sequence can be partially mutated to decrease miRNA binding affinity and as such result in reduced downmodulation of the polynucleotide. In essence, the degree of match or mis-match between the miRNA binding site and the miRNA seed can act as a rheostat to more finely tune the ability of the miRNA to modulate protein expression. In addition, mutation in the non-seed region of a miRNA binding site can also impact the ability of a miRNA to modulate protein expression.

In one embodiment, a miRNA sequence can be incorporated into the loop of a stem loop.

In another embodiment, a miRNA seed sequence can be incorporated in the loop of a stem loop and a miRNA binding site can be incorporated into the 5′ or 3′ stem of the stem loop.

In one embodiment the miRNA sequence in the 5′ UTR can be used to stabilize a polynucleotide of the invention described herein.

In another embodiment, a miRNA sequence in the 5′ UTR of a polynucleotide of the invention can be used to decrease the accessibility of the site of translation initiation such as, but not limited to a start codon. See, e.g., Matsuda et al., PLOS One. 2010 11 (5): e15057; incorporated herein by reference in its entirety, which used antisense locked nucleic acid (LNA) oligonucleotides and exon-junction complexes (EJCs) around a start codon (−4 to +37 where the A of the AUG codons is +1) to decrease the accessibility to the first start codon (AUG). Matsuda showed that altering the sequence around the start codon with an LNA or EJC affected the efficiency, length and structural stability of a polynucleotide. A polynucleotide of the invention can comprise a miRNA sequence, instead of the LNA or EJC sequence described by Matsuda et al, near the site of translation initiation to decrease the accessibility to the site of translation initiation. The site of translation initiation can be prior to, after or within the miRNA sequence. As a non-limiting example, the site of translation initiation can be located within a miRNA sequence such as a seed sequence or binding site.

In some embodiments, a polynucleotide of the invention can include at least one miRNA to dampen the antigen presentation by antigen presenting cells. The miRNA can be the complete miRNA sequence, the miRNA seed sequence, the miRNA sequence without the seed, or a combination thereof. As a non-limiting example, a miRNA incorporated into a polynucleotide of the invention can be specific to the hematopoietic system. As another non-limiting example, a miRNA incorporated into a polynucleotide of the invention to dampen antigen presentation is miR-142-3p.

In some embodiments, a polynucleotide of the invention can include at least one miRNA to dampen expression of the encoded polypeptide in a tissue or cell of interest. As a non-limiting example, a polynucleotide of the invention can include at least one miR-142-3p binding site, miR-142-3p seed sequence, miR-142-3p binding site without the seed, miR-142-5p binding site, miR-142-5p seed sequence, miR-142-5p binding site without the seed, miR-146 binding site, miR-146 seed sequence and/or miR-146 binding site without the seed sequence.

In some embodiments, a polynucleotide of the invention can comprise at least one miRNA binding site in the 3′UTR to selectively degrade mRNA therapeutics in the immune cells to subdue unwanted immunogenic reactions caused by therapeutic delivery. As a non-limiting example, the miRNA binding site can make a polynucleotide of the invention more unstable in antigen presenting cells. Non-limiting examples of these miRNAs include miR-142-5p, miR-142-3p, miR-146a-5p, and miR-146-3p.

In one embodiment, a polynucleotide of the invention comprises at least one miRNA sequence in a region of the polynucleotide that can interact with a RNA binding protein.

In some embodiments, the polynucleotide of the invention (e.g., a RNA, e.g., an mRNA) comprising (i) a sequence-optimized nucleotide sequence (e.g., an ORF) encoding a checkpoint cancer vaccine comprising (i) one or more IDO antigenic peptides and (ii) one or more PD-L1 antigenic peptides and (ii) a miRNA binding site (e.g., a miRNA binding site that binds to miR-142) and/or a miRNA binding site that binds to miR-126.

### IVT Polynucleotide Architecture

In some embodiments, the polynucleotide of the present disclosure comprising an mRNA encoding a checkpoint cancer vaccine comprising (i) one or more IDO antigenic peptides and (ii) one or more PD-L1 antigenic peptides is an IVT polynucleotide. Traditionally, the basic components of an mRNA molecule include at least a coding region, a 5′UTR, a 3′UTR, a 5′ cap and a poly-A tail. The IVT polynucleotides of the present disclosure can function as mRNA but are distinguished from wild-type mRNA in their functional and/or structural design features which serve, e.g., to overcome existing problems of effective polypeptide production using nucleic-acid based therapeutics.

The primary construct of an IVT polynucleotide comprises a first region of linked nucleotides that is flanked by a first flanking region and a second flaking region. This first region can include, but is not limited to, the encoded a checkpoint cancer vaccine. The first flanking region can include a sequence of linked nucleosides which function as a 5′ untranslated region (UTR) such as the 5′ UTR of any of the nucleic acids encoding the native 5′ UTR of the polypeptide or a non-native 5′UTR such as, but not limited to, a heterologous 5′ UTR or a synthetic 5′ UTR. The IVT encoding a checkpoint cancer vaccine comprising (i) one or more IDO antigenic peptides and (ii) one or more PD-L1 antigenic peptides can comprise at its 5 terminus a signal sequence region encoding one or more signal sequences. The flanking region can comprise a region of linked nucleotides comprising one or more complete or incomplete 5′ UTRs sequences. The flanking region can also comprise a 5′ terminal cap. The second flanking region can comprise a region of linked nucleotides comprising one or more complete or incomplete 3′ UTRs which can encode the native 3′ UTR of a checkpoint cancer vaccine or a non-native 3′ UTR such as, but not limited to, a heterologous 3′ UTR or a synthetic 3′ UTR. The flanking region can also comprise a 3′ tailing sequence. The 3′ tailing sequence can be, but is not limited to, a polyA tail, a polyA-G quartet and/or a stem loop sequence.

Additional and exemplary features of IVT polynucleotide architecture are disclosed in International PCT application WO 2017/201325, filed on 18 May 2017, the entire contents of which are hereby incorporated by reference.

### 5′UTR and 3′ UTR

A UTR can be homologous or heterologous to the coding region in a polynucleotide. In some embodiments, the UTR is homologous to the ORF encoding the checkpoint cancer vaccine comprising (i) one or more IDO antigenic peptides and (ii) one or more PD-L1 antigenic peptides

In some embodiments, the polynucleotide comprises two or more 5′ UTRs or functional fragments thereof, each of which has the same or different nucleotide sequences. In some embodiments, the polynucleotide comprises two or more 3′ UTRs or functional fragments thereof, each of which has the same or different nucleotide sequences.

In some embodiments, the 5′ UTR or functional fragment thereof, 3′ UTR or functional fragment thereof, or any combination thereof is sequence optimized.

In some embodiments, the 5′UTR or functional fragment thereof, 3′ UTR or functional fragment thereof, or any combination thereof comprises at least one chemically modified nucleobase, e.g., N1-methylpseudouracil or 5-methoxyuracil.

UTRs can have features that provide a regulatory role, e.g., increased or decreased stability, localization and/or translation efficiency. A polynucleotide comprising a UTR can be administered to a cell, tissue, or organism, and one or more regulatory features can be measured using routine methods. In some embodiments, a functional fragment of a 5′ UTR or 3′ UTR comprises one or more regulatory features of a full length 5′ or 3′ UTR, respectively.

Natural 5′UTRs bear features that play roles in translation initiation. They harbor signatures like Kozak sequences that are commonly known to be involved in the process by which the ribosome initiates translation of many genes. Kozak sequences have the consensus CCR(A/G)CCAUGG, where R is a purine (adenine or guanine) three bases upstream of the start codon (AUG), which is followed by another ‘G’. 5′ UTRs also have been known to form secondary structures that are involved in elongation factor binding.

By engineering the features typically found in abundantly expressed genes of specific target organs, one can enhance the stability and protein production of a polynucleotide. For example, introduction of 5′ UTR of liver-expressed mRNA, such as albumin, serum amyloid A, Apolipoprotein A/B/E, transferrin, alpha fetoprotein, erythropoietin, or Factor VIII, can enhance expression of polynucleotides in hepatic cell lines or liver. Likewise, use of 5′UTR from other tissue-specific mRNA to improve expression in that tissue is possible for muscle (e.g., MyoD, Myosin, Myoglobin, Myogenin, Herculin), for endothelial cells (e.g., Tie-1, CD36), for myeloid cells (e.g., C/EBP, AML1, G-CSF, GM-CSF, CD11b, MSR, Fr-1, i-NOS), for leukocytes (e.g., CD45, CD18), for adipose tissue (e.g., CD36, GLUT4, ACRP30, adiponectin) and for lung epithelial cells (e.g., SP-A/B/C/D).

In some embodiments, UTRs are selected from a family of transcripts whose proteins share a common function, structure, feature or property. For example, an encoded polypeptide can belong to a family of proteins (i.e., that share at least one function, structure, feature, localization, origin, or expression pattern), which are expressed in a particular cell, tissue or at some time during development. The UTRs from any of the genes or mRNA can be swapped for any other UTR of the same or different family of proteins to create a new polynucleotide.

In some embodiments, the 5′ UTR and the 3′ UTR can be heterologous. In some embodiments, the 5′ UTR can be derived from a different species than the 3′ UTR. In some embodiments, the 3′ UTR can be derived from a different species than the 5′ UTR.

Co-owned International Patent Application No. PCT/US2014/021522 (Publ. No. WO/2014/164253, incorporated herein by reference in its entirety) provides a listing of exemplary UTRs that can be utilized in the polynucleotide of the present invention as flanking regions to an ORF.

Additional exemplary UTRs of the application include, but are not limited to, one or more 5′UTR and/or 3′UTR derived from the nucleic acid sequence of: a globin, such as an α- or β-globin (e.g., a Xenopus, mouse, rabbit, or human globin); a strong Kozak translational initiation signal; a CYBA (e.g., human cytochrome b-245 α polypeptide); an albumin (e.g., human albumin7); a HSD17B4 (hydroxysteroid (17-β) dehydrogenase); a virus (e.g., a tobacco etch virus (TEV), a Venezuelan equine encephalitis virus (VEEV), a Dengue virus, a cytomegalovirus (CMV) (e.g., CMV immediate early 1 (IE1)), a hepatitis virus (e.g., hepatitis B virus), a sindbis virus, or a PAV barley yellow dwarf virus); a heat shock protein (e.g., hsp70); a translation initiation factor (e.g., elF4G); a glucose transporter (e.g., hGLUT1 (human glucose transporter 1)); an actin (e.g., human a or β actin); a GAPDH; a tubulin; a histone; a citric acid cycle enzyme; a topoisomerase (e.g., a 5′UTR of a TOP gene lacking the 5′ TOP motif (the oligopyrimidine tract)); a ribosomal protein Large 32 (L32); a ribosomal protein (e.g., human or mouse ribosomal protein, such as, for example, rps9); an ATP synthase (e.g., ATP5A1 or the β subunit of mitochondrial H⁺-ATP synthase); a growth hormone e (e.g., bovine (bGH) or human (hGH)); an elongation factor (e.g., elongation factor 1 α1 (EEF1A1)); a manganese superoxide dismutase (MnSOD); a myocyte enhancer factor 2A (MEF2A); a β-F1-ATPase, a creatine kinase, a myoglobin, a granulocyte-colony stimulating factor (G-CSF); a collagen (e.g., collagen type I, alpha 2 (Col1A2), collagen type I, alpha 1 (Col1A1), collagen type VI, alpha 2 (Col6A2), collagen type VI, alpha 1 (Col6A1)); a ribophorin (e.g., ribophorin I (RPNI)); a low density lipoprotein receptor-related protein (e.g., LRP1); a cardiotrophin-like cytokine factor (e.g., Nnt1); calreticulin (Calr); a procollagen-lysine, 2-oxoglutarate 5-dioxygenase 1 (Plod1); and a nucleobindin (e.g., Nucb1).

In some embodiments, the 5′ UTR is selected from the group consisting of a β-globin 5′ UTR; a 5′UTR containing a strong Kozak translational initiation signal; a cytochrome b-245 α polypeptide (CYBA) 5′ UTR; a hydroxysteroid (17-β) dehydrogenase (HSD17B4) 5′ UTR; a Tobacco etch virus (TEV) 5′ UTR; a Venezuelan equine encephalitis virus (TEEV) 5′ UTR; a 5′ proximal open reading frame of rubella virus (RV) RNA encoding nonstructural proteins; a Dengue virus (DEN) 5′ UTR; a heat shock protein 70 (Hsp70) 5′ UTR; a eIF4G 5′ UTR; a GLUT1 5′ UTR; functional fragments thereof and any combination thereof.

In some embodiments, the 3′ UTR is selected from the group consisting of a β-globin 3′ UTR; a CYBA 3′ UTR; an albumin 3′ UTR; a growth hormone (GH) 3′ UTR; a VEEV 3′ UTR; a hepatitis B virus (HBV) 3′ UTR; α-globin 3′UTR; a DEN 3′ UTR; a PAV barley yellow dwarf virus (BYDV-PAV) 3′ UTR; an elongation factor 1 α1 (EEF1A1) 3′ UTR; a manganese superoxide dismutase (MnSOD) 3′ UTR; a β subunit of mitochondrial H(+)-ATP synthase (β-mRNA) 3′ UTR; a GLUT1 3′ UTR; a MEF2A 3′ UTR; a β-F1-ATPase 3′ UTR; functional fragments thereof and combinations thereof.

Wild-type UTRs derived from any gene or mRNA can be incorporated into the polynucleotides of the invention. In some embodiments, a UTR can be altered relative to a wild type or native UTR to produce a variant UTR, e.g., by changing the orientation or location of the UTR relative to the ORF; or by inclusion of additional nucleotides, deletion of nucleotides, swapping or transposition of nucleotides. In some embodiments, variants of 5′ or 3′ UTRs can be utilized, for example, mutants of wild type UTRs, or variants wherein one or more nucleotides are added to or removed from a terminus of the UTR.

Additionally, one or more synthetic UTRs can be used in combination with one or more non-synthetic UTRs. See, e.g., Mandal and Rossi, Nat. Protoc. 2013 8(3):568-82, the contents of which are incorporated herein by reference in their entirety.

UTRs or portions thereof can be placed in the same orientation as in the transcript from which they were selected or can be altered in orientation or location. Hence, a 5′ and/or 3′ UTR can be inverted, shortened, lengthened, or combined with one or more other 5′ UTRs or 3′ UTRs.

In some embodiments, the polynucleotide comprises multiple UTRs, e.g., a double, a triple or a quadruple 5′ UTR or 3′ UTR. For example, a double UTR comprises two copies of the same UTR either in series or substantially in series. For example, a double beta-globin 3′UTR can be used (see US2010/0129877, the contents of which are incorporated herein by reference in its entirety).

In certain embodiments, the 5′ UTR and/or 3′ UTR sequence of the invention comprises a nucleotide sequence at least about 60%, at least about 70%, at least about 80%, at least about 90%, at least about 95%, at least about 96%, at least about 97%, at least about 98%, at least about 99%, or about 100% identical to a sequence selected from the group consisting of 5′ UTR sequences comprising any of the 5′ UTR or 3′ UTR sequences disclosed herein (e.g., in Table 3A or Table 3B), and any combination thereof.

The polynucleotides of the invention can comprise combinations of features. For example, the ORF can be flanked by a 5′ UTR that comprises a strong Kozak translational initiation signal and/or a 3′ UTR comprising an oligo (dT) sequence for templated addition of a poly-A tail. A 5′ UTR can comprise a first polynucleotide fragment and a second polynucleotide fragment from the same and/or different UTRs (see, e.g., US2010/0293625, herein incorporated by reference in its entirety).

Other non-UTR sequences can be used as regions or subregions within the polynucleotides of the invention. For example, introns or portions of intron sequences can be incorporated into the polynucleotides of the invention. Incorporation of intronic sequences can increase protein production as well as polynucleotide expression levels. In some embodiments, the polynucleotide of the invention comprises an internal ribosome entry site (IRES) instead of or in addition to a UTR (see, e.g., Yakubov et al., Biochem. Biophys. Res. Commun. 2010 394(1):189-193, the contents of which are incorporated herein by reference in their entirety). In some embodiments, the polynucleotide comprises an IRES instead of a 5′ UTR sequence. In some embodiments, the polynucleotide comprises an ORF and a viral capsid sequence. In some embodiments, the polynucleotide comprises a synthetic 5′ UTR in combination with a non-synthetic 3′ UTR.

In some embodiments, the UTR can also include at least one translation enhancer polynucleotide, translation enhancer element, or translational enhancer elements (collectively, “TEE,” which refers to nucleic acid sequences that increase the amount of polypeptide or protein produced from a polynucleotide. As a non-limiting example, the TEE can be located between the transcription promoter and the start codon. In some embodiments, the 5′ UTR comprises a TEE.

In one aspect, a TEE is a conserved element in a UTR that can promote translational activity of a nucleic acid such as, but not limited to, cap-dependent or cap-independent translation.

### a. 5′ UTR Sequences

5′ UTR sequences are important for ribosome recruitment to the mRNA and have been reported to play a role in translation (Hinnebusch A, et al., (2016) Science, 352:6292: 1413-6).

Disclosed herein, inter alia, is a polynucleotide, e.g., mRNA, comprising an open reading frame encoding a checkpoint cancer vaccine comprising (i) one or more IDO antigenic peptides and (ii) one or more PD-L1 antigenic peptides (e.g., as described herein) encoding a polypeptide, wherein the polynucleotide has a 5′ UTR that confers an increased half-life, increased expression and/or increased activity of the polypeptide encoded by said polynucleotide, or of the polynucleotide itself. In an embodiment, a polynucleotide disclosed herein comprises: (a) a 5′-UTR (e.g., as provided in Table 3A or a variant or fragment thereof); (b) a coding region comprising a stop element (e.g., as described herein); and (c) a 3′-UTR (e.g., as described herein), and LNP compositions comprising the same. In an embodiment, the polynucleotide comprises a 5′-UTR comprising a sequence provided in Table 3A or a variant or fragment thereof (e.g., a functional variant or fragment thereof).

In an embodiment, the polynucleotide having a 5′ UTR sequence provided in Table 3A or a variant or fragment thereof, has an increase in the half-life of the polynucleotide, e.g., about 1.5-20-fold increase in half-life of the polynucleotide. In an embodiment, the increase in half-life is about 1.5, 2, 3, 4, 5, 6, 7, 8, 9, 10, 11, 12, 13, 14, 15, 16, 17, 18, 19, or 20-fold, or more. In an embodiment, the increase in half life is about 1.5-fold or more. In an embodiment, the increase in half life is about 2-fold or more. In an embodiment, the increase in half life is about 3-fold or more. In an embodiment, the increase in half life is about 4-fold or more. In an embodiment, the increase in half life is about 5-fold or more.

In an embodiment, the polynucleotide having a 5′ UTR sequence provided in Table 3A or a variant or fragment thereof, results in an increased level and/or activity, e.g., output, of the polypeptide encoded by the polynucleotide. In an embodiment, the 5′ UTR results in about 1.5-20-fold increase in level and/or activity, e.g., output, of the polypeptide encoded by the polynucleotide. In an embodiment, the increase in level and/or activity is about 1.5, 2, 3, 4, 5, 6, 7, 8, 9, 10, 11, 12, 13, 14, 15, 16, 17, 18, 19, or 20-fold, or more. In an embodiment, the increase in level and/or activity is about 1.5-fold or more. In an embodiment, the increase in level and/or activity is about 2-fold or more. In an embodiment, the increase in level and/or activity is about 3-fold or more. In an embodiment, the increase in level and/or activity is about 4-fold or more. In an embodiment, the increase in level and/or activity is about 5-fold or more.

In an embodiment, the increase is compared to an otherwise similar polynucleotide which does not have a 5′ UTR, has a different 5′ UTR, or does not have a 5′ UTR described in Table 3A or a variant or fragment thereof.

In an embodiment, the increase in half-life of the polynucleotide is measured according to an assay that measures the half-life of a polynucleotide, e.g., an assay described herein.

In an embodiment, the increase in level and/or activity, e.g., output, of the polypeptide encoded by the polynucleotide is measured according to an assay that measures the level and/or activity of a polypeptide, e.g., an assay described herein.

In an embodiment, the 5′ UTR comprises a sequence provided in Table 3A or a sequence with at least 80%, 85%, 90%, 95%, 96%, 97%, 98%, 99% or 100% identity to a 5′ UTR sequence provided in Table 3A, or a variant or a fragment thereof. In an embodiment, the 5′ UTR comprises a sequence with at least 80%, 85%, 90%, 95%, 96%, 97%, 98%, 99% or 100% identity to SEQ ID NO: 50, SEQ ID NO: 51, SEQ ID NO: 52, SEQ ID NO: 53, SEQ ID NO: 54, SEQ ID NO: 55, SEQ ID NO: 56, SEQ ID NO: 57, SEQ ID NO: 58, SEQ ID NO: 60, SEQ ID NO: 61, SEQ ID NO: 62, SEQ ID NO: 63, SEQ ID NO: 64, SEQ ID NO: 65, SEQ ID NO: 66, SEQ ID NO: 67, SEQ ID NO: 68, SEQ ID NO: 69, SEQ ID NO: 70, SEQ ID NO: 71, SEQ ID NO: 72, SEQ ID NO: 73, SEQ ID NO: 74, SEQ ID NO: 75, SEQ ID NO: 76, SEQ ID NO: 77, SEQ ID NO: 78, or SEQ ID NO: 79.

In an embodiment, the 5′ UTR comprises a sequence with at least 80%, 85%, 90%, 95%, 96%, 97%, 98%, 99%, or 100% identity to SEQ ID NO: 50. In an embodiment, the 5′ UTR comprises a sequence with at least 80%, 85%, 90%, 95%, 96%, 97%, 98%, 99%, or 100% identity to SEQ ID NO: 51. In an embodiment, the 5′ UTR comprises a sequence with at least 80%, 85%, 90%, 95%, 96%, 97%, 98%, 99%, or 100% identity to SEQ ID NO: 52. In an embodiment, the 5′ UTR comprises a sequence with at least 80%, 85%, 90%, 95%, 96%, 97%, 98%, 99%, or 100% identity to SEQ ID NO: 53. In an embodiment, the 5′ UTR comprises a sequence with at least 80%, 85%, 90%, 95%, 96%, 97%, 98%, 99%, or 100% identity to SEQ ID NO: 54. In an embodiment, the 5′ UTR comprises a sequence with at least 80%, 85%, 90%, 95%, 96%, 97%, 98%, 99%, or 100% identity to SEQ ID NO: 55. In an embodiment, the 5′ UTR comprises a sequence with at least 80%, 85%, 90%, 95%, 96%, 97%, 98%, 99%, or 100% identity to SEQ ID NO: 56. In an embodiment, the 5′ UTR comprises a sequence with at least 80%, 85%, 90%, 95%, 96%, 97%, 98%, 99%, or 100% identity to SEQ ID NO: 57. In an embodiment, the 5′ UTR comprises a sequence with at least 80%, 85%, 90%, 95%, 96%, 97%, 98%, 99%, or 100% identity to SEQ ID NO: 58. In an embodiment, the 5′ UTR comprises a sequence with at least 80%, 85%, 90%, 95%, 96%, 97%, 98%, 99%, or 100% identity to SEQ ID NO: 59. In an embodiment, the 5′ UTR comprises a sequence with at least 80%, 85%, 90%, 95%, 96%, 97%, 98%, 99%, or 100% identity to SEQ ID NO: 60. In an embodiment, the 5′ UTR comprises a sequence with at least 80%, 85%, 90%, 95%, 96%, 97%, 98%, 99%, or 100% identity to SEQ ID NO: 61. In an embodiment, the 5′ UTR comprises a sequence with at least 80%, 85%, 90%, 95%, 96%, 97%, 98%, 99%, or 100% identity to SEQ ID NO: 62. In an embodiment, the 5′ UTR comprises a sequence with at least 80%, 85%, 90%, 95%, 96%, 97%, 98%, 99%, or 100% identity to SEQ ID NO: 63. In an embodiment, the 5′ UTR comprises a sequence with at least 80%, 85%, 90%, 95%, 96%, 97%, 98%, 99%, or 100% identity to SEQ ID NO: 64. In an embodiment, the 5′ UTR comprises a sequence with at least 80%, 85%, 90%, 95%, 96%, 97%, 98%, 99%, or 100% identity to SEQ ID NO: 65. In an embodiment, the 5′ UTR comprises a sequence with at least 80%, 85%, 90%, 95%, 96%, 97%, 98%, 99%, or 100% identity to SEQ ID NO: 66. In an embodiment, the 5′ UTR comprises a sequence with at least 80%, 85%, 90%, 95%, 96%, 97%, 98%, 99%, or 100% identity to SEQ ID NO: 67. In an embodiment, the 5′ UTR comprises a sequence with at least 80%, 85%, 90%, 95%, 96%, 97%, 98%, 99%, or 100% identity to SEQ ID NO: 68. In an embodiment, the 5′ UTR comprises a sequence with at least 80%, 85%, 90%, 95%, 96%, 97%, 98%, 99%, or 100% identity to SEQ ID NO: 69. In an embodiment, the 5′ UTR comprises a sequence with at least 80%, 85%, 90%, 95%, 96%, 97%, 98%, 99%, or 100% identity to SEQ ID NO: 70. In an embodiment, the 5′ UTR comprises a sequence with at least 80%, 85%, 90%, 95%, 96%, 97%, 98%, 99%, or 100% identity to SEQ ID NO: 71. In an embodiment, the 5′ UTR comprises a sequence with at least 80%, 85%, 90%, 95%, 96%, 97%, 98%, 99%, or 100% identity to SEQ ID NO: 72. In an embodiment, the 5′ UTR comprises a sequence with at least 80%, 85%, 90%, 95%, 96%, 97%, 98%, 99%, or 100% identity to SEQ ID NO: 73. In an embodiment, the 5′ UTR comprises a sequence with at least 80%, 85%, 90%, 95%, 96%, 97%, 98%, 99%, or 100% identity to SEQ ID NO: 74. In an embodiment, the 5′ UTR comprises a sequence with at least 80%, 85%, 90%, 95%, 96%, 97%, 98%, 99%, or 100% identity to SEQ ID NO: 75. In an embodiment, the 5′ UTR comprises a sequence with at least 80%, 85%, 90%, 95%, 96%, 97%, 98%, 99%, or 100% identity to SEQ ID NO: 76. In an embodiment, the 5′ UTR comprises a sequence with at least 80%, 85%, 90%, 95%, 96%, 97%, 98%, 99%, or 100% identity to SEQ ID NO: 77. In an embodiment, the 5′ UTR comprises a sequence with at least 80%, 85%, 90%, 95%, 96%, 97%, 98%, 99%, or 100% identity to SEQ ID NO: 78. In an embodiment, the 5′ UTR comprises a sequence with at least 80%, 85%, 90%, 95%, 96%, 97%, 98%, 99%, or 100% identity to SEQ ID NO: 79.

In an embodiment, a 5′ UTR sequence provided in Table 3A has an additional first nucleotide which is an A. In an embodiment, a 5′ UTR sequence provided in Table 3A has an additional first nucleotide which is a G.

TABLE 3A
5′ UTR sequences

| SEQ | | |
|---|---|---|
| ID | Sequence | |
| NO: | Name | Sequence |
| 50 | A1 | GGAAAUCGCAAAAUUUGCUCUUCGCGUUAGA |
| | | UUUCUUUUAGUUUUCUCGCAACUAGCAAGCU |
| | | UUUUGUUCUCGCC |
| 51 | A5 | GGAAAUCCCCACAACCGCCUCAUAUCCAGGC |
| | | UCAAGAAUAGAGCUCAGUGUUUUGUUGUUUA |
| | | AUCAUUCCGACGUGUUUUGCGAUAUUCGCGC |
| | | AAAGCAGCCAGUCGCGCGCUUGCUUUUAAGU |
| | | AGAGUUGUUUUUCCACCCGUUUGCCAGGCAU |
| | | CUUUAAUUUAACAUAUUUUUAUUUUUCAGGC |
| | | UAACCUACGCCGCCACC |
| 52 | A6 | GGAAAUAAGAGAGAAAAGAAGAGUAAGAAGA |
| | | AAUAUAAGAUCUCCCUGAGCUUCAGGGAGCC |
| | | CCGGCGCCGCCACC |
| 53 | A7 | GGAAACCCCCCACCCCCGUAAGAGAGAAAAG |
| | | AAGAGUAAGAAGAAAUAUAAGAUCUCCCUGA |
| | | GCUUCAGGGAGCCCCGGCGCCGCCACC |
| 54 | A8 | GGAGAACUUCCGCUUCCGUUGGCGCAAGCGC |
| | | UUUCAUUUUUUCUGCUACCGUGACUAAG |
| 55 | A9 | GGAAAUAAGAGAGAAAAGAAGAGUAAGAAGA |
| | | AAUAUAAGAGCCACC |
| 56 | A11 | GGAAAUAAGAGAGAAAAGAAGAGUAAGAAGA |
| | (Reference) | AAUAUAAGACCCCGGCGCCGCCACC |
| 57 | A2 | GGAAAUCGCAAAAUUUGCUCUUCGCGUUAGA |
| | | UUUCUUUUAGUUUUCUCGCAACUAGCAAGCU |
| | | UUUUGUUCUCGCCGCCGCC |
| 58 | A3 | GGAAAUCGCAAAAUUUUCUUUUCGCGUUAGA |
| | | UUUCUUUUAGUUUUCUUUCAACUAGCAAGCU |
| | | UUUUGUUCUCGCCGCCGCC |
| 59 | A4 | G G A A A U C G C A A A A (N₂)ₓ |
| | | (N₃)ₓ C U (N₄)ₓ (N₅)ₓ C G C G U |
| | | U A G A U U U C U U U U A G U U |
| | | U U C U N₆ N₇ C A A C U A G C A |
| | | A G C U U U U U G U U C U C G C |
| | | C (N₈ C C)ₓ |
| | | (N₂)ₓ is a uracil and x is an |
| | | integer from 0 to 5, e.g., |
| | | wherein x = 3 or 4; |
| | | (N₃)ₓ is a guanine and x is an |
| | | integer from 0 to 1; |
| | | (N₄)ₓ is a cytosine and x is |
| | | an integer from 0 to 1; |
| | | (N₅)ₓ is a uracil and x is an |
| | | integer from 0 to 5, e.g., |
| | | wherein x = 2 or 3; |
| | | N₆ is a uracil or cytosine; |
| | | N₇ is a uracil or guanine; |
| | | N₈ is adenine or guanine and |
| | | x is an integer from 0 to 1. |
| 60 | A27 | GGAAAAUUUUAGCCUGGAACGUUAGAUAACU |
| | | GUCCUGUUGUCUUUAUAUACUUGGUCCCCAA |
| | | GUAGUUUGUCUUCCAAA |
| 61 | A12 | GGAAACUUUAUUUAGUGUUACUUUAUUUUCU |
| | | GUUUAUUUGUGUUUCUUCAGUGGGUUUGUUC |
| | | UAAUUUCCUUGGCCGCC |
| 62 | A13 | GGAAAAUCUGUAUUAGGUUGGCGUGUUCUUU |
| | | GGUCGGUUGUUAGUAUUGUUGUUGAUUCGUU |
| | | UGUGGUCGGUUGCCGCC |
| 63 | A14 | GGAAAAUUAUUAACAUCUUGGUAUUCUCGAU |
| | | AACCAUUCGUUGGAUUUUAUUGUAUUCGUAG |
| | | UUUGGGUUCCUGCCGCC |
| 64 | A15 | GGAAAUUAUUAUUAUUUCUAGCUACAAUUUA |
| | | UCAUUGUAUUAUUUUAGCUAUUCAUCAUUAU |
| | | UUACUUGGUGAUCAACA |
| 65 | A16 | GGAAAUAGGUUGUUAACCAAGUUCAAGCCUA |
| | | AUAAGCUUGGAUUCUGGUGACUUGCUUCACC |
| | | GUUGGCGGGCACCGAUC |
| 66 | A17 | GGAAAUCGUAGAGAGUCGUACUUAGUACAUA |
| | | UCGACUAUCGGUGGACACCAUCAAGAUUAUA |
| | | AACCAGGCCAGA |
| 67 | A18 | GGAAACCCGCCCAAGCGACCCCAACAUAUCA |
| | | GCAGUUGCCCAAUCCCAACUCCCAACACAAU |
| | | CCCCAAGCAACGCCGCC |
| 68 | A19 | GGAAAGCGAUUGAAGGCGUCUUUUCAACUAC |
| | | UCGAUUAAGGUUGGGUAUCGUCGUGGGACUU |
| | | GGAAAUUUGUUGUUUCC |
| 69 | A20 | GGAAACUAAUCGAAAUAAAAGAGCCCCGUAC |
| | | UCUUUUAUUUCUAUUAGGUUAGGAGCCUUAG |
| | | CAUUUGUAUCUUAGGUA |
| 70 | A21 | GGAAAUGUGAUUUCCAGCAACUUCUUUUGAA |
| | | UAUAUUGAAUUCCUAAUUCAAAGCGAACAAA |
| | | UCUACAAGCCAUAUACC |
| 71 | A22 | GGAAAUCGUAGAGAGUCGUACUUACGUGGUC |
| | | GCCAUUGCAUAGCGCGCGAAAGCAACAGGAA |
| | | CAAGAACGCGCC |
| 72 | A23 | GGAAAUCGUAGAGAGUCGUACUUAGAAUAAA |
| | | CAGAGUCGGGUCGACUUGUCUCUGAUACUAC |
| | | GACGUCACAAUC |
| 73 | A24 | GGAAAAUUUGCCUUCGGAGUUGCGUAUCCUG |
| | | AACUGCCCAGCCUCCUGAUAUACAACUGUUC |
| | | CGCUUAUUCGGGCCGCC |
| 74 | A25 | GGAAAUCUGAGCAGGAAUCCUUUGUGCAUUG |
| | | AAGACUUUAGAUUCCUCUCUGCGGUAGACGU |
| | | GCACUUAUAAGUAUUUG |
| 75 | A26 | GGAAAGCGAUUGAAGGCGUCUUUUCAACUAC |
| | | UCGAUUAAGGUUGGGUAUCGUCGUGGGACUU |
| | | GGAAAUUUGUUGCCACC |
| 76 | A28 | GGAAAUUUUUUUUUGAUAUUAUAAGAGUUUU |
| | | UUUUUGAUAUUAAGAAAAUUUUUUUUUGAUA |
| | | UUAGAAGAGUAAGAAGAAAUAUAAGACCCCG |
| | | GCGCCGCCACC |
| 77 | A29 | GGAAAUAAGAGAGAAAAGAAGAGUAAGAAGA |
| | | AAUAUAAGAGCCAAAAAAAAAAAACC |
| 78 | A30 | GGAAAUCUCCCUGAGCUUCAGGGAGUAAGAG |
| | | AGAAAAGAAGAGUAAGAAGAAAUAUAAGACC |
| | | CCGGCGCCGCCACC |
| | A31 | GCCRCC, wherein R = A or G |
| 79 | A32 | GGAAAUCGCAAAAUUUGCUCUUCGCGUUAGA |
| | | UUUCUUUUAGUUUUCUCGCAACUAGCAAGCU |
| | | UUUUGUUCUCGCC |

In an embodiment, the 5′ UTR comprises a variant of SEQ ID NO: 50. In an embodiment, the variant of SEQ ID NO: 50 comprises a nucleic acid sequence of Formula A:

(SEQ ID NO: 59)
G G A A A U C G C A A A A (N2)X (N3)X C U (N4)X
(N5)X C G C G U U A G A U U U C U U U U A G U U
U U C U N6 N7 C A A C U A G C A A G C U U U U U
G U U C U C G CC (N8 C C)x,

wherein:
(N2)x is a uracil and x is an integer from 0 to 5, e.g., wherein x=3 or 4;
(N3)x is a guanine and x is an integer from 0 to 1;
(N4)x is a cytosine and x is an integer from 0 to 1;
(N5)x is a uracil and x is an integer from 0 to 5, e.g., wherein x=2 or 3;
N6 is a uracil or cytosine;
N7 is a uracil or guanine;
N8 is adenine or guanine and x is an integer from 0 to 1.

In an embodiment (N2)x is a uracil and x is 0. In an embodiment (N2)x is a uracil and x is 1. In an embodiment (N2)x is a uracil and x is 2. In an embodiment (N2)x is a uracil and x is 3. In an embodiment, (N2)x is a uracil and x is 4. In an embodiment (N2)x is a uracil and x is 5.

In an embodiment, (N3)x is a guanine and x is 0. In an embodiment, (N3)x is a guanine and x is 1.

In an embodiment, (N4)x is a cytosine and x is 0. In an embodiment, (N4)x is a cytosine and x is 1.

In an embodiment (N5)x is a uracil and x is 0. In an embodiment (N5)x is a uracil and x is 1. In an embodiment (N5)x is a uracil and x is 2. In an embodiment (N5)x is a uracil and x is 3. In an embodiment, (N5)x is a uracil and x is 4. In an embodiment (N5)x is a uracil and x is 5.

In an embodiment, N6 is a uracil. In an embodiment, N6 is a cytosine.

In an embodiment, N7 is a uracil. In an embodiment, N7 is a guanine.

In an embodiment, N8 is an adenine and x is 0. In an embodiment, N8 is an adenine and x is 1.

In an embodiment, N8 is a guanine and x is 0. In an embodiment, N8 is a guanine and x is 1.

In an embodiment, the 5′ UTR comprises a variant of SEQ ID NO: 50. In an embodiment, the variant of SEQ ID NO: 50 comprises a sequence with at least 50%, 60%, 70%, 80%, 85%, 90%, 95%, 96%, 97%, 98%, or 99% identity to SEQ ID NO: 50. In an embodiment, the variant of SEQ ID NO: 50 comprises a sequence with at least 50% identity to SEQ ID NO: 50. In an embodiment, the variant of SEQ ID NO: 50 comprises a sequence with at least 60% identity to SEQ ID NO: 50. In an embodiment, the variant of SEQ ID NO: 50 comprises a sequence with at least 70% identity to SEQ ID NO: 50. In an embodiment, the variant of SEQ ID NO: 50 comprises a sequence with at least 80% identity to SEQ ID NO: 50. In an embodiment, the variant of SEQ ID NO: 50 comprises a sequence with at least 90% identity to SEQ ID NO: 50. In an embodiment, the variant of SEQ ID NO: 50 comprises a sequence with at least 95% identity to SEQ ID NO: 50. In an embodiment, the variant of SEQ ID NO: 50 comprises a sequence with at least 96% identity to SEQ ID NO: 50. In an embodiment, the variant of SEQ ID NO: 50 comprises a sequence with at least 97% identity to SEQ ID NO: 50. In an embodiment, the variant of SEQ ID NO: 50 comprises a sequence with at least 98% identity to SEQ ID NO: 50. In an embodiment, the variant of SEQ ID NO: 50A comprises a sequence with at least 99% identity to SEQ ID NO: 50.

In an embodiment, the variant of SEQ ID NO: 50 comprises a uridine content of at least 5%, 10%, 20%, 30%, 40%, 50%, 60%, 70%, or 80%. In an embodiment, the variant of SEQ ID NO: 50 comprises a uridine content of at least 5%. In an embodiment, the variant of SEQ ID NO: 50 comprises a uridine content of at least 10%. In an embodiment, the variant of SEQ ID NO: 50 comprises a uridine content of at least 20%. In an embodiment, the variant of SEQ ID NO: 50 comprises a uridine content of at least 30%. In an embodiment, the variant of SEQ ID NO: 50 comprises a uridine content of at least 40%. In an embodiment, the variant of SEQ ID NO: 50 comprises a uridine content of at least 50%. In an embodiment, the variant of SEQ ID NO: 50 comprises a uridine content of at least 60%. In an embodiment, the variant of SEQ ID NO: 50 comprises a uridine content of at least 70%. In an embodiment, the variant of SEQ ID NO: 50 comprises a uridine content of at least 80%.

In an embodiment, the variant of SEQ ID NO: 50 comprises at least 2, 3, 4, 5, 6 or 7 consecutive uridines (e.g., a polyuridine tract). In an embodiment, the polyuridine tract in the variant of SEQ ID NO: 50 comprises at least 1-7, 2-7, 3-7, 4-7, 5-7, 6-7, 1-6, 1-5, 1-4, 1-3, 1-2, 2-6, or 3-5 consecutive uridines.

In an embodiment, the polyuridine tract in the variant of SEQ ID NO: 50 comprises 4 consecutive uridines. In an embodiment, the polyuridine tract in the variant of SEQ ID NO: 50 comprises 5 consecutive uridines.

In an embodiment, the variant of SEQ ID NO: 50 comprises 1, 2, 3, 4, 5, 6, 7, 8, 9, 10, 11, 12, 13, 14, or 15 polyuridine tracts. In an embodiment, the variant of SEQ ID NO: 50 comprises 3 polyuridine tracts. In an embodiment, the variant of SEQ ID NO: 50 comprises 4 polyuridine tracts. In an embodiment, the variant of SEQ ID NO: 50 comprises 5 polyuridine tracts.

In an embodiment, one or more of the polyuridine tracts are adjacent to a different polyuridine tract. In an embodiment, each of, e.g., all, the polyuridine tracts are adjacent to each other, e.g., all of the polyuridine tracts are contiguous.

In an embodiment, one or more of the polyuridine tracts are separated by 1, 2, 3, 4, 5, 6, 7, 8, 9, 10, 11, 2, 13, 14, 15, 16, 17, 18, 19, 20, 30, 40, 50, or 60 nucleotides. In an embodiment, each of, e.g., all of, the polyuridine tracts are separated by 1, 2, 3, 4, 5, 6, 7, 8, 9, 10, 11, 2, 13, 14, 15, 16, 17, 18, 19, 20, 30, 40, 50, or 60 nucleotides.

In an embodiment, a first polyuridine tract and a second polyuridine tract are adjacent to each other.

In an embodiment, a subsequent, e.g., third, fourth, fifth, sixth, seventh, eighth, ninth, or tenth, polyuridine tract is separated by 1, 2, 3, 4, 5, 6, 7, 8, 9, 10, 11, 2, 13, 14, 15, 16, 17, 18, 19, 20, 30, 40, 50, or 60 nucleotides from the first polyuridine tract, the second polyuridine tract, or any one of the subsequent polyuridine tracts.

In an embodiment, a first polyuridine tract is separated by 1, 2, 3, 4, 5, 6, 7, 8, 9, 10, 11, 2, 13, 14, 15, 16, 17, 18, 19, 20, 30, 40, 50 or 60 nucleotides from a subsequent polyuridine tract, e.g., a second, third, fourth, fifth, sixth or seventh, eighth, ninth, or tenth polyuridine tract. In an embodiment, one or more of the subsequent polyuridine tracts are adjacent to a different polyuridine tract.

In an embodiment, the 5′ UTR comprises a Kozak sequence, e.g., a GCCRCC nucleotide sequence, wherein R is an adenine or guanine. In an embodiment, the Kozak sequence is disposed at the 3′ end of the 5′ ‘UTR sequence.

In an aspect, the polynucleotide (e.g., mRNA) comprising an open reading frame encoding a checkpoint cancer vaccine comprising one or more IDO antigenic peptides and one or more PD-L1 antigenic peptides (e.g., SEQ ID NO: 300, 301, or 302) and comprising a 5′ UTR sequence disclosed herein is formulated as an LNP. In an embodiment, the LNP composition comprises: (i) an ionizable lipid, e.g., an amino lipid; (ii) a sterol or other structural lipid; (iii) a non-cationic helper lipid or phospholipid; and (iv) a PEG-lipid.

### b. 3′ UTR Sequences

3′UTR sequences have been shown to influence translation, half-life, and subcellular localization of mRNAs (Mayr C., Cold Spring Harb Persp Biol 2019 Oct. 1; 11(10):a034728).

Disclosed herein, inter alia, is a polynucleotide, e.g., mRNA, comprising an open reading frame encoding a checkpoint cancer vaccine comprising one or more IDO antigenic peptides and one or more PD-L1 antigenic peptides (e.g., SEQ ID NO: 300, 301, or 302), which polynucleotide has a 3′ UTR that confers an increased half-life, increased expression and/or increased activity of the polypeptide encoded by said polynucleotide, or of the polynucleotide itself. In an embodiment, a polynucleotide disclosed herein comprises: (a) a 5′-UTR (e.g., as described herein); (b) a coding region comprising a stop element (e.g., as described herein); and (c) a 3′-UTR (e.g., as provided in Table 3B or a variant or fragment thereof), and LNP compositions comprising the same. In an embodiment, the polynucleotide comprises a 3′-UTR comprising a sequence provided in Table 3B or a variant or fragment thereof.

In an embodiment, the polynucleotide having a 3′ UTR sequence provided in Table 3B or a variant or fragment thereof, results in an increased half-life of the polynucleotide, e.g., about 1.5-10-fold increase in half-life of the polynucleotide. In an embodiment, the increase in half-life is about 1.5, 2, 3, 4, 5, 6, 7, 8, 9, or 10-fold, or more. In an embodiment, the increase in half-life is about 1.5-fold or more. In an embodiment, the increase in half-life is about 2-fold or more. In an embodiment, the increase in half-life is about 3-fold or more. In an embodiment, the increase in half-life is about 4-fold or more. In an embodiment, the increase in half-life is about 5-fold or more. In an embodiment, the increase in half-life is about 6-fold or more. In an embodiment, the increase in half-life is about 7-fold or more. In an embodiment, the increase in half-life is about 8-fold. In an embodiment, the increase in half-life is about 9-fold or more. In an embodiment, the increase in half-life is about 10-fold or more.

In an embodiment, the polynucleotide having a 3′ UTR sequence provided in Table 3B or a variant or fragment thereof, results in a polynucleotide with a mean half-life score of greater than 10.

In an embodiment, the polynucleotide having a 3′ UTR sequence provided in Table 3B or a variant or fragment thereof, results in an increased level and/or activity, e.g., output, of the polypeptide encoded by the polynucleotide.

In an embodiment, the increase is compared to an otherwise similar polynucleotide which does not have a 3′ UTR, has a different 3′ UTR, or does not have a 3′ UTR of Table 3B or a variant or fragment thereof.

In an embodiment, the polynucleotide comprises a 3′ UTR sequence provided in Table 3B or a sequence with at least 80%, 85%, 90%, 95%, 96%, 97%, 98%, 99% or 100% identity to a 3′ UTR sequence provided in Table 3B, or a fragment thereof. In an embodiment, the 3′ UTR comprises a sequence with at least 80%, 85%, 90%, 95%, 96%, 97%, 98%, 99% or 100% identity to SEQ ID NO: 100, SEQ ID NO: 101, SEQ ID NO: 102, SEQ ID NO: 103, SEQ ID NO: 104, SEQ ID NO: 105, SEQ ID NO: 106, SEQ ID NO: 107, SEQ ID NO: 108, SEQ ID NO: 109, SEQ ID NO: 110, SEQ ID NO: 111, SEQ ID NO: 112, SEQ ID NO: 113, SEQ ID NO: 114, SEQ ID NO:115, SEQ ID NO:136, SEQ ID NO: 137, SEQ ID NO: 138, SEQ ID NO: 139, SEQ ID NO: 140, or SEQ ID NO: 141.

In an embodiment, the 3′ UTR comprises the sequence of SEQ ID NO: 100, or a sequence with at least 80%, 85%, 90%, 95%, 96%, 97%, 98%, 99% or 100% identity to SEQ ID NO: 100. In an embodiment, the 3′ UTR comprises the sequence of SEQ ID NO: 101, or a sequence with at least 80%, 85%, 90%, 95%, 96%, 97%, 98%, 99% or 100% identity to SEQ ID NO: 101. In an embodiment, the 3′ UTR comprises the sequence of SEQ ID NO: 102, or a sequence with at least 80%, 85%, 90%, 95%, 96%, 97%, 98%, 99% or 100% identity to SEQ ID NO: 102. In an embodiment, the 3′ UTR comprises the sequence of SEQ ID NO: 103, or a sequence with at least 80%, 85%, 90%, 95%, 96%, 97%, 98%, 99% or 100% identity to SEQ ID NO: 103. In an embodiment, the 3′ UTR comprises the sequence of SEQ ID NO: 104, or a sequence with at least 80%, 85%, 90%, 95%, 96%, 97%, 98%, 99% or 100% identity to SEQ ID NO: 104. In an embodiment, the 3′ UTR comprises the sequence of SEQ ID NO: 105, or a sequence with at least 80%, 85%, 90%, 95%, 96%, 97%, 98%, 99% or 100% identity to SEQ ID NO: 105. In an embodiment, the 3′ UTR comprises the sequence of SEQ ID NO: 106, or a sequence with at least 80%, 85%, 90%, 95%, 96%, 97%, 98%, 99% or 100% identity to SEQ ID NO: 106. In an embodiment, the 3′ UTR comprises the sequence of SEQ ID NO: 107, or a sequence with at least 80%, 85%, 90%, 95%, 96%, 97%, 98%, 99% or 100% identity to SEQ ID NO: 107. In an embodiment, the 3′ UTR comprises the sequence of SEQ ID NO: 108, or a sequence with at least 80%, 85%, 90%, 95%, 96%, 97%, 98%, 99% or 100% identity to SEQ ID NO: 108. In an embodiment, the 3′ UTR comprises the sequence of SEQ ID NO: 109, or a sequence with at least 80%, 85%, 90%, 95%, 96%, 97%, 98%, 99% or 100% identity to SEQ ID NO: 109. In an embodiment, the 3′ UTR comprises the sequence of SEQ ID NO: 110, or a sequence with at least 80%, 85%, 90%, 95%, 96%, 97%, 98%, 99% or 100% identity to SEQ ID NO: 110. In an embodiment, the 3′ UTR comprises the sequence of SEQ ID NO: 111, or a sequence with at least 80%, 85%, 90%, 95%, 96%, 97%, 98%, 99% or 100% identity to SEQ ID NO: 111. In an embodiment, the 3′ UTR comprises the sequence of SEQ ID NO: 112, or a sequence with at least 80%, 85%, 90%, 95%, 96%, 97%, 98%, 99% or 100% identity to SEQ ID NO: 112. In an embodiment, the 3′ UTR comprises the sequence of SEQ ID NO: 113, or a sequence with at least 80%, 85%, 90%, 95%, 96%, 97%, 98%, 99% or 100% identity to SEQ ID NO: 113. In an embodiment, the 3′ UTR comprises the sequence of SEQ ID NO: 114, or a sequence with at least 80%, 85%, 90%, 95%, 96%, 97%, 98%, 99% or 100% identity to SEQ ID NO: 114. In an embodiment, the 3′ UTR comprises the sequence of SEQ ID NO: 115, or a sequence with at least 80%, 85%, 90%, 95%, 96%, 97%, 98%, 99% or 100% identity to SEQ ID NO: 115. In an embodiment, the 3′ UTR comprises the sequence of SEQ ID NO: 136, or a sequence with at least 80%, 85%, 90%, 95%, 96%, 97%, 98%, 99% or 100% identity to SEQ ID NO: 136. In an embodiment, the 3′ UTR comprises the sequence of SEQ ID NO: 137, or a sequence with at least 80%, 85%, 90%, 95%, 96%, 97%, 98%, 99% or 100% identity to SEQ ID NO: 137. In an embodiment, the 3′ UTR comprises the sequence of SEQ ID NO: 138, or a sequence with at least 80%, 85%, 90%, 95%, 96%, 97%, 98%, 99% or 100% identity to SEQ ID NO: 138. In an embodiment, the 3′ UTR comprises the sequence of SEQ ID NO: 139, or a sequence with at least 80%, 85%, 90%, 95%, 96%, 97%, 98%, 99% or 100% identity to SEQ ID NO: 139. In an embodiment, the 3′ UTR comprises the sequence of SEQ ID NO: 140, or a sequence with at least 80%, 85%, 90%, 95%, 96%, 97%, 98%, 99% or 100% identity to SEQ ID NO: 140. In an embodiment, the 3′ UTR comprises the sequence of SEQ ID NO: 141, or a sequence with at least 80%, 85%, 90%, 95%, 96%, 97%, 98%, 99% or 100% identity to SEQ ID NO: 141.

TABLE 3B
3′ UTR sequences

| SEQ ID | | |
|---|---|---|
| NO | Sequence Name | Sequence |
| 100 | B1 | UAAUAGUAAGCUGGAGCCUCCUGAGAGACCUGUGUGAACU |
| | | AUUGAGAAGAUCGGAACAGCUCCUUACUCUGAGGAAGUU |
| | | GGUACCCCCGUGGUCUUUGAAUAAAGUCUGAGUGGGCGGC |
| 101 | B3 | UAAUAGUAAGCUGGAGCCUCACUCUCCUCUCCAUCCCGUA |
| | | UCCAGGCUGUGAAUUUUUCAAGGAAUAUAAAGAUCGGGA |
| | | UGUACCCCCGUGGUCUUUGAAUAAAGUCUGAGUGGGCGGC |
| 102 | B4 | UGAUAGUAAGCUGGAGCCUCUAGUGACGGCAACAGGGCUU |
| | | GGUUUUUCCUUGUUGUGAAAUCGACAUCUCUGAAGACAG |
| | | GGUACCCCCGUGGUCUUUGAAUAAAGUCUGAGUGGGCGGC |
| 103 | B5 | UGAUAGUAAGCUGGAGCCUCCUUCCAUCUAGUCACAAAGA |
| | | CUCCUUCGUCCCCAGUUGCCGUCUAGGAUUGGGCCUCCCA |
| | | GUACCCCCGUGGUCUUUGAAUAAAGUCUGAGUGGGCGGC |
| 104 | B6 | UGAUAGUAAGCUGGAGCCUCCCAUAACAUGACAUAUCUGG |
| | | AUUUUGUGCUUAGAACCUUAAAUUGGAAGCAUUCUUAAU |
| | | UGUACCCCCGUGGUCUUUGAAUAAAGUCUGAGUGGGCGGC |
| 105 | B7 | UAAUAGUAAGCUGGAGCCUCCGGAAAACUAAAAUAGAGA |
| | | UAUUUCAAGAUUUUAUAAUUUUCAAAGACCUUUGAAAUA |
| | | UUGUACCCCCGUGGUCUUUGAAUAAAGUCUGAGUGGGCGG |
| | | C |
| 106 | B8 | UAAUAGUAAGCUGGAGCCUCUACACAUUGCUUCUAGUUGG |
| | | CAGAAAUAAUUGAUUAAAAGACCAGAAACUGUGAUAACU |
| | | GGUACCCCCGUGGUCUUUAAAUAAAGUCUAAGUGGGCGGC |
| 107 | B9 | UGAUAAUAGGCUGGAGCCUCGGUGGCCAUGCUUCUUGCCC |
| | | CUUGGGCCUCCCCCCAGCCCCUCCUCCCCUUCCUGCACCCG |
| | | UACCCCCGUGGUCUUUGAAUAAAGUCUGAGUGGGGGC |
| 108 | B10 | UGAUAAUAGGCUGGAGCCUCGGUGGCCUAGCUUCUUGCCC |
| | | CUUGGGCCUCCCCCCAGCCCCUCCUCCCCUUCCUGCACCCG |
| | | UACCCCCGUGGUCUUUGAAUAAAGUCUGAGUGGGGGC |
| 109 | B11 | UGAUAAUAGGCUGGAGCCUCGGUGGCCAUGCUUCUUGCCC |
| | | CUUGGGCCUCCCCCCAGCCCCUCCUCCCCUUCCUGCACCCG |
| | | UACCCCCCAAACACCAUUGUCACACUCCAGUGGUCUUUGA |
| | | AUAAAGUCUGAGUGGGGGC |
| 110 | B12 | UGAUAAUAGGCUGGAGCCUCGGUGGCCUAGCUUCUUGCCC |
| | | CUUGGGCCUCCCCCCAGCCCCUCCUCCCCUUCCUGCACCCG |
| | | UACCCCCCAAACACCAUUGUCACACUCCAGUGGUCUUUGA |
| | | AUAAAGUCUGAGUGGGCGGC |
| 111 | B13 | UGAUAAUAGGCUGGAGCCUCGGUGGCCUAGCUUCUUGCCC |
| | | CUUGGGCCUCCCCCCAGCCCCUCCUCCCCUUCCUGCACCCG |
| | | UACCCCCUCCAUAAAGUAGGAAACACUACAGUGGUCUUUG |
| | | AAUAAAGUCUGAGUGGGGGC |
| 112 | B14 | UGAUAAUAGGCUGGAGCCUCGGUGGCCUAGCUUCUUGCCC |
| | | CUUGGGCCUCCCCCCAGCCCCUCCUCCCCUUCCUGCACCCG |
| | | UACCCCCCGCAUUAUUACUCACGGUACGAGUGGUCUUUGA |
| | | AUAAAGUCUGAGUGGGCGGC |
| 113 | B15 | UGAUAAUAGUCCAUAAAGUAGGAAACACUACAGCUGGAG |
| | | CCUCGGUGGCCAUGCUUCUUGCCCCUUGGGCCUCCCCCCA |
| | | GCCCCUCCUCCCCUUCCUGCACCCGUACCCCCCGCAUUAUU |
| | | ACUCACGGUACGAGUGGUCUUUGAAUAAAGUCUGAGUGG |
| | | GCGGC |
| 114 | B16 | UGAUAAUAGUCCAUAAAGUAGGAAACACUACAGCUGGAG |
| | | CCUCGGUGGCCUAGCUUCUUGCCCCUUGGGCCUCCAUAAA |
| | | GUAGGAAACACUACAUCCCCCCAGCCCCUCCUCCCCUUCCU |
| | | GCACCCGUACCCCCUCCAUAAAGUAGGAAACACUACAGUG |
| | | GUCUUUGAAUAAAGUCUGAGUGGGCGGC |
| 115 | B2 | CUGAGAGACCUGUGUGAACUAUUGAGAAGAUCGGAACAG |
| | | CUCCUUACUCUGAGGAAGUUG |
| 116 | B17 | UGAUAAUAGGCUGGAGCCUCUCACACACCUCUGCCCCUUG |
| | | GGCCUCCCACUCCCAUGGCUCUGGGCGGUCCAGAAGGAGC |
| | | GUACCCCCGUGGUCUUUGAAUAAAGUCUGAGUGGGCGGC |
| 117 | B18 | UGAUAAUAGGCUGGAGCCUCCACCGCGUUAUCCGUUCCUC |
| | | GUAGGCUGGUCCUGGGGAACGGGUCGGCGGGUACCCCCGU |
| | | GGUCUUUGAAUAAAGUCUGAGUGGGOGGC |
| 118 | B19 | UGAUAAUAGGCUGGAGCCUCUGCCCGGCAACGGCCAGGUC |
| | | UGUGCCAAGUGUUUGCUGACGCAACCCCCACUGGCUGGGG |
| | | CUUGGUCAUGGGCCAUCAGCGCGUGCGUGGAACCUUUUCG |
| | | GCUCCUCUGCCGAUCCAUACUGCGGAACUCCUAGCCGCUU |
| | | GUUUUGCUCGCAGCAGGUCUGGAGCAAACAUUAUCGGGAC |
| | | UGAUAACUCUGUUGUCCUGUACCCCCGUGGUCUUUGAAUA |
| | | AAGUCUGAGUGGGCGGC |
| 119 | B20 | UGAUAAUAGGCUGGAGCCUCGGUGGCCUAGCUUCUUGCCC |
| | | CUUGGGCCUCCCCCCAGCCCCUCCUCCCCUUCCUGCACCCG |
| | | UACCCUUUUUUUUUUUUUUUUUUUCUUCUUUUUUUUUU |
| | | UUCUUUUUUUUUUUUCUUUCUUUUUUUCUUUUUUUUUCU |
| | | UUUUUUUUUCUUUUUUUUUUUUUUUUCCGUGGUCUUUG |
| | | AAUAAAGUCUGAGUGGGCGGC |
| 120 | B21 | UGAUAAUAGGCUGGAGCCUCGGUGGCCUAGCUUCUUGCCC |
| | | CUUGGGCCUCCCCCCAGCCCCUCCUCCCCUUCCUGCACCCG |
| | | UACCCUUUUUUUUUUUUUUUUUUUUUUUUUUUUUUUUUU |
| | | UUUUUUUUUUUUUUUUUUUUUUUUUUUUUUUUUUUUUUU |
| | | UUUUUUUUUUUUUUUUUUUUUUUUUUUCCGUGGUCUUUG |
| | | AAUAAAGUCUGAGUGGGCGGC |
| 133 | B22 | UAAGUCUCCAUAAAGUAGGAAACACUACAGCUGGAGCCUC |
| | | GGUGGCCUAGCUUCUUGCCCCUUGGGCCUCCAUAAAGUAG |
| | | GAAACACUACAUCCCCCCAGCCCCUCCUCCCCUUCCUGCAC |
| | | CCGUACCCCCUCCAUAAAGUAGGAAACACUACAGUGGUCU |
| | | UUGAAUAAAGUCUGAGUGGGCGGC |
| 134 | B23 | UAAAGCGCUGGAGCCUCGGUGGCCUAGCUUCUUGCCCCUU |
| | | GGGCCUCCCCCCAGCCCCUCCUCCCCUUCCUGCACCCGUAC |
| | | CCCCUCCAUAAAGUAGGAAACACUACAGUGGUCUUUGAAU |
| | | AAAGUCUGAGUGGGCGGC |
| 135 | B24 | UAAAGCUCCCCGGGGUCCAUAAAGUAGGAAACACUACAGC |
| | | UGGAGCCUCCUGAGAGACCUGUGUGAACUAUUGAGAAGA |
| | | UCGGAACAGCUCCUUACUCUGAGGAAGUUGUCCAUAAAGU |
| | | AGGAAACACUACAGUACCCCCUCCAUAAAGUAGGAAACAC |
| | | UACAGUGGUCUUUGAAUAAAGUCUGAGUGGGCGGC |
| 136 | B25 | UAAUAGUAAACCUCACUCACGGCCACAUUGAGUGCCAGGC |
| | | UCCGGGCUGGUUUAUAGUAGUGUAGAGCAUUGCAGCACU |
| | | UAGACUGGGGUGCUGUAGUCUUUAUUGUAGUCUUUCCAC |
| | | AUACCUGAUAAUUCUUAGAUAAUUUCUUAUUUUAAUUCC |
| | | AUAAAGUAGGAAACACUACAUAAAUCUCCAUAAAGUAGG |
| | | AAACACUACAUAUUCUUCCAUAAAGUAGGAAACACUACAU |
| | | AGGCU |
| 137 | B26 | GCCUCCACCGCGUUAUCCGUUCCUCGUAGGCUGGUCCUGG |
| | | GGAACGGGUCGGCGGGUACCCCCGUGGUCUUUGAAUAAAG |
| | | UCUGAGUGGGCGGC |
| 138 | B27 | CACCGCGUUAUCCGUUCCUCGUAGGCUGGUCCUGGGGAAC |
| | | GGGUCGGCGGGCCUCGGUGGCCUAGCUUCUUGCCCCUUGG |
| | | GCCCACCGCGUUAUCCGUUCCUCGUAGGCUGGUCCUGGGG |
| | | AACGGGUCGGCGGUCCCCCCAGCCCCUCCUCCCCUUCCUGC |
| | | ACCCGUACCCCCCACCGCGUUAUCCGUUCCUCGUAGGCUG |
| | | GUCCUGGGGAACGGGUCGGCGGGUGGUCUUUGAAUAAAG |
| | | UCUGAGUGGGCGGC |
| 139 | B28 | UAAAGCUCCCCGGGGGCCUCGGUGGCCUAGCUUCUUGCCC |
| | | CUUGGGCCUCCCCCCAGCCCCUCCUCCCCUUCCUGCACCCG |
| | | UACCCCCGUGGUCUUUGAAUAAAGUCUGAGUGGGCGGC |
| 140 | B29 | UAAGUCUCCGCUGGAGCCUCGGUGGCCUAGCUUCUUGCCC |
| | | CUUGGGCCUCCCCCCAGCCCCUCCUCCCCUUCCUGCACCCG |
| | | UACCCCCGUGGUCUUUGAAUAAAGUCUGAGUGGGCGGC |
| 141 | B30 | UAAGCCCCUCCGGGGGCCUCGGUGGCCUAGCUUCUUGCCC |
| | | CUUGGGCCUCCCCCCAGCCCCUCCUCCCCUUCCUGCACCCG |
| | | UACCCCCGUGGUCUUUGAAUAAAGUCUGAGUGGGCGGC |

In an aspect, disclosed herein is a polynucleotide encoding a polypeptide, wherein the polynucleotide comprises: (a) a 5′-UTR, e.g., as described herein; (b) a coding region comprising a stop element (e.g., as described herein); and (c) a 3′-UTR (e.g., as described herein).

In an aspect, an LNP composition comprising a polynucleotide comprising an open reading frame encoding a checkpoint cancer vaccine comprising (i) one or more IDO antigenic peptides and (ii) one or more PD-L1 antigenic peptides (e.g., SEQ ID NO: 6, 300, 301, or 302) and comprising a 3′ UTR disclosed herein comprises: (i) an ionizable lipid, e.g., an amino lipid; (ii) a sterol or other structural lipid; (iii) a non-cationic helper lipid or phospholipid; and (iv) a PEG-lipid.

### Regions Having a 5′ Cap

The disclosure also includes a polynucleotide that comprises both a 5′ Cap and a polynucleotide of the present invention (e.g., a polynucleotide comprising a nucleotide sequence encoding a checkpoint cancer vaccine).

The 5′ cap structure of a natural mRNA is involved in nuclear export, increasing mRNA stability and binds the mRNA Cap Binding Protein (CBP), which is responsible for mRNA stability in the cell and translation competency through the association of CBP with poly(A) binding protein to form the mature cyclic mRNA species. The cap further assists the removal of 5′ proximal introns during mRNA splicing.

Endogenous mRNA molecules can be 5′-end capped generating a 5′-ppp-5′-triphosphate linkage between a terminal guanosine cap residue and the 5′-terminal transcribed sense nucleotide of the mRNA molecule. This 5′-guanylate cap can then be methylated to generate an N7-methyl-guanylate residue. The ribose sugars of the terminal and/or ante-terminal transcribed nucleotides of the 5′ end of the mRNA can optionally also be 2′-O-methylated. 5′-decapping through hydrolysis and cleavage of the guanylate cap structure can target a nucleic acid molecule, such as an mRNA molecule, for degradation.

In some embodiments, the polynucleotides of the present invention (e.g., a polynucleotide comprising a nucleotide sequence encoding a checkpoint cancer vaccine) incorporate a cap moiety.

In some embodiments, polynucleotides of the present invention (e.g., a polynucleotide comprising a nucleotide sequence encoding a checkpoint cancer vaccine) comprise a non-hydrolyzable cap structure preventing decapping and thus increasing mRNA half-life. Because cap structure hydrolysis requires cleavage of 5′-ppp-5′ phosphorodiester linkages, modified nucleotides can be used during the capping reaction. For example, a Vaccinia Capping Enzyme from New England Biolabs (Ipswich, MA) can be used with α-thio-guanosine nucleotides according to the manufacturer's instructions to create a phosphorothioate linkage in the 5′-ppp-5′ cap. Additional modified guanosine nucleotides can be used such as α-methyl-phosphonate and seleno-phosphate nucleotides.

Additional modifications include, but are not limited to, 2′-O-methylation of the ribose sugars of 5′-terminal and/or 5′-anteterminal nucleotides of the polynucleotide (as mentioned above) on the 2′-hydroxyl group of the sugar ring. Multiple distinct 5′-cap structures can be used to generate the 5′-cap of a nucleic acid molecule, such as a polynucleotide that functions as an mRNA molecule. Cap analogs, which herein are also referred to as synthetic cap analogs, chemical caps, chemical cap analogs, or structural or functional cap analogs, differ from natural (i.e., endogenous, wild-type or physiological) 5′-caps in their chemical structure, while retaining cap function. Cap analogs can be chemically (i.e., non-enzymatically) or enzymatically synthesized and/or linked to the polynucleotides of the invention.

For example, the Anti-Reverse Cap Analog (ARCA) cap contains two guanines linked by a 5′-5′-triphosphate group, wherein one guanine contains an N7 methyl group as well as a 3′-O-methyl group (i.e., N7,3′-O-dimethyl-guanosine-5′-triphosphate-5′-guanosine (m7G-3′mppp-G; which can equivalently be designated 3′ O-Me-m7G(5′)ppp(5′) G). The 3′-O atom of the other, unmodified, guanine becomes linked to the 5′-terminal nucleotide of the capped polynucleotide. The N7- and 3′-O-methlyated guanine provides the terminal moiety of the capped polynucleotide.

Another exemplary cap is mCAP, which is similar to ARCA but has a 2′-O-methyl group on guanosine (i.e., N7,2′-O-dimethyl-guanosine-5′-triphosphate-5′-guanosine, m7Gm-ppp-G).

Another exemplary cap is m⁷G-ppp-Gm-A (i.e., N7,guanosine-5′-triphosphate-2′-O-dimethyl-guanosine-adenosine).

In some embodiments, the cap is a dinucleotide cap analog. As a non-limiting example, the dinucleotide cap analog can be modified at different phosphate positions with a boranophosphate group or a phosphoroselenoate group such as the dinucleotide cap analogs described in U.S. Pat. No. 8,519,110, the contents of which are herein incorporated by reference in its entirety.

In another embodiment, the cap is a cap analog is a N7-(4-chlorophenoxyethyl) substituted dinucleotide form of a cap analog known in the art and/or described herein. Non-limiting examples of a N7-(4-chlorophenoxyethyl) substituted dinucleotide form of a cap analog include a N7-(4-chlorophenoxyethyl)-G(5′)ppp(5′)G and a N7-(4-chlorophenoxyethyl)-m3′-OG(5′)ppp(5′)G cap analog (See, e.g., the various cap analogs and the methods of synthesizing cap analogs described in Kore et al. Bioorganic & Medicinal Chemistry 2013 21:4570-4574; the contents of which are herein incorporated by reference in its entirety). In another embodiment, a cap analog of the present invention is a 4-chloro/bromophenoxyethyl analog.

While cap analogs allow for the concomitant capping of a polynucleotide or a region thereof, in an in vitro transcription reaction, up to 20% of transcripts can remain uncapped. This, as well as the structural differences of a cap analog from an endogenous 5′-cap structures of nucleic acids produced by the endogenous, cellular transcription machinery, can lead to reduced translational competency and reduced cellular stability.

Polynucleotides of the invention (e.g., a polynucleotide comprising a nucleotide sequence encoding a checkpoint cancer vaccine comprising (i) one or more IDO antigenic peptides and (ii) one or more PD-L1 antigenic peptides) can also be capped post-manufacture (whether IVT or chemical synthesis), using enzymes, to generate more authentic 5′-cap structures. As used herein, the phrase “more authentic” refers to a feature that closely mirrors or mimics, either structurally or functionally, an endogenous or wild type feature. That is, a “more authentic” feature is better representative of an endogenous, wild-type, natural or physiological cellular function and/or structure as compared to synthetic features or analogs, etc., of the prior art, or which outperforms the corresponding endogenous, wild-type, natural or physiological feature in one or more respects. Non-limiting examples of more authentic 5′cap structures of the present invention are those that, among other things, have enhanced binding of cap binding proteins, increased half-life, reduced susceptibility to 5′ endonucleases and/or reduced 5′ decapping, as compared to synthetic 5′ cap structures known in the art (or to a wild-type, natural or physiological 5′ cap structure). For example, recombinant Vaccinia Virus Capping Enzyme and recombinant 2′-O-methyltransferase enzyme can create a canonical 5′-5′-triphosphate linkage between the 5′-terminal nucleotide of a polynucleotide and a guanine cap nucleotide wherein the cap guanine contains an N7 methylation and the 5′-terminal nucleotide of the mRNA contains a 2′-O-methyl. Such a structure is termed the Cap1 structure. This cap results in a higher translational-competency and cellular stability and a reduced activation of cellular pro-inflammatory cytokines, as compared, e.g., to other 5′ cap analog structures known in the art. Cap structures include, but are not limited to, 7 mG(5′)ppp(5′)N1pN2p (cap 0), 7mG(5′)ppp(5′)N1mpN2p (cap 1), and 7mG(5′)-ppp(5′) N1mpN2mp (cap 2). Cap 1 is sometimes referred to as Cap C1 herein. In some embodiments, Cap C1 can optionally include an additional G at the 3′ end of the cap. In some embodiments, in Cap C1, N2 may comprise the first nucleotide of a 5′ UTR.

As a non-limiting example, capping chimeric polynucleotides post-manufacture can be more efficient as nearly 100% of the chimeric polynucleotides can be capped. This is in contrast to ˜80% efficiency when a cap analog is linked to a chimeric polynucleotide during an in vitro transcription reaction.

According to the present invention, 5′ terminal caps can include endogenous caps or cap analogs. According to the present invention, a 5′ terminal cap can comprise a guanine analog. Useful guanine analogs include, but are not limited to, inosine, N1-methyl-guanosine, 2′fluoro-guanosine, 7-deaza-guanosine, 8-oxo-guanosine, 2-amino-guanosine, LNA-guanosine, and 2-azido-guanosine.

Also provided herein are exemplary caps including those that can be used in co-transcriptional capping methods for ribonucleic acid (RNA) synthesis, using RNA polymerase, e.g., wild type RNA polymerase or variants thereof, e.g., such as those variants described herein. In one embodiment, caps can be added when RNA is produced in a “one-pot” reaction, without the need for a separate capping reaction. Thus, the methods, in some embodiments, comprise reacting a polynucleotide template with an RNA polymerase variant, nucleoside triphosphates, and a cap analog under in vitro transcription reaction conditions to produce RNA transcript.

A cap analog may be, for example, a dinucleotide cap, a trinucleotide cap, or a tetranucleotide cap. In some embodiments, a cap analog is a dinucleotide cap. In some embodiments, a cap analog is a trinucleotide cap. In some embodiments, a cap analog is a tetranucleotide cap. As used here the term “cap” includes the inverted G nucleotide and can comprise one or more additional nucleotides 3′ of the inverted G nucleotide, e.g., 1, 2, or more nucleotides 3′ of the inverted G nucleotide and 5′ to the 5′ UTR, e.g., a 5′ UTR described herein.

Exemplary caps comprise a sequence of GG, GA, or GGA, wherein the underlined, italicized G is an in inverted G nucleotide followed by a 5′-5′-triphosphate group.

A trinucleotide cap, in some embodiments, comprises a compound of formula (I)

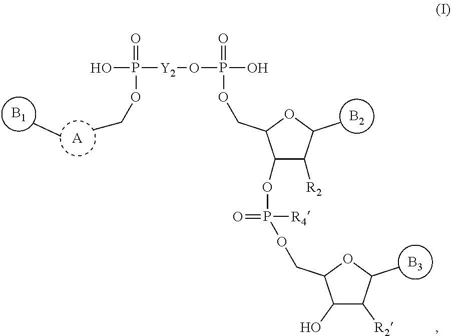

or a stereoisomer, tautomer or salt thereof, wherein

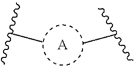

is

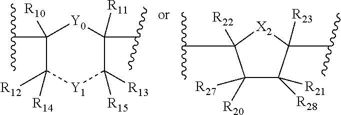

ring B1 is a modified or unmodified Guanine;
ring B2 and ring B3 each independently is a nucleobase or a modified nucleobase;
X2 is O, S(O) p, NR24 or CR25R26 in which p is 0, 1, or 2;
Y0 is O or CR6R7;
Y1 is O, S(O)n, CR6R⁷, or NR8, in which n is 0, 1, or 2;
each - - - is a single bond or absent, wherein when each - - - is a single bond, Yi is O, S(O)n, CR6R7, or NR8; and when each - - - is absent, Y1 is void;
Y2 is (OP(O)R4)m in which m is 0, 1, or 2, or —O—(CR40R41)u-Q0-(CR42R43) v-, in which Q0 is a bond, O, S(O)r, NR44, or CR45R46, r is 0, 1, or 2, and each of u and v independently is 1, 2, 3 or 4;
each R2 and R2′ independently is halo, LNA, or OR3;
each R3 independently is H, C1-C6 alkyl, C2-C6 alkenyl, or C2-C6 alkynyl and R3, when being C1-C6 alkyl, C2-C6 alkenyl, or C2-C6 alkynyl, is optionally substituted with one or more of halo, OH and C1-C6 alkoxyl that is optionally substituted with one or more OH or OC(O)—C1-C6 alkyl;
each R4 and R4′ independently is H, halo, C1-C6 alkyl, OH, SH, SeH, or BH3-;
each of R6, R7, and R8, independently, is -Q1-T1, in which Q1 is a bond or C1-C3 alkyl linker optionally substituted with one or more of halo, cyano, OH and C1-C6 alkoxy, and T1 is H, halo, OH, COOH, cyano, or Rs1, in which Rs1 is C1-C3 alkyl, C2-C6 alkenyl, C2-C6 alkynyl, C1-C6 alkoxyl, C(O)O—C1-C6 alkyl, C3-C8 cycloalkyl, C6-C10 aryl, NR31R32, (NR31R32R33)+, 4 to 12-membered heterocycloalkyl, or 5- or 6-membered heteroaryl, and Rs1 is optionally substituted with one or more substituents selected from the group consisting of halo, OH, oxo, C1-C6 alkyl, COOH, C(O)O—C1-C6 alkyl, cyano, C1-C6 alkoxyl, NR31R32, (NR31R32R33)+, C3-C8 cycloalkyl, C6-C10 aryl, 4 to 12-membered heterocycloalkyl, and 5- or 6-membered heteroaryl;
each of R10, R11, R12, R13 R14, and R15, independently, is -Q2-T2, in which Q2 is a bond or C1-C3 alkyl linker optionally substituted with one or more of halo, cyano, OH and C1-C6 alkoxy, and T2 is H, halo, OH, NH2, cyano, NO2, N3, Rs2, or ORs2, in which Rs2 is C1-C6 alkyl, C2-C6 alkenyl, C2-C6 alkynyl, C3-C8 cycloalkyl, C6-C10 aryl, NHC(O)—C1-C6 alkyl, NR31R32, (NR31R32R33)+, 4 to 12-membered heterocycloalkyl, or 5- or 6-membered heteroaryl, and Rs2 is optionally substituted with one or more substituents selected from the group consisting of halo, OH, oxo, C1-C6 alkyl, COOH, C(O)O—C1-C6 alkyl, cyano, C1-C6 alkoxyl, NR31R32, (NR31R32R33)+, C3-C8 cycloalkyl, C6-C10 aryl, 4 to 12-membered heterocycloalkyl, and 5- or 6-membered heteroaryl; or alternatively R12 together with R14 is oxo, or R13 together with R15 is oxo,
each of R20, R21, R22, and R23 independently is -Q3-T3, in which Q3 is a bond or C1-C3 alkyl linker optionally substituted with one or more of halo, cyano, OH and C₁-C₆ alkoxy, and T3 is H, halo, OH, NH2, cyano, NO2, N3, RS3, or ORS3, in which RS3 is C1-C6 alkyl, C2-C6 alkenyl, C2-C6 alkynyl, C3-C8 cycloalkyl, C6-C10 aryl, NHC(O)—C1-C6 alkyl, mono-C1-C6 alkylamino, di-C1-C6 alkylamino, 4 to 12-membered heterocycloalkyl, or 5- or 6-membered heteroaryl, and Rs3 is optionally substituted with one or more substituents selected from the group consisting of halo, OH, oxo, C1-C6 alkyl, COOH, C(O)O—C1-C6 alkyl, cyano, C1-C6 alkoxyl, amino, mono-C1-C6 alkylamino, di-C1-C6 alkylamino, C3-C8 cycloalkyl, C6-C10 aryl, 4 to 12-membered heterocycloalkyl, and 5- or 6-membered heteroaryl;
each of R24, R25, and R26 independently is H or C1-C6 alkyl;
each of R27 and R28 independently is H or OR29; or R27 and R28 together form O—R30-O; each R29 independently is H, C1-C6 alkyl, C2-C6 alkenyl, or C2-C6 alkynyl and R29, when being C1-C6 alkyl, C2-C6 alkenyl, or C2-C6 alkynyl, is optionally substituted with one or more of halo, OH and C1-C6 alkoxyl that is optionally substituted with one or more OH or OC(O)—C1-C6 alkyl;
R30 is C1-C6 alkylene optionally substituted with one or more of halo, OH and C1-C6 alkoxyl;
each of R31, R32, and R33, independently is H, C1-C6 alkyl, C3-C8 cycloalkyl, C6-C10 aryl, 4 to 12-membered heterocycloalkyl, or 5- or 6-membered heteroaryl;
each of R40, R41, R42, and R43 independently is H, halo, OH, cyano, N3, OP(O)R47R48, or C1-C6 alkyl optionally substituted with one or more OP(O)R47R48, or one R41 and one R43, together with the carbon atoms to which they are attached and Q0, form C4-C10 cycloalkyl, 4- to 14-membered heterocycloalkyl, C6-C10 aryl, or 5- to 14-membered heteroaryl, and each of the cycloalkyl, heterocycloalkyl, phenyl, or 5- to 6-membered heteroaryl is optionally substituted with one or more of OH, halo, cyano, N3, oxo, OP(O)R47R48, C1-C6 alkyl, C1-C6 haloalkyl, COOH, C(O)O—C1-C6 alkyl, C1-C6 alkoxyl, C1-C6 haloalkoxyl, amino, mono-C1-C6 alkylamino, and di-C1-C6 alkylamino; R44 is H, C1-C6 alkyl, or an amine protecting group;

each of R45 and R46 independently is H, OP(O)R47R48, or C1-C6 alkyl optionally substituted with one or more OP(O)R47R48, and each of R47 and R48, independently is H, halo, C1-C6 alkyl, OH, SH, SeH, or BH3.

It should be understood that a cap analog, as provided herein, may include any of the cap analogs described in international publication WO 2017/066797, published on 20 Apr. 2017, incorporated by reference herein in its entirety.

In some embodiments, the B2 middle position can be a non-ribose molecule, such as arabinose.

In some embodiments R² is ethyl-based.

Thus, in some embodiments, a trinucleotide cap comprises the following structure:

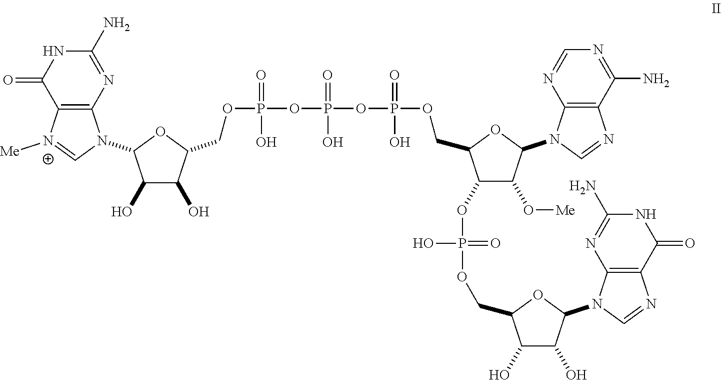

In other embodiments, a trinucleotide cap comprises the following structure:

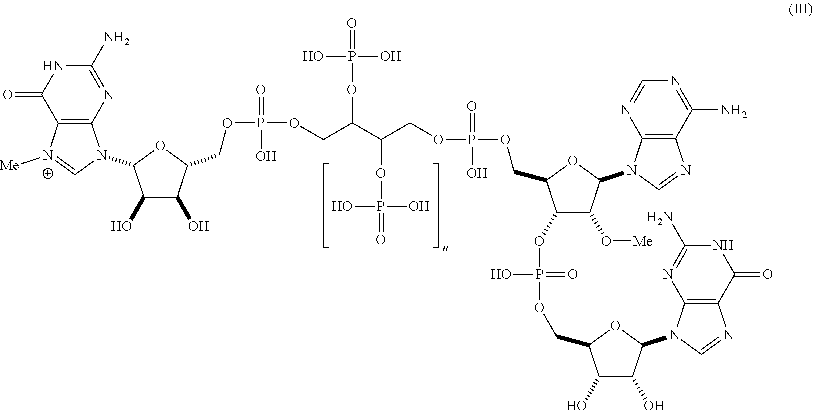

In yet other embodiments, a trinucleotide cap comprises the following structure:

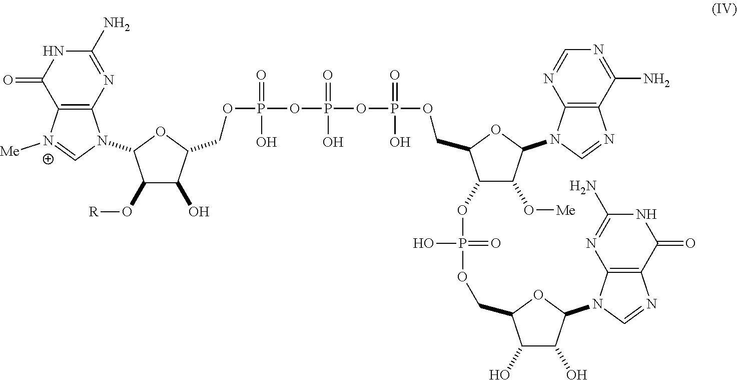

In still other embodiments, a trinucleotide cap comprises the following structure:

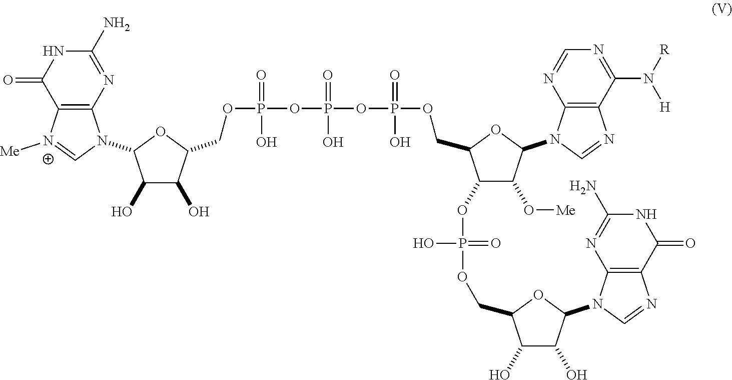

In some embodiments, R is an alkyl (e.g., C₁-C₆ alkyl). In some embodiments, R is a methyl group (e.g., C1 alkyl). In some embodiments, R is an ethyl group (e.g., C2 alkyl).

A dinucleotide cap, in some embodiments, comprises a compound of formula (I-b)

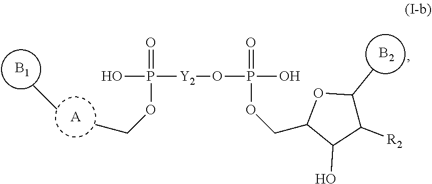

or a stereoisomer, tautomer or salt thereof, wherein

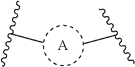

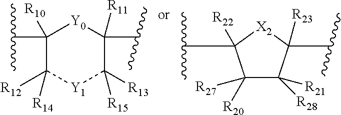

ring B1 is a modified or unmodified Guanine;
ring B2 is a nucleobase or a modified nucleobase;
X2 is O, S(O) p, NR24 or CR25R26 in which p is 0, 1, or 2;
Y0 is O or CR6R7;
Y1 is O, S(O)n, CR6R7, or NR8, in which n is 0, 1, or 2;
each - - - is a single bond or absent, wherein when each - - - is a single bond, Y1 is O, S(O)n, CR6R7, or NR8; and when each - - - is absent, Y1 is void;
Y2 is (OP(O)R4)m in which m is 0, 1, or 2, or —O—(CR40R41) u-Q0-(CR42R43) v-, in which Q0 is a bond, O, S(O)r, NR44, or CR45R46, r is 0, 1, or 2, and each of u and v independently is 1, 2, 3 or 4; R2 is halo, LNA, or OR3;
each R3 independently is H, C1-C6 alkyl, C2-C6 alkenyl, or C2-C6 alkynyl and R3, when being C1-C6 alkyl, C2-C6 alkenyl, or C2-C6 alkynyl, is optionally substituted with one or more of halo, OH and C1-C6 alkoxyl that is optionally substituted with one or more OH or OC(O)—C1-C6 alkyl;
R4 is H, halo, C1-C6 alkyl, OH, SH, SeH, or BH3-;
each of R6, R7, and R8, independently, is -Q1-T1, in which Q1 is a bond or C1-C3 alkyl linker optionally substituted with one or more of halo, cyano, OH and C1-C6 alkoxy, and T1 is H, halo, OH, COOH, cyano, or Rs1, in which Rs1 is C1-C3 alkyl, C2-C6 alkenyl, C2-C6 alkynyl, C1-C6 alkoxyl, C(O)O—C1-C6 alkyl, C3-C8 cycloalkyl, C6-C10 aryl, NR31R32, (NR31R32R33)+, 4 to 12-membered heterocycloalkyl, or 5- or 6-membered heteroaryl, and Rs1 is optionally substituted with one or more substituents selected from the group consisting of halo, OH, oxo, C1-C6 alkyl, COOH, C(O)O—C1-C6 alkyl, cyano, C1-C6 alkoxyl, NR31R32, (NR31R32R33)+, C3-C8 cycloalkyl, C6-C10 aryl, 4 to 12-membered heterocycloalkyl, and 5- or 6-membered heteroaryl;
each of R10, R11, R12, R13 R14, and R15, independently, is -Q2-T2, in which Q2 is a bond or C1-C3 alkyl linker optionally substituted with one or more of halo, cyano, OH and C1-C6 alkoxy, and T2 is H, halo, OH, NH2, cyano, NO2, N3, Rs2, or ORs2, in which Rs2 is C1-C6 alkyl, C2-C6 alkenyl, C2-C6 alkynyl, C3-C8 cycloalkyl, C6-C10 aryl, NHC(O)—C1-C6 alkyl, NR31R32, (NR31R32R33)+, 4 to 12-membered heterocycloalkyl, or 5- or 6-membered heteroaryl, and Rs2 is optionally substituted with one or more substituents selected from the group consisting of halo, OH, oxo, C1-C6 alkyl, COOH, C(O)O—C1-C6 alkyl, cyano, C1-C6 alkoxyl, NR31R32, (NR31R32R33)+, C3-C8 cycloalkyl, C6-C10 aryl, 4 to 12-membered heterocycloalkyl, and 5- or 6-membered heteroaryl; or alternatively R12 together with R14 is oxo, or R13 together with R15 is oxo,
each of R20, R21, R22, and R23 independently is -Q3-T3, in which Q3 is a bond or C1-C3 alkyl linker optionally substituted with one or more of halo, cyano, OH and C1-C6 alkoxy, and T3 is H, halo, OH, NH2, cyano, NO2, N3, RS3, or ORS3, in which RS3 is C1-C6 alkyl, C2-C6 alkenyl, C2-C6 alkynyl, C3-C8 cycloalkyl, C6-C10 aryl, NHC(O)—C1-C6 alkyl, mono-C1-C6 alkylamino, di-C1-C6 alkylamino, 4 to 12-membered heterocycloalkyl, or 5- or 6-membered heteroaryl, and Rs3 is optionally substituted with one or more substituents selected from the group consisting of halo, OH, oxo, C1-C6 alkyl, COOH, C(O)O—C1-C6 alkyl, cyano, C1-C6 alkoxyl, amino, mono-C1-C6 alkylamino, di-C1-C6 alkylamino, C3-C8 cycloalkyl, C6-C10 aryl, 4 to 12-membered heterocycloalkyl, and 5- or 6-membered heteroaryl; each of R24, R25, and R26 independently is H or C1-C6 alkyl;

each of R27 and R28 independently is H or OR29; or R27 and R28 together form O—R30-O; each R29 independently is H, C1-C6 alkyl, C2-C6 alkenyl, or C2-C6 alkynyl and R29, when being C1-C6 alkyl, C2-C6 alkenyl, or C2-C6 alkynyl, is optionally substituted with one or more of halo, OH and C1-C6 alkoxyl that is optionally substituted with one or more OH or OC(O)—C1-C6 alkyl;

R30 is C1-C6 alkylene optionally substituted with one or more of halo, OH and C1-C6 alkoxyl; each of R31, R32, and R33, independently is H, C1-C6 alkyl, C3-C8 cycloalkyl, C6-C10 aryl, 4 to 12-membered heterocycloalkyl, or 5- or 6-membered heteroaryl;

each of R40, R41, R42, and R43 independently is H, halo, OH, cyano, N3, OP(O)R47R48, or C1-C6 alkyl optionally substituted with one or more OP(O)R47R48, or one R41 and one R43, together with the carbon atoms to which they are attached and Q0, form C4-C10 cycloalkyl, 4- to 14-membered heterocycloalkyl, C6-C10 aryl, or 5- to 14-membered heteroaryl, and each of the cycloalkyl, heterocycloalkyl, phenyl, or 5- to 6-membered heteroaryl is optionally substituted with one or more of OH, halo, cyano, N3, oxo, OP(O)R47R48, C1-C6 alkyl, C1-C6 haloalkyl, COOH, C(O)O—C1-C6 alkyl, C1-C6 alkoxyl, C1-C6 haloalkoxyl, amino, mono-C1-C6 alkylamino, and di-C1-C6 alkylamino; R44 is H, C1-C6 alkyl, or an amine protecting group;

each of R45 and R46 independently is H, OP(O)R47R48, or C1-C6 alkyl optionally substituted with one or more OP(O)R47R48, and each of R47 and R48, independently is H, halo, C1-C6 alkyl, OH, SH, SeH, or BH3.

Thus, in some embodiments, a dinucleotide cap comprises the following structure:

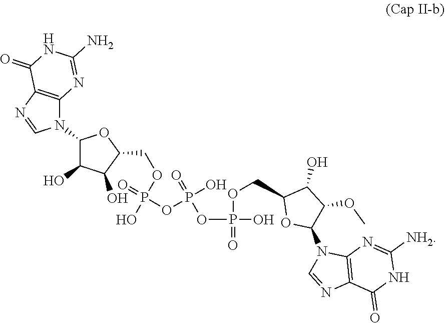 [SMILES: CO[C@H]1[C@@H](O)[C@H](COP(=O)(O)OP(=O)(O)OP(=O)(O)OC[C@H]2O[C@@H](n3cnc4c(=O)[nH]c(N)nc43)[C@H](O)[C@@H]2O)O[C@@H]1n1cnc2c(=O)[nH]c(N)nc21]

A trinucleotide cap, in some embodiments, comprises a sequence selected from the following sequences: GAA, GAC, GAG, GAU, GCA, GCC, GCG, GCU, GGA, GGC, GGG, GGU, GUA, GUC, GUG, and GUU. In some embodiments, a trinucleotide cap comprises GAA. In some embodiments, a trinucleotide cap comprises GAC. In some embodiments, a trinucleotide cap comprises GAG. In some embodiments, a trinucleotide cap comprises GAU. In some embodiments, a trinucleotide cap comprises GCA. In some embodiments, a trinucleotide cap comprises GCC. In some embodiments, a trinucleotide cap comprises GCG. In some embodiments, a trinucleotide cap comprises GCU. In some embodiments, a trinucleotide cap comprises GGA. In some embodiments, a trinucleotide cap comprises GGC. In some embodiments, a trinucleotide cap comprises GGG. In some embodiments, a trinucleotide cap comprises GGU. In some embodiments, a trinucleotide cap comprises GUA. In some embodiments, a trinucleotide cap comprises GUC. In some embodiments, a trinucleotide cap comprises GUG. In some embodiments, a trinucleotide cap comprises GUU.

In some embodiments, a trinucleotide cap comprises a sequence selected from the following sequences: m⁷GpppApA, m⁷GpppApC, m⁷GpppApG, m⁷GpppApU, m⁷GpppCpA, m⁷GpppCpC, m⁷GpppCpG, m⁷GpppCpU, m⁷GpppGpA, m⁷GpppGpC, m⁷GpppGpG, m⁷GpppGpU, m⁷GpppUpA, m⁷GpppUpC, m⁷GpppUpG, and m⁷GpppUpU.

In some embodiments, a trinucleotide cap comprises m⁷GpppApA. In some embodiments, a trinucleotide cap comprises m⁷GpppApC. In some embodiments, a trinucleotide cap comprises m⁷GpppApG. In some embodiments, a trinucleotide cap comprises m⁷GpppApU. In some embodiments, a trinucleotide cap comprises m⁷GpppCpA. In some embodiments, a trinucleotide cap comprises m⁷GpppCpC. In some embodiments, a trinucleotide cap comprises m⁷GpppCpG. In some embodiments, a trinucleotide cap comprises m⁷GpppCpU. In some embodiments, a trinucleotide cap comprises m⁷GpppGpA. In some embodiments, a trinucleotide cap comprises m⁷GpppGpC. In some embodiments, a trinucleotide cap comprises m⁷GpppGpG. In some embodiments, a trinucleotide cap comprises m⁷GpppGpU. In some embodiments, a trinucleotide cap comprises m⁷GpppUpA. In some embodiments, a trinucleotide cap comprises m⁷GpppUpC. In some embodiments, a trinucleotide cap comprises m⁷GpppUpG. In some embodiments, a trinucleotide cap comprises m⁷GpppUpU.

A trinucleotide cap, in some embodiments, comprises a sequence selected from the following sequences: m⁷G_{3′OMe}pppApA, m⁷G_{3′OMe}pppApC, m⁷G_{3′OMe}pppApG, m⁷G_{3′OMe}pppApU, m⁷G_{3′OMe}pppCpA, m⁷G_{3′OMe}pppCpC, m⁷G_{3′OMe}pppCpG, m⁷G_{3′OMe}pppCPU, m⁷G_{3′OMe}pppGpA, m⁷G_{3′OMe}pppGpC, m⁷G_{3′OMe}pppGpG, m⁷G_{3′OMe}pppGpU, m⁷G_{3′OMe}pppUpA, m⁷G_{3′OMe}pppUpC, m⁷G_{3′OMe}pppUpG, and m⁷G_{3′OMe}pppUpU.

In some embodiments, a trinucleotide cap comprises m⁷G_{3′OMe}pppApA. In some embodiments, a trinucleotide cap comprises m⁷G_{3′OMe}pppApC. In some embodiments, a trinucleotide cap comprises m⁷G_{3′OMe}pppApG. In some embodiments, a trinucleotide cap comprises m⁷G_{3′OMe}pppApU. In some embodiments, a trinucleotide cap comprises m⁷G_{3′OMe}pppCpA. In some embodiments, a trinucleotide cap comprises m⁷G_{3′OMe}pppCpC. In some embodiments, a trinucleotide cap comprises m⁷G_{3′OMe}pppCpG. In some embodiments, a trinucleotide cap comprises m⁷G_{3′OMe}pppCpU. In some embodiments, a trinucleotide cap comprises m⁷G_{3′OMe}pppGpA. In some embodiments, a trinucleotide cap comprises m⁷G_{3′OMe}pppGpC. In some embodiments, a trinucleotide cap comprises m⁷G_{3′OMe}pppGpG. In some embodiments, a trinucleotide cap comprises m⁷G_{3′OMe}pppGpU. In some embodiments, a trinucleotide cap comprises m⁷G_{3′OMe}pppUpA. In some embodiments, a trinucleotide cap comprises m⁷G_{3′OMe}pppUpC. In some embodiments, a trinucleotide cap comprises m⁷G_{3′OMe}pppUpG. In some embodiments, a trinucleotide cap comprises m⁷G_{3′OMe}pppUpU.

A trinucleotide cap, in other embodiments, comprises a sequence selected from the following sequences: m⁷G_{3′OMe}pppA_{2′OMe}pA, m⁷G_{3′OMe}pppA_{2′OMe}pC, m⁷G_{3′OMe}pppA_{2′OMe}pG, m⁷G_{3′OMe}pppA_{2′OMe}pU, m⁷G_{3′OMe}ppp_{2′OMe}pA, m⁷G_{3′OMe}pppC_{2′OMe}pC, m⁷G_{3′OMe}pppC_{2′OMe}pG, m⁷G_{3′OMe}pppC_{2′OMe}pU, m⁷G_{3′OMe}pppG_{2′OMe}pA, m⁷G_{3′OMe}pppG_{2′OMe}pC, m⁷G_{3′OMe}pppG_{2′OMe}pG, m⁷G_{3′OMe}ppp_{2′OMe}pU, m⁷G_{3′OMe}pppU_{2′OMe}pA, m⁷G_{3′OMe}pppU_{2′OMe}pC, m⁷G_{3′OMe}pppU_{2′OMe}pG, and m⁷G_{3′OMe}pppU_{2′OMe}pU.

In some embodiments, a trinucleotide cap comprises m⁷G_{3′OMe}pppA_{2′OMe}pA. In some embodiments, a trinucleotide cap comprises m⁷G_{3′OMe}pppA_{2′OMe}pC. In some embodiments, a trinucleotide cap comprises m⁷G_{3′OMe}pppA_{2′OMe}pG. In some embodiments, a trinucleotide cap comprises m⁷G_{3′OMe}pppA_{2′OMe}pU. In some embodiments, a trinucleotide cap comprises m⁷G_{3′OMe}pppC_{2′OMe}pA. In some embodiments, a trinucleotide cap comprises m⁷G_{3′OMe}pppC_{2′OMe}pC. In some embodiments, a trinucleotide cap comprises m⁷G_{3′OMe}pppC_{2′OMe}pG. In some embodiments, a trinucleotide cap comprises m⁷G_{3′OMe}pppC_{2′OMe}pU. In some embodiments, a trinucleotide cap comprises m⁷G_{3′OMe}pppG_{2′OMe}pA. In some embodiments, a trinucleotide cap comprises m⁷G_{3′OMe}pppG_{2′OMe}pC. In some embodiments, a trinucleotide cap comprises m⁷G_{3′OMe}pppG_{2′OMe}pG. In some embodiments, a trinucleotide cap comprises m⁷G_{3′OMe}pppG_{2′OMe}pU. In some embodiments, a trinucleotide cap comprises m⁷G_{3′OMe}pppU_{2′OMe}pA. In some embodiments, a trinucleotide cap comprises m⁷G_{3′OMe}pppU_{2′OMe}pC. In some embodiments, a trinucleotide cap comprises m⁷G_{3′OMe}pppU_{2′OMe}pG. In some embodiments, a trinucleotide cap comprises m⁷G_{3′OMe}pppU_{2′OMe}pU.

A trinucleotide cap, in still other embodiments, comprises a sequence selected from the following sequences: m⁷GpppA_{2′OMe}pA, m⁷GpppA_{2′OMe}pC, m⁷GpppA_{2′OMe}pG, m⁷GpppA_{2′OMe}pU, m⁷GpppCA_{2′OMe}pA, m⁷GpppC_{2′OMe}pC, m⁷GpppC_{2′OMe}pG, m⁷GpppC_{2′OMe}pU, m⁷GpppG_{2′OMe}pA, m⁷GpppG_{2′OMe}pC, m⁷GpppG_{2′OMe}pG, m⁷GpppG_{2′OMe}pU, m⁷GpppU_{2′OMe}pA, m⁷GpppU_{2′OMe}pC, m⁷GpppU_{2′OMe}pG, and m⁷GpppU_{2′OMe}pU.

In some embodiments, a trinucleotide cap comprises m⁷GpppA_{2′OMe}pA. In some embodiments, a trinucleotide cap comprises m⁷GpppA_{2′OMe}pC. In some embodiments, a trinucleotide cap comprises m⁷GpppA_{2′OMe}pG. In some embodiments, a trinucleotide cap comprises m⁷GpppA_{2′OMe}pU. In some embodiments, a trinucleotide cap comprises m⁷GpppC_{2′OMe}pA. In some embodiments, a trinucleotide cap comprises m⁷GpppC_{2′OMe}pC. In some embodiments, a trinucleotide cap comprises m⁷GpppC_{2′OMe}pG. In some embodiments, a trinucleotide cap comprises m⁷GpppC_{2′OMe}pU. In some embodiments, a trinucleotide cap comprises m⁷GpppG_{2′OMe}pA. In some embodiments, a trinucleotide cap comprises m⁷GpppG_{2′OMe}pC. In some embodiments, a trinucleotide cap comprises m⁷GpppG_{2′OMe}pG. In some embodiments, a trinucleotide cap comprises m⁷GpppG_{2′OMe}pU. In some embodiments, a trinucleotide cap comprises m⁷GpppU_{2′OMe}pA. In some embodiments, a trinucleotide cap comprises m⁷GpppU_{2′OMe}pC. In some embodiments, a trinucleotide cap comprises m⁷GpppU_{2′OMe}pG. In some embodiments, a trinucleotide cap comprises m⁷GpppU_{2′OMe}pU.

In some embodiments, a trinucleotide cap comprises m⁷Gpppm⁶A_{2′OMe}pG. In some embodiments, a trinucleotide cap comprises m⁷Gpppe⁶A_{2′OMe}pG.

In some embodiments, a trinucleotide cap comprises GAG. In some embodiments, a trinucleotide cap comprises GCG. In some embodiments, a trinucleotide cap comprises GUG. In some embodiments, a trinucleotide cap comprises GGG.

In some embodiments, a trinucleotide cap comprises any one of the following structures:

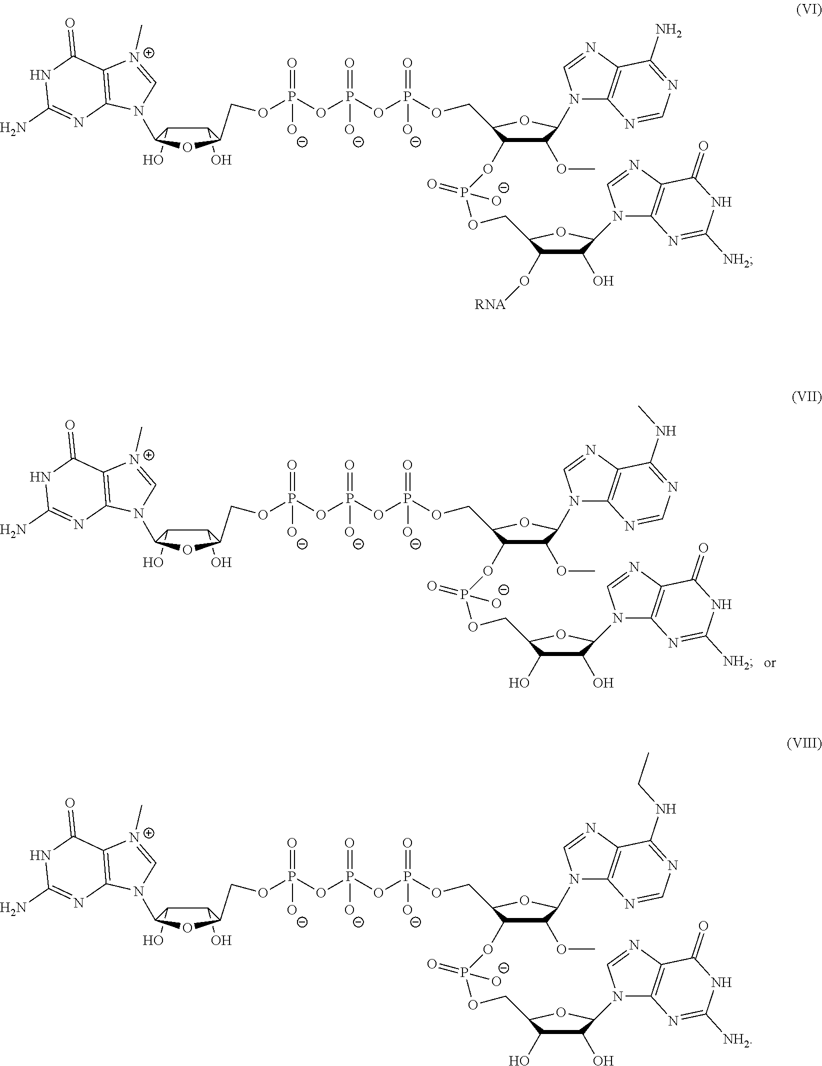

In some embodiments, the cap analog comprises a tetranucleotide cap. In some embodiments, the tetranucleotide cap comprises a trinucleotide as set forth above. In some embodiments, the tetranucleotide cap comprises m⁷GpppN₁N₂N₃, where N₁, N₂, and N₃ are optional (i.e., can be absent or one or more can be present) and are independently a natural, a modified, or an unnatural nucleoside base. In some embodiments, ^{m7}G is further methylated, e.g., at the 3′ position. In some embodiments, the ^{m7}G comprises an O-methyl at the 3′ position. In some embodiments N₁, N₂, and N₃ if present, optionally, are independently an adenine, a uracil, a guanidine, a thymine, or a cytosine. In some embodiments, one or more (or all) of N₁, N₂, and N₃, if present, are methylated, e.g., at the 2′ position. In some embodiments, one or more (or all) of N₁, N₂, and N₃, if present have an O-methyl at the 2′ position. In some embodiments, the tetranucleotide cap comprises the following structure:

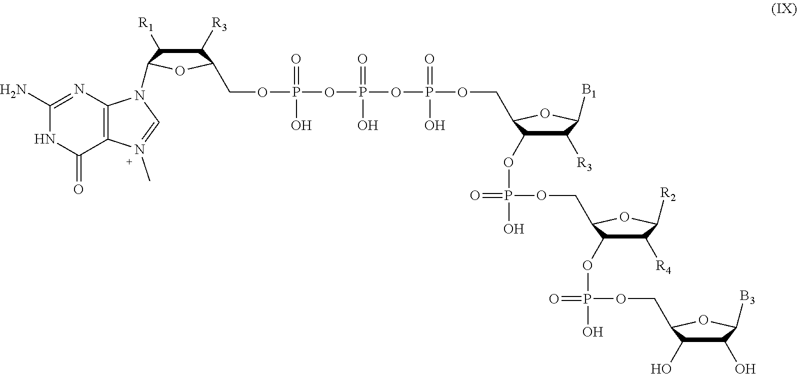

wherein B₁, B₂, and B₃ are independently a natural, a modified, or an unnatural nucleoside based; and R₁, R₂, R₃, and R₄ are independently OH or O-methyl. In some embodiments, R₃ is O-methyl and R₄ is OH. In some embodiments, R₃ and R₄ are O-methyl. In some embodiments, R₄ is O-methyl. In some embodiments, R₁ is OH, R₂ is OH, R₃ is O-methyl, and R₄ is OH. In some embodiments, R₁ is OH, R₂ is OH, R₃ is O-methyl, and R₄ is O-methyl. In some embodiments, at least one of R₁ and R₂ is O-methyl, R₃ is O-methyl, and R₄ is OH. In some embodiments, at least one of R₁ and R₂ is O-methyl, R₃ is O-methyl, and R₄ is O-methyl.

In some embodiments, B₁, B₃, and B₃ are natural nucleoside bases. In some embodiments, at least one of B₁, B₂, and B₃ is a modified or unnatural base. In some embodiments, at least one of B₁, B₂, and B₃ is N6-methyladenine. In some embodiments, B1 is adenine, cytosine, thymine, or uracil. In some embodiments, B₁ is adenine, B₂ is uracil, and B₃ is adenine. In some embodiments, R₁ and R₂ are OH, R₃ and R₄ are O-methyl, B₁ is adenine, B₂ is uracil, and B₃ is adenine.

In some embodiments the tetranucleotide cap comprises a sequence selected from the following sequences: GAAA, GACA, GAGA, GAUA, GCAA, GCCA, GCGA, GCUA, GGAA, GGCA, GGGA, GGUA, GUCA, and GUUA. In some embodiments the tetranucleotide cap comprises a sequence selected from the following sequences: GAAG, GACG, GAGG, GAUG, GCAG, GCCG, GCGG, GCUG, GGAG, GGCG, GGGG, GGUG, GUCG, GUGG, and GUUG. In some embodiments the tetranucleotide cap comprises a sequence selected from the following sequences: GAAU, GACU, GAGU, GAUU, GCAU, GCCU, GCGU, GCUU, GGAU, GGCU, GGGU, GGUU, GUAU, GUCU, GUGU, and GUUU.

In some embodiments the tetranucleotide cap comprises a sequence selected from the following sequences: GAAC, GACC, GAGC, GAUC, GCAC, GCCC, GCGC, GCUC, GGAC, GGCC, GGGC, GGUC, GUAC, GUCC, GUGC, and GUUC.

A tetranucleotide cap, in some embodiments, comprises a sequence selected from the following sequences: m⁷G_{2′OMe}pppApApN, m⁷G_{3′OMe}pppApCpN, m⁷G_{3′OMe}pppApGpN, m⁷G_{3′OMe}pppApUpN, m⁷G_{3′OMe}pppCpApN, m⁷G_{3′OMe}pppCpCpN, m⁷G_{3′OMe}pppCpGpN, m⁷G_{3′OMe}pppCpUpN, m⁷G_{3′OMe}pppGpApN, m⁷G_{3′OMe}pppGpCpN, m⁷G_{3′OMe}pppGpGpN, m⁷G_{3′OMe}pppGpUpN, m⁷G_{3′OMe}pppUpApN, m⁷G_{3′OMe}pppUpCpN, m⁷G_{3′OMe}pppUpGpN, and m⁷G_{3′OMe}pppUpUpN, where N is a natural, a modified, or an unnatural nucleoside base.

A tetranucleotide cap, in other embodiments, comprises a sequence selected from the following sequences: m⁷G_{3′OMe}pppA_{2′OMe}pApN, m⁷G_{3′OMe}pppA_{2′OMe}pCpN, m⁷G_{3′OMe}pppA_{2′OMe}pGpN, m⁷G_{3′OMe}pppA_{2′OMe}pUpN, m⁷G_{3′OMe}pppC_{2′OMe}pApN, m⁷G_{3′OMe}pppC_{2′OMe}pCpN, m⁷G_{3′OMe}pppC_{2′OMe}pGpN, m⁷G_{3′OMe}pppC_{2′OMe}pUpN, m⁷G_{3′OMe}pppG_{2′OMe}pApN, m⁷G_{3′OMe}pppG_{2′OMe}pCpN, m⁷G_{3′OMe}pppG_{2′OMe}pGpN, m⁷G_{3′OMe}pppG_{2′OMe}pUpN, m⁷G_{3′OMe}pppU_{2′OMe}pApN, m⁷G_{3′OMe}pppU_{2′OMe}pCpN, m⁷G_{3′OMe}pppU_{2′OMe}pGpN, and m⁷G_{2′OMe}pUpN, where N is a natural, a modified, or an unnatural nucleoside base.

A tetranucleotide cap, in still other embodiments, comprises a sequence selected from the following sequences: m⁷GpppA_{2′OMe}pApN, m⁷GpppA_{2′OMe}pCpN, m⁷GpppA_{2′OMe}pGpN, m⁷GpppA_{2′OMe}pUpN, m⁷GpppC_{2′OMe}pApN, m⁷GpppC_{2′OMe}pCpN, m⁷GpppC_{2′OMe}pGpN, m′GpppC_{2′OMe}pUpN, m′GppG_{2′OMe}pApN, m′GpppG_{2′OMe}pCpN, m′GpppG_{2′OMe}pGpN, m⁷GpppG_{2′OMe}pUpN, m⁷GpppU_{2′OMe}pApN, m⁷GpppU_{2′OMe}pCpN, m⁷GpppU_{2′OMe}pGpN, and m⁷GpppU_{2′OMe}pUpN, where N is a natural, a modified, or an unnatural nucleoside base.

A tetranucleotide cap, in other embodiments, comprises a sequence selected from the following sequences: m⁷G_{3′OMe}pppA_{2′OMe}pA_{2′OMe}pN, m⁷G_{3′OMe}pppA_{2′OMe}pC_{2′OMe}pN, m⁷G_{3′OMe}pppA_{2′OMe}pG_{3′OMe}pN, m⁷G_{3′OMe}pppA_{2′OMe}pU_{2′OMe}pN, m⁷G_{3′OMe}pppC_{2′OMe}pA_{2′OMe}pN, m⁷G_{3′OMe}pppC_{2′OMe}pC_{2′OMe}pN, m⁷G_{3′OMe}pppC_{2′OMe}pG_{2′OMe}pN, m⁷G_{3′OMe}pppC_{2′OMe}pU_{2′OMe}, m⁷G_{3′OMe}pppG_{2′OMe}pA_{2′OMe}pN, m⁷G_{3′OMe}pppG_{2′OMe}pC_{2′OMe}N, m⁷G_{3′OMe}pppG_{2′OMe}pG_{2′OMe}pN, m⁷G_{3′OMe}pppG_{2′OMe}pU_{2′OMe}pN, m⁷G_{3′OMe}pppU_{2′OMe}pA_{2′OMe}pN, m⁷G_{3′OMe}pppU_{2′OMe}pC_{2′OMe}pN, m⁷G_{3′OMe}pppU_{2′OMe}pG_{2′OMe}pN, and m⁷G_{3′OMe}pppU_{2′OMe}pU_{2′OMe}pN, where N is a natural, a modified, or an unnatural nucleoside base.

A tetranucleotide cap, in still other embodiments, comprises a sequence selected from the following sequences: m⁷GpppA_{2′OMe}pA_{2′OMe}pN, m⁷GpppA_{2′OMe}pC_{2′OMe}pN, m⁷GpppA_{2′OMe}pG_{2′OMe}pN, m⁷GpppA_{2′OMe}pU_{2′OMe}pN, m⁷GpppC_{2′OMe}pA_{2′OMe}pN, m⁷GpppC_{2′OMe}pC_{2′OMe}pN, m⁷GpppC_{2′OMe}pG_{2′OMe}pN, m⁷GpppC_{2′OMe}pU_{2′OMe}pN, m⁷GpppG_{2′OMe}pA_{2′OMe}pN, m⁷GpppG_{2′OMe}pC_{2′OMe}pN, m⁷GpppG_{2′OMe}pG_{2′OMe}pN, m⁷GpppG_{2′OMe}pU_{2′OMe}pN, m⁷GpppU_{2′OMe}pA_{2′OMe}pN, m⁷GpppU_{2′OMe}pC_{2′OMe}pN, m⁷GpppU_{2′OMe}pG_{2′OMe}pN, and m⁷GpppU_{2′OMe}pU_{2′OMe}pN, where N is a natural, a modified, or an unnatural nucleoside base. In some embodiments, a tetranucleotide cap comprises GGAG. In some embodiments, a tetranucleotide cap comprises the following structure:

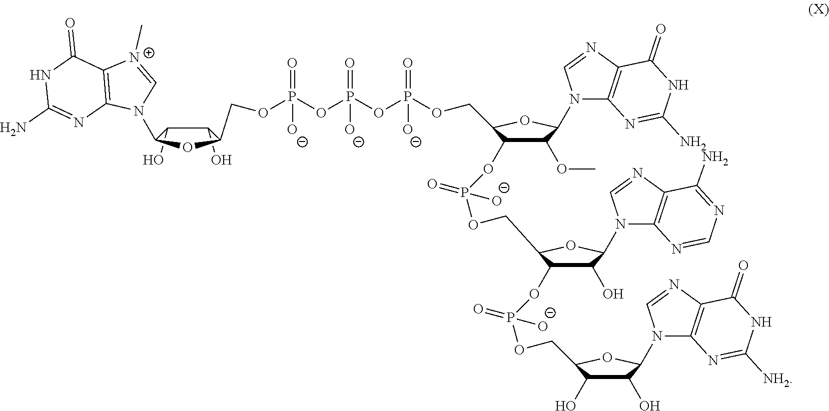

### Tails, e.g., Poly A Tails

In some embodiments, the polynucleotides of the present disclosure (e.g., a polynucleotide comprising a nucleotide sequence encoding a checkpoint cancer vaccine comprising (i) one or more IDO antigenic peptides and (ii) one or more PD-L1 antigenic peptides) further comprise a tail, e.g., a poly-A tail. In further embodiments, terminal groups on the poly-A tail can be incorporated for stabilization. In other embodiments, a poly-A tail comprises des-3′ hydroxyl tails.

During RNA processing, a long chain of adenine nucleotides (poly-A tail) can be added to a polynucleotide such as an mRNA molecule to increase stability. Immediately after transcription, the 3′ end of the transcript can be cleaved to free a 3′ hydroxyl. Then poly-A polymerase adds a chain of adenine nucleotides to the RNA. The process, called polyadenylation, adds a poly-A tail that can be between, for example, approximately 80 to approximately 250 residues long, including approximately 80, 90, 100, 110, 120, 130, 140, 150, 160, 170, 180, 190, 200, 210, 220, 230, 240 or 250 residues long. In one embodiment, the poly-A tail is 100 nucleotides in length (SEQ ID NO:502). aaaaaaaaaa aaaaaaaaaa aaaaaaaaaa aaaaaaaaaa aaaaaaaaaa aaaaaaaaaa aaaaaaaaaa aaaaaaaaaa aaaaaaaaaa aaaaaaaaaa (SEQ ID NO: 502)

PolyA tails can also be added after the construct is exported from the nucleus.

According to the present invention, terminal groups on the poly A tail can be incorporated for stabilization. Polynucleotides of the present invention can include des-3′ hydroxyl tails. They can also include structural moieties or 2′-Omethyl modifications as taught by Junjie Li, et al. (Current Biology, Vol. 15, 1501-1507 Aug. 23, 2005, the contents of which are incorporated herein by reference in its entirety).

The polynucleotides of the present invention can be designed to encode transcripts with alternative polyA tail structures including histone mRNA. According to Norbury, “Terminal uridylation has also been detected on human replication-dependent histone mRNAs. The turnover of these mRNAs is thought to be important for the prevention of potentially toxic histone accumulation following the completion or inhibition of chromosomal DNA replication. These mRNAs are distinguished by their lack of a 3′ poly(A) tail, the function of which is instead assumed by a stable stem-loop structure and its cognate stem-loop binding protein (SLBP); the latter carries out the same functions as those of PABP on polyadenylated mRNAs” (Norbury, “Cytoplasmic RNA: a case of the tail wagging the dog,” Nature Reviews Molecular Cell Biology; AOP, published online 29 Aug. 2013; doi: 10.1038/nrm3645) the contents of which are incorporated herein by reference in its entirety.

Unique poly-A tail lengths provide certain advantages to the polynucleotides of the present invention. Generally, the length of a poly-A tail, when present, is greater than 30 nucleotides (SEQ ID NO: 711) in length. In another embodiment, the poly-A tail is greater than 35 nucleotides in length (SEQ ID NO: 712) (e.g., at least or greater than about 35, 40, 45, 50, 55, 60, 70, 80, 90, 100, 120, 140, 160, 180, 200, 250, 300, 350, 400, 450, 500, 600, 700, 800, 900, 1,000, 1,100, 1,200, 1,300, 1,400, 1,500, 1,600, 1,700, 1,800, 1,900, 2,000, 2,500, and 3,000 nucleotides).

In some embodiments, the polynucleotide or region thereof includes from about 30 to about 3,000 nucleotides (e.g., from 30 to 50, from 30 to 100, from 30 to 250, from 30 to 500, from 30 to 750, from 30 to 1,000, from 30 to 1,500, from 30 to 2,000, from 30 to 2,500, from 50 to 100, from 50 to 250, from 50 to 500, from 50 to 750, from 50 to 1,000, from 50 to 1,500, from 50 to 2,000, from 50 to 2,500, from 50 to 3,000, from 100 to 500, from 100 to 750, from 100 to 1,000, from 100 to 1,500, from 100 to 2,000, from 100 to 2,500, from 100 to 3,000, from 500 to 750, from 500 to 1,000, from 500 to 1,500, from 500 to 2,000, from 500 to 2,500, from 500 to 3,000, from 1,000 to 1,500, from 1,000 to 2,000, from 1,000 to 2,500, from 1,000 to 3,000, from 1,500 to 2,000, from 1,500 to 2,500, from 1,500 to 3,000, from 2,000 to 3,000, from 2,000 to 2,500, and from 2,500 to 3,000).

In some embodiments, the poly-A tail is designed relative to the length of the overall polynucleotide or the length of a particular region of the polynucleotide. This design can be based on the length of a coding region, the length of a particular feature or region or based on the length of the ultimate product expressed from the polynucleotides.

In this context, the poly-A tail can be 10, 20, 30, 40, 50, 60, 70, 80, 90, or 100% greater in length than the polynucleotide or feature thereof. The poly-A tail can also be designed as a fraction of the polynucleotides to which it belongs. In this context, the poly-A tail can be 10, 20, 30, 40, 50, 60, 70, 80, or 90% or more of the total length of the construct, a construct region or the total length of the construct minus the poly-A tail. Further, engineered binding sites and conjugation of polynucleotides for Poly-A binding protein can enhance expression.

Additionally, multiple distinct polynucleotides can be linked together via the PABP (Poly-A binding protein) through the 3′-end using modified nucleotides at the 3′-terminus of the poly-A tail. Transfection experiments can be conducted in relevant cell lines at and protein production can be assayed by ELISA at 12 hr, 24 hr, 48 hr, 72 hr and day 7 post-transfection.

In some embodiments, the polynucleotides of the present invention are designed to include a polyA-G Quartet region. The G-quartet is a cyclic hydrogen bonded array of four guanine nucleotides that can be formed by G-rich sequences in both DNA and RNA. In this embodiment, the G-quartet is incorporated at the end of the poly-A tail. The resultant polynucleotide is assayed for stability, protein production and other parameters including half-life at various time points. It has been discovered that the polyA-G quartet results in protein production from an mRNA equivalent to at least 75% of that seen using a poly-A tail of 120 nucleotides alone (SEQ ID NO:503).

(SEQ ID NO: 503)
Aaaaaaaaaa aaaaaaaaaa aaaaaaaaaa aaaaaaaaaa
aaaaaaaaaa aaaaaaaaaa aaaaaaaaaa aaaaaaaaaa
aaaaaaaaaa aaaaaaaaaa aaaaaaaaaa aaaaaaaaaa

In some embodiments, the polyA tail comprises an alternative nucleoside, e.g., inverted thymidine. PolyA tails comprising an alternative nucleoside, e.g., inverted thymidine, may be generated as described herein. For instance, mRNA constructs may be modified by ligation to stabilize the poly(A) tail. Ligation may be performed using 0.5-1.5 mg/mL mRNA (5′ Cap1, 3′ A100), 50 mM Tris-HCl pH 7.5, 10 mM MgCl2, 1 mM TCEP, 1000 units/mL T4 RNA Ligase 1, 1 mM ATP, 20% w/v polyethylene glycol 8000, and 5:1 molar ratio of modifying oligo to mRNA. Modifying oligo has a sequence of 5′-phosphate-AAAAAAAAAAAAAAAAAAAA-(inverted deoxythymidine (idT) (SEQ ID NO:209)) (see below). Ligation reactions are mixed and incubated at room temperature (˜22° C.) for, e.g., 4 hours. Stable tail mRNA are purified by, e.g., dT purification, reverse phase purification, hydroxyapatite purification, ultrafiltration into water, and sterile filtration. The resulting stable tail-containing mRNAs contain the following structure at the 3′end, starting with the polyA region: A100-UCUAGAAAAAAAAAAAAAAAAAAAA-inverted deoxythymidine (SEQ ID NO:211).

Modifying oligo to stabilize tail (5′-phosphate-AAAAAAAAAAAAAAAAAAAA-(inverted deoxythymidine) (SEQ ID NO:209)) (see below):

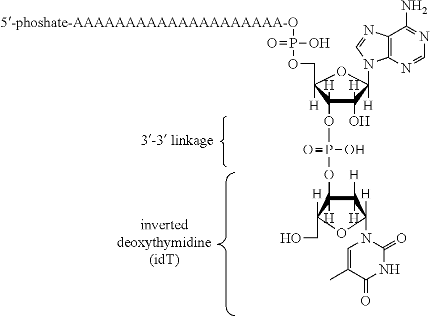

### Start Codon Region

The invention also includes a polynucleotide that comprises both a start codon region and a polynucleotide described herein (e.g., a polynucleotide comprising a nucleotide sequence encoding a checkpoint cancer vaccine comprising (i) one or more IDO antigenic peptides and (ii) one or more PD-L1 antigenic peptides). In some embodiments, the polynucleotides of the present invention can have regions that are analogous to or function like a start codon region.

In some embodiments, the translation of a polynucleotide can initiate on a codon that is not the start codon AUG. Translation of the polynucleotide can initiate on an alternative start codon such as, but not limited to, ACG, AGG, AAG, CTG/CUG, GTG/GUG, ATA/AUA, ATT/AUU, TTG/UUG (see Touriol et al. Biology of the Cell 95 (2003) 169-178 and Matsuda and Mauro PloS ONE, 2010 5:11; the contents of each of which are herein incorporated by reference in its entirety).

As a non-limiting example, the translation of a polynucleotide begins on the alternative start codon ACG. As another non-limiting example, polynucleotide translation begins on the alternative start codon CTG or CUG. As another non-limiting example, the translation of a polynucleotide begins on the alternative start codon GTG or GUG.

Nucleotides flanking a codon that initiates translation such as, but not limited to, a start codon or an alternative start codon, are known to affect the translation efficiency, the length and/or the structure of the polynucleotide. (See, e.g., Matsuda and Mauro PloS ONE, 2010 5:11; the contents of which are herein incorporated by reference in its entirety). Masking any of the nucleotides flanking a codon that initiates translation can be used to alter the position of translation initiation, translation efficiency, length and/or structure of a polynucleotide.

In some embodiments, a masking agent can be used near the start codon or alternative start codon to mask or hide the codon to reduce the probability of translation initiation at the masked start codon or alternative start codon. Non-limiting examples of masking agents include antisense locked nucleic acids (LNA) polynucleotides and exon-junction complexes (EJCs) (See, e.g., Matsuda and Mauro describing masking agents LNA polynucleotides and EJCs (PloS ONE, 2010 5:11); the contents of which are herein incorporated by reference in its entirety).

In another embodiment, a masking agent can be used to mask a start codon of a polynucleotide to increase the likelihood that translation will initiate on an alternative start codon. In some embodiments, a masking agent can be used to mask a first start codon or alternative start codon to increase the chance that translation will initiate on a start codon or alternative start codon downstream to the masked start codon or alternative start codon.

In some embodiments, a start codon or alternative start codon can be located within a perfect complement for a miRNA binding site. The perfect complement of a miRNA binding site can help control the translation, length and/or structure of the polynucleotide similar to a masking agent. As a non-limiting example, the start codon or alternative start codon can be located in the middle of a perfect complement for a miRNA binding site. The start codon or alternative start codon can be located after the first nucleotide, second nucleotide, third nucleotide, fourth nucleotide, fifth nucleotide, sixth nucleotide, seventh nucleotide, eighth nucleotide, ninth nucleotide, tenth nucleotide, eleventh nucleotide, twelfth nucleotide, thirteenth nucleotide, fourteenth nucleotide, fifteenth nucleotide, sixteenth nucleotide, seventeenth nucleotide, eighteenth nucleotide, nineteenth nucleotide, twentieth nucleotide, or twenty-first nucleotide.

In another embodiment, the start codon of a polynucleotide can be removed from the polynucleotide sequence to have the translation of the polynucleotide begin on a codon that is not the start codon. Translation of the polynucleotide can begin on the codon following the removed start codon or on a downstream start codon or an alternative start codon. In a non-limiting example, the start codon ATG or AUG is removed as the first 3 nucleotides of the polynucleotide sequence to have translation initiate on a downstream start codon or alternative start codon. The polynucleotide sequence where the start codon was removed can further comprise at least one masking agent for the downstream start codon and/or alternative start codons to control or attempt to control the initiation of translation, the length of the polynucleotide and/or the structure of the polynucleotide.

### Stop Codon Region

The invention also includes a polynucleotide that comprises both a stop codon region and a polynucleotide described herein (e.g., a polynucleotide comprising a nucleotide sequence encoding a checkpoint cancer vaccine comprising (i) one or more IDO antigenic peptides and (ii) one or more PD-L1 antigenic peptides). In some embodiments, the polynucleotides of the present invention can include at least two stop codons before the 3′ untranslated region (UTR). The stop codon can be selected from TGA, TAA and TAG in the case of DNA, or from UGA, UAA and UAG in the case of RNA. In some embodiments, the polynucleotides of the present invention include the stop codon TGA in the case or DNA, or the stop codon UGA in the case of RNA, and one additional stop codon. In a further embodiment the addition stop codon can be TAA or UAA. In another embodiment, the polynucleotides of the present invention include three consecutive stop codons, four stop codons, or more.

### 3′ Stabilizing Region

In some embodiments, the polynucleotides of the present disclosure (e.g., a polynucleotide comprising a nucleotide sequence encoding a checkpoint cancer vaccine comprising (i) one or more IDO antigenic peptides and (ii) one or more PD-L1 antigenic peptides) further comprise a 3′ stabilizing region. In an embodiment, the polynucleotide comprises: (a) a 5′-UTR (e.g., as described herein); (b) a coding region comprising an open reading frame (ORF) (e.g., as described herein); (c) a 3′-UTR (e.g., as described herein), and (d) a 3′ stabilizing region. Also disclosed herein are LNP compositions comprising the same.

In an embodiment, the polynucleotide comprises a 3′ stabilizing region, e.g., a stabilized tail (e.g., as described herein). A polynucleotide containing a 3′-stabilizing region (e.g., a 3′-stabilizing region including an alternative nucleobase, sugar, and/or backbone) may be particularly effective for use in therapeutic compositions, because they may benefit from increased stability, high expression levels.

In an embodiment, the 3′ stabilizing region comprises a poly A tail, e.g., a poly A tail comprising 80-150 (SEQ ID NO: 713), e.g., 120, adenines (SEQ ID NO: 370). In an embodiment, the poly A tail comprises a UCUAG sequence. In an embodiment, the poly A tail comprises about 80-120, e.g., 100, adenines upstream of UCUAG. In an embodiment, the poly A tail comprises about 1-40, e.g., 20, adenines downstream of UCUAG.

In an embodiment, the 3′ stabilizing region comprises at least one alternative nucleoside. In an embodiment, the alternative nucleoside is an inverted thymidine (idT). In an embodiment, the alternative nucleoside is disposed at the 3′ end of the 3′ stabilizing region.

In an embodiment, the 3′ stabilizing region comprises a structure of Formula VII:

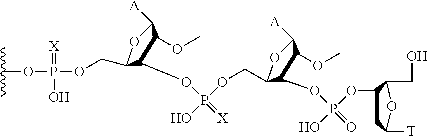

or a salt thereof, wherein each X is independently O or S, and A represents adenine and T represents Thymine.

In an aspect, disclosed herein is an LNP composition comprising a polynucleotide (e.g., an mRNA) which encodes a checkpoint cancer vaccine comprising (i) one or more IDO antigenic peptides and (ii) one or more PD-L1 antigenic peptides (e.g., described herein), wherein the polynucleotide comprises: (a) a 5′-UTR (e.g., as described herein); (b) a coding region comprising a stop element (e.g., as described herein); (c) a 3′-UTR (e.g., as described herein) and; (d) a 3′ stabilizing region (e.g., as described herein).

In an embodiment, the LNP composition comprises: (i) an ionizable lipid (e.g., an amino lipid); (ii) a sterol or other structural lipid; (iii) a non-cationic helper lipid or phospholipid; and (iv) a PEG-lipid.

### Methods of Making Polynucleotides

The present disclosure also provides methods for making a polynucleotide disclosed herein or a complement thereof. In some aspects, a polynucleotide (e.g., an mRNA) disclosed herein encoding a checkpoint cancer vaccine comprising (i) one or more IDO antigenic peptides and (ii) one or more PD-L1 antigenic peptides can be constructed using in vitro transcription.

In other aspects, a polynucleotide (e.g., an mRNA) disclosed herein encoding a checkpoint cancer vaccine can be constructed by chemical synthesis using an oligonucleotide synthesizer. In other aspects, a polynucleotide (e.g., an mRNA) disclosed herein encoding a checkpoint cancer vaccine is made by using a host cell. In certain aspects, a polynucleotide (e.g., an mRNA) disclosed herein encoding a checkpoint cancer vaccine is made by one or more combination of the IVT, chemical synthesis, host cell expression, or any other methods known in the art.

Naturally occurring nucleosides, non-naturally occurring nucleosides, or combinations thereof, can totally or partially naturally replace occurring nucleosides present in the candidate nucleotide sequence and can be incorporated into a sequence-optimized nucleotide sequence (e.g., an mRNA) encoding a checkpoint cancer vaccine comprising (i) one or more IDO antigenic peptides and (ii) one or more PD-L1 antigenic peptides. The resultant mRNAs can then be examined for their ability to produce protein and/or produce a therapeutic outcome.

While RNA can be made synthetically using methods well known in the art, in one embodiment an RNA transcript (e.g., mRNA transcript) is synthesized by contacting a DNA template with a RNA polymerase (e.g., a T7 RNA polymerase or a T7 RNA polymerase variant) under conditions that result in the production of RNA transcript.

In some aspects, the present disclosure provides methods of performing an IVT (in vitro transcription) reaction, comprising contacting a DNA template with the RNA polymerase (e.g., a T7 RNA polymerase, such as a T7 RNA polymerase variant) in the presence of nucleoside triphosphates and buffer under conditions that result in the production of RNA transcripts.

Other aspects of the present disclosure provide capping methods, e.g., co-transcriptional capping methods or other methods known in the art. In one embodiment, a capping method comprises reacting a polynucleotide template with a T7 RNA polymerase variant, nucleoside triphosphates, and a cap analog under in vitro transcription reaction conditions to produce RNA transcript.

IVT conditions typically require a purified linear DNA template containing a promoter, nucleoside triphosphates, a buffer system that includes dithiothreitol (DTT) and magnesium ions, and a RNA polymerase. The exact conditions used in the transcription reaction depend on the amount of RNA needed for a specific application. Typical IVT reactions are performed by incubating a DNA template with a RNA polymerase and nucleoside triphosphates, including GTP, ATP, CTP, and UTP (or nucleotide analogs) in a transcription buffer. A RNA transcript having a 5′ terminal guanosine triphosphate is produced from this reaction.

A deoxyribonucleic acid (DNA) is simply a nucleic acid template for RNA polymerase. A DNA template may include a polynucleotide encoding a checkpoint cancer vaccine comprising (i) one or more IDO antigenic peptides and (ii) one or more PD-L1 antigenic peptides (e.g., disclosed herein). A DNA template, in some embodiments, includes a RNA polymerase promoter (e.g., a T7 RNA polymerase promoter) located 5′ from and operably linked to polynucleotide encoding a checkpoint cancer vaccine. A DNA template may also include a nucleotide sequence encoding a polyadenylation (polyA) tail located at the 3′ end of the polynucleotide.

Polypeptides of interest include, but are not limited to, biologics, antibodies, antigens (vaccines), and therapeutic proteins. The term “protein” encompasses peptides.

A RNA transcript, in some embodiments, is the product of an IVT reaction and, as will be understood by one of ordinary skill in the art, the DNA template for making an RNA molecule is known based on base complementarity. A RNA transcript, in some embodiments, is a messenger RNA (mRNA) that includes a nucleotide sequence encoding a polypeptide of interest linked to a polyA tail. In some embodiments, the mRNA is modified mRNA (mmRNA), which includes at least one modified nucleotide.

A nucleotide includes a nitrogenous base, a five-carbon sugar (ribose or deoxyribose), and at least one phosphate group. Nucleotides include nucleoside monophosphates, nucleoside diphosphates, and nucleoside triphosphates. A nucleoside monophosphate (NMP) includes a nucleobase linked to a ribose and a single phosphate; a nucleoside diphosphate (NDP) includes a nucleobase linked to a ribose and two phosphates; and a nucleoside triphosphate (NTP) includes a nucleobase linked to a ribose and three phosphates. Nucleotide analogs are compounds that have the general structure of a nucleotide or are structurally similar to a nucleotide. Nucleotide analogs, for example, include an analog of the nucleobase, an analog of the sugar and/or an analog of the phosphate group(s) of a nucleotide.

A nucleoside includes a nitrogenous base and a 5-carbon sugar. Thus, a nucleoside plus a phosphate group yields a nucleotide. Nucleoside analogs are compounds that have the general structure of a nucleoside or are structurally similar to a nucleoside. Nucleoside analogs, for example, include an analog of the nucleobase and/or an analog of the sugar of a nucleoside.

It should be understood that the term “nucleotide” includes naturally-occurring nucleotides, synthetic nucleotides and modified nucleotides, unless indicated otherwise. Examples of naturally-occurring nucleotides used for the production of RNA, e.g., in an IVT reaction, as provided herein include adenosine triphosphate (ATP), guanosine triphosphate (GTP), cytidine triphosphate (CTP), uridine triphosphate (UTP), and 5-methyluridine triphosphate (m5UTP). In some embodiments, adenosine diphosphate (ADP), guanosine diphosphate (GDP), cytidine diphosphate (CDP), and/or uridine diphosphate (UDP) are used.

Examples of nucleotide analogs include, but are not limited to, antiviral nucleotide analogs, phosphate analogs (soluble or immobilized, hydrolyzable or non-hydrolyzable), dinucleotide, trinucleotide, tetranucleotide, e.g., a cap analog, or a precursor/substrate for enzymatic capping (vaccinia or ligase), a nucleotide labeled with a functional group to facilitate ligation/conjugation of cap or 5′ moiety (IRES), a nucleotide labeled with a 5′ PO4 to facilitate ligation of cap or 5′ moiety, or a nucleotide labeled with a functional group/protecting group that can be chemically or enzymatically cleaved. Examples of antiviral nucleotide/nucleoside analogs include, but are not limited, to Ganciclovir, Entecavir, Telbivudine, Vidarabine and Cidofovir.

Modified nucleotides may include modified nucleobases. For example, a RNA transcript (e.g., mRNA transcript) of the present disclosure may include a modified nucleobase selected from pseudouridine (ψ), 1-methylpseudouridine (m1ψ), 1-ethylpseudouridine, 2-thiouridine, 4′-thiouridine, 2-thio-1-methyl-1-deaza-pseudouridine, 2-thio-1-methyl-pseudouridine, 2-thio-5-aza-uridine, 2-thio-dihydropseudouridine, 2-thio-dihydrouridine, 2-thio-pseudouridine, 4-methoxy-2-thio-pseudouridine, 4-methoxy-pseudouridine, 4-thio-1-methyl-pseudouridine, 4-thio-pseudouridine, 5-aza-uridine, dihydropseudouridine, 5-methyluridine, 5-methoxyuridine (mo5U) and 2′-O-methyl uridine. In some embodiments, a RNA transcript (e.g., mRNA transcript) includes a combination of at least two (e.g., 2, 3, 4, or more) of the foregoing modified nucleobases.

The nucleoside triphosphates (NTPs) as provided herein may comprise unmodified or modified ATP, modified or unmodified UTP, modified or unmodified GTP, and/or modified or unmodified CTP. In some embodiments, NTPs of an IVT reaction comprise unmodified ATP. In some embodiments, NTPs of an IVT reaction comprise modified ATP. In some embodiments, NTPs of an IVT reaction comprise unmodified UTP. In some embodiments, NTPs of an IVT reaction comprise modified UTP. In some embodiments, NTPs of an IVT reaction comprise unmodified GTP. In some embodiments, NTPs of an IVT reaction comprise modified GTP. In some embodiments, NTPs of an IVT reaction comprise unmodified CTP. In some embodiments, NTPs of an IVT reaction comprise modified CTP.

The concentration of nucleoside triphosphates and cap analog present in an IVT reaction may vary. In some embodiments, NTPs and cap analog are present in the reaction at equimolar concentrations. In some embodiments, the molar ratio of cap analog (e.g., trinucleotide cap) to nucleoside triphosphates in the reaction is greater than 1:1. For example, the molar ratio of cap analog to nucleoside triphosphates in the reaction may be 2:1, 3:1, 4:1, 5:1, 6:1, 7:1, 8:1, 9:1, 10:1, 15:1, 20:1, 25:1, 50:1, or 100:1. In some embodiments, the molar ratio of cap analog (e.g., trinucleotide cap) to nucleoside triphosphates in the reaction is less than 1:1. For example, the molar ratio of cap analog (e.g., trinucleotide cap) to nucleoside triphosphates in the reaction may be 1:2, 1:3, 1:4, 1:5, 1:6, 1:7, 1:8, 1:9, 1:10, 1:15, 1:20, 1:25, 1:50, or 1:100.

The composition of NTPs in an IVT reaction may also vary. For example, ATP may be used in excess of GTP, CTP and UTP. As a non-limiting example, an IVT reaction may include 7.5 millimolar GTP, 7.5 millimolar CTP, 7.5 millimolar UTP, and 3.75 millimolar ATP. The same IVT reaction may include 3.75 millimolar cap analog (e.g., trinucleotide cap). In some embodiments, the molar ratio of G: C: U: A: cap is 1:1:1:0.5:0.5. In some embodiments, the molar ratio of G: C: U: A: cap is 1:1:0.5:1:0.5. In some embodiments, the molar ratio of G: C: U: A: cap is 1:0.5:1:1:0.5. In some embodiments, the molar ratio of G: C: U: A: cap is 0.5:1:1:1:0.5.

In some embodiments, a RNA transcript (e.g., mRNA transcript) includes a modified nucleobase selected from pseudouridine (ψ), 1-methylpseudouridine (m1ψ), 5-methoxyuridine (mo5U), 5-methylcytidine (m5C), α-thio-guanosine and α-thio-adenosine. In some embodiments, a RNA transcript (e.g., mRNA transcript) includes a combination of at least two (e.g., 2, 3, 4 or more) of the foregoing modified nucleobases.

In some embodiments, a RNA transcript (e.g., mRNA transcript) includes pseudouridine (ψ). In some embodiments, a RNA transcript (e.g., mRNA transcript) includes 1-methylpseudouridine (m1ψ). In some embodiments, a RNA transcript (e.g., mRNA transcript) includes 5-methoxyuridine (mo⁵U). In some embodiments, a RNA transcript (e.g., mRNA transcript) includes 5-methylcytidine (m⁵C). In some embodiments, a RNA transcript (e.g., mRNA transcript) includes α-thio-guanosine. In some embodiments, a RNA transcript (e.g., mRNA transcript) includes α-thio-adenosine.

In some embodiments, the polynucleotide (e.g., RNA polynucleotide, such as mRNA polynucleotide) is uniformly modified (e.g., fully modified, modified throughout the entire sequence) for a particular modification. For example, a polynucleotide can be uniformly modified with 1-methylpseudouridine (m¹ψ), meaning that all uridine residues in the mRNA sequence are replaced with 1-methylpseudouridine (m¹ψ). Similarly, a polynucleotide can be uniformly modified for any type of nucleoside residue present in the sequence by replacement with a modified residue such as any of those set forth above. Alternatively, the polynucleotide (e.g., RNA polynucleotide, such as mRNA polynucleotide) may not be uniformly modified (e.g., partially modified, part of the sequence is modified). Each possibility represents a separate embodiment of the present invention.

In some embodiments, the buffer system contains tris. The concentration of tris used in an IVT reaction, for example, may be at least 10 mM, at least 20 mM, at least 30 mM, at least 40 mM, at least 50 mM, at least 60 mM, at least 70 mM, at least 80 mM, at least 90 mM, at least 100 mM or at least 110 mM phosphate. In some embodiments, the concentration of phosphate is 20-60 mM or 10-100 mM.

In some embodiments, the buffer system contains dithiothreitol (DTT). The concentration of DTT used in an IVT reaction, for example, may be at least 1 mM, at least 5 mM, or at least 50 mM. In some embodiments, the concentration of DTT used in an IVT reaction is 1-50 mM or 5-50 mM. In some embodiments, the concentration of DTT used in an IVT reaction is 5 mM.

In some embodiments, the buffer system contains magnesium. In some embodiments, the molar ratio of NTP to magnesium ions (Mg²⁺; e.g., MgCl2) present in an IVT reaction is 1:1 to 1:5. For example, the molar ratio of NTP to magnesium ions may be 1:1, 1:2, 1:3, 1:4 or 1:5.

In some embodiments, the molar ratio of NTP plus cap analog (e.g., trinucleotide cap, such as GAG) to magnesium ions (Mg²⁺; e.g., MgCl2) present in an IVT reaction is 1:1 to 1:5. For example, the molar ratio of NTP+trinucleotide cap (e.g., GAG) to magnesium ions may be 1:1, 1:2, 1:3, 1:4, or 1:5.

In some embodiments, the buffer system contains Tris-HCl, spermidine (e.g., at a concentration of 1-30 mM), TRITON® X-100 (polyethylene glycol p-(1,1,3,3-tetramethylbutyl)-phenyl ether) and/or polyethylene glycol (PEG).

The addition of nucleoside triphosphates (NTPs) to the 3′ end of a growing RNA strand is catalyzed by a polymerase, such as T7 RNA polymerase, for example, any one or more of the T7 RNA polymerase variants (e.g., G47A) of the present disclosure. In some embodiments, the RNA polymerase (e.g., T7 RNA polymerase variant) is present in a reaction (e.g., an IVT reaction) at a concentration of 0.01 mg/ml to 1 mg/ml. For example, the RNA polymerase may be present in a reaction at a concentration of 0.01 mg/mL, 0.05 mg/ml, 0.1 mg/ml, 0.5 mg/ml, or 1.0 mg/ml.

In some embodiments, the polynucleotide of the present disclosure is an IVT polynucleotide. Traditionally, the basic components of an mRNA molecule include at least a coding region, a 5′UTR, a 3′UTR, a 5′ cap and a poly-A tail. The IVT polynucleotides of the present disclosure can function as mRNA but are distinguished from wild-type mRNA in their functional and/or structural design features which serve, e.g., to overcome existing problems of effective polypeptide production using nucleic-acid based therapeutics.

The primary construct of an IVT polynucleotide comprises a first region of linked nucleotides that is flanked by a first flanking region and a second flaking region. This first region can include, but is not limited to, the encoded checkpoint cancer vaccine. The first flanking region can include a sequence of linked nucleosides which function as a 5′ untranslated region (UTR) such as the 5′ UTR of any of the nucleic acids encoding the native 5′ UTR of the polypeptide or a non-native 5′UTR such as, but not limited to, a heterologous 5′ UTR or a synthetic 5′ UTR. The IVT encoding a checkpoint cancer vaccine therapeutic payload or prophylactic payload can comprise at its 5 terminus a signal sequence region encoding one or more signal sequences. The flanking region can comprise a region of linked nucleotides comprising one or more complete or incomplete 5′ UTRs sequences. The flanking region can also comprise a 5′ terminal cap. The second flanking region can comprise a region of linked nucleotides comprising one or more complete or incomplete 3′ UTRs which can encode the native 3′ UTR of a checkpoint cancer vaccine therapeutic payload or prophylactic payload, or a non-native 3′ UTR such as, but not limited to, a heterologous 3′ UTR or a synthetic 3′ UTR. The flanking region can also comprise a 3′ tailing sequence. The 3′ tailing sequence can be, but is not limited to, a polyA tail, a polyA-G quartet and/or a stem loop sequence.

Exemplary methods of making a polynucleotide disclosed herein include: in vitro transcription enzymatic synthesis and chemical synthesis which are disclosed in International PCT application WO 2017/201325, filed on 18 May 2017, the entire contents of which are hereby incorporated by reference.

### Chemical Synthesis

Standard methods can be applied to synthesize an isolated polynucleotide sequence encoding an isolated polypeptide of interest, such as a polynucleotide of the invention (e.g., a polynucleotide comprising a nucleotide sequence encoding a checkpoint cancer vaccine comprising (i) one or more IDO antigenic peptides and (ii) one or more PD-L1 antigenic peptides). For example, a single DNA or RNA oligomer containing a codon-optimized nucleotide sequence coding for the particular isolated polypeptide can be synthesized. In other aspects, several small oligonucleotides coding for portions of the desired polypeptide can be synthesized and then ligated. In some aspects, the individual oligonucleotides typically contain 5′ or 3′ overhangs for complementary assembly.

A polynucleotide disclosed herein (e.g., a RNA, e.g., an mRNA) can be chemically synthesized using chemical synthesis methods and potential nucleobase substitutions known in the art. See, for example, International Publication Nos. WO2014093924, WO2013052523; WO2013039857, WO2012135805, WO2013151671; U.S. Publ. No. US20130115272; or U.S. Pat. Nos. U.S. Pat. No. 8,999,380 or U.S. Pat. No. 8,710,200, all of which are herein incorporated by reference in their entireties.

### Purification

In other aspects, a polynucleotide (e.g., an mRNA) disclosed herein encoding a checkpoint cancer vaccine comprising (i) one or more IDO antigenic peptides and (ii) one or more PD-L1 antigenic peptides can be purified. Purification of the polynucleotides (e.g., mRNA) encoding a checkpoint cancer vaccine described herein can include, but is not limited to, polynucleotide clean-up, quality assurance and quality control. Clean-up can be performed by methods known in the arts such as, but not limited to, AGENCOURT® beads (Beckman Coulter Genomics, Danvers, MA), poly-T beads, LNATM oligo-T capture probes (EXIQON® Inc, Vedbaek, Denmark) or HPLC based purification methods such as, but not limited to, strong anion exchange HPLC, weak anion exchange HPLC, reverse phase HPLC (RP-HPLC), and hydrophobic interaction HPLC (HIC-HPLC). The term “purified” when used in relation to a polynucleotide such as a “purified polynucleotide” refers to one that is separated from at least one contaminant. As used herein, a “contaminant” is any substance which makes another unfit, impure or inferior. Thus, a purified polynucleotide (e.g., DNA and RNA) is present in a form or setting different from that in which it is found in nature, or a form or setting different from that which existed prior to subjecting it to a treatment or purification method.

In some embodiments, purification of a polynucleotide (e.g., mRNA) encoding a checkpoint cancer vaccine of the disclosure removes impurities that can reduce or remove an unwanted immune response, e.g., reducing cytokine activity.

In some embodiments, the polynucleotide (e.g., mRNA) encoding a checkpoint cancer vaccine of the disclosure is purified prior to administration using column chromatography (e.g., strong anion exchange HPLC, weak anion exchange HPLC, reverse phase HPLC (RP-HPLC), and hydrophobic interaction HPLC (HIC-HPLC), or (LCMS)). In some embodiments, a column chromatography (e.g., strong anion exchange HPLC, weak anion exchange HPLC, reverse phase HPLC (RP-HPLC), and hydrophobic interaction HPLC (HIC-HPLC), or (LCMS)) purified polynucleotide, which encodes a checkpoint cancer vaccine as disclosed herein increases expression of the checkpoint cancer vaccine compared to polynucleotides encoding the checkpoint cancer vaccine purified by a different purification method.

In some embodiments, a column chromatography (e.g., strong anion exchange HPLC, weak anion exchange HPLC, reverse phase HPLC (RP-HPLC), and hydrophobic interaction HPLC (HIC-HPLC), or (LCMS)) purified polynucleotide encodes a checkpoint cancer vaccine comprising (i) one or more IDO antigenic peptides and (ii) one or more PD-L1 antigenic peptides.

In some embodiments, the purified polynucleotide is at least about 80% pure, at least about 85% pure, at least about 90% pure, at least about 95% pure, at least about 96% pure, at least about 97% pure, at least about 98% pure, at least about 99% pure, or about 100% pure.

A quality assurance and/or quality control check can be conducted using methods such as, but not limited to, gel electrophoresis, UV absorbance, or analytical HPLC.

In another embodiment, the polynucleotides can be sequenced by methods including, but not limited to reverse-transcriptase-PCR.

### Chemical Modifications of Polynucleotides

The present disclosure provides for modified nucleosides and nucleotides of a nucleic acid (e.g., RNA nucleic acids, such as mRNA nucleic acids). A “nucleoside” refers to a compound containing a sugar molecule (e.g., a pentose or ribose) or a derivative thereof in combination with an organic base (e.g., a purine or pyrimidine) or a derivative thereof (also referred to herein as “nucleobase”). A “nucleotide” refers to a nucleoside, including a phosphate group. Modified nucleotides may by synthesized by any useful method, such as, for example, chemically, enzymatically, or recombinantly, to include one or more modified or non-natural nucleosides. Nucleic acids can comprise a region or regions of linked nucleosides. Such regions may have variable backbone linkages. The linkages can be standard phosphodiester linkages, in which case the nucleic acids would comprise regions of nucleotides.

Modified nucleotide base pairing encompasses not only the standard adenosine-thymine, adenosine-uracil, or guanosine-cytosine base pairs, but also base pairs formed between nucleotides and/or modified nucleotides comprising non-standard or modified bases, wherein the arrangement of hydrogen bond donors and hydrogen bond acceptors permits hydrogen bonding between a non-standard base and a standard base or between two complementary non-standard base structures, such as, for example, in those nucleic acids having at least one chemical modification. One example of such non-standard base pairing is the base pairing between the modified nucleotide inosine and adenine, cytosine or uracil. Any combination of base/sugar or linker may be incorporated into nucleic acids of the present disclosure.

In some embodiments, modified nucleobases in nucleic acids (e.g., RNA nucleic acids, such as mRNA nucleic acids) comprise N1-methyl-pseudouridine (m1ψ), 1-ethyl-pseudouridine (e1ψ), 5-methoxy-uridine (mo5U), 5-methyl-cytidine (m5C), and/or pseudouridine (w). In some embodiments, modified nucleobases in nucleic acids (e.g., RNA nucleic acids, such as mRNA nucleic acids) comprise 5-methoxymethyl uridine, 5-methylthio uridine, 1-methoxymethyl pseudouridine, 5-methyl cytidine, and/or 5-methoxy cytidine. In some embodiments, the polyribonucleotide includes a combination of at least two (e.g., 2, 3, 4 or more) of any of the aforementioned modified nucleobases, including but not limited to chemical modifications.

In some embodiments, a RNA nucleic acid of the disclosure comprises N1-methyl-pseudouridine (m1ψ) substitutions at one or more or all uridine positions of the nucleic acid.

In some embodiments, a RNA nucleic acid of the disclosure comprises N1-methyl-pseudouridine (m1ψ) substitutions at one or more or all uridine positions of the nucleic acid and 5-methyl cytidine substitutions at one or more or all cytidine positions of the nucleic acid.

In some embodiments, a RNA nucleic acid of the disclosure comprises pseudouridine (ψ) substitutions at one or more or all uridine positions of the nucleic acid.

In some embodiments, a RNA nucleic acid of the disclosure comprises pseudouridine (ψ) substitutions at one or more or all uridine positions of the nucleic acid and 5-methyl cytidine substitutions at one or more or all cytidine positions of the nucleic acid.

In some embodiments, a RNA nucleic acid of the disclosure comprises uridine at one or more or all uridine positions of the nucleic acid.

In some embodiments, nucleic acids (e.g., RNA nucleic acids, such as mRNA nucleic acids) are uniformly modified (e.g., fully modified, modified throughout the entire sequence) for a particular modification. For example, a nucleic acid can be uniformly modified with N1-methyl-pseudouridine, meaning that all uridine residues in the mRNA sequence are replaced with N1-methyl-pseudouridine. Similarly, a nucleic acid can be uniformly modified for any type of nucleoside residue present in the sequence by replacement with a modified residue such as those set forth above.

The nucleic acids of the present disclosure may be partially or fully modified along the entire length of the molecule. For example, one or more or all or a given type of nucleotide (e.g., purine or pyrimidine, or any one or more or all of A, G, U, C) may be uniformly modified in a nucleic acid of the disclosure, or in a predetermined sequence region thereof (e.g., in the mRNA including or excluding the polyA tail). In some embodiments, all nucleotides X in a nucleic acid of the present disclosure (or in a sequence region thereof) are modified nucleotides, wherein X may be any one of nucleotides A, G, U, C, or any one of the combinations A+G, A+U, A+C, G+U, G+C, U+C, A+G+U, A+G+C, G+U+C or A+G+C.

The nucleic acid may contain from about 1% to about 100% modified nucleotides (either in relation to overall nucleotide content, or in relation to one or more types of nucleotide, i.e., any one or more of A, G, U or C) or any intervening percentage (e.g., from 1% to 20%, from 1% to 25%, from 1% to 50%, from 1% to 60%, from 1% to 70%, from 1% to 80%, from 1% to 90%, from 1% to 95%, from 10% to 20%, from 10% to 25%, from 10% to 50%, from 10% to 60%, from 10% to 70%, from 10% to 80%, from 10% to 90%, from 10% to 95%, from 10% to 100%, from 20% to 25%, from 20% to 50%, from 20% to 60%, from 20% to 70%, from 20% to 80%, from 20% to 90%, from 20% to 95%, from 20% to 100%, from 50% to 60%, from 50% to 70%, from 50% to 80%, from 50% to 90%, from 50% to 95%, from 50% to 100%, from 70% to 80%, from 70% to 90%, from 70% to 95%, from 70% to 100%, from 80% to 90%, from 80% to 95%, from 80% to 100%, from 90% to 95%, from 90% to 100%, and from 95% to 100%). It will be understood that any remaining percentage is accounted for by the presence of unmodified A, G, U, or C.

The nucleic acids may contain at a minimum 1% and at maximum 100% modified nucleotides, or any intervening percentage, such as at least 5% modified nucleotides, at least 10% modified nucleotides, at least 25% modified nucleotides, at least 50% modified nucleotides, at least 80% modified nucleotides, or at least 90% modified nucleotides. For example, the nucleic acids may contain a modified pyrimidine such as a modified uracil or cytosine. In some embodiments, at least 5%, at least 10%, at least 25%, at least 50%, at least 80%, at least 90% or 100% of the uracil in the nucleic acid is replaced with a modified uracil (e.g., a 5-substituted uracil). The modified uracil can be replaced by a compound having a single unique structure, or can be replaced by a plurality of compounds having different structures (e.g., 2, 3, 4 or more unique structures). In some embodiments, at least 5%, at least 10%, at least 25%, at least 50%, at least 80%, at least 90% or 100% of the cytosine in the nucleic acid is replaced with a modified cytosine (e.g., a 5-substituted cytosine). The modified cytosine can be replaced by a compound having a single unique structure, or can be replaced by a plurality of compounds having different structures (e.g., 2, 3, 4 or more unique structures).

### Pharmaceutical Compositions

The present disclosure provides pharmaceutical formulations comprising any of the LNP compositions disclosed herein, e.g., an LNP composition comprising a polynucleotide comprising an mRNA encoding a checkpoint cancer vaccine comprising (i) one or more IDO antigenic peptides and (ii) one or more PD-L1 antigenic peptides.

In some embodiments, the composition or formulation can contain a polynucleotide comprising a sequence optimized nucleic acid sequence disclosed herein which encodes a checkpoint cancer vaccine comprising (i) one or more IDO antigenic peptides and (ii) one or more PD-L1 antigenic peptides. In some embodiments, the composition or formulation can contain a polynucleotide (e.g., a RNA, e.g., an mRNA) comprising a polynucleotide (e.g., an ORF) having significant sequence identity to a sequence optimized nucleic acid sequence disclosed herein which encodes a checkpoint cancer vaccine. In some embodiments, the polynucleotide further comprises a miRNA binding site, e.g., a miRNA binding site that binds miR-126, miR-142, miR-144, miR-146, miR-150, miR-155, miR-16, miR-21, miR-223, miR-24, miR-27, and miR-26a.

In some embodiments of the disclosure, the polynucleotides are formulated in compositions and complexes in combination with one or more pharmaceutically acceptable excipients. Pharmaceutical compositions can optionally comprise one or more additional active substances, e.g. therapeutically and/or prophylactically active substances. Pharmaceutical compositions of the present disclosure can be sterile and/or pyrogen-free. General considerations in the formulation and/or manufacture of pharmaceutical agents can be found, for example, in Remington: The Science and Practice of Pharmacy 21st ed., Lippincott Williams & Wilkins, 2005.

In some embodiments, compositions are administered to humans, human patients, or subjects. For the purposes of the present disclosure, the phrase “active ingredient” generally refers to polynucleotides to be delivered as described herein.

Although the descriptions of pharmaceutical compositions provided herein are principally directed to pharmaceutical compositions which are suitable for administration to humans, it will be understood by the skilled artisan that such compositions are generally suitable for administration to any other animal, e.g., to non-human animals, e.g. non-human mammals. Modification of pharmaceutical compositions suitable for administration to humans in order to render the compositions suitable for administration to various animals is well understood, and the ordinarily skilled veterinary pharmacologist can design and/or perform such modification with merely ordinary, if any, experimentation. Subjects to which administration of the pharmaceutical compositions is contemplated include, but are not limited to, humans and/or other primates; mammals.

In some embodiments, the polynucleotide of the present disclosure is formulated for intramuscular delivery.

Formulations of the pharmaceutical compositions described herein can be prepared by any method known or hereafter developed in the art of pharmacology. In general, such preparatory methods include the step of bringing the active ingredient into association with an excipient and/or one or more other accessory ingredients, and then, if necessary and/or desirable, dividing, shaping and/or packaging the product into a desired single- or multi-dose unit.

Relative amounts of the active ingredient, the pharmaceutically acceptable excipient, and/or any additional ingredients in a pharmaceutical composition in accordance with the disclosure will vary, depending upon the identity, size, and/or condition of the subject treated and further depending upon the route by which the composition is to be administered. By way of example, the composition can comprise between 0.1% and 100%, e.g., between 0.5% and 50%, between 1% and 30%, between 5% and 80%, or at least 80% (w/w) active ingredient.

### Formulations

The polynucleotide comprising an mRNA encoding a checkpoint cancer vaccine, of the disclosure can be formulated using one or more excipients.

The present invention provides pharmaceutical formulations that comprise a polynucleotide described herein (e.g., a polynucleotide comprising a nucleotide sequence encoding a checkpoint cancer vaccine comprising (i) one or more IDO antigenic peptides and (ii) one or more PD-L1 antigenic peptides). The polynucleotides described herein can be formulated using one or more excipients to: (1) increase stability; (2) increase cell transfection; (3) permit the sustained or delayed release (e.g., from a depot formulation of the polynucleotide); (4) alter the biodistribution (e.g., target the polynucleotide to specific tissues or cell types); (5) increase the translation of encoded protein in vivo; and/or (6) alter the release profile of encoded protein in vivo. In some embodiments, the pharmaceutical formulation further comprises a delivery agent comprising, e.g., a compound having the Formula (I); or a compound having the Formula (III), (IV), (V), or (VI), or any combination thereof. In some embodiments, the delivery agent comprises an ionizable amino lipid, a helper lipid (e.g., DSPC), a sterol (e.g., Cholesterol), and a PEG lipid (e.g., PEG-DMG), e.g., with a mole ratio in the range of about (i) 40-50 mol % ionizable amino lipid, optionally 45-50 mol % ionizable amino lipid, for example, 45-46 mol %, 46-47 mol %, 47-48 mol %, 48-49 mol %, or 49-50 mol % for example about 45 mol %, 45.5 mol %, 46 mol %, 46.5 mol %, 47 mol %, 47.5 mol %, 48 mol %, 48.5 mol %, 49 mol %, or 49.5 mol %; (ii) 30-45 mol % sterol (e.g., cholesterol), optionally 35-42 mol % sterol, for example, 30-31 mol %, 31-32 mol %, 32-33 mol %, 33-34 mol %, 35-35 mol %, 35-36 mol %, 36-37 mol %, 37-38 mol %, 38-39 mol %, or 39-40 mol %, or 40-42 mol % sterol; (iii) 5-15 mol % helper lipid (e.g., DSPC), optionally 10-15 mol % helper lipid, for example, 5-6 mol %, 6-7 mol %, 7-8 mol %, 8-9 mol %, 9-10 mol %, 10-11 mol %, 11-12 mol %, 12-13 mol %, 13-14 mol %, or 14-15 mol % helper lipid; and (iv) 1-5% PEG lipid, optionally 1-5 mol % PEG lipid, for example 1.5 to 2.5 mol %, 1-2 mol %, 2-3 mol %, 3-4 mol %, or 4-5 mol % PEG lipid.

A pharmaceutically acceptable excipient, as used herein, includes, but are not limited to, any and all solvents, dispersion media, or other liquid vehicles, dispersion or suspension aids, diluents, granulating and/or dispersing agents, surface active agents, isotonic agents, thickening or emulsifying agents, preservatives, binders, lubricants or oil, coloring, sweetening or flavoring agents, stabilizers, antioxidants, antimicrobial or antifungal agents, osmolality adjusting agents, pH adjusting agents, buffers, chelants, cyoprotectants, and/or bulking agents, as suited to the particular dosage form desired. Various excipients for formulating pharmaceutical compositions and techniques for preparing the composition are known in the art (see Remington: The Science and Practice of Pharmacy, 21st Edition, A. R. Gennaro (Lippincott, Williams & Wilkins, Baltimore, MD, 2006; incorporated herein by reference in its entirety).

Exemplary diluents include, but are not limited to, calcium or sodium carbonate, calcium phosphate, calcium hydrogen phosphate, sodium phosphate, lactose, sucrose, cellulose, microcrystalline cellulose, kaolin, mannitol, sorbitol, etc., and/or combinations thereof.

Exemplary surface active agents and/or emulsifiers include, but are not limited to, natural emulsifiers (e.g., acacia, agar, alginic acid, sodium alginate, tragacanth, chondrux, cholesterol, xanthan, pectin, gelatin, egg yolk, casein, wool fat, cholesterol, wax, and lecithin), sorbitan fatty acid esters (e.g., polyoxyethylene sorbitan monooleate [TWEEN®80], sorbitan monopalmitate [SPAN®40], glyceryl monooleate, polyoxyethylene esters, polyethylene glycol fatty acid esters (e.g., CREMOPHOR®), polyoxyethylene ethers (e.g., polyoxyethylene lauryl ether [BRIJ®30]), PLUORINC®F 68, POLOXAMER®188, etc. and/or combinations thereof.

Exemplary binding agents include, but are not limited to, starch, gelatin, sugars (e.g., sucrose, glucose, dextrose, dextrin, molasses, lactose, lactitol, mannitol), amino acids (e.g., glycine), natural and synthetic gums (e.g., acacia, sodium alginate), ethylcellulose, hydroxyethylcellulose, hydroxypropyl methylcellulose, etc., and combinations thereof.

Oxidation is a potential degradation pathway for mRNA, especially for liquid mRNA Formulations. In order to prevent oxidation, antioxidants can be added to the Formulations. Exemplary antioxidants include, but are not limited to, alpha tocopherol, ascorbic acid, ascorbyl palmitate, benzyl alcohol, butylated hydroxyanisole, m-cresol, methionine, butylated hydroxytoluene, monothioglycerol, sodium or potassium metabisulfite, propionic acid, propyl gallate, sodium ascorbate, etc., and combinations thereof.

Exemplary chelating agents include, but are not limited to, ethylenediaminetetraacetic acid (EDTA), citric acid monohydrate, disodium edetate, fumaric acid, malic acid, phosphoric acid, sodium edetate, tartaric acid, trisodium edetate, etc., and combinations thereof.

Exemplary antimicrobial or antifungal agents include, but are not limited to, benzalkonium chloride, benzethonium chloride, methyl paraben, ethyl paraben, propyl paraben, butyl paraben, benzoic acid, hydroxybenzoic acid, potassium or sodium benzoate, potassium or sodium sorbate, sodium propionate, sorbic acid, etc., and combinations thereof.

Exemplary preservatives include, but are not limited to, vitamin A, vitamin C, vitamin E, beta-carotene, citric acid, ascorbic acid, butylated hydroxyanisol, ethylenediamine, sodium lauryl sulfate (SLS), sodium lauryl ether sulfate (SLES), etc., and combinations thereof.

In some embodiments, the pH of polynucleotide solutions is maintained between pH 5 and pH 8 to improve stability. Exemplary buffers to control pH can include, but are not limited to sodium phosphate, sodium citrate, sodium succinate, histidine (or histidine-HCl), sodium malate, sodium carbonate, etc., and/or combinations thereof.

Exemplary lubricating agents include, but are not limited to, magnesium stearate, calcium stearate, stearic acid, silica, talc, malt, hydrogenated vegetable oils, polyethylene glycol, sodium benzoate, sodium or magnesium lauryl sulfate, etc., and combinations thereof.

The pharmaceutical composition or Formulation described here can contain a cryoprotectant to stabilize a polynucleotide described herein during freezing. Exemplary cryoprotectants include, but are not limited to mannitol, sucrose, trehalose, lactose, glycerol, dextrose, etc., and combinations thereof.

The pharmaceutical composition or formulation described here can contain a bulking agent in lyophilized polynucleotide formulations to yield a “pharmaceutically elegant” cake, stabilize the lyophilized polynucleotides during long-term (e.g., 36 month) storage. Exemplary bulking agents of the present invention can include, but are not limited to sucrose, trehalose, mannitol, glycine, lactose, raffinose, and combinations thereof.

In some embodiments, the pharmaceutical composition or Formulation further comprises a delivery agent. The delivery agent of the present disclosure can include, without limitation, liposomes, lipid nanoparticles, lipidoids, polymers, lipoplexes, microvesicles, exosomes, peptides, proteins, cells transfected with polynucleotides, hyaluronidase, nanoparticle mimics, nanotubes, conjugates, and combinations thereof.

Formulations of the disclosure can include one or more excipients, each in an amount that together increases the stability of the polynucleotide, increases cell transfection by the polynucleotide, increases the expression of polynucleotides encoded protein, and/or alters the release profile of polynucleotide encoded proteins. Further, the polynucleotides of the present disclosure can be formulated using self-assembled nucleic acid nanoparticles.

Formulations of the pharmaceutical compositions described herein can be prepared by any method known or hereafter developed in the art of pharmacology. In general, such preparatory methods include the step of associating the active ingredient with an excipient and/or one or more other accessory ingredients.

A pharmaceutical composition in accordance with the present disclosure can be prepared, packaged, and/or sold in bulk, as a single unit dose, and/or as a plurality of single unit doses. As used herein, a “unit dose” refers to a discrete amount of the pharmaceutical composition comprising a predetermined amount of the active ingredient. The amount of the active ingredient is generally equal to the dosage of the active ingredient which would be administered to a subject and/or a convenient fraction of such a dosage such as, for example, one-half or one-third of such a dosage.

Relative amounts of the active ingredient, the pharmaceutically acceptable excipient, and/or any additional ingredients in a pharmaceutical composition in accordance with the present disclosure can vary, depending upon the identity, size, and/or condition of the subject being treated and further depending upon the route by which the composition is to be administered. For example, the composition can comprise between 0.1% and 99% (w/w) of the active ingredient. By way of example, the composition can comprise between 0.1% and 100%, e.g., between 0.5 and 50%, between 1-30%, between 5-80%, at least 80% (w/w) active ingredient.

In some embodiments, the formulations described herein contain at least one polynucleotide. As a non-limiting example, the formulations contain 1, 2, 3, 4, or 5 polynucleotides.

The use of a conventional excipient medium can be contemplated within the scope of the present disclosure, except insofar as any conventional excipient medium can be incompatible with a substance or its derivatives, such as by producing any undesirable biological effect or otherwise interacting in a deleterious manner with any other component(s) of the pharmaceutical composition.

In some embodiments, the particle size of the lipid nanoparticle is increased and/or decreased. The change in particle size can be able to help counter biological reaction such as, but not limited to, inflammation or can increase the biological effect of the modified mRNA delivered to mammals.

Pharmaceutically acceptable excipients used in the manufacture of pharmaceutical compositions include, but are not limited to, inert diluents, surface active agents and/or emulsifiers, preservatives, buffering agents, lubricating agents, and/or oils. Such excipients can optionally be included in the pharmaceutical formulations of the disclosure.

In some embodiments, the polynucleotides is administered in or with, formulated in or delivered with nanostructures that can sequester molecules such as cholesterol. Non-limiting examples of these nanostructures and methods of making these nanostructures are described in US Patent Publication No. US20130195759. Exemplary structures of these nanostructures are shown in US Patent Publication No. US20130195759, and can include a core and a shell surrounding the core.

### Equivalents and Scope

Those skilled in the art will recognize, or be able to ascertain using no more than routine experimentation, many equivalents to the specific embodiments in accordance with the disclosure described herein. The scope of the present disclosure is not intended to be limited to the Description below, but rather is as set forth in the appended claims.

In the claims, articles such as “a,” “an,” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The disclosure includes embodiments in which exactly one member of the group is present in, employed in, or otherwise relevant to a given product or process. The disclosure includes embodiments in which more than one, or all of the group members are present in, employed in, or otherwise relevant to a given product or process.

It is also noted that the term “comprising” is intended to be open and permits but does not require the inclusion of additional elements or steps. When the term “comprising” is used herein, the term “consisting of” is thus also encompassed and disclosed.

Where ranges are given, endpoints are included. Furthermore, it is to be understood that unless otherwise indicated or otherwise evident from the context and understanding of one of ordinary skill in the art, values that are expressed as ranges can assume any specific value or subrange within the stated ranges in different embodiments of the disclosure, to the tenth of the unit of the lower limit of the range, unless the context clearly dictates otherwise.

All cited sources, for example, references, publications, databases, database entries, and art cited herein, are incorporated into this application by reference, even if not expressly stated in the citation. In case of conflicting statements of a cited source and the instant application, the statement in the instant application shall control.

### EXAMPLES

The disclosure will be more fully understood by reference to the following examples. They should not, however, be construed as limiting the scope of the disclosure. It is understood that the examples and embodiments described herein are for illustrative purposes only and that various modifications or changes in light thereof will be suggested to persons skilled in the art and are to be included within the spirit and purview of this application and scope of the appended claims.

### Example 1: Immunogenicity of Checkpoint Cancer Vaccine

A messenger RNA (mRNA) vaccine designed to generate antigen-specific T cell responses to an IDO1 and PD-L1 epitope in human leukocyte antigen (HLA) serotype A 02:01 (HLA-A*02:01) transgenic mice was tested for immunogenicity. The vaccine encodes an octamer comprised of (PD-L1₆₋₃₀+IDO1₁₉₂₋₂₁₆) repeated four times. The vaccine was formulated compound 25-based lipid nanoparticles (LNPs).

Mice were vaccinated with 6 μg of the vaccine on Study Days 1 and 8. Spleens were harvested on Study Day 15 and splenocytes were re-stimulated ex vivo with overlapping peptides corresponding to individual epitopes encoded in the vaccine.

Positive responses were detected by IFNγ enzyme-linked immune absorbent spot (ELISpot) and intracellular cytokine staining (ICS) and flow cytometry. Immune responses were detected to both PD-L1 and IDO1 by IFNγ ELISpot in all HLA-A*02:01 animals evaluated. PD-L1-specific CD8+IFNγ+ T cell responses were observed in all HLA-A*02:01 animals, and one HLA-A*02:01 animal showed IDO1-specific CD8+IFNγ+ T cell responses.

The totality of the data indicates that the IDO1/PD-L1 antigen concatemer mRNA vaccine generates an IDO1/PD-L1-specific T cell response in the HLA-A*02:01 transgenic mouse T cell immunogenicity model.

### Example 1A. Vaccine Design

The sequences of the two epitopes in the mRNA vaccine and peptides used to perform re-stimulations (including the full length 25mer, the predicted minimal sequence, and 4 overlapping 15mer-peptides for each) are listed in Table 1.

TABLE 1
IDO1/PD-L1 antigen concatemer Messenger RNA
Vaccine Epitopes and Restimulation Peptides

| | | Restimulation |
|---|---|---|
| | Epitope | Peptide |
| Epitope | Sequence | Sequences |
| IDO1_{192- 216} | ERDTLLKALLEIAS | ERDTLLKALLEIASCLEKALQ |
| | CLEKALQVFHQ | VFHQALLEIASCLERDTLLK |
| | (SEQ ID NO: 1) | ALLEIASCLKALLEIASCLEK |
| | | ALEIASCLEKALQVFHQRDT |
| | | LLKALLEIASCL |
| | | (SEQ ID NO: 714) |
| PD-L1₆₋₃₀ | VFIFMTYWHLLNAF | VFIFMTYWHLLNAFTVTVPK |
| | TVTVPKDLYVV | DLYVVLLNAFTVTVVFIFMT |
| | (SEQ ID NO: 3) | YWHLLNAFTTYWHLLNAFT |
| | | VTVPKLNAFTVTVPKDLYVV |
| | | FMTYWHLLNAFTVTVAFTV |
| | | TV (SEQ ID NO: 715) |

The mRNA drug substances were manufactured as described in Richner et al (Richner et al 2017). The mRNAs were formulated in an LNP. The negative control is a lipid nanoparticle (LNP), which contains a non-translated Factor IX sequence (NT-FIX). All test articles are formulated with the same composition and in the same final buffer (100 mM Tris, 7% w/v PG, 1 mM DTPA, pH 7.5). Formulation details are listed in Table 2.

TABLE 2
Formulation details for two test articles

| | mRNA | | | |
|---|---|---|---|---|
| | Concentration | | | |
| Test Material | (mg/ml) | Size (nm) | DPI | EE % |
| NT-FIX (FID 18295.2) | 0.28 | 82 | 0.15 | 93 |
| IDO1- | 0.36 | 88 | 0.12 | 89 |
| PDL1_alt25merX4 | | | | |
| (FID 18297.1) | | | | |

Abbreviations: DS, drug substance; EE, encapsulation efficiency; IDO1, Indoleamine 2,3-Dioxygenase 1; mRNA, messenger RNA; NT-FIX, nontranslatable factor XI; PDI, polydispersity index; PDL1, programmed death-ligand 1 (PD-L1).

The mRNA vaccine dose included 6 μg of mRNA formulated in compound 25-based LNP in a total dose volume of 100 μL. All test articles were diluted in PBS before injection.

### Mice

Female HLA-A*02:01 mice (OR1159, Taconic strain #9659-F; age: 6-8 weeks at delivery; average weight: 21.3 g at study initiation), female BALB/c mice (OR1160, Taconic strain #BALB/cAnNTac; age: 6-8 weeks at delivery; average weight 19.8 g at study initiation) and female C57BL/6 mice (Taconic strain #B6nTAC; age 6-8 weeks at delivery; average weight 18.7 g at study initiation) were purchased from Taconic Laboratories (Hudson, NY).

Just prior to dose initiation, female HLA-A*02:01 mice were randomly assigned into groups (n=4 in Group 1 and n=6 in Groups 2-9) according to body weight in a range of 18.33-25.73 grams (mean 21.29 g, median 21.04 g).

### Example 1B. Study Design

Just prior to dose initiation, animals were randomly assigned into groups (n=6 per group, within each group n=3 per mouse strain tested) according to body weight in a range of 16.67-22.28 g (mean 19.77 g, median 19.73 g) for the BALB/c mice (mouse #1-3 per group) and in a range of 16.45-20.27 g (mean 18.67 g, median 18.72 g) for the C57BL/6 mice (mouse #4-6 per group). Data related to Groups 2 through 4 and Groups 6 through 9 are not presented in this report.

Mice were treated according to the study design presented in Table 3.

TABLE 3
Treatment Regimen for Studies OR1159 and OR1160

| | | | | ROA, | Time of |
|---|---|---|---|---|---|
| | | | Individual | Volume, | Tissue |
| | | mRNA | mRNA Dose | Treatment | Harvest |
| Group^{a} | mRNA/Treatment^{b} | Ratio | (μg)^{c} | Day | (Post-dose) |
| 1 | NT FIX | N/A | 6 | IM, 100 | 7 Days |
| 5 | IDO1- | N/A | 6 | μL (50 μL | post second |
| | PDL1_25mer_altX4 | | | per leg), | dose (Study |
| | | | | Study Days 1 | Day 15) |
| | | | | and 8 | |

Abbreviations: IDO1, Indoleamine 2,3-Dioxygenase 1; IM, intramuscular; mRNA, messenger RNA; N/A, not applicable; NT FIX, non-translatable factor IX; PDL1, programmed death-ligand 1 (PD-L1); ROA, route of administration.
^{a}In Study OR1159 (female HLA-A*02:01 mice) group size was n = 4 in Group 1 and n = 6 in Groups 2-9. In Study OR1160 (wildtype female C57BL/6 and BALB/c mice) group size was n = 6 with n = 3 mice per mouse strain tested.
^{b}mRNAs were formulated in compound 25-containing lipid nanoparticles in 100 mM Tris, 7% w/v PG, 1 mM DTPA, pH 7.5 buffer.
^{c}Total mRNA dose was 6 μg.

### Example 1C. Immunization, Tissue Harvesting and Processing

The mRNAs, individually formulated in LNPs, were diluted to the target dose level in PBS and administered intramuscularly (50 μL administered into each hind leg per dose) on Study Days 1 and 8 with mRNA-4359, the IDO1/PD-L1 concatemer mRNA vaccine formulated in compound-based LNPs (High Throughput Operations lot FID 18297, 6 μg dose, n=4 in Group 1 and n=6 in Groups 2-9, n=3 mice per strain tested, and n=6 per group. Spleens were harvested on Study Day 15, and single cell suspensions were prepared per standard procedure. Briefly, spleens were manually disrupted using the plunger of a 5 mL syringe through a 70 μL cell strainer sitting inside a 50 mL conical tube. The cell strainer was washed multiple times with medium during mechanical disruption collecting all of the wash media into the 50 mL conical tube. The cell suspension was spun down at 300×g for 10 minutes at 4° C., supernatant poured off of the pellet and the cells were resuspended in 3 mL of ammonium-chloride-potassium lysing buffer for red blood cell lysis. Lysis was performed at room temperature for 3 minutes. After the 3-minute lysis, 12 mL of RPMI-1640 containing 10% FBS was added to each tube, the suspension spun down at 300×g for 10 minutes at 4° C. Supernatant was poured off and the cells resuspended in 3 mL of RPMI-1640 containing 10% FBS. The cell suspension was then filtered through a 70 μL cell strainer into a new 50 mL conical tube. A wash step was performed by rinsing the original 50 mL conical tube with 10 mL RPMI-1640 containing 10% FBS through the cell strainer and collecting into a new 50 mL conical tube. Cell suspensions were then counted and stored at 370C, 5% CO2, until staining.

Mouse IFNγ ELISpot-plus kit (Mabtech, 3321-4APT-2) was used to analyze antigen-specific immune responses. The ELISpot kit includes IFNγ precoated plates, biotinylated detection monoclonal antibody (R4-6A2), streptavidin-ALP, and BCIP/NBT-plus substrate. Splenocytes were processed to a cell suspension, red blood cells were removed, and viable cells were counted. The ELISpot plates were blocked with complete media for 2 hours at 37° C. and 5% CO₂ prior to plating cells. Cells were plated (4× 10⁵ cells/well) with paired stimulant peptides (2 μg/mL; Table 1), a negative (vehicle/medium) control, or a positive (50 ng/mL phorbol myristate acetate, [Sigma-Aldrich, P8139] and 1 μg/mL ionomycin [Sigma-Aldrich, I0634]) control (added to 1 well each), and plates were incubated at 37° C. and 5% CO₂ for 36 hours. After the incubation, cells were washed from the membrane using 5 washes of PBS. Anti-mouse IFNγ detection antibody R4-6A2 (100 μL/well; 1 μg/mL) was added to the membranes, and plates were incubated at 37° C. and 5% CO₂ for 2 hours. Membranes were then washed 5 times with PBS before adding the streptavidin-ALP for 1 hour at room temperature. Following the 1-hour incubation, membranes were washed 5 times with PBS, followed by 3 washes with PBS before adding BCIP/NBT-plus substrate. Membranes were incubated for 4 minutes in BCIP/NBT-plus substrate to develop the plates before stopping the reaction by rinsing the plates in cold tap water.

Spleen cells were counted, normalized, stained for viability (Fixable Viability Dye, 4), blocked with TruStain fcX™ (anti-mouse CD16/32) antibody (BioLegend, #101319), and stained with fluorescent antibodies diluted in staining buffer as detailed in Table 4. Intracellular staining was performed with FoxP3/Transcription Factor Staining Buffer Set (Thermo Fisher Scientific, #005523). Samples were otherwise fixed with Intracellular Fixation buffer (Thermo Fisher Scientific, #88-8824-00). Stained samples were analyzed on an Attune NxT Flow Cytometer (Invitrogen).

TABLE 4
Flow Cytometry Staining Antibodies and Dilutions Used

| Antibody | Fluorophore | Clone | Isotype | Dilution |
|---|---|---|---|---|
| CD44 | FITC | IM7 | Rat IgG2b | 200 |
| CD8b | Percp Cy5.5 | YTS156.7.7 | Rat IgG2b | 300 |
| CD4 | APC | GK1.5 | Rat IgG2b | 300 |
| Foxp3 | AF700 | MF-14 | Rat IgG2b | 200 |
| Fixable | eFluor 780 | | | 800 |
| Viability Dye | | | | |
| CD107a | BV421 | 1D4B | Rat IgG2a | 200 |
| CD62L | BV510 | MEL-14 | Rat IgG2a | 200 |
| TNF alpha | BV650 | MP6-XT22 | Rat IgG1 | 200 |
| IL-2 | PE | JES6-5H4 | Rat IgG2b | 200 |
| IFN | PE/Cy7 | XMG1.2 | Rat IgG2b | 400 |
| gamma | | | | |

### IFNγ ELISpot Analysis

Spot forming units were enumerated using a CTL ImmunoSpot® Analyzer. Background subtraction was performed by subtracting vehicle-only spots from each well. Statistical significance for the ELISpot results was calculated by the Mann-Whitney test and flow cytometry results by Two-way ANOVA using Graphpad Prism software. Parameters applied were a 2-tailed distribution. P values equal to or less than 0.05 were considered to be significant: 0.05≥P>0.01, *significant; 0.01≥P>0.001, **very significant; 0.001>P, ***highly significant.

### Flow Cytometry Analysis

All flow cytometry analyses were performed using FlowJo (version 10, FlowJo; Ashland, OR). Compensation was adjusted manually in this software using compensation controls and applied equally to all samples within a stain. The compensation matrices were similar between each stain but were adjusted for each stain according to the single-color controls.

### Example 1D. Results

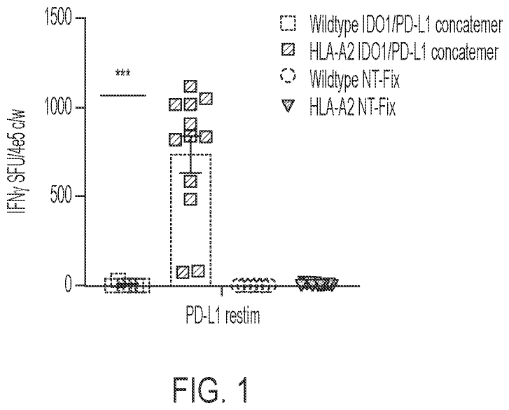

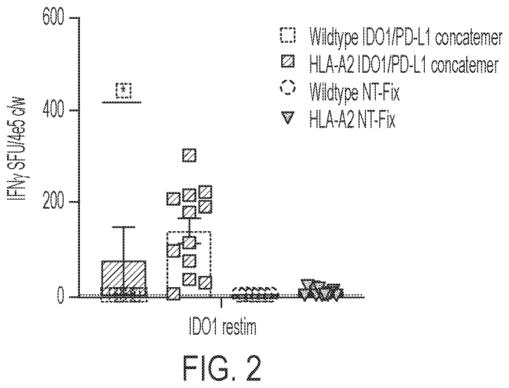

IFNγ ELISpot responses were compared between the splenocytes of HLA-A*02:01 and wildtype mice vaccinated with IDO1/PDL1 mRNA formulated in compound 25-based LNPs by re-stimulating cells ex vivo with overlapping peptides corresponding to the IDO1 and PD-L1 sequences encoded by the mRNA vaccine or vehicle-only. Background subtraction was performed by subtracting vehicle-only spots from each well. Responses were detected to IDO1 and PD-L1 epitopes in the vaccine. Additionally, epitope specific responses detected in HLA-A*02:01 animals are significantly greater than those detected in wildtype animals as determined by Mann-Whitney t-test; FIG. 1 and FIG. 2). Minimal responses were detected to vehicle-only.

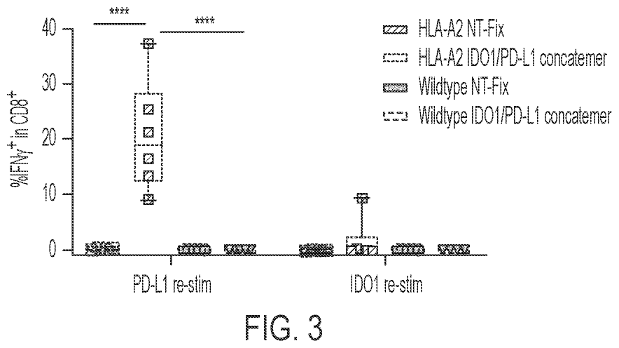

CD8+IFNγ+ T cell responses were also measured in response to ex vivo re-stimulation with vehicle-only, IDO1, and PD-L1 peptides by ICS and flow cytometry (FIG. 3). Background subtraction was performed by subtracting vehicle-only responses from the corresponding sample. PD-L1-specific CD8 T cell responses were detected in IDO1/PD-L1 concatemer treated animals. Additionally, PD-L1-specific responses detected in HLA-A*02:01 animals are significantly greater than those detected in wildtype animals as determined by Mann-Whitney t-test. No significant IDO1-specific CD8+IFNγ+ T cell responses were detected with this assay.

In a subsequent pharmacology study, robust IDO1-specific T cell responses were induced by the checkpoint cancer vaccine. Notably, these responses were detectable irrespective of the HLA-A*02:01 transgene (data not shown).

### Example 1E. Conclusions

The IDO1/PD-L1 antigen concatemer mRNA vaccine was designed to generate T cell responses. HLA-A*02:01 restricted immune responses were evaluated by comparing IDO1/PD-L1-specific responses in HLA-A*02:01 transgenic and wildtype mice after vaccination with the IDO1/PD-L1 concatemer mRNA vaccine. A significantly higher quantity of IDO1 and PD-L1 specific responses were observed by IFNγ ELISpot in HLA-A*02:01 compared to wildtype animals indicating the antigen-specific responses are also mediated by the human HLA. Flow cytometry-based analysis indicated PD-L1-specific responses were driven by CD8+IFNγ+ T cells. Together, these data demonstrate that the IDO1/PD-L1 antigen concatemer mRNA vaccine elicits IDO1/PD-L1 specific T cell responses in the HLA-A*02:01 transgenic mouse immunogenicity model.

### Example 2. Immunostimulatory/Immunodynamic Modeling to Support the First in Human Dose Selection of an mRNA Encoding a Checkpoint Cancer Vaccine

This example describes semi-mechanistic mathematical modeling to identify a dosage range for an mRNA encoding a checkpoint cancer vaccine against PD-L1 and IDO, two targets frequently upregulated in cancers. This modeling framework, also called IS/ID modeling, describes the immune response stimulated by vaccination (IS), and the resulting immune response dynamics (ID).

Vaccine drug development lacks traditional concentration versus time profiles commonly used as a framework in standard PK/PD modeling approaches. Accordingly, vaccine dose selection for first in human (FIH) trials often involves selection of a relevant animal model that would enable proper assessment of safety, toxicology, and dose-response through model-informed drug development (MIDD) and toxicity data assessments. In addition, the selected dose must also be therapeutically relevant as the study is conducted in oncology patients and not healthy subjects.

However, in the context of the checkpoint cancer vaccine directed to IDO1 and PD-L1, there are no relevant animal models to evaluate on-target toxicity of T-cells activated against either PD-L1 or IDO1. The absence of a clinically relevant model was primarily due to the following: (a) a high binding specificity of T cell receptors (TCRs) to the peptide-MHC complex is required for immune response; (b) the presence of high polymorphisms and low prevalence of MHC binding alleles within and across all species; (c) a predicted low probability of MHC binding peptides in non-human primates; and (d) an inability of animal models to mimic the pharmacological activity that is observed in humans. Furthermore, traditional toxicity studies would be limited to the toxicity of the LNP-only and not the antigen-specific on-target T cell toxicity.

Accordingly, to assess toxicity, other programs were leveraged that used the same LNP formulation to help define LNP toxicity and clinical safety data was obtained from previously published clinical safety data on a peptide-based vaccine directed to PD-L1 and IDO1. Leveraging the published literature data of the peptide-based vaccine against the PD-L1 and IDO1 a modeling framework capable of predicting a therapeutically relevant FIH dose was established.

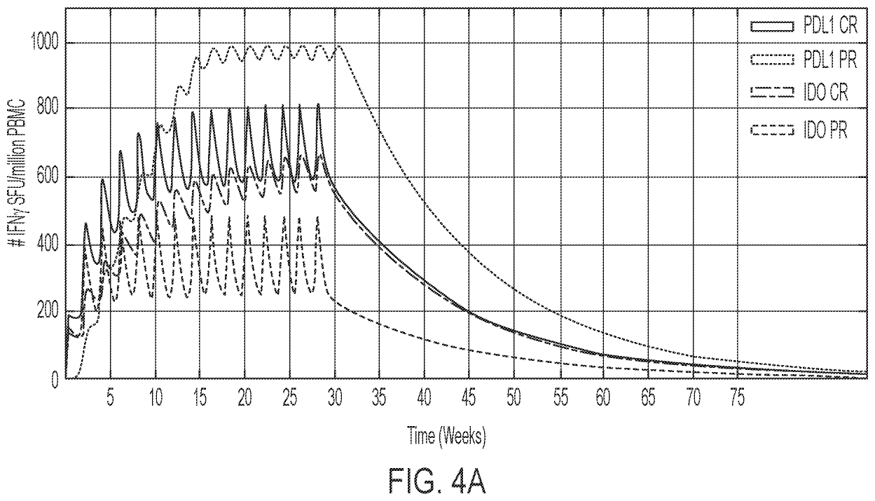

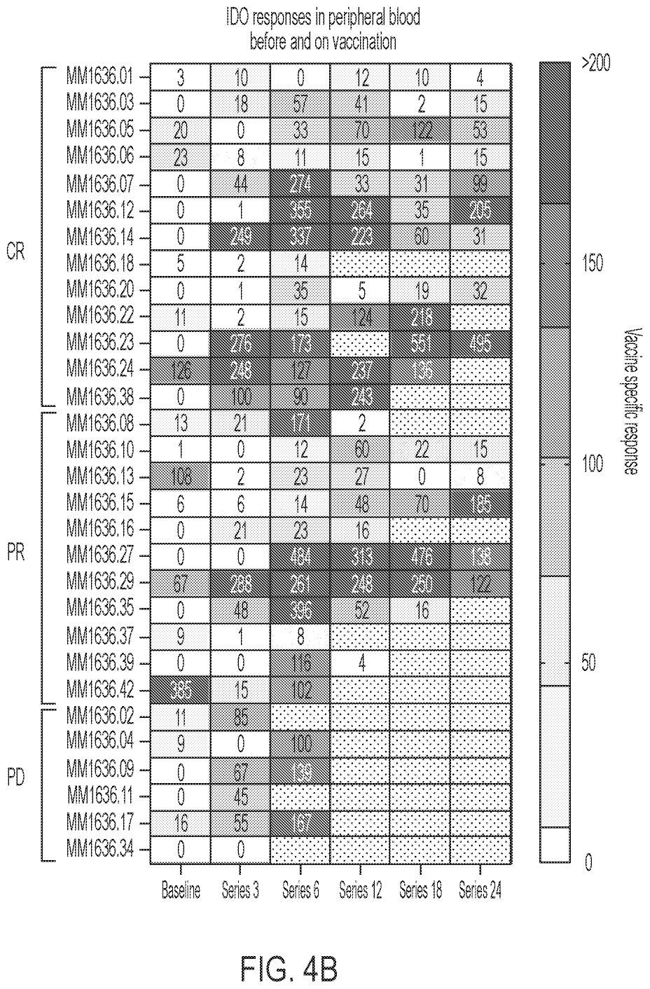

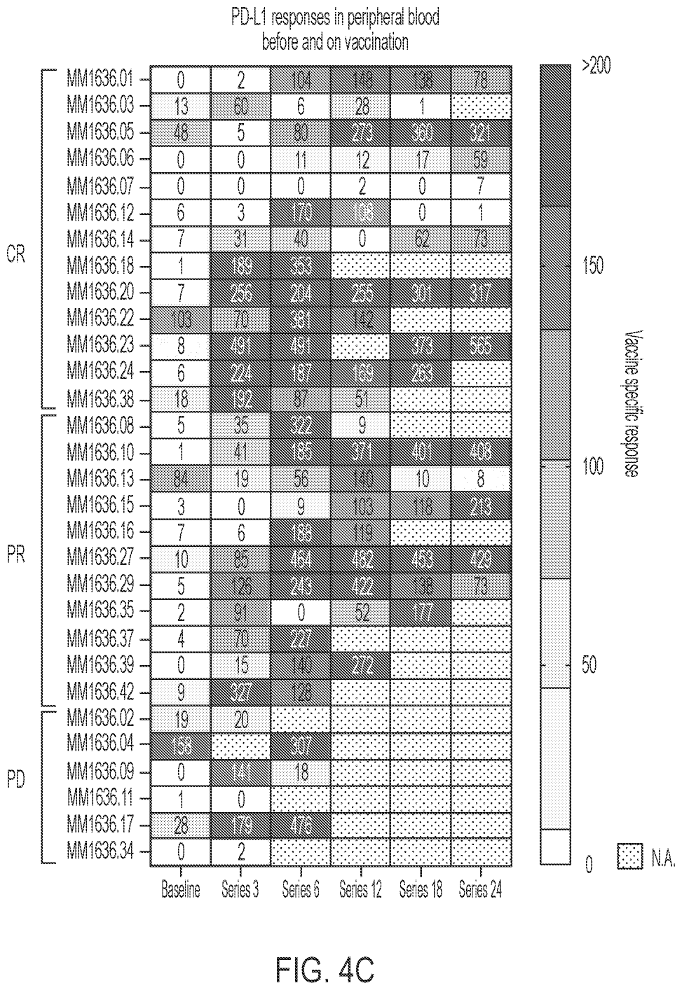

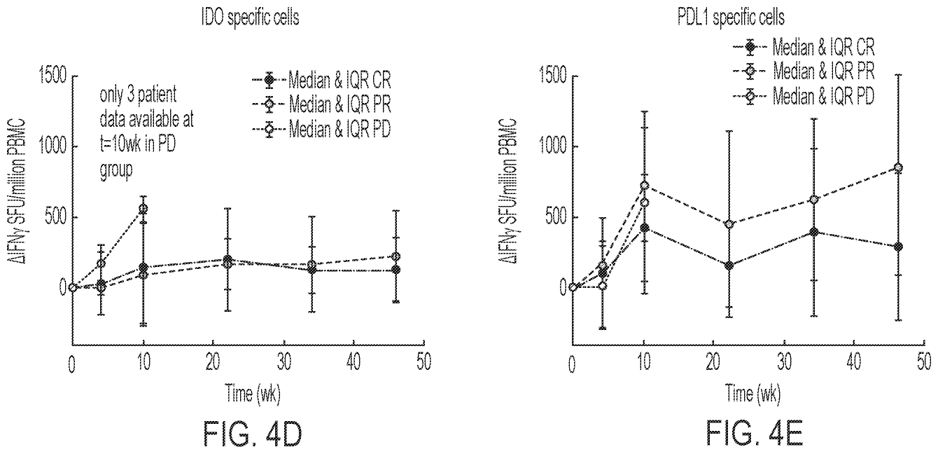

Similar to traditional PK/PD modeling, the first step in developing a model was to mathematically describe the underlying mechanisms of immune stimulation and dynamics of the response. In a reference study using a peptide vaccine comprised of a single PD-L1 peptide and a single IDO1 peptide, oncology patients were given 15 vaccine doses every 2 weeks and their immune responses were measured. As shown in FIG. 4A, the patients' immune responses were measured by looking at their T-cell responses up to 75 weeks following vaccination as depicted by the number of IFNγ Spot Forming Units (SFU) normalized to 1 million peripheral blood mononuclear cells (PBMCs) over time.

The antigen specific T-cell response stratified by patient was digitized and scaled to 1 million PBMCs (FIGS. 4B and 4C). Data was explored by response category (FIGS. 4D and 4E) and a modeling framework was constructed around the digitized data.

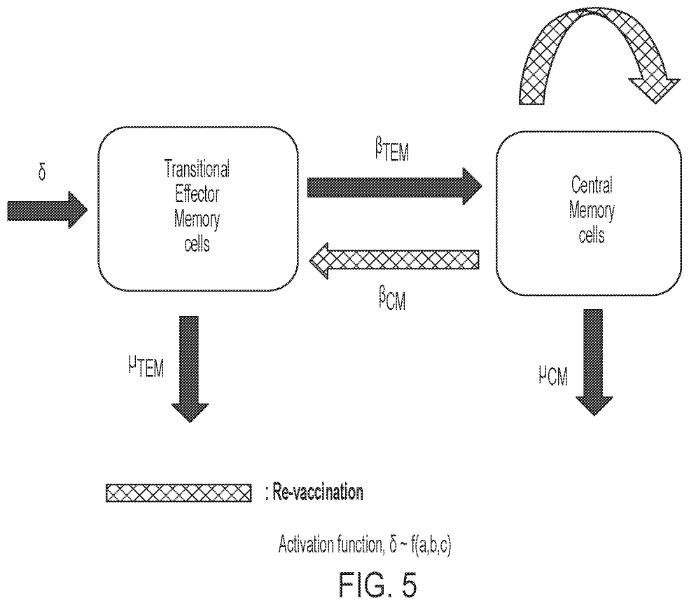

In the model, based on a previously published IS/ID model (FIG. 5), Transitional Effector Memory (TEM) Cells were brought into the system upon vaccination as represented by the activation function, δ, which is a function of magnitude (a), delay (b), and time (c) activation (FIG. 5). As shown in FIG. 5, the TEM cells were formed by conversion of central memory cells into TEM cells on revaccination (B_{CM}). The TEM were cells diminished either due to natural death (μ_{TEM}) or by their conversion into longer-lived central memory cells (β_{TEM}). Central memory cells are longer-lived and are made by TEM differentiation (β_{TEM}) or by replication (RCM). Central memory cells were also removed from their pool by conversion to TEM cells (B_{CM}) upon revaccination or death (μ_{CM}). The model is depicted by the formula:

$$d ( TEM ) / dt = δ ( Activation Function ) - {β}_{TEM} * TEM - {μ}_{TEM} * TEM + CM * {β}_{CM}$$

$$d ( CM ) / dt = {β}_{TEM} * TEM - ( CM * {β}_{CM} - CM * RCM + μ CM * CM ) upon revaccination$$

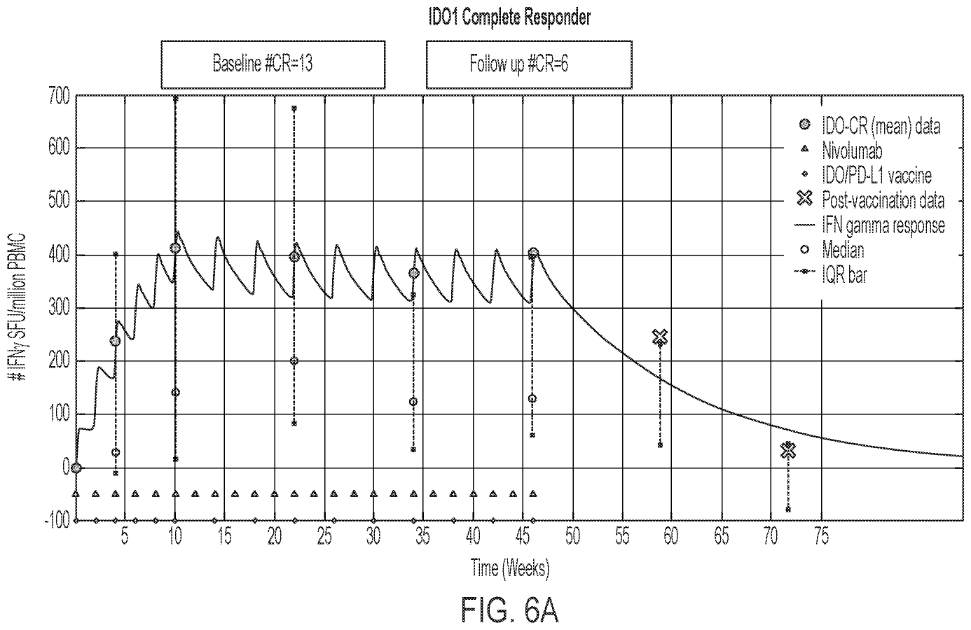

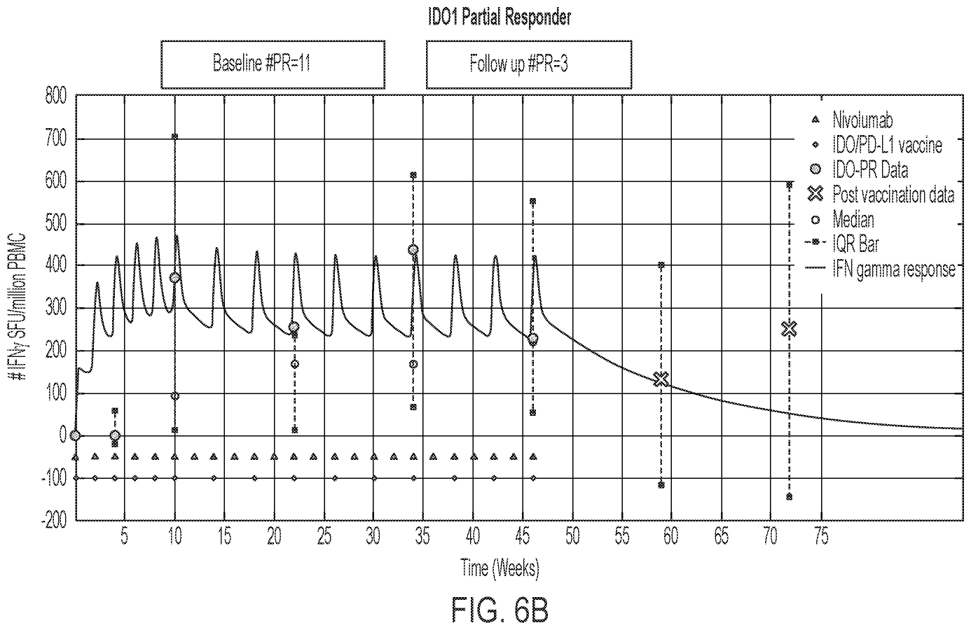

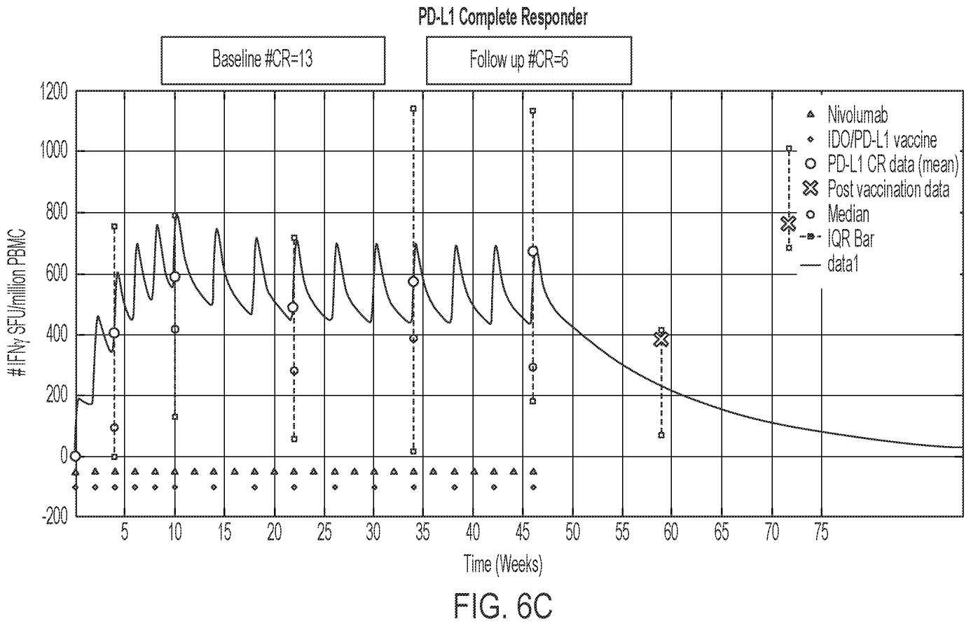

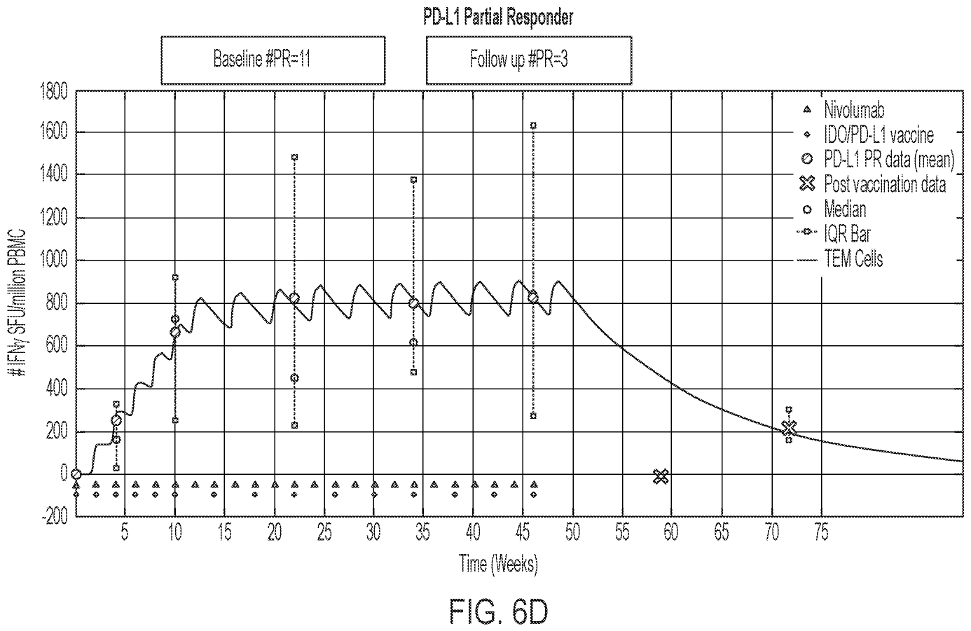

Once an IS/ID model was generated, it was qualified against the published peptide-based vaccine digitized data (FIGS. 6A-6D). Briefly, the model was fitted against mean IDO1 (FIGS. 6A and 6B) or PD-L1 (FIGS. 6C and 6D) T-cell response data separated by response groups, complete responders (FIGS. 6A and 6C) and partial responders (FIGS. 6B and 6D). As shown in FIGS. 6A-6D, the model adequately fit the mean published data up to 72 weeks post-treatment.

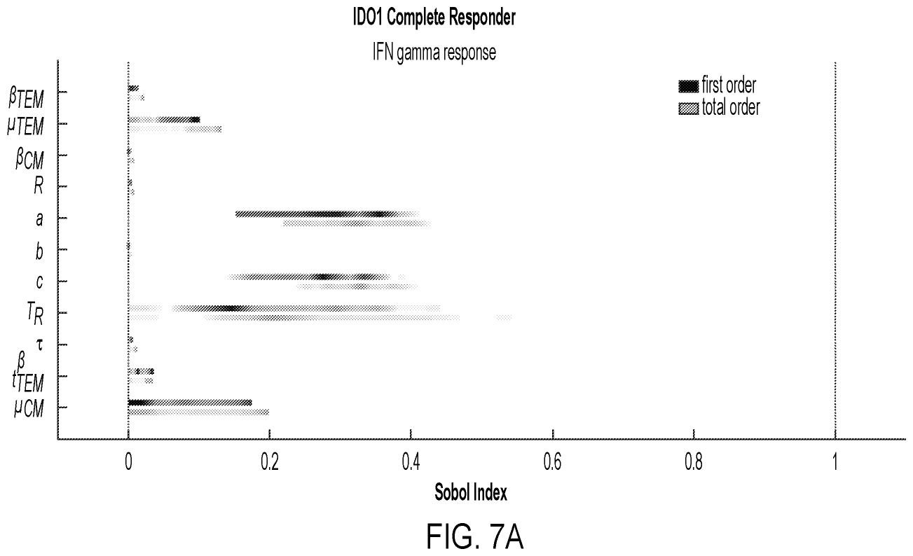

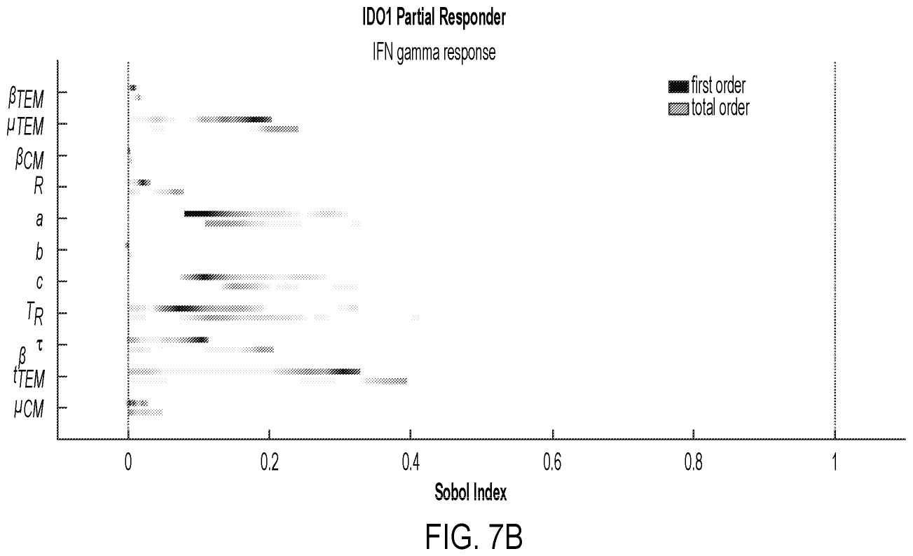

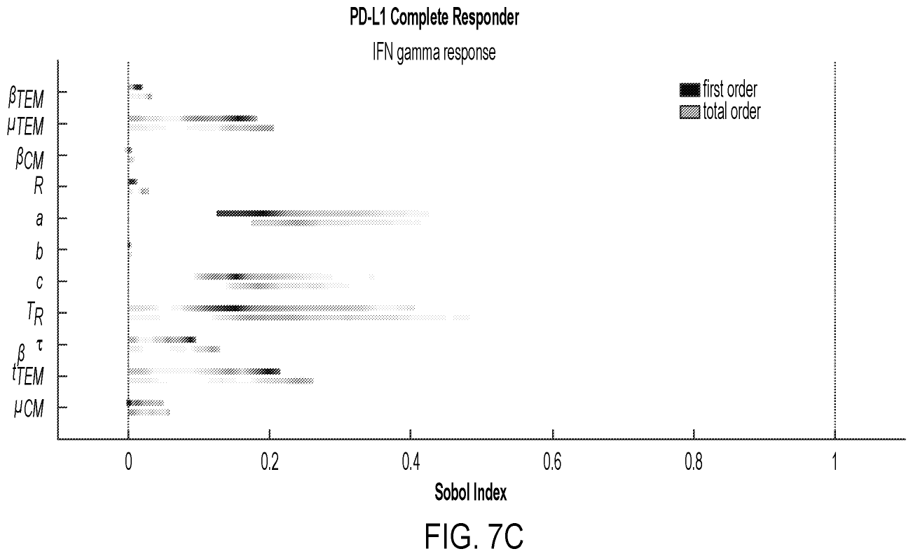

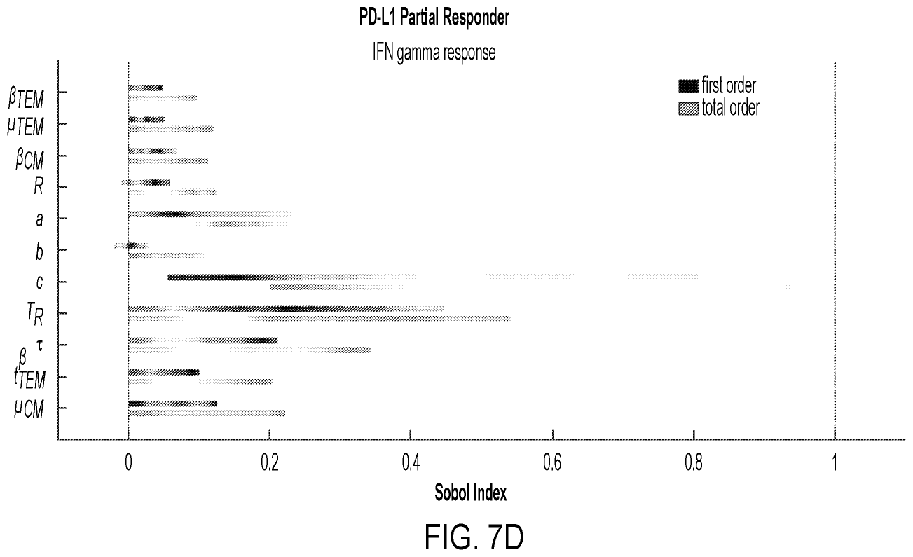

Next, a parameter sensitivity analysis was conducted to further qualify the model, as shown in FIGS. 7A-7D. A single model parameterization was taken as a baseline and varied by a range of 0.5 to 2× to measure an effective outcome due to the variation. Both a first-order sobol index and a total-order sobol index were used as measures of sensitivity. The first-order sobol measured sensitivity of the parameter when it was varied alone, and the total-order sobol measures sensitivity of a parameter when it is varied as well as with all other parameters. As shown in FIGS. 7A-7D, different color intensities are used to represent the frequency of observing the corresponding sobol index over the full-time course. A darker color means a high frequency of observing the corresponding sobol index, and a lighter color means lower frequency of the sobol index.

The global sensitivity analysis showed appropriate levels of sensitivity to all estimated parameters and showed that the magnitude of the sensitivity parameters was similar across the two phenotypes (CP/PR) except for a high sensitivity of ‘b’ (time of activation) and conversion to TEM from CM cells (BCM) for the PD-L1 CR patient fit (FIGS. 7A-7D). The high sensitivity for the PD-L1 CR patient fit was likely due to the large difference in observed response between week 4 and week 10 in this subset of patients, which requires a longer time to reach peak influx of TEM cells.

Another sensitive parameter was the time of revaccination (TR) suggesting that different regimens may lead to different T-cell responses (FIGS. 7A-7D). Without wishing to be bound by theory, a higher sensitivity in the time to revaccination parameter suggests it may be possible to optimize the dosing schedule (FIGS. 7A-7D).

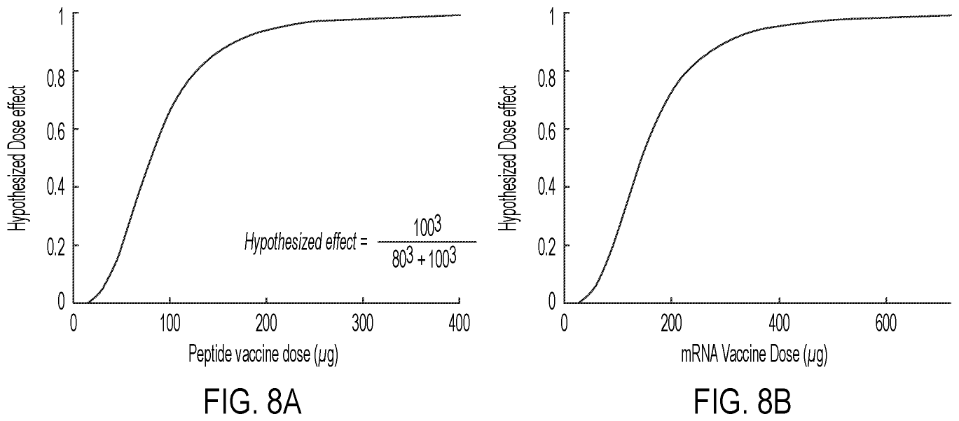

Once the model was qualified and a sensitivity analysis was conducted, various simulations were performed. The predicted dose-effect curve for the peptide-based vaccine is shown in FIG. 8A, and the equivalent curve for the mRNA vaccine are shown in FIG. 8B. The results shown in FIGS. 8A and 8B suggested that an equivalent 180 μg dose of the mRNA encoding the checkpoint cancer vaccine could elicit a similar T-cell response compared to the peptide-based vaccine. This is based on certain modeling assumptions including: (a) a mRNA vaccine should be targeted to elicit the same response as seen in the peptide-based vaccine trial; (b) that there is perfect efficiency in molecule internalization by APCs; (c) that each mRNA molecule has 4 PD-L1 and 4 IDO1 sequences as designed; and (d) each mRNA molecule produces approximately 17 moles of peptide.

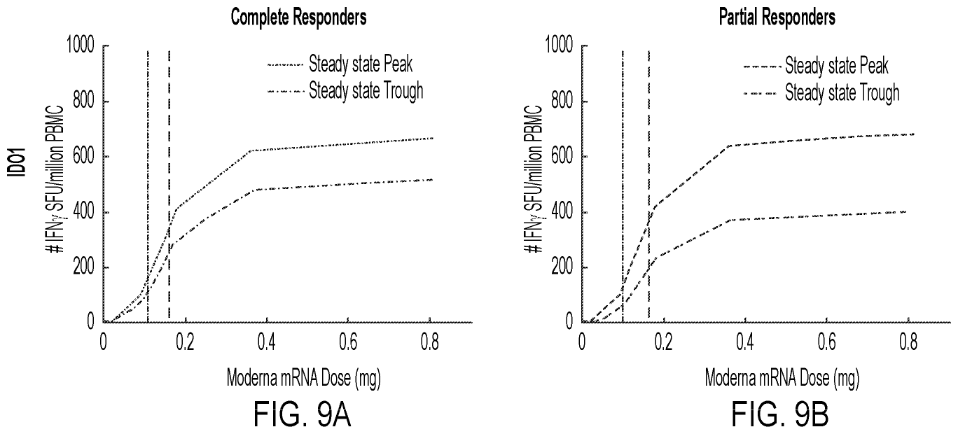

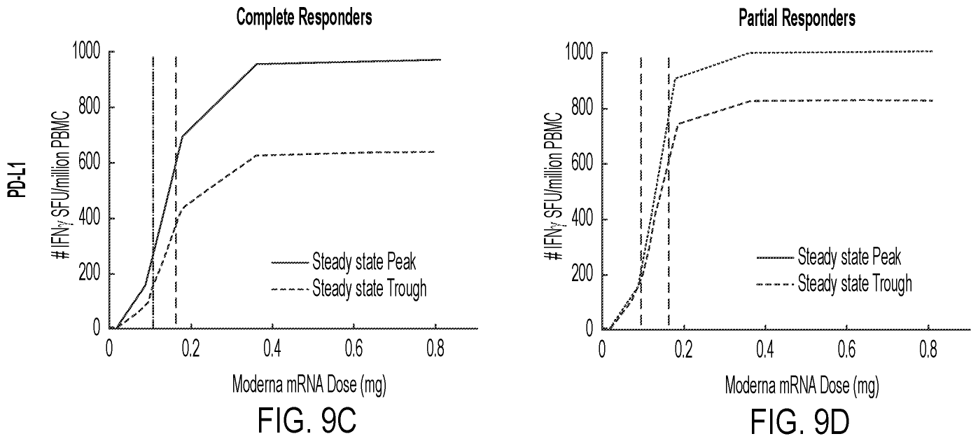

Once the dose-effect relationship was established, dose-effect simulations were conducted to determine potential differences in response and peptide types (FIGS. 9A-9D). Although the model, as shown in FIGS. 8A and 8B, predicted that a 180 μg dose could likely elicit a robust T-cell response, in the absence of pre-clinical data, following evaluation of several relevant clinical considerations a 100 μg dose was identified as one that could theoretically provide therapeutic response but allow for exploration and investigation of the dose-response curve for both therapeutic effect and adverse events.

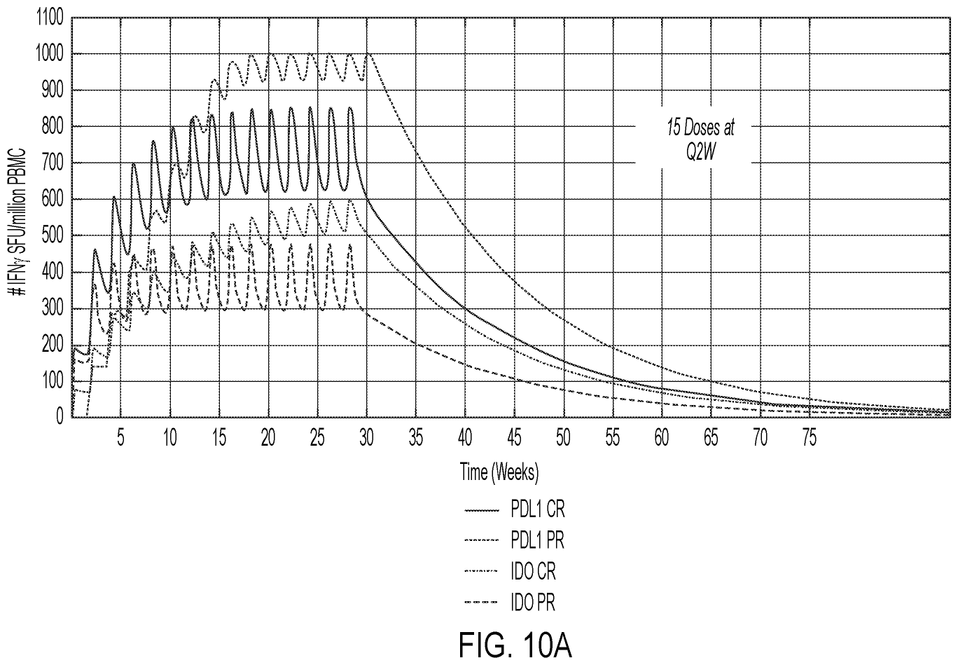

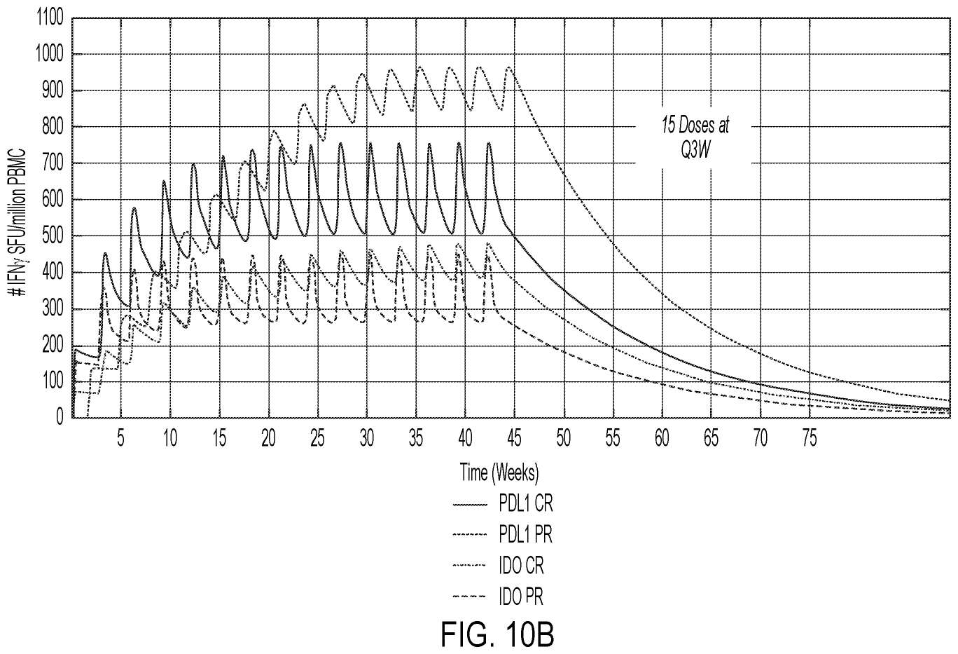

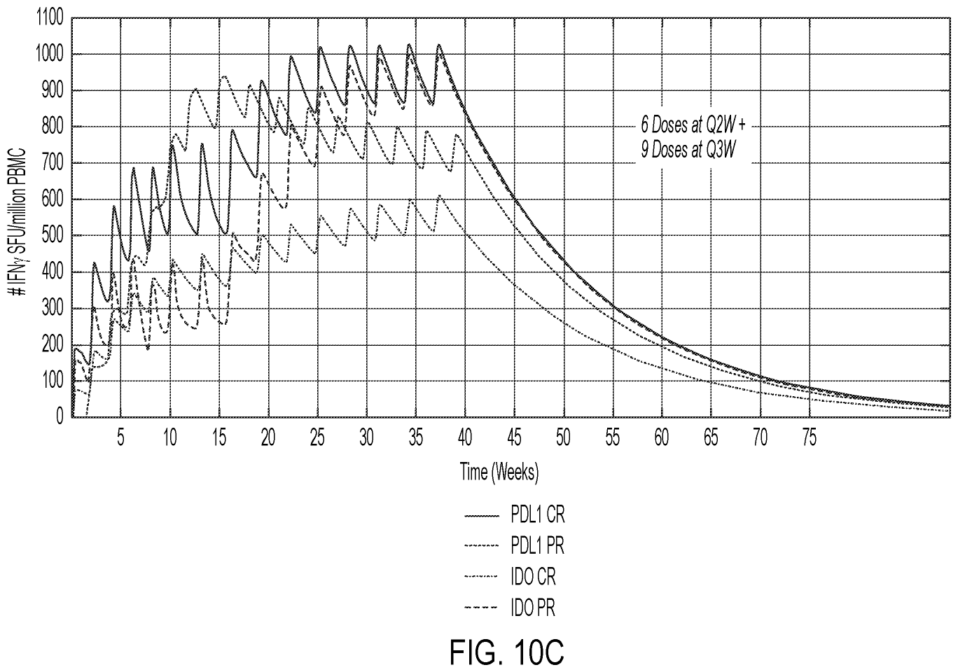

Finally, to model the effect of repeat dosing, several feasible dosing regimen simulations were conducted to estimate T-cell response following various potential vaccination schedules (FIGS. 10A-10C). Based on these preliminary simulations, a 15-dose QW3 regimen was predicted to have a longer response in comparison to the other simulated scenarios (FIGS. 10A-10C).

### Example 3. First in Human, Phase ½ Open-Label Study of an mRNA Encoding a Checkpoint Cancer Vaccine

This study will evaluate the safety and tolerability of a checkpoint cancer vaccine alone or in combination with pembrolizumab in patients with locally advanced or metastatic solid tumors. This study will include the following treatment arms:

Arm 1a includes adults with locally advanced or metastatic cancer (e.g., cutaneous melanoma, non-small cell lung carcinoma [NSCLC], non-muscle invasive bladder cancer, head and neck squamous cell carcinoma, microsatellite stable colorectal cancer, basal cell carcinoma, or triple negative breast cancer). Dose levels are to be determined by modified continuous reassessment method modeling, preliminary dose levels are 50 μg, 100 μg, 200 μg, 400 μg, and 1000 μg. The checkpoint cancer vaccine will be administered intramuscularly once every 3 weeks (Day 1 of each cycle) for 9 cycles. The starting dose at 100 μg will be administered to Arm 1a participants.

Arm 1b and pharmacodynamic (PD) Arm Groups 1 and 2 include adults with locally advanced or metastatic, and checkpoint inhibitor refractory melanoma or locally advanced or metastatic, and checkpoint inhibitor refractory NSCLC. Arm 1b will be initiated sequentially at a checkpoint cancer vaccine dose level that had been declared safe in Arm 1a based upon the totality of the data and at least one dose level less than then current level which was explored in Arm 1a. Pembrolizumab dosing is planned in combination with the checkpoint cancer vaccine only and is to be administered at 400 mg as a 30-minute intravenous infusion once every 6 weeks (e.g., on Day 1 of every other cycle; Cycle 1, Cycle 3, Cycle 5, Cycle 7, and Cycle 9) during the 9-cycle Treatment Period. Participants in the PD Arm will receive the checkpoint cancer vaccine in combination with pembrolizumab.

Arm 2 includes adults who have not received any prior therapy for this cancer in this setting (e.g., first line therapy) with locally advanced or metastatic melanoma (Cohort 1) or with locally advanced or metastatic NSCLC with a PD-L1 tumor proportion score≥1% (Cohort 2). Once the MTD/recommended dose for expansion has been declared for a combination therapy in the Dose Confirmation Arm (Arm 1b), the Dose Expansion Arm will begin. Participants in Arm 2 cohorts were to be treated on Day 1 of 21-day cycles for nine cycles (9 total doses of the checkpoint cancer vaccine; 5 total doses of pembrolizumab).

The safety and tolerability of the checkpoint cancer vaccine alone or in combination with pembrolizumab will be evaluated by a variety of readouts. Primary outcome measures will include the number of patents with dose-limiting toxicities and number and type of adverse events. Secondary endpoints will include objective response rate (ORR), disease control rate (DCR), duration of response (DOR), and progression free survival (PFS), e.g., based on Response Evaluation Criteria in Solid Tumors Version 1.1 (RECIST). Additional outcome measures will include percent change from baseline in T cell profile in the periphery and in the tumor (e.g., as measured by changes in CD3+CD8+, CD3+CD4+ and CD3+CD4+Foxp3+ (e.g., measured by flow cytometry in peripheral blood and by immunohistochemistry in tumor).

### OTHER EMBODIMENTS

It is to be understood that while the present disclosure has been described in conjunction with the detailed description thereof, the foregoing description is intended to illustrate and not limit the scope of the present disclosure, which is defined by the scope of the appended claims. Other aspects, advantages, and alterations are within the scope of the following claims. All references described herein are incorporated by reference in their entireties.

What is claimed is:

1. A lipid nanoparticle (LNP) composition comprising a polynucleotide comprising an mRNA which encodes a checkpoint cancer vaccine comprising (i) one or more (e.g., 1, 2, 3, 4, or more) Indoleamine-pyrrole 2,3-dioxygenase (IDO) antigenic peptides and (ii) one or more (e.g., 1, 2, 3, 4, or more) programmed death-ligand 1 (PD-L1) antigenic peptides.

2. A lipid nanoparticle (LNP) composition for immunomodulation, e.g., for inducing an immune response and/or breaking immune tolerance (e.g., stimulating T effector cells), the composition comprising a polynucleotide comprising an mRNA which encodes a checkpoint cancer vaccine comprising (i) one or more (e.g., 1, 2, 3, 4, or more) IDO antigenic peptides and (ii) one or more (e.g., 1, 2, 3, 4, or more) PD-L1 antigenic peptides.

3. A lipid nanoparticle composition, for stimulating T effector cells, the composition comprising a polynucleotide comprising an mRNA which encodes a checkpoint cancer vaccine comprising (i) one or more (e.g., 1, 2, 3, 4, or more) IDO antigenic peptides and (ii) one or more (e.g., 1, 2, 3, 4, or more) PD-L1 antigenic peptides.

4. An mRNA construct comprising a polynucleotide which encodes a checkpoint cancer vaccine comprising (i) one or more (e.g., 1, 2, 3, 4, or more) IDO antigenic peptides and (ii) one or more (e.g., 1, 2, 3, 4, or more) PD-L1 antigenic peptides.

5. The LNP composition or mRNA construct of any of the preceding claims, wherein the IDO antigenic peptide comprises a fragment of a naturally occurring IDO molecule, or a variant thereof.

6. The LNP composition or mRNA construct of any of the preceding claims, wherein the IDO antigenic peptide is derived from IDO1 or IDO2.

7. The LNP composition or mRNA construct of claim 6, wherein the IDO antigenic peptide is derived from IDO1.

8. The LNP composition or mRNA construct of any of the preceding claims, wherein the IDO antigenic peptide comprises an amino acid sequence having at least 85%, 90%, 95%, 96%, 97%, 98%, 99% or 100% identity to the sequence of SEQ ID NO: 1.

9. The LNP composition or mRNA construct of any of the preceding claims, wherein the polynucleotide encoding the IDO antigenic fragment comprises a nucleotide sequence having at least 60%, 65%, 70%, 75%, 80%, 85%, 90%, 95%, 96%, 97%, 98%, 99% or 100% identity to the sequence of SEQ ID NO: 2, or an antigenic fragment thereof.

10. The LNP composition or mRNA construct of any of the preceding claims, wherein the PD-L1 antigenic peptide comprises a fragment of a naturally occurring PD-L1 molecule, or a variant thereof.

11. The LNP composition or mRNA construct of any of the preceding claims, wherein the PD-L1 antigenic peptide comprises an amino acid sequence having at least 85%, 90%, 95%, 96%, 97%, 98%, 99% or 100% identity to the sequence of SEQ ID NO: 3.

12. The LNP composition or mRNA construct of any of the preceding claims, wherein the polynucleotide encoding the PD-L1 antigenic fragment comprises a nucleotide sequence having at least 60%, 65%, 70%, 75%, 80%, 85%, 90%, 95%, 96%, 97%, 98%, 99% or 100% identity to the sequence of SEQ ID NO: 4, or an antigenic fragment thereof.

13. The LNP composition or mRNA construct of any of the preceding claims, wherein the checkpoint cancer vaccine comprises two IDO antigenic peptides and two PD-L1 antigenic peptides.

14. The LNP composition or mRNA construct of any one of claims 1-12, wherein the checkpoint cancer vaccine comprises three IDO antigenic peptides and three PD-L1 antigenic peptides.

15. The LNP composition or mRNA construct of any one of claims 1-12, wherein the checkpoint cancer vaccine comprises four IDO antigenic peptides and four PD-L1 antigenic peptides.

16. The LNP composition or mRNA construct of any of the preceding claims, wherein the checkpoint cancer vaccine comprises alternating IDO and PD-L1 antigenic peptides.

17. The LNP composition or mRNA construct of claim 15 or 16, wherein the checkpoint cancer vaccine comprises (i) an IDO antigenic peptide, (ii) a PD-L1 antigenic peptide, (iii) an IDO antigenic peptide, (iv) a PD-L1 antigenic peptide, (v) an IDO antigenic peptide, (vi) a PD-L1 antigenic peptide, (vii) an IDO antigenic peptide, and (viii) a PD-L1 antigenic peptide.

18. The LNP composition or mRNA construct of any one of claims 15-17, wherein the alternating IDO and PD-L1 antigenic peptides comprise an amino acid sequence having at least 85%, 90%, 95%, 96%, 97%, 98%, 99% or 100% identity to an IDO amino acid sequence provided in Table 1A, e.g., SEQ ID NO: 5, or an antigenic fragment thereof.

19. The LNP composition or mRNA construct of any one of claims 16-18, wherein the polynucleotide encoding the alternating IDO and PD-L1 antigenic peptides comprises a nucleotide sequence having at least 50%, 55%, 60%, 65%, 70%, 75%, 80%, 85%, 90%, 95%, 96%, 97%, 98%, 99% or 100% identity to the sequence of SEQ ID NO: 6, 300, 301, or 302, or an antigenic fragment thereof.

20. The LNP composition or mRNA construct of any of the preceding claims, which results in:
(i) stimulation of T effector cells;
(ii) cytotoxic T cell-mediated killing of suppressive immune and tumor cells that overexpress PD-L1 or IDO; and/or
(iii) induction of an anti-tumor immune response.

21. The LNP composition or mRNA construct of any of the preceding claims, which results in amelioration or delay of cancer progression, e.g., as described herein, in a subject.

22. The LNP composition or mRNA construct of any one of the preceding claims, wherein the polynucleotide comprising an mRNA encoding the checkpoint cancer vaccine comprising one or more (e.g., 1, 2, 3, 4, or more) Indoleamine-pyrrole 2,3-dioxygenase (IDO) antigenic peptides and (ii) one or more (e.g., 1, 2, 3, 4, or more) programmed death-ligand 1 (PD-L1) antigenic peptides, comprises at least one chemical modification.

23. The LNP composition or mRNA construct of claim 22, wherein the chemical modification is N1-methylpseudouridine

24. The LNP composition of any one of the preceding claims, wherein the LNP composition comprises: (i) an ionizable lipid, e.g., an amino lipid; (ii) a sterol or other structural lipid; (iii) a non-cationic helper lipid or phospholipid; and (iv) a PEG-lipid.

25. The LNP composition of claim 24, wherein the ionizable lipid comprises Compound 25.

26. The LNP composition of claim 24, wherein the PEG lipid is PEG DMG.

27. The LNP composition of claim 24, wherein the LNP composition comprises (i) Compound 25, (ii) cholesterol, (iii) DSPC, and (iv) PEG DMG.

28. A pharmaceutical composition comprising the LNP composition or mRNA construct of any one of claims 1-27.

29. A method of modulating, e.g., stimulating, an immune response in a subject, comprising administering to the subject in need thereof an effective amount of an LNP composition comprising a polynucleotide comprising an mRNA which encodes a checkpoint cancer vaccine comprising one or more (e.g., 1, 2, 3, 4, or more) Indoleamine-pyrrole 2,3-dioxygenase (IDO) antigenic peptides and (ii) one or more (e.g., 1, 2, 3, 4, or more) programmed death-ligand 1 (PD-L1) antigenic peptides.

30. A method of stimulating T effector cells in a subject, comprising administering to the subject an effective amount of an LNP composition comprising a polynucleotide comprising an mRNA which encodes a checkpoint cancer vaccine comprising one or more (e.g., 1, 2, 3, 4, or more) Indoleamine-pyrrole 2,3-dioxygenase (IDO) antigenic peptides and (ii) one or more (e.g., 1, 2, 3, 4, or more) programmed death-ligand 1 (PD-L1) antigenic peptides.

31. A method of treating, or preventing, a cancer, or a symptom thereof, comprising administering to the subject in need thereof an effective amount of an LNP composition comprising a polynucleotide comprising an mRNA which encodes a checkpoint cancer vaccine comprising one or more (e.g., 1, 2, 3, 4, or more) Indoleamine-pyrrole 2,3-dioxygenase (IDO) antigenic peptides and (ii) one or more (e.g., 1, 2, 3, 4, or more) programmed death-ligand 1 (PD-L1) antigenic peptides.

32. The method of claim 31, wherein the cancer is a locally advanced or metastatic solid tumor.

33. The method of claim 31, wherein the cancer is a melanoma.

34. The method of claim 33, wherein the melanoma is a cutaneous melanoma.

35. The method of claim 34, wherein the cutaneous melanoma is a 1 L cutaneous melanoma stage IIIB+.

36. The method of claim 31, wherein the cancer is a NSCLC.

37. The method of claim 36, wherein the NSCLC is a 1 L NSCLC.

38. The method of claim 31, wherein the cancer is a bladder cancer.

39. The method of claim 38, wherein bladder cancer is a non-muscle invasive bladder cancer.

40. The method of claim 31, wherein the cancer is a head and neck cancer.

41. The method of claim 40, wherein the head and neck cancer is a head and neck squamous cell carcinoma.

42. The method of claim 31, wherein the cancer is a colorectal cancer.

43. The method of claim 42, wherein the colorectal cancer is a microsatellite stable colorectal cancer.

44. The method of claim 31, wherein the cancer is a basal cell carcinoma.

45. The method of claim 31, wherein the cancer is a breast cancer.

46. The method of claim 45, wherein the breast cancer is a triple negative breast cancer.

47. The method of any of claims 29-46, wherein the mRNA encoding the checkpoint cancer vaccine comprises the nucleic acid sequence of SEQ ID NO: 300, 301, or 302.

48. The method of any one of claims 29-47, wherein the LNP composition comprises: (i) an ionizable lipid, e.g., an amino lipid; (ii) a sterol or other structural lipid; (iii) a non-cationic helper lipid or phospholipid; and (iv) a PEG-lipid.

49. The method of claim 48, wherein the ionizable lipid comprises Compound 25.

50. The method of claim 48, wherein the LNP composition comprises (i) Compound 25, (ii) cholesterol, (iii) DSPC, and (iv) PEG DMG.

51. The method of claim 48, wherein the method further comprises administering a checkpoint inhibitor selected from the group consisting of anti-PD-1 and anti-CTLA4 to the subject.

52. The method of claim 51, wherein the checkpoint inhibitor comprises pembrolizumab.

53. The method of any one of claims 29-52, wherein the LNP composition is administered at a dose of 100 μg to about 1 mg.

54. The method of claim 53, wherein the LNP composition is administered at a dose of 50 μg to 150 μg, 150 μg to 250 μg, 250 μg to 350 μg, 350 μg to 450 μg, 450 μg to 550 μg, 550 μg to 650 μg, 650 μg to 750 μg, 750 μg to 850 μg, 850 μg to 950 μg, or 950 μg to 1 mg.

55. The method of claim 54, wherein the LNP composition is administered at a dose of 50 μg, 100 μg, 200 μg, 300 μg, 400 μg, 500 μg, 600 μg, 700 μg, 800 μg, 900 μg, or 1 mg.

56. The method of any one of claims 29-55, wherein the LNP composition is administered intramuscularly.

57. The method of any one of claims 29-56, wherein the LNP composition is administered once every three weeks.


SEQUENCE LISTING (716 sequences)

SEQ ID NO: 1 - protein, 25 aa, synthetic construct
ERDTLLKALLEIASCLEKALQVFHQ

SEQ ID NO: 2 - RNA, 75 nt, synthetic construct
  source 1..75: mol_type = other RNA
gagcgggataccctcctgaaggctctgctggagatcgcaagctgcctggagaaagccctccaggtgttccaccaa

SEQ ID NO: 3 - protein, 25 aa, synthetic construct
VFIFMTYWHLLNAFTVTVPKDLYVV

SEQ ID NO: 4 - RNA, 75 nt, synthetic construct
  source 1..75: mol_type = other RNA
gtgttcatcttcatgacatactggcatctgctgaacgcctttaccgtgaccgtacccaaggacctgtacgtggtg

SEQ ID NO: 5 - protein, 200 aa, synthetic construct
ERDTLLKALLEIASCLEKALQVFHQVFIFMTYWHLLNAFTVTVPKDLYVVERDTLLKALLEIASCLEKALQVFHQVFIFMTYWHLLNAFTVTVPKDLYVVERDTLLKALLEIASCLEKALQVFHQVFIFMTYWHLLNAFTVTVPKDLYVVERDTLLKALLEIASCLEKALQVFHQVFIFMTYWHLLNAFTVTVPKDLYVV

SEQ ID NO: 6 - RNA, 603 nt, synthetic construct
  source 1..603: mol_type = other RNA
  modified_base 2: mod_base = OTHER; note = N1-methylpseudouracil
  modified_base 12: mod_base = OTHER; note = N1-methylpseudouracil
  modified_base 17: mod_base = OTHER; note = N1-methylpseudouracil
  modified_base 20: mod_base = OTHER; note = N1-methylpseudouracil
  modified_base 27: mod_base = OTHER; note = N1-methylpseudouracil
  modified_base 29: mod_base = OTHER; note = N1-methylpseudouracil
  modified_base 32: mod_base = OTHER; note = N1-methylpseudouracil
  modified_base 38: mod_base = OTHER; note = N1-methylpseudouracil
  modified_base 46: mod_base = OTHER; note = N1-methylpseudouracil
  modified_base 50: mod_base = OTHER; note = N1-methylpseudouracil
  modified_base 62: mod_base = OTHER; note = N1-methylpseudouracil
  modified_base 68: mod_base = OTHER; note = N1-methylpseudouracil
  modified_base 70: mod_base = OTHER; note = N1-methylpseudouracil
  modified_base 71: mod_base = OTHER; note = N1-methylpseudouracil
  modified_base 80: mod_base = OTHER; note = N1-methylpseudouracil
  modified_base 82: mod_base = OTHER; note = N1-methylpseudouracil
  modified_base 83: mod_base = OTHER; note = N1-methylpseudouracil
  modified_base 86: mod_base = OTHER; note = N1-methylpseudouracil
  modified_base 88: mod_base = OTHER; note = N1-methylpseudouracil
  modified_base 89: mod_base = OTHER; note = N1-methylpseudouracil
  modified_base 92: mod_base = OTHER; note = N1-methylpseudouracil
  modified_base 97: mod_base = OTHER; note = N1-methylpseudouracil
  modified_base 100: mod_base = OTHER; note = N1-methylpseudouracil
  modified_base 105: mod_base = OTHER; note = N1-methylpseudouracil
  modified_base 107: mod_base = OTHER; note = N1-methylpseudouracil
  modified_base 110: mod_base = OTHER; note = N1-methylpseudouracil
  modified_base 118: mod_base = OTHER; note = N1-methylpseudouracil
  modified_base 119: mod_base = OTHER; note = N1-methylpseudouracil
  modified_base 120: mod_base = OTHER; note = N1-methylpseudouracil
  modified_base 125: mod_base = OTHER; note = N1-methylpseudouracil
  modified_base 131: mod_base = OTHER; note = N1-methylpseudouracil
  modified_base 143: mod_base = OTHER; note = N1-methylpseudouracil
  modified_base 145: mod_base = OTHER; note = N1-methylpseudouracil
  modified_base 149: mod_base = OTHER; note = N1-methylpseudouracil
  modified_base 152: mod_base = OTHER; note = N1-methylpseudouracil
  modified_base 162: mod_base = OTHER; note = N1-methylpseudouracil
  modified_base 167: mod_base = OTHER; note = N1-methylpseudouracil
  modified_base 170: mod_base = OTHER; note = N1-methylpseudouracil
  modified_base 179: mod_base = OTHER; note = N1-methylpseudouracil
  modified_base 182: mod_base = OTHER; note = N1-methylpseudouracil
  modified_base 188: mod_base = OTHER; note = N1-methylpseudouracil
  modified_base 193: mod_base = OTHER; note = N1-methylpseudouracil
  modified_base 196: mod_base = OTHER; note = N1-methylpseudouracil
  modified_base 200: mod_base = OTHER; note = N1-methylpseudouracil
  modified_base 212: mod_base = OTHER; note = N1-methylpseudouracil
  modified_base 218: mod_base = OTHER; note = N1-methylpseudouracil
  modified_base 220: mod_base = OTHER; note = N1-methylpseudouracil
  modified_base 221: mod_base = OTHER; note = N1-methylpseudouracil
  modified_base 222: mod_base = OTHER; note = N1-methylpseudouracil
  modified_base 230: mod_base = OTHER; note = N1-methylpseudouracil
  modified_base 232: mod_base = OTHER; note = N1-methylpseudouracil
  modified_base 233: mod_base = OTHER; note = N1-methylpseudouracil
  modified_base 236: mod_base = OTHER; note = N1-methylpseudouracil
  modified_base 237: mod_base = OTHER; note = N1-methylpseudouracil
  modified_base 238: mod_base = OTHER; note = N1-methylpseudouracil
  modified_base 239: mod_base = OTHER; note = N1-methylpseudouracil
  modified_base 242: mod_base = OTHER; note = N1-methylpseudouracil
  modified_base 247: mod_base = OTHER; note = N1-methylpseudouracil
  modified_base 249: mod_base = OTHER; note = N1-methylpseudouracil
  modified_base 250: mod_base = OTHER; note = N1-methylpseudouracil
  modified_base 257: mod_base = OTHER; note = N1-methylpseudouracil
  modified_base 259: mod_base = OTHER; note = N1-methylpseudouracil
  modified_base 260: mod_base = OTHER; note = N1-methylpseudouracil
  modified_base 267: mod_base = OTHER; note = N1-methylpseudouracil
  modified_base 268: mod_base = OTHER; note = N1-methylpseudouracil
  modified_base 269: mod_base = OTHER; note = N1-methylpseudouracil
  modified_base 270: mod_base = OTHER; note = N1-methylpseudouracil
  modified_base 273: mod_base = OTHER; note = N1-methylpseudouracil
  modified_base 275: mod_base = OTHER; note = N1-methylpseudouracil
  modified_base 281: mod_base = OTHER; note = N1-methylpseudouracil
  modified_base 291: mod_base = OTHER; note = N1-methylpseudouracil
  modified_base 292: mod_base = OTHER; note = N1-methylpseudouracil
  modified_base 293: mod_base = OTHER; note = N1-methylpseudouracil
  modified_base 295: mod_base = OTHER; note = N1-methylpseudouracil
  modified_base 299: mod_base = OTHER; note = N1-methylpseudouracil
  modified_base 302: mod_base = OTHER; note = N1-methylpseudouracil
  modified_base 317: mod_base = OTHER; note = N1-methylpseudouracil
  modified_base 320: mod_base = OTHER; note = N1-methylpseudouracil
  modified_base 329: mod_base = OTHER; note = N1-methylpseudouracil
  modified_base 332: mod_base = OTHER; note = N1-methylpseudouracil
  modified_base 333: mod_base = OTHER; note = N1-methylpseudouracil
  modified_base 338: mod_base = OTHER; note = N1-methylpseudouracil
  modified_base 346: mod_base = OTHER; note = N1-methylpseudouracil
  modified_base 348: mod_base = OTHER; note = N1-methylpseudouracil
  modified_base 350: mod_base = OTHER; note = N1-methylpseudouracil
  modified_base 362: mod_base = OTHER; note = N1-methylpseudouracil
  modified_base 368: mod_base = OTHER; note = N1-methylpseudouracil
  modified_base 370: mod_base = OTHER; note = N1-methylpseudouracil
  modified_base 371: mod_base = OTHER; note = N1-methylpseudouracil
  modified_base 380: mod_base = OTHER; note = N1-methylpseudouracil
  modified_base 382: mod_base = OTHER; note = N1-methylpseudouracil
  modified_base 383: mod_base = OTHER; note = N1-methylpseudouracil
  modified_base 386: mod_base = OTHER; note = N1-methylpseudouracil
  modified_base 388: mod_base = OTHER; note = N1-methylpseudouracil
  modified_base 389: mod_base = OTHER; note = N1-methylpseudouracil
  modified_base 390: mod_base = OTHER; note = N1-methylpseudouracil
  modified_base 392: mod_base = OTHER; note = N1-methylpseudouracil
  modified_base 396: mod_base = OTHER; note = N1-methylpseudouracil
  modified_base 397: mod_base = OTHER; note = N1-methylpseudouracil
  modified_base 400: mod_base = OTHER; note = N1-methylpseudouracil
  modified_base 407: mod_base = OTHER; note = N1-methylpseudouracil
  modified_base 410: mod_base = OTHER; note = N1-methylpseudouracil
  modified_base 418: mod_base = OTHER; note = N1-methylpseudouracil
  modified_base 419: mod_base = OTHER; note = N1-methylpseudouracil
  modified_base 425: mod_base = OTHER; note = N1-methylpseudouracil
  modified_base 431: mod_base = OTHER; note = N1-methylpseudouracil
  modified_base 442: mod_base = OTHER; note = N1-methylpseudouracil
  modified_base 443: mod_base = OTHER; note = N1-methylpseudouracil
  modified_base 445: mod_base = OTHER; note = N1-methylpseudouracil
  modified_base 449: mod_base = OTHER; note = N1-methylpseudouracil
  modified_base 452: mod_base = OTHER; note = N1-methylpseudouracil
  modified_base 467: mod_base = OTHER; note = N1-methylpseudouracil
  modified_base 469: mod_base = OTHER; note = N1-methylpseudouracil
  modified_base 470: mod_base = OTHER; note = N1-methylpseudouracil
  modified_base 479: mod_base = OTHER; note = N1-methylpseudouracil
  modified_base 482: mod_base = OTHER; note = N1-methylpseudouracil
  modified_base 488: mod_base = OTHER; note = N1-methylpseudouracil
  modified_base 489: mod_base = OTHER; note = N1-methylpseudouracil
  modified_base 496: mod_base = OTHER; note = N1-methylpseudouracil
  modified_base 498: mod_base = OTHER; note = N1-methylpseudouracil
  modified_base 500: mod_base = OTHER; note = N1-methylpseudouracil
  modified_base 512: mod_base = OTHER; note = N1-methylpseudouracil
  modified_base 518: mod_base = OTHER; note = N1-methylpseudouracil
  modified_base 520: mod_base = OTHER; note = N1-methylpseudouracil
  modified_base 521: mod_base = OTHER; note = N1-methylpseudouracil
  modified_base 525: mod_base = OTHER; note = N1-methylpseudouracil
  modified_base 530: mod_base = OTHER; note = N1-methylpseudouracil
  modified_base 532: mod_base = OTHER; note = N1-methylpseudouracil
  modified_base 533: mod_base = OTHER; note = N1-methylpseudouracil
  modified_base 534: mod_base = OTHER; note = N1-methylpseudouracil
  modified_base 536: mod_base = OTHER; note = N1-methylpseudouracil
  modified_base 538: mod_base = OTHER; note = N1-methylpseudouracil
  modified_base 539: mod_base = OTHER; note = N1-methylpseudouracil
  modified_base 540: mod_base = OTHER; note = N1-methylpseudouracil
  modified_base 542: mod_base = OTHER; note = N1-methylpseudouracil
  modified_base 547: mod_base = OTHER; note = N1-methylpseudouracil
  modified_base 550: mod_base = OTHER; note = N1-methylpseudouracil
  modified_base 557: mod_base = OTHER; note = N1-methylpseudouracil
  modified_base 560: mod_base = OTHER; note = N1-methylpseudouracil
  modified_base 568: mod_base = OTHER; note = N1-methylpseudouracil
  modified_base 569: mod_base = OTHER; note = N1-methylpseudouracil
  modified_base 575: mod_base = OTHER; note = N1-methylpseudouracil
  modified_base 581: mod_base = OTHER; note = N1-methylpseudouracil
  modified_base 591: mod_base = OTHER; note = N1-methylpseudouracil
  modified_base 593: mod_base = OTHER; note = N1-methylpseudouracil
  modified_base 595: mod_base = OTHER; note = N1-methylpseudouracil
  modified_base 599: mod_base = OTHER; note = N1-methylpseudouracil
  modified_base 602: mod_base = OTHER; note = N1-methylpseudouracil
atggagcgggataccctcctgaaggctctgctggagatcgcaagctgcctggagaaagccctccaggtgttccaccaagtgttcatcttcatgacatactggcatctgctgaacgcctttaccgtgaccgtacccaaggacctgtacgtggtggagagagataccctgctgaaagccctgctggaaatcgcctcgtgcctggagaaggccctgcaggtgtttcaccaggtgttcattttcatgacctattggcacctgttgaacgcttttactgtgaccgtgccaaaggatttatacgtggtggagcgggacacactgctgaaggccctgcttgagatcgccagctgtctcgagaaagccctgcaagtgttccaccaggtattcatctttatgacttactggcacctgctcaacgccttcaccgtgacagtgcccaaggacttgtacgtggtcgagcgggacaccctgttaaaggccctgctggagattgccagctgtctggagaaggcactgcaggtgttccatcaggtgtttatctttatgacctactggcacctcctgaacgccttcacagtgaccgtgcccaaggatctctacgtggtg

SEQ ID NO: 7-49 - skipped (43 entries)

SEQ ID NO: 50 - RNA, 75 nt, synthetic construct
  source 1..75: mol_type = other RNA
ggaaatcgcaaaatttgctcttcgcgttagatttcttttagttttctcgcaactagcaagctttttgttctcgcc

SEQ ID NO: 51 - RNA, 203 nt, synthetic construct
  source 1..203: mol_type = other RNA
ggaaatccccacaaccgcctcatatccaggctcaagaatagagctcagtgttttgttgtttaatcattccgacgtgttttgcgatattcgcgcaaagcagccagtcgcgcgcttgcttttaagtagagttgtttttccacccgtttgccaggcatctttaatttaacatatttttatttttcaggctaacctacgccgccacc

SEQ ID NO: 52 - RNA, 76 nt, synthetic construct
  source 1..76: mol_type = other RNA
ggaaataagagagaaaagaagagtaagaagaaatataagatctccctgagcttcagggagccccggcgccgccacc

SEQ ID NO: 53 - RNA, 89 nt, synthetic construct
  source 1..89: mol_type = other RNA
ggaaaccccccacccccgtaagagagaaaagaagagtaagaagaaatataagatctccctgagcttcagggagccccggcgccgccacc

SEQ ID NO: 54 - RNA, 59 nt, synthetic construct
  source 1..59: mol_type = other RNA
ggagaacttccgcttccgttggcgcaagcgctttcattttttctgctaccgtgactaag

SEQ ID NO: 55 - RNA, 46 nt, synthetic construct
  source 1..46: mol_type = other RNA
ggaaataagagagaaaagaagagtaagaagaaatataagagccacc

SEQ ID NO: 56 - RNA, 56 nt, synthetic construct
  source 1..56: mol_type = other RNA
  modified_base 6: mod_base = OTHER; note = N1-methylpseudouracil
  modified_base 24: mod_base = OTHER; note = N1-methylpseudouracil
  modified_base 34: mod_base = OTHER; note = N1-methylpseudouracil
  modified_base 36: mod_base = OTHER; note = N1-methylpseudouracil
ggaaataagagagaaaagaagagtaagaagaaatataagaccccggcgccgccacc

SEQ ID NO: 57 - RNA, 81 nt, synthetic construct
  source 1..81: mol_type = other RNA
ggaaatcgcaaaatttgctcttcgcgttagatttcttttagttttctcgcaactagcaagctttttgttctcgccgccgcc

SEQ ID NO: 58 - RNA, 81 nt, synthetic construct
  source 1..81: mol_type = other RNA
ggaaatcgcaaaattttcttttcgcgttagatttcttttagttttctttcaactagcaagctttttgttctcgccgccgcc

SEQ ID NO: 59 - RNA, 83 nt, synthetic construct
  source 1..83: mol_type = other RNA
  misc_difference 14..18: note = This region may encompass 0-5 residues
  misc_difference 19: note = This region may be present or absent
  misc_difference 22: note = This region may be present or absent
  misc_difference 23..27: note = This region may encompass 0-5 residues
  misc_difference 81..83: note = This region may be present or absent
ggaaatcgcaaaatttttgctctttttcgcgttagatttcttttagttttctykcaactagcaagctttttgttctcgccrcc

SEQ ID NO: 60 - RNA, 79 nt, synthetic construct
  source 1..79: mol_type = other RNA
ggaaaattttagcctggaacgttagataactgtcctgttgtctttatatacttggtccccaagtagtttgtcttccaaa

SEQ ID NO: 61 - RNA, 79 nt, synthetic construct
  source 1..79: mol_type = other RNA
ggaaactttatttagtgttactttattttctgtttatttgtgtttcttcagtgggtttgttctaatttccttggccgcc

SEQ ID NO: 62 - RNA, 79 nt, synthetic construct
  source 1..79: mol_type = other RNA
ggaaaatctgtattaggttggcgtgttctttggtcggttgttagtattgttgttgattcgtttgtggtcggttgccgcc

SEQ ID NO: 63 - RNA, 79 nt, synthetic construct
  source 1..79: mol_type = other RNA
ggaaaattattaacatcttggtattctcgataaccattcgttggattttattgtattcgtagtttgggttcctgccgcc

SEQ ID NO: 64 - RNA, 79 nt, synthetic construct
  source 1..79: mol_type = other RNA
ggaaattattattatttctagctacaatttatcattgtattattttagctattcatcattatttacttggtgatcaaca

SEQ ID NO: 65 - RNA, 79 nt, synthetic construct
  source 1..79: mol_type = other RNA
ggaaataggttgttaaccaagttcaagcctaataagcttggattctggtgacttgcttcaccgttggcgggcaccgatc

SEQ ID NO: 66 - RNA, 74 nt, synthetic construct
  source 1..74: mol_type = other RNA
ggaaatcgtagagagtcgtacttagtacatatcgactatcggtggacaccatcaagattataaaccaggccaga

SEQ ID NO: 67 - RNA, 79 nt, synthetic construct
  source 1..79: mol_type = other RNA
ggaaacccgcccaagcgaccccaacatatcagcagttgcccaatcccaactcccaacacaatccccaagcaacgccgcc

SEQ ID NO: 68 - RNA, 79 nt, synthetic construct
  source 1..79: mol_type = other RNA
ggaaagcgattgaaggcgtcttttcaactactcgattaaggttgggtatcgtcgtgggacttggaaatttgttgtttcc

SEQ ID NO: 69 - RNA, 79 nt, synthetic construct
  source 1..79: mol_type = other RNA
ggaaactaatcgaaataaaagagccccgtactcttttatttctattaggttaggagccttagcatttgtatcttaggta

SEQ ID NO: 70 - RNA, 79 nt, synthetic construct
  source 1..79: mol_type = other RNA
ggaaatgtgatttccagcaacttcttttgaatatattgaattcctaattcaaagcgaacaaatctacaagccatatacc

SEQ ID NO: 71 - RNA, 74 nt, synthetic construct
  source 1..74: mol_type = other RNA
ggaaatcgtagagagtcgtacttacgtggtcgccattgcatagcgcgcgaaagcaacaggaacaagaacgcgcc

SEQ ID NO: 72 - RNA, 74 nt, synthetic construct
  source 1..74: mol_type = other RNA
ggaaatcgtagagagtcgtacttagaataaacagagtcgggtcgacttgtctctgatactacgacgtcacaatc

SEQ ID NO: 73 - RNA, 79 nt, synthetic construct
  source 1..79: mol_type = other RNA
ggaaaatttgccttcggagttgcgtatcctgaactgcccagcctcctgatatacaactgttccgcttattcgggccgcc

SEQ ID NO: 74 - RNA, 79 nt, synthetic construct
  source 1..79: mol_type = other RNA
ggaaatctgagcaggaatcctttgtgcattgaagactttagattcctctctgcggtagacgtgcacttataagtatttg

SEQ ID NO: 75 - RNA, 79 nt, synthetic construct
  source 1..79: mol_type = other RNA
ggaaagcgattgaaggcgtcttttcaactactcgattaaggttgggtatcgtcgtgggacttggaaatttgttgccacc

SEQ ID NO: 76 - RNA, 104 nt, synthetic construct
  source 1..104: mol_type = other RNA
ggaaatttttttttgatattataagagtttttttttgatattaagaaaatttttttttgatattagaagagtaagaagaaatataagaccccggcgccgccacc

SEQ ID NO: 77 - RNA, 57 nt, synthetic construct
  source 1..57: mol_type = other RNA
ggaaataagagagaaaagaagagtaagaagaaatataagagccaaaaaaaaaaaacc

SEQ ID NO: 78 - RNA, 76 nt, synthetic construct
  source 1..76: mol_type = other RNA
ggaaatctccctgagcttcagggagtaagagagaaaagaagagtaagaagaaatataagaccccggcgccgccacc

SEQ ID NO: 79 - RNA, 75 nt, synthetic construct; identical to SEQ ID NO: 50
  source 1..75: mol_type = other RNA

SEQ ID NO: 80 - RNA, 42 nt, synthetic construct
  source 1..42: mol_type = other RNA
gggagacaagcttggcattccggtactgttggtaaagccacc

SEQ ID NO: 81 - RNA, 47 nt, synthetic construct
  source 1..47: mol_type = other RNA
gggaattaacagagaaaagaagagtaagaagaaatataagagccacc

SEQ ID NO: 82 - RNA, 47 nt, synthetic construct
  source 1..47: mol_type = other RNA
gggaaattagacagaaaagaagagtaagaagaaatataagagccacc

SEQ ID NO: 83 - RNA, 47 nt, synthetic construct
  source 1..47: mol_type = other RNA
gggaaataagagagtaaagaacagtaagaagaaatataagagccacc

SEQ ID NO: 84 - RNA, 47 nt, synthetic construct
  source 1..47: mol_type = other RNA
gggaaaaaagagagaaaagaagactaagaagaaatataagagccacc

SEQ ID NO: 85 - RNA, 47 nt, synthetic construct
  source 1..47: mol_type = other RNA
gggaaataagagagaaaagaagagtaagaagatatataagagccacc

SEQ ID NO: 86 - RNA, 47 nt, synthetic construct
  source 1..47: mol_type = other RNA
gggaaataagagacaaaacaagagtaagaagaaatataagagccacc

SEQ ID NO: 87 - RNA, 47 nt, synthetic construct
  source 1..47: mol_type = other RNA
gggaaattagagagtaaagaacagtaagtagaattaaaagagccacc

SEQ ID NO: 88 - RNA, 47 nt, synthetic construct
  source 1..47: mol_type = other RNA
gggaaataagagagaatagaagagtaagaagaaatataagagccacc

SEQ ID NO: 89 - RNA, 47 nt, synthetic construct
  source 1..47: mol_type = other RNA
gggaaataagagagaaaagaagagtaagaagaaaattaagagccacc

SEQ ID NO: 90 - RNA, 47 nt, synthetic construct
  source 1..47: mol_type = other RNA
gggaaataagagagaaaagaagagtaagaagaaatttaagagccacc

SEQ ID NO: 91 - RNA, 92 nt, synthetic construct
  source 1..92: mol_type = other RNA
tcaagcttttggaccctcgtacagaagctaatacgactcactatagggaaataagagagaaaagaagagtaagaagaaatataagagccacc

SEQ ID NO: 92-99 - skipped (8 entries)

SEQ ID NO: 100 - RNA, 119 nt, synthetic construct
  source 1..119: mol_type = other RNA
taatagtaagctggagcctcctgagagacctgtgtgaactattgagaagatcggaacagctccttactctgaggaagttggtacccccgtggtctttgaataaagtctgagtgggcggc

SEQ ID NO: 101 - RNA, 119 nt, synthetic construct
  source 1..119: mol_type = other RNA
taatagtaagctggagcctcactctcctctccatcccgtatccaggctgtgaatttttcaaggaatataaagatcgggatgtacccccgtggtctttgaataaagtctgagtgggcggc

SEQ ID NO: 102 - RNA, 119 nt, synthetic construct
  source 1..119: mol_type = other RNA
tgatagtaagctggagcctctagtgacggcaacagggcttggtttttccttgttgtgaaatcgacatctctgaagacagggtacccccgtggtctttgaataaagtctgagtgggcggc

SEQ ID NO: 103 - RNA, 119 nt, synthetic construct
  source 1..119: mol_type = other RNA
tgatagtaagctggagcctccttccatctagtcacaaagactccttcgtccccagttgccgtctaggattgggcctcccagtacccccgtggtctttgaataaagtctgagtgggcggc

SEQ ID NO: 104 - RNA, 119 nt, synthetic construct
  source 1..119: mol_type = other RNA
tgatagtaagctggagcctcccataacatgacatatctggattttgtgcttagaaccttaaattggaagcattcttaattgtacccccgtggtctttgaataaagtctgagtgggcggc

SEQ ID NO: 105 - RNA, 119 nt, synthetic construct
  source 1..119: mol_type = other RNA
taatagtaagctggagcctccggaaaactaaaatagagatatttcaagattttataattttcaaagacctttgaaatattgtacccccgtggtctttgaataaagtctgagtgggcggc

SEQ ID NO: 106 - RNA, 119 nt, synthetic construct
  source 1..119: mol_type = other RNA
taatagtaagctggagcctctacacattgcttctagttggcagaaataattgattaaaagaccagaaactgtgataactggtacccccgtggtctttaaataaagtctaagtgggcggc

SEQ ID NO: 107 - RNA, 119 nt, synthetic construct
  source 1..119: mol_type = other RNA
tgataataggctggagcctcggtggccatgcttcttgccccttgggcctccccccagcccctcctccccttcctgcacccgtacccccgtggtctttgaataaagtctgagtgggcggc

SEQ ID NO: 108 - RNA, 119 nt, synthetic construct
  source 1..119: mol_type = other RNA
  modified_base 1: mod_base = OTHER; note = N1-methylpseudouracil
  modified_base 4: mod_base = OTHER; note = N1-methylpseudouracil
  modified_base 7: mod_base = OTHER; note = N1-methylpseudouracil
  modified_base 12: mod_base = OTHER; note = N1-methylpseudouracil
  modified_base 19: mod_base = OTHER; note = N1-methylpseudouracil
  modified_base 23: mod_base = OTHER; note = N1-methylpseudouracil
  modified_base 28: mod_base = OTHER; note = N1-methylpseudouracil
  modified_base 32: mod_base = OTHER; note = N1-methylpseudouracil
  modified_base 33: mod_base = OTHER; note = N1-methylpseudouracil
  modified_base 35: mod_base = OTHER; note = N1-methylpseudouracil
  modified_base 36: mod_base = OTHER; note = N1-methylpseudouracil
  modified_base 42: mod_base = OTHER; note = N1-methylpseudouracil
  modified_base 43: mod_base = OTHER; note = N1-methylpseudouracil
  modified_base 49: mod_base = OTHER; note = N1-methylpseudouracil
  modified_base 62: mod_base = OTHER; note = N1-methylpseudouracil
  modified_base 65: mod_base = OTHER; note = N1-methylpseudouracil
  modified_base 70: mod_base = OTHER; note = N1-methylpseudouracil
  modified_base 71: mod_base = OTHER; note = N1-methylpseudouracil
  modified_base 74: mod_base = OTHER; note = N1-methylpseudouracil
  modified_base 82: mod_base = OTHER; note = N1-methylpseudouracil
  modified_base 90: mod_base = OTHER; note = N1-methylpseudouracil
  modified_base 93: mod_base = OTHER; note = N1-methylpseudouracil
  modified_base 95: mod_base = OTHER; note = N1-methylpseudouracil
  modified_base 96: mod_base = OTHER; note = N1-methylpseudouracil
  modified_base 97: mod_base = OTHER; note = N1-methylpseudouracil
  modified_base 101: mod_base = OTHER; note = N1-methylpseudouracil
  modified_base 106: mod_base = OTHER; note = N1-methylpseudouracil
  modified_base 108: mod_base = OTHER; note = N1-methylpseudouracil
  modified_base 112: mod_base = OTHER; note = N1-methylpseudouracil
tgataataggctggagcctcggtggcctagcttcttgccccttgggcctccccccagcccctcctccccttcctgcacccgtacccccgtggtctttgaataaagtctgagtgggcggc

SEQ ID NO: 109 - RNA, 141 nt, synthetic construct
  source 1..141: mol_type = other RNA
tgataataggctggagcctcggtggccatgcttcttgccccttgggcctccccccagcccctcctccccttcctgcacccgtaccccccaaacaccattgtcacactccagtggtctttgaataaagtctgagtgggcggc

SEQ ID NO: 110 - RNA, 141 nt, synthetic construct
  source 1..141: mol_type = other RNA
tgataataggctggagcctcggtggcctagcttcttgccccttgggcctccccccagcccctcctccccttcctgcacccgtaccccccaaacaccattgtcacactccagtggtctttgaataaagtctgagtgggcggc

SEQ ID NO: 111 - RNA, 142 nt, synthetic construct
  source 1..142: mol_type = other RNA
tgataataggctggagcctcggtggcctagcttcttgccccttgggcctccccccagcccctcctccccttcctgcacccgtaccccctccataaagtaggaaacactacagtggtctttgaataaagtctgagtgggcggc

SEQ ID NO: 112 - RNA, 141 nt, synthetic construct
  source 1..141: mol_type = other RNA
tgataataggctggagcctcggtggcctagcttcttgccccttgggcctccccccagcccctcctccccttcctgcacccgtaccccccgcattattactcacggtacgagtggtctttgaataaagtctgagtgggcggc

SEQ ID NO: 113 - RNA, 164 nt, synthetic construct
  source 1..164: mol_type = other RNA
tgataatagtccataaagtaggaaacactacagctggagcctcggtggccatgcttcttgccccttgggcctccccccagcccctcctccccttcctgcacccgtaccccccgcattattactcacggtacgagtggtctttgaataaagtctgagtgggcggc

SEQ ID NO: 114 - RNA, 188 nt, synthetic construct
  source 1..188: mol_type = other RNA
tgataatagtccataaagtaggaaacactacagctggagcctcggtggcctagcttcttgccccttgggcctccataaagtaggaaacactacatccccccagcccctcctccccttcctgcacccgtaccccctccataaagtaggaaacactacagtggtctttgaataaagtctgagtgggcggc

SEQ ID NO: 115 - RNA, 60 nt, synthetic construct
  source 1..60: mol_type = other RNA
ctgagagacctgtgtgaactattgagaagatcggaacagctccttactctgaggaagttg

SEQ ID NO: 116 - RNA, 119 nt, synthetic construct
  source 1..119: mol_type = other RNA
tgataataggctggagcctctcacacacctctgccccttgggcctcccactcccatggctctgggcggtccagaaggagcgtacccccgtggtctttgaataaagtctgagtgggcggc

SEQ ID NO: 117 - RNA, 109 nt, synthetic construct
  source 1..109: mol_type = other RNA
tgataataggctggagcctccaccgcgttatccgttcctcgtaggctggtcctggggaacgggtcggcgggtacccccgtggtctttgaataaagtctgagtgggcggc

SEQ ID NO: 118 - RNA, 257 nt, synthetic construct
  source 1..257: mol_type = other RNA
tgataataggctggagcctctgcccggcaacggccaggtctgtgccaagtgtttgctgacgcaacccccactggctggggcttggtcatgggccatcagcgcgtgcgtggaaccttttcggctcctctgccgatccatactgcggaactcctagccgcttgttttgctcgcagcaggtctggagcaaacattatcgggactgataactctgttgtcctgtacccccgtggtctttgaataaagtctgagtgggcggc

SEQ ID NO: 119 - RNA, 219 nt, synthetic construct
  source 1..219: mol_type = other RNA
tgataataggctggagcctcggtggcctagcttcttgccccttgggcctccccccagcccctcctccccttcctgcacccgtaccctttttttttttttttttttcttcttttcttttttttcttttttttttttctttctttttttctttttttttcttttcttttttcttttttttttttttttccgtggtctttgaataaagtctgagtgggcggc

SEQ ID NO: 120 - RNA, 219 nt, synthetic construct
  source 1..219: mol_type = other RNA
tgataataggctggagcctcggtggcctagcttcttgccccttgggcctccccccagcccctcctccccttcctgcacccgtacccttttttttttttttttttttttttttttttttttttttttttttttttttttttttttttttttttttttttttttttttttttttttttttttttttttccgtggtctttgaataaagtctgagtgggcggc

SEQ ID NO: 121-132 - skipped (12 entries)

SEQ ID NO: 133 - RNA, 185 nt, synthetic construct
  source 1..185: mol_type = other RNA
taagtctccataaagtaggaaacactacagctggagcctcggtggcctagcttcttgccccttgggcctccataaagtaggaaacactacatccccccagcccctcctccccttcctgcacccgtaccccctccataaagtaggaaacactacagtggtctttgaataaagtctgagtgggcggc

SEQ ID NO: 134 - RNA, 139 nt, synthetic construct
  source 1..139: mol_type = other RNA
taaagcgctggagcctcggtggcctagcttcttgccccttgggcctccccccagcccctcctccccttcctgcacccgtaccccctccataaagtaggaaacactacagtggtctttgaataaagtctgagtgggcggc

SEQ ID NO: 135 - RNA, 194 nt, synthetic construct
  source 1..194: mol_type = other RNA
taaagctccccggggtccataaagtaggaaacactacagctggagcctcctgagagacctgtgtgaactattgagaagatcggaacagctccttactctgaggaagttgtccataaagtaggaaacactacagtaccccctccataaagtaggaaacactacagtggtctttgaataaagtctgagtgggcggc

SEQ ID NO: 136 - RNA, 241 nt, synthetic construct
  source 1..241: mol_type = other RNA
taatagtaaacctcactcacggccacattgagtgccaggctccgggctggtttatagtagtgtagagcattgcagcacttagactggggtgctgtagtctttattgtagtctttccacatacctgataattcttagataatttcttattttaattccataaagtaggaaacactacataaatctccataaagtaggaaacactacatattcttccataaagtaggaaacactacataggct

SEQ ID NO: 137 - RNA, 94 nt, synthetic construct
  source 1..94: mol_type = other RNA
gcctccaccgcgttatccgttcctcgtaggctggtcctggggaacgggtcggcgggtacccccgtggtctttgaataaagtctgagtgggcggc

SEQ ID NO: 138 - RNA, 254 nt, synthetic construct
  source 1..254: mol_type = other RNA
caccgcgttatccgttcctcgtaggctggtcctggggaacgggtcggcgggcctcggtggcctagcttcttgccccttgggcccaccgcgttatccgttcctcgtaggctggtcctggggaacgggtcggcggtccccccagcccctcctccccttcctgcacccgtaccccccaccgcgttatccgttcctcgtaggctggtcctggggaacgggtcggcgggtggtctttgaataaagtctgagtgggcggc

SEQ ID NO: 139 - RNA, 119 nt, synthetic construct
  source 1..119: mol_type = other RNA
taaagctccccgggggcctcggtggcctagcttcttgccccttgggcctccccccagcccctcctccccttcctgcacccgtacccccgtggtctttgaataaagtctgagtgggcggc

SEQ ID NO: 140 - RNA, 119 nt, synthetic construct
  source 1..119: mol_type = other RNA
taagtctccgctggagcctcggtggcctagcttcttgccccttgggcctccccccagcccctcctccccttcctgcacccgtacccccgtggtctttgaataaagtctgagtgggcggc

SEQ ID NO: 141 - RNA, 119 nt, synthetic construct
  source 1..119: mol_type = other RNA
taagcccctccgggggcctcggtggcctagcttcttgccccttgggcctccccccagcccctcctccccttcctgcacccgtacccccgtggtctttgaataaagtctgagtgggcggc

SEQ ID NO: 142 - skipped

SEQ ID NO: 143 - RNA, 22 nt, unidentified
  source 1..22: mol_type = unassigned RNA
tgtcagtttgtcaaatacccca

SEQ ID NO: 144-198 - skipped (55 entries)

SEQ ID NO: 199 - RNA, 164 nt, unidentified; identical to SEQ ID NO: 113
  source 1..164: mol_type = unassigned RNA

SEQ ID NO: 200 - RNA, 87 nt, unidentified
  source 1..87: mol_type = unassigned RNA
gacagtgcagtcacccataaagtagaaagcactactaacagcactggagggtgtagtgtttcctactttatggatgagtgtactgtg

SEQ ID NO: 201 - RNA, 23 nt, unidentified
  source 1..23: mol_type = unassigned RNA
tgtagtgtttcctactttatgga

SEQ ID NO: 202 - RNA, 23 nt, unidentified
  source 1..23: mol_type = unassigned RNA
tccataaagtaggaaacactaca

SEQ ID NO: 203 - RNA, 21 nt, unidentified
  source 1..21: mol_type = unassigned RNA
cataaagtagaaagcactact

SEQ ID NO: 204 - RNA, 21 nt, unidentified
  source 1..21: mol_type = unassigned RNA
agtagtgctttctactttatg

SEQ ID NO: 205 - RNA, 85 nt, unidentified
  source 1..85: mol_type = unassigned RNA
cgctggcgacgggacattattacttttggtacgcgctgtgacacttcaaactcgtaccgtgagtaataatgcgccgtccacggca

SEQ ID NO: 206 - RNA, 22 nt, unidentified
  source 1..22: mol_type = unassigned RNA
tcgtaccgtgagtaataatgcg

SEQ ID NO: 207 - RNA, 22 nt, unidentified
  source 1..22: mol_type = unassigned RNA
cgcattattactcacggtacga

SEQ ID NO: 208 - RNA, 21 nt, unidentified
  source 1..21: mol_type = unassigned RNA
cattattacttttggtacgcg

SEQ ID NO: 209 - DNA, 21 bp, synthetic construct
  source 1..21: mol_type = other DNA
  modified_base 1: mod_base = OTHER; note = 5'-phosphate nucleotide
  modified_base 21: mod_base = OTHER; note = inverted deoxythymidine
  misc_feature 1..20: note = RNA
  misc_feature 21: note = DNA
aaaaaaaaaaaaaaaaaaaat

SEQ ID NO: 210 - skipped

SEQ ID NO: 211 - DNA, 126 bp, synthetic construct
  source 1..126: mol_type = other DNA
  modified_base 126: mod_base = OTHER; note = inverted deoxythymidine
  misc_feature 1..125: note = RNA
  misc_feature 126: note = DNA
aaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaatctagaaaaaaaaaaaaaaaaaaaat

SEQ ID NO: 212-219 - skipped (8 entries)

SEQ ID NO: 220 - RNA, 133 nt, unidentified
  source 1..133: mol_type = unassigned RNA
gctggagcctcggtggccatgcttcttgccccttgggcctccccccagcccctcctccccttcctgcacccgtaccccctccataaagtaggaaacactacagtggtctttgaataaagtctgagtgggcggc

SEQ ID NO: 221 - RNA, 22 nt, unidentified
  source 1..22: mol_type = unassigned RNA
cctctgaaattcagttcttcag

SEQ ID NO: 222 - RNA, 22 nt, unidentified
  source 1..22: mol_type = unassigned RNA
tgagaactgaattccatgggtt

SEQ ID NO: 223 - RNA, 22 nt, unidentified
  source 1..22: mol_type = unassigned RNA
ctcctacatattagcattaaca

SEQ ID NO: 224 - RNA, 23 nt, unidentified
  source 1..23: mol_type = unassigned RNA
ttaatgctaatcgtgataggggt

SEQ ID NO: 225 - RNA, 22 nt, unidentified
  source 1..22: mol_type = unassigned RNA
ccagtattaactgtgctgctga

SEQ ID NO: 226 - RNA, 22 nt, unidentified
  source 1..22: mol_type = unassigned RNA
tagcagcacgtaaatattggcg

SEQ ID NO: 227 - RNA, 21 nt, unidentified
  source 1..21: mol_type = unassigned RNA
caacaccagtcgatgggctgt

SEQ ID NO: 228 - RNA, 22 nt, unidentified
  source 1..22: mol_type = unassigned RNA
tagcttatcagactgatgttga

SEQ ID NO: 229 - skipped

SEQ ID NO: 230 - RNA, 22 nt, unidentified
  source 1..22: mol_type = unassigned RNA
cgtgtatttgacaagctgagtt

SEQ ID NO: 231 - RNA, 22 nt, unidentified
  source 1..22: mol_type = unassigned RNA
tggctcagttcagcaggaacag

SEQ ID NO: 232 - RNA, 22 nt, unidentified
  source 1..22: mol_type = unassigned RNA
tgcctactgagctgatatcagt

SEQ ID NO: 233 - RNA, 21 nt, unidentified
  source 1..21: mol_type = unassigned RNA
ttcacagtggctaagttccgc

SEQ ID NO: 234 - RNA, 22 nt, unidentified
  source 1..22: mol_type = unassigned RNA
agggcttagctgcttgtgagca

SEQ ID NO: 235 - RNA, 141 nt, unidentified
  source 1..141: mol_type = unassigned RNA
tgataataggctggagcctcggtggccatgcttcttgccccttgggcctccccccagcccctcctccccttcctgcacccgtaccccccgcattattactcacggtacgagtggtctttgaataaagtctgagtgggcggc

SEQ ID NO: 236 - RNA, 119 nt, unidentified; identical to SEQ ID NO: 107
  source 1..119: mol_type = unassigned RNA

SEQ ID NO: 237 - RNA, 142 nt, unidentified
  source 1..142: mol_type = unassigned RNA
tgataataggctggagcctcggtggccatgcttcttgccccttgggcctccccccagcccctcctccccttcctgcacccgtaccccctccataaagtaggaaacactacagtggtctttgaataaagtctgagtgggcggc

SEQ ID NO: 238 - skipped

SEQ ID NO: 239 - RNA, 23 nt, unidentified
  source 1..23: mol_type = unassigned RNA
ttaatgctaattgtgataggggt

SEQ ID NO: 240 - RNA, 23 nt, unidentified
  source 1..23: mol_type = unassigned RNA
acccctatcacaattagcattaa

SEQ ID NO: 241 - RNA, 188 nt, unidentified
  source 1..188: mol_type = unassigned RNA
tgataatagtccataaagtaggaaacactacagctggagcctcggtggccatgcttcttgccccttgggcctccataaagtaggaaacactacatccccccagcccctcctccccttcctgcacccgtaccccctccataaagtaggaaacactacagtggtctttgaataaagtctgagtgggcggc

SEQ ID NO: 242 - RNA, 140 nt, unidentified
  source 1..140: mol_type = unassigned RNA
tgataataggctggagcctcggtggccatgcttcttgccccttgggcctccccccagcccctcctccccttcctgcacccgtacccccagtagtgctttctactttatggtggtctttgaataaagtctgagtgggcggc

SEQ ID NO: 243 - RNA, 182 nt, unidentified
  source 1..182: mol_type = unassigned RNA
tgataatagagtagtgctttctactttatggctggagcctcggtggccatgcttcttgccccttgggccagtagtgctttctactttatgtccccccagcccctcctccccttcctgcacccgtacccccagtagtgctttctactttatggtggtctttgaataaagtctgagtgggcggc

SEQ ID NO: 244 - RNA, 184 nt, unidentified
  source 1..184: mol_type = unassigned RNA
tgataatagagtagtgctttctactttatggctggagcctcggtggccatgcttcttgccccttgggcctccataaagtaggaaacactacatccccccagcccctcctccccttcctgcacccgtacccccagtagtgctttctactttatggtggtctttgaataaagtctgagtgggcggc

SEQ ID NO: 245 - RNA, 142 nt, unidentified
  source 1..142: mol_type = unassigned RNA
tgataataggctggagcctcggtggccatgcttcttgccccttgggcctccccccagcccctcctccccttcctgcacccgtacccccacccctatcacaattagcattaagtggtctttgaataaagtctgagtgggcggc

SEQ ID NO: 246 - RNA, 188 nt, unidentified
  source 1..188: mol_type = unassigned RNA
tgataatagacccctatcacaattagcattaagctggagcctcggtggccatgcttcttgccccttgggccacccctatcacaattagcattaatccccccagcccctcctccccttcctgcacccgtacccccacccctatcacaattagcattaagtggtctttgaataaagtctgagtgggcggc

SEQ ID NO: 247 - RNA, 188 nt, unidentified
  source 1..188: mol_type = unassigned RNA
tgataatagacccctatcacaattagcattaagctggagcctcggtggccatgcttcttgccccttgggcctccataaagtaggaaacactacatccccccagcccctcctccccttcctgcacccgtacccccacccctatcacaattagcattaagtggtctttgaataaagtctgagtgggcggc

SEQ ID NO: 248 - RNA, 142 nt, unidentified
  source 1..142: mol_type = unassigned RNA
tgataatagtccataaagtaggaaacactacagctggagcctcggtggccatgcttcttgccccttgggcctccccccagcccctcctccccttcctgcacccgtacccccgtggtctttgaataaagtctgagtgggcggc

SEQ ID NO: 249 - RNA, 142 nt, unidentified
  source 1..142: mol_type = unassigned RNA
tgataataggctggagcctcggtggctccataaagtaggaaacactacacatgcttcttgccccttgggcctccccccagcccctcctccccttcctgcacccgtacccccgtggtctttgaataaagtctgagtgggcggc

SEQ ID NO: 250 - RNA, 142 nt, unidentified
  source 1..142: mol_type = unassigned RNA
tgataataggctggagcctcggtggccatgcttcttgccccttgggcctccataaagtaggaaacactacatccccccagcccctcctccccttcctgcacccgtacccccgtggtctttgaataaagtctgagtgggcggc

SEQ ID NO: 251 - RNA, 70 nt, unidentified
  source 1..70: mol_type = unassigned RNA
gggaaataagagtccataaagtaggaaacactacaagaaaagaagagtaagaagaaatataagagccacc

SEQ ID NO: 252 - RNA, 70 nt, unidentified
  source 1..70: mol_type = unassigned RNA
gggaaataagagagaaaagaagagtaatccataaagtaggaaacactacagaagaaatataagagccacc

SEQ ID NO: 253 - RNA, 70 nt, unidentified
  source 1..70: mol_type = unassigned RNA
gggaaataagagagaaaagaagagtaagaagaaatataatccataaagtaggaaacactacagagccacc

SEQ ID NO: 254 - RNA, 23 nt, unidentified
  source 1..23: mol_type = unassigned RNA
acccctatcacaattagcattaa

SEQ ID NO: 255 - RNA, 181 nt, synthetic construct
  source 1..181: mol_type = other RNA
tgataatagagtagtgctttctactttatggctggagcctcggtggccatgcttcttgccccttgggccagtagtgctttctactttatgtccccccagcccctctccccttcctgcacccgtacccccagtagtgctttctactttatggtggtctttgaataaagtctgagtgggcggc

SEQ ID NO: 256 - RNA, 142 nt, synthetic construct
  source 1..142: mol_type = other RNA
tgataataggctggagcctcggtggccatgcttcttgccccttccataaagtaggaaacactacatgggcctccccccagcccctcctccccttcctgcacccgtacccccgtggtctttgaataaagtctgagtgggcggc

SEQ ID NO: 257 - RNA, 142 nt, synthetic construct
  source 1..142: mol_type = other RNA
tgataataggctggagcctcggtggccatgcttcttgccccttgggcctccccccagtccataaagtaggaaacactacacccctcctccccttcctgcacccgtacccccgtggtctttgaataaagtctgagtgggcggc

SEQ ID NO: 258 - RNA, 142 nt, synthetic construct
  source 1..142: mol_type = other RNA
tgataataggctggagcctcggtggccatgcttcttgccccttgggcctccccccagcccctcctccccttctccataaagtaggaaacactacactgcacccgtacccccgtggtctttgaataaagtctgagtgggcggc

SEQ ID NO: 259 - RNA, 142 nt, synthetic construct
  source 1..142: mol_type = other RNA
tgataataggctggagcctcggtggccatgcttcttgccccttgggcctccccccagcccctcctccccttcctgcacccgtacccccgtggtctttgaataaagttccataaagtaggaaacactacactgagtgggcggc

SEQ ID NO: 260 - RNA, 164 nt, synthetic construct
  source 1..164: mol_type = other RNA
tgataatagtccataaagtaggaaacactacagctggagcctcggtggcctagcttcttgccccttgggcctccccccagcccctcctccccttcctgcacccgtaccccccgcattattactcacggtacgagtggtctttgaataaagtctgagtgggcggc

SEQ ID NO: 261 - RNA, 119 nt, synthetic construct; identical to SEQ ID NO: 108
  source 1..119: mol_type = other RNA

SEQ ID NO: 262 - RNA, 141 nt, synthetic construct; identical to SEQ ID NO: 112
  source 1..141: mol_type = other RNA

SEQ ID NO: 263 - RNA, 188 nt, synthetic construct; identical to SEQ ID NO: 114
  source 1..188: mol_type = other RNA

SEQ ID NO: 264 - RNA, 142 nt, synthetic construct
  source 1..142: mol_type = other RNA
tgataatagtccataaagtaggaaacactacagctggagcctcggtggcctagcttcttgccccttgggcctccccccagcccctcctccccttcctgcacccgtacccccgtggtctttgaataaagtctgagtgggcggc

SEQ ID NO: 265 - RNA, 142 nt, synthetic construct
  source 1..142: mol_type = other RNA
tgataataggctggagcctcggtggctccataaagtaggaaacactacactagcttcttgccccttgggcctccccccagcccctcctccccttcctgcacccgtacccccgtggtctttgaataaagtctgagtgggcggc

SEQ ID NO: 266 - RNA, 142 nt, synthetic construct
  source 1..142: mol_type = other RNA
tgataataggctggagcctcggtggcctagcttcttgccccttgggcctccataaagtaggaaacactacatccccccagcccctcctccccttcctgcacccgtacccccgtggtctttgaataaagtctgagtgggcggc

SEQ ID NO: 267 - RNA, 142 nt, synthetic construct
  source 1..142: mol_type = other RNA
tgataataggctggagcctcggtggcctagcttcttgccccttgggcctccccccagcccctcctccccttcctgcacccgtacccccacccctatcacaattagcattaagtggtctttgaataaagtctgagtgggcggc

SEQ ID NO: 268 - RNA, 188 nt, synthetic construct
  source 1..188: mol_type = other RNA
tgataatagacccctatcacaattagcattaagctggagcctcggtggcctagcttcttgccccttgggccacccctatcacaattagcattaatccccccagcccctcctccccttcctgcacccgtacccccacccctatcacaattagcattaagtggtctttgaataaagtctgagtgggcggc

SEQ ID NO: 269 - RNA, 188 nt, synthetic construct
  source 1..188: mol_type = other RNA
tgataatagacccctatcacaattagcattaagctggagcctcggtggcctagcttcttgccccttgggcctccataaagtaggaaacactacatccccccagcccctcctccccttcctgcacccgtacccccacccctatcacaattagcattaagtggtctttgaataaagtctgagtgggcggc

SEQ ID NO: 270 - skipped

SEQ ID NO: 271 - RNA, 47 nt, synthetic construct
  source 1..47: mol_type = other RNA
aggaaataagagagaaaagaagagtaagaagaaatataagagccacc

SEQ ID NO: 272 - RNA, 57 nt, synthetic construct
  source 1..57: mol_type = other RNA
  modified_base 7: mod_base = OTHER; note = N1-methylpseudouracil
  modified_base 25: mod_base = OTHER; note = N1-methylpseudouracil
  modified_base 35: mod_base = OTHER; note = N1-methylpseudouracil
  modified_base 37: mod_base = OTHER; note = N1-methylpseudouracil
gggaaataagagagaaaagaagagtaagaagaaatataagaccccggcgccgccacc

SEQ ID NO: 273 - RNA, 57 nt, synthetic construct
  source 1..57: mol_type = other RNA
  modified_base 7: mod_base = OTHER; note = N1-methylpseudouracil
  modified_base 25: mod_base = OTHER; note = N1-methylpseudouracil
  modified_base 35: mod_base = OTHER; note = N1-methylpseudouracil
  modified_base 37: mod_base = OTHER; note = N1-methylpseudouracil
aggaaataagagagaaaagaagagtaagaagaaatataagaccccggcgccgccacc

SEQ ID NO: 274 - RNA, 47 nt, synthetic construct
  source 1..47: mol_type = other RNA
gggagatcagagagaaaagaagagtaagaagaaatataagagccacc

SEQ ID NO: 275-299 - skipped (25 entries)

SEQ ID NO: 300 - RNA, 778 nt, synthetic construct
  source 1..778: mol_type = other RNA
  modified_base 6: mod_base = OTHER; note = N1-methylpseudouracil
  modified_base 24: mod_base = OTHER; note = N1-methylpseudouracil
  modified_base 34: mod_base = OTHER; note = N1-methylpseudouracil
  modified_base 36: mod_base = OTHER; note = N1-methylpseudouracil
  modified_base 58: mod_base = OTHER; note = N1-methylpseudouracil
  modified_base 68: mod_base = OTHER; note = N1-methylpseudouracil
  modified_base 73: mod_base = OTHER; note = N1-methylpseudouracil
  modified_base 76: mod_base = OTHER; note = N1-methylpseudouracil
  modified_base 83: mod_base = OTHER; note = N1-methylpseudouracil
  modified_base 85: mod_base = OTHER; note = N1-methylpseudouracil
  modified_base 88: mod_base = OTHER; note = N1-methylpseudouracil
  modified_base 94: mod_base = OTHER; note = N1-methylpseudouracil
  modified_base 102: mod_base = OTHER; note = N1-methylpseudouracil
  modified_base 106: mod_base = OTHER; note = N1-methylpseudouracil
  modified_base 118: mod_base = OTHER; note = N1-methylpseudouracil
  modified_base 124: mod_base = OTHER; note = N1-methylpseudouracil
  modified_base 126: mod_base = OTHER; note = N1-methylpseudouracil
  modified_base 127: mod_base = OTHER; note = N1-methylpseudouracil
  modified_base 136: mod_base = OTHER; note = N1-methylpseudouracil
  modified_base 138: mod_base = OTHER; note = N1-methylpseudouracil
  modified_base 139: mod_base = OTHER; note = N1-methylpseudouracil
  modified_base 142: mod_base = OTHER; note = N1-methylpseudouracil
  modified_base 144: mod_base = OTHER; note = N1-methylpseudouracil
  modified_base 145: mod_base = OTHER; note = N1-methylpseudouracil
  modified_base 148: mod_base = OTHER; note = N1-methylpseudouracil
  modified_base 153: mod_base = OTHER; note = N1-methylpseudouracil
  modified_base 156: mod_base = OTHER; note = N1-methylpseudouracil
  modified_base 161: mod_base = OTHER; note = N1-methylpseudouracil
  modified_base 163: mod_base = OTHER; note = N1-methylpseudouracil
  modified_base 166: mod_base = OTHER; note = N1-methylpseudouracil
  modified_base 174: mod_base = OTHER; note = N1-methylpseudouracil
  modified_base 175: mod_base = OTHER; note = N1-methylpseudouracil
  modified_base 176: mod_base = OTHER; note = N1-methylpseudouracil
  modified_base 181: mod_base = OTHER; note = N1-methylpseudouracil
  modified_base 187: mod_base = OTHER; note = N1-methylpseudouracil
  modified_base 199: mod_base = OTHER; note = N1-methylpseudouracil
  modified_base 201: mod_base = OTHER; note = N1-methylpseudouracil
  modified_base 205: mod_base = OTHER; note = N1-methylpseudouracil
  modified_base 208: mod_base = OTHER; note = N1-methylpseudouracil
  modified_base 218: mod_base = OTHER; note = N1-methylpseudouracil
  modified_base 223: mod_base = OTHER; note = N1-methylpseudouracil
  modified_base 226: mod_base = OTHER; note = N1-methylpseudouracil
  modified_base 235: mod_base = OTHER; note = N1-methylpseudouracil
  modified_base 238: mod_base = OTHER; note = N1-methylpseudouracil
  modified_base 244: mod_base = OTHER; note = N1-methylpseudouracil
  modified_base 249: mod_base = OTHER; note = N1-methylpseudouracil
  modified_base 252: mod_base = OTHER; note = N1-methylpseudouracil
  modified_base 256: mod_base = OTHER; note = N1-methylpseudouracil
  modified_base 268: mod_base = OTHER; note = N1-methylpseudouracil
  modified_base 274: mod_base = OTHER; note = N1-methylpseudouracil
  modified_base 276: mod_base = OTHER; note = N1-methylpseudouracil
  modified_base 277: mod_base = OTHER; note = N1-methylpseudouracil
  modified_base 278: mod_base = OTHER; note = N1-methylpseudouracil
  modified_base 286: mod_base = OTHER; note = N1-methylpseudouracil
  modified_base 288: mod_base = OTHER; note = N1-methylpseudouracil
  modified_base 289: mod_base = OTHER; note = N1-methylpseudouracil
  modified_base 292: mod_base = OTHER; note = N1-methylpseudouracil
  modified_base 293: mod_base = OTHER; note = N1-methylpseudouracil
  modified_base 294: mod_base = OTHER; note = N1-methylpseudouracil
  modified_base 295: mod_base = OTHER; note = N1-methylpseudouracil
  modified_base 298: mod_base = OTHER; note = N1-methylpseudouracil
  modified_base 303: mod_base = OTHER; note = N1-methylpseudouracil
  modified_base 305: mod_base = OTHER; note = N1-methylpseudouracil
  modified_base 306: mod_base = OTHER; note = N1-methylpseudouracil
  modified_base 313: mod_base = OTHER; note = N1-methylpseudouracil
  modified_base 315: mod_base = OTHER; note = N1-methylpseudouracil
  modified_base 316: mod_base = OTHER; note = N1-methylpseudouracil
  modified_base 323: mod_base = OTHER; note = N1-methylpseudouracil
  modified_base 324: mod_base = OTHER; note = N1-methylpseudouracil
  modified_base 325: mod_base = OTHER; note = N1-methylpseudouracil
  modified_base 326: mod_base = OTHER; note = N1-methylpseudouracil
  modified_base 329: mod_base = OTHER; note = N1-methylpseudouracil
  modified_base 331: mod_base = OTHER; note = N1-methylpseudouracil
  modified_base 337: mod_base = OTHER; note = N1-methylpseudouracil
  modified_base 347: mod_base = OTHER; note = N1-methylpseudouracil
  modified_base 348: mod_base = OTHER; note = N1-methylpseudouracil
  modified_base 349: mod_base = OTHER; note = N1-methylpseudouracil
  modified_base 351: mod_base = OTHER; note = N1-methylpseudouracil
  modified_base 355: mod_base = OTHER; note = N1-methylpseudouracil
  modified_base 358: mod_base = OTHER; note = N1-methylpseudouracil
  modified_base 373: mod_base = OTHER; note = N1-methylpseudouracil
  modified_base 376: mod_base = OTHER; note = N1-methylpseudouracil
  modified_base 385: mod_base = OTHER; note = N1-methylpseudouracil
  modified_base 388: mod_base = OTHER; note = N1-methylpseudouracil
  modified_base 389: mod_base = OTHER; note = N1-methylpseudouracil
  modified_base 394: mod_base = OTHER; note = N1-methylpseudouracil
  modified_base 402: mod_base = OTHER; note = N1-methylpseudouracil
  modified_base 404: mod_base = OTHER; note = N1-methylpseudouracil
  modified_base 406: mod_base = OTHER; note = N1-methylpseudouracil
  modified_base 418: mod_base = OTHER; note = N1-methylpseudouracil
  modified_base 424: mod_base = OTHER; note = N1-methylpseudouracil
  modified_base 426: mod_base = OTHER; note = N1-methylpseudouracil
  modified_base 427: mod_base = OTHER; note = N1-methylpseudouracil
  modified_base 436: mod_base = OTHER; note = N1-methylpseudouracil
  modified_base 438: mod_base = OTHER; note = N1-methylpseudouracil
  modified_base 439: mod_base = OTHER; note = N1-methylpseudouracil
  modified_base 442: mod_base = OTHER; note = N1-methylpseudouracil
  modified_base 444: mod_base = OTHER; note = N1-methylpseudouracil
  modified_base 445: mod_base = OTHER; note = N1-methylpseudouracil
  modified_base 446: mod_base = OTHER; note = N1-methylpseudouracil
  modified_base 448: mod_base = OTHER; note = N1-methylpseudouracil
  modified_base 452: mod_base = OTHER; note = N1-methylpseudouracil
  modified_base 453: mod_base = OTHER; note = N1-methylpseudouracil
  modified_base 456: mod_base = OTHER; note = N1-methylpseudouracil
  modified_base 463: mod_base = OTHER; note = N1-methylpseudouracil
  modified_base 466: mod_base = OTHER; note = N1-methylpseudouracil
  modified_base 474: mod_base = OTHER; note = N1-methylpseudouracil
  modified_base 475: mod_base = OTHER; note = N1-methylpseudouracil
  modified_base 481: mod_base = OTHER; note = N1-methylpseudouracil
  modified_base 487: mod_base = OTHER; note = N1-methylpseudouracil
  modified_base 498: mod_base = OTHER; note = N1-methylpseudouracil
  modified_base 499: mod_base = OTHER; note = N1-methylpseudouracil
  modified_base 501: mod_base = OTHER; note = N1-methylpseudouracil
  modified_base 505: mod_base = OTHER; note = N1-methylpseudouracil
  modified_base 508: mod_base = OTHER; note = N1-methylpseudouracil
  modified_base 523: mod_base = OTHER; note = N1-methylpseudouracil
  modified_base 525: mod_base = OTHER; note = N1-methylpseudouracil
  modified_base 526: mod_base = OTHER; note = N1-methylpseudouracil
  modified_base 535: mod_base = OTHER; note = N1-methylpseudouracil
  modified_base 538: mod_base = OTHER; note = N1-methylpseudouracil
  modified_base 544: mod_base = OTHER; note = N1-methylpseudouracil
  modified_base 545: mod_base = OTHER; note = N1-methylpseudouracil
  modified_base 552: mod_base = OTHER; note = N1-methylpseudouracil
  modified_base 554: mod_base = OTHER; note = N1-methylpseudouracil
  modified_base 556: mod_base = OTHER; note = N1-methylpseudouracil
  modified_base 568: mod_base = OTHER; note = N1-methylpseudouracil
  modified_base 574: mod_base = OTHER; note = N1-methylpseudouracil
  modified_base 576: mod_base = OTHER; note = N1-methylpseudouracil
  modified_base 577: mod_base = OTHER; note = N1-methylpseudouracil
  modified_base 581: mod_base = OTHER; note = N1-methylpseudouracil
  modified_base 586: mod_base = OTHER; note = N1-methylpseudouracil
  modified_base 588: mod_base = OTHER; note = N1-methylpseudouracil
  modified_base 589: mod_base = OTHER; note = N1-methylpseudouracil
  modified_base 590: mod_base = OTHER; note = N1-methylpseudouracil
  modified_base 592: mod_base = OTHER; note = N1-methylpseudouracil
  modified_base 594: mod_base = OTHER; note = N1-methylpseudouracil
  modified_base 595: mod_base = OTHER; note = N1-methylpseudouracil
  modified_base 596: mod_base = OTHER; note = N1-methylpseudouracil
  modified_base 598: mod_base = OTHER; note = N1-methylpseudouracil
  modified_base 603: mod_base = OTHER; note = N1-methylpseudouracil
  modified_base 606: mod_base = OTHER; note = N1-methylpseudouracil
  modified_base 613: mod_base = OTHER; note = N1-methylpseudouracil
  modified_base 616: mod_base = OTHER; note = N1-methylpseudouracil
  modified_base 624: mod_base = OTHER; note = N1-methylpseudouracil
  modified_base 625: mod_base = OTHER; note = N1-methylpseudouracil
  modified_base 631: mod_base = OTHER; note = N1-methylpseudouracil
  modified_base 637: mod_base = OTHER; note = N1-methylpseudouracil
  modified_base 647: mod_base = OTHER; note = N1-methylpseudouracil
  modified_base 649: mod_base = OTHER; note = N1-methylpseudouracil
  modified_base 651: mod_base = OTHER; note = N1-methylpseudouracil
  modified_base 655: mod_base = OTHER; note = N1-methylpseudouracil
  modified_base 658: mod_base = OTHER; note = N1-methylpseudouracil
  modified_base 660: mod_base = OTHER; note = N1-methylpseudouracil
  modified_base 663: mod_base = OTHER; note = N1-methylpseudouracil
  modified_base 666: mod_base = OTHER; note = N1-methylpseudouracil
  modified_base 671: mod_base = OTHER; note = N1-methylpseudouracil
  modified_base 678: mod_base = OTHER; note = N1-methylpseudouracil
  modified_base 682: mod_base = OTHER; note = N1-methylpseudouracil
  modified_base 687: mod_base = OTHER; note = N1-methylpseudouracil
  modified_base 691: mod_base = OTHER; note = N1-methylpseudouracil
  modified_base 692: mod_base = OTHER; note = N1-methylpseudouracil
  modified_base 694: mod_base = OTHER; note = N1-methylpseudouracil
  modified_base 695: mod_base = OTHER; note = N1-methylpseudouracil
  modified_base 701: mod_base = OTHER; note = N1-methylpseudouracil
  modified_base 702: mod_base = OTHER; note = N1-methylpseudouracil
  modified_base 708: mod_base = OTHER; note = N1-methylpseudouracil
  modified_base 721: mod_base = OTHER; note = N1-methylpseudouracil
  modified_base 724: mod_base = OTHER; note = N1-methylpseudouracil
  modified_base 729: mod_base = OTHER; note = N1-methylpseudouracil
  modified_base 730: mod_base = OTHER; note = N1-methylpseudouracil
  modified_base 733: mod_base = OTHER; note = N1-methylpseudouracil
  modified_base 741: mod_base = OTHER; note = N1-methylpseudouracil
  modified_base 749: mod_base = OTHER; note = N1-methylpseudouracil
  modified_base 752: mod_base = OTHER; note = N1-methylpseudouracil
  modified_base 754: mod_base = OTHER; note = N1-methylpseudouracil
  modified_base 755: mod_base = OTHER; note = N1-methylpseudouracil
  modified_base 756: mod_base = OTHER; note = N1-methylpseudouracil
  modified_base 760: mod_base = OTHER; note = N1-methylpseudouracil
  modified_base 765: mod_base = OTHER; note = N1-methylpseudouracil
  modified_base 767: mod_base = OTHER; note = N1-methylpseudouracil
  modified_base 771: mod_base = OTHER; note = N1-methylpseudouracil
ggaaataagagagaaaagaagagtaagaagaaatataagaccccggcgccgccaccatggagcgggataccctcctgaaggctctgctggagatcgcaagctgcctggagaaagccctccaggtgttccaccaagtgttcatcttcatgacatactggcatctgctgaacgcctttaccgtgaccgtacccaaggacctgtacgtggtggagagagataccctgctgaaagccctgctggaaatcgcctcgtgcctggagaaggccctgcaggtgtttcaccaggtgttcattttcatgacctattggcacctgttgaacgcttttactgtgaccgtgccaaaggatttatacgtggtggagcgggacacactgctgaaggccctgcttgagatcgccagctgtctcgagaaagccctgcaagtgttccaccaggtattcatctttatgacttactggcacctgctcaacgccttcaccgtgacagtgcccaaggacttgtacgtggtcgagcgggacaccctgttaaaggccctgctggagattgccagctgtctggagaaggcactgcaggtgttccatcaggtgtttatctttatgacctactggcacctcctgaacgccttcacagtgaccgtgcccaaggatctctacgtggtgtgataataggctggagcctcggtggcctagcttcttgccccttgggcctccccccagcccctcctccccttcctgcacccgtacccccgtggtctttgaataaagtctgagtgggcggc

SEQ ID NO: 301 - RNA, 779 nt, synthetic construct
  source 1..779: mol_type = other RNA
  modified_base 7: mod_base = OTHER; note = N1-methylpseudouracil
  modified_base 25: mod_base = OTHER; note = N1-methylpseudouracil
  modified_base 35: mod_base = OTHER; note = N1-methylpseudouracil
  modified_base 37: mod_base = OTHER; note = N1-methylpseudouracil
  modified_base 59: mod_base = OTHER; note = N1-methylpseudouracil
  modified_base 69: mod_base = OTHER; note = N1-methylpseudouracil
  modified_base 74: mod_base = OTHER; note = N1-methylpseudouracil
  modified_base 77: mod_base = OTHER; note = N1-methylpseudouracil
  modified_base 84: mod_base = OTHER; note = N1-methylpseudouracil
  modified_base 86: mod_base = OTHER; note = N1-methylpseudouracil
  modified_base 89: mod_base = OTHER; note = N1-methylpseudouracil
  modified_base 95: mod_base = OTHER; note = N1-methylpseudouracil
  modified_base 103: mod_base = OTHER; note = N1-methylpseudouracil
  modified_base 107: mod_base = OTHER; note = N1-methylpseudouracil
  modified_base 119: mod_base = OTHER; note = N1-methylpseudouracil
  modified_base 125: mod_base = OTHER; note = N1-methylpseudouracil
  modified_base 127: mod_base = OTHER; note = N1-methylpseudouracil
  modified_base 128: mod_base = OTHER; note = N1-methylpseudouracil
  modified_base 137: mod_base = OTHER; note = N1-methylpseudouracil
  modified_base 139: mod_base = OTHER; note = N1-methylpseudouracil
  modified_base 140: mod_base = OTHER; note = N1-methylpseudouracil
  modified_base 143: mod_base = OTHER; note = N1-methylpseudouracil
  modified_base 145: mod_base = OTHER; note = N1-methylpseudouracil
  modified_base 146: mod_base = OTHER; note = N1-methylpseudouracil
  modified_base 149: mod_base = OTHER; note = N1-methylpseudouracil
  modified_base 154: mod_base = OTHER; note = N1-methylpseudouracil
  modified_base 157: mod_base = OTHER; note = N1-methylpseudouracil
  modified_base 162: mod_base = OTHER; note = N1-methylpseudouracil
  modified_base 164: mod_base = OTHER; note = N1-methylpseudouracil
  modified_base 167: mod_base = OTHER; note = N1-methylpseudouracil
  modified_base 175: mod_base = OTHER; note = N1-methylpseudouracil
  modified_base 176: mod_base = OTHER; note = N1-methylpseudouracil
  modified_base 177: mod_base = OTHER; note = N1-methylpseudouracil
  modified_base 182: mod_base = OTHER; note = N1-methylpseudouracil
  modified_base 188: mod_base = OTHER; note = N1-methylpseudouracil
  modified_base 200: mod_base = OTHER; note = N1-methylpseudouracil
  modified_base 202: mod_base = OTHER; note = N1-methylpseudouracil
  modified_base 206: mod_base = OTHER; note = N1-methylpseudouracil
  modified_base 209: mod_base = OTHER; note = N1-methylpseudouracil
  modified_base 219: mod_base = OTHER; note = N1-methylpseudouracil
  modified_base 224: mod_base = OTHER; note = N1-methylpseudouracil
  modified_base 227: mod_base = OTHER; note = N1-methylpseudouracil
  modified_base 236: mod_base = OTHER; note = N1-methylpseudouracil
  modified_base 239: mod_base = OTHER; note = N1-methylpseudouracil
  modified_base 245: mod_base = OTHER; note = N1-methylpseudouracil
  modified_base 250: mod_base = OTHER; note = N1-methylpseudouracil
  modified_base 253: mod_base = OTHER; note = N1-methylpseudouracil
  modified_base 257: mod_base = OTHER; note = N1-methylpseudouracil
  modified_base 269: mod_base = OTHER; note = N1-methylpseudouracil
  modified_base 275: mod_base = OTHER; note = N1-methylpseudouracil
  modified_base 277: mod_base = OTHER; note = N1-methylpseudouracil
  modified_base 278: mod_base = OTHER; note = N1-methylpseudouracil
  modified_base 279: mod_base = OTHER; note = N1-methylpseudouracil
  modified_base 287: mod_base = OTHER; note = N1-methylpseudouracil
  modified_base 289: mod_base = OTHER; note = N1-methylpseudouracil
  modified_base 290: mod_base = OTHER; note = N1-methylpseudouracil
  modified_base 293: mod_base = OTHER; note = N1-methylpseudouracil
  modified_base 294: mod_base = OTHER; note = N1-methylpseudouracil
  modified_base 295: mod_base = OTHER; note = N1-methylpseudouracil
  modified_base 296: mod_base = OTHER; note = N1-methylpseudouracil
  modified_base 299: mod_base = OTHER; note = N1-methylpseudouracil
  modified_base 304: mod_base = OTHER; note = N1-methylpseudouracil
  modified_base 306: mod_base = OTHER; note = N1-methylpseudouracil
  modified_base 307: mod_base = OTHER; note = N1-methylpseudouracil
  modified_base 314: mod_base = OTHER; note = N1-methylpseudouracil
  modified_base 316: mod_base = OTHER; note = N1-methylpseudouracil
  modified_base 317: mod_base = OTHER; note = N1-methylpseudouracil
  modified_base 324: mod_base = OTHER; note = N1-methylpseudouracil
  modified_base 325: mod_base = OTHER; note = N1-methylpseudouracil
  modified_base 326: mod_base = OTHER; note = N1-methylpseudouracil
  modified_base 327: mod_base = OTHER; note = N1-methylpseudouracil
  modified_base 330: mod_base = OTHER; note = N1-methylpseudouracil
  modified_base 332: mod_base = OTHER; note = N1-methylpseudouracil
  modified_base 338: mod_base = OTHER; note = N1-methylpseudouracil
  modified_base 348: mod_base = OTHER; note = N1-methylpseudouracil
  modified_base 349: mod_base = OTHER; note = N1-methylpseudouracil
  modified_base 350: mod_base = OTHER; note = N1-methylpseudouracil
  modified_base 352: mod_base = OTHER; note = N1-methylpseudouracil
  modified_base 356: mod_base = OTHER; note = N1-methylpseudouracil
  modified_base 359: mod_base = OTHER; note = N1-methylpseudouracil
  modified_base 374: mod_base = OTHER; note = N1-methylpseudouracil
  modified_base 377: mod_base = OTHER; note = N1-methylpseudouracil
  modified_base 386: mod_base = OTHER; note = N1-methylpseudouracil
  modified_base 389: mod_base = OTHER; note = N1-methylpseudouracil
  modified_base 390: mod_base = OTHER; note = N1-methylpseudouracil
  modified_base 395: mod_base = OTHER; note = N1-methylpseudouracil
  modified_base 403: mod_base = OTHER; note = N1-methylpseudouracil
  modified_base 405: mod_base = OTHER; note = N1-methylpseudouracil
  modified_base 407: mod_base = OTHER; note = N1-methylpseudouracil
  modified_base 419: mod_base = OTHER; note = N1-methylpseudouracil
  modified_base 425: mod_base = OTHER; note = N1-methylpseudouracil
  modified_base 427: mod_base = OTHER; note = N1-methylpseudouracil
  modified_base 428: mod_base = OTHER; note = N1-methylpseudouracil
  modified_base 437: mod_base = OTHER; note = N1-methylpseudouracil
  modified_base 439: mod_base = OTHER; note = N1-methylpseudouracil
  modified_base 440: mod_base = OTHER; note = N1-methylpseudouracil
  modified_base 443: mod_base = OTHER; note = N1-methylpseudouracil
  modified_base 445: mod_base = OTHER; note = N1-methylpseudouracil
  modified_base 446: mod_base = OTHER; note = N1-methylpseudouracil
  modified_base 447: mod_base = OTHER; note = N1-methylpseudouracil
  modified_base 449: mod_base = OTHER; note = N1-methylpseudouracil
  modified_base 453: mod_base = OTHER; note = N1-methylpseudouracil
  modified_base 454: mod_base = OTHER; note = N1-methylpseudouracil
  modified_base 457: mod_base = OTHER; note = N1-methylpseudouracil
  modified_base 464: mod_base = OTHER; note = N1-methylpseudouracil
  modified_base 467: mod_base = OTHER; note = N1-methylpseudouracil
  modified_base 475: mod_base = OTHER; note = N1-methylpseudouracil
  modified_base 476: mod_base = OTHER; note = N1-methylpseudouracil
  modified_base 482: mod_base = OTHER; note = N1-methylpseudouracil
  modified_base 488: mod_base = OTHER; note = N1-methylpseudouracil
  modified_base 499: mod_base = OTHER; note = N1-methylpseudouracil
  modified_base 500: mod_base = OTHER; note = N1-methylpseudouracil
  modified_base 502: mod_base = OTHER; note = N1-methylpseudouracil
  modified_base 506: mod_base = OTHER; note = N1-methylpseudouracil
  modified_base 509: mod_base = OTHER; note = N1-methylpseudouracil
  modified_base 524: mod_base = OTHER; note = N1-methylpseudouracil
  modified_base 526: mod_base = OTHER; note = N1-methylpseudouracil
  modified_base 527: mod_base = OTHER; note = N1-methylpseudouracil
  modified_base 536: mod_base = OTHER; note = N1-methylpseudouracil
  modified_base 539: mod_base = OTHER; note = N1-methylpseudouracil
  modified_base 545: mod_base = OTHER; note = N1-methylpseudouracil
  modified_base 546: mod_base = OTHER; note = N1-methylpseudouracil
  modified_base 553: mod_base = OTHER; note = N1-methylpseudouracil
  modified_base 555: mod_base = OTHER; note = N1-methylpseudouracil
  modified_base 557: mod_base = OTHER; note = N1-methylpseudouracil
  modified_base 569: mod_base = OTHER; note = N1-methylpseudouracil
  modified_base 575: mod_base = OTHER; note = N1-methylpseudouracil
  modified_base 577: mod_base = OTHER; note = N1-methylpseudouracil
  modified_base 578: mod_base = OTHER; note = N1-methylpseudouracil
  modified_base 582: mod_base = OTHER; note = N1-methylpseudouracil
  modified_base 587: mod_base = OTHER; note = N1-methylpseudouracil
  modified_base 589: mod_base = OTHER; note = N1-methylpseudouracil
  modified_base 590: mod_base = OTHER; note = N1-methylpseudouracil
  modified_base 591: mod_base = OTHER; note = N1-methylpseudouracil
  modified_base 593: mod_base = OTHER; note = N1-methylpseudouracil
  modified_base 595: mod_base = OTHER; note = N1-methylpseudouracil
  modified_base 596: mod_base = OTHER; note = N1-methylpseudouracil
  modified_base 597: mod_base = OTHER; note = N1-methylpseudouracil
  modified_base 599: mod_base = OTHER; note = N1-methylpseudouracil
  modified_base 604: mod_base = OTHER; note = N1-methylpseudouracil
  modified_base 607: mod_base = OTHER; note = N1-methylpseudouracil
  modified_base 614: mod_base = OTHER; note = N1-methylpseudouracil
  modified_base 617: mod_base = OTHER; note = N1-methylpseudouracil
  modified_base 625: mod_base = OTHER; note = N1-methylpseudouracil
  modified_base 626: mod_base = OTHER; note = N1-methylpseudouracil
  modified_base 632: mod_base = OTHER; note = N1-methylpseudouracil
  modified_base 638: mod_base = OTHER; note = N1-methylpseudouracil
  modified_base 648: mod_base = OTHER; note = N1-methylpseudouracil
  modified_base 650: mod_base = OTHER; note = N1-methylpseudouracil
  modified_base 652: mod_base = OTHER; note = N1-methylpseudouracil
  modified_base 656: mod_base = OTHER; note = N1-methylpseudouracil
  modified_base 659: mod_base = OTHER; note = N1-methylpseudouracil
  modified_base 661: mod_base = OTHER; note = N1-methylpseudouracil
  modified_base 664: mod_base = OTHER; note = N1-methylpseudouracil
  modified_base 667: mod_base = OTHER; note = N1-methylpseudouracil
  modified_base 672: mod_base = OTHER; note = N1-methylpseudouracil
  modified_base 679: mod_base = OTHER; note = N1-methylpseudouracil
  modified_base 683: mod_base = OTHER; note = N1-methylpseudouracil
  modified_base 688: mod_base = OTHER; note = N1-methylpseudouracil
  modified_base 692: mod_base = OTHER; note = N1-methylpseudouracil
  modified_base 693: mod_base = OTHER; note = N1-methylpseudouracil
  modified_base 695: mod_base = OTHER; note = N1-methylpseudouracil
  modified_base 696: mod_base = OTHER; note = N1-methylpseudouracil
  modified_base 702: mod_base = OTHER; note = N1-methylpseudouracil
  modified_base 703: mod_base = OTHER; note = N1-methylpseudouracil
  modified_base 709: mod_base = OTHER; note = N1-methylpseudouracil
  modified_base 722: mod_base = OTHER; note = N1-methylpseudouracil
  modified_base 725: mod_base = OTHER; note = N1-methylpseudouracil
  modified_base 730: mod_base = OTHER; note = N1-methylpseudouracil
  modified_base 731: mod_base = OTHER; note = N1-methylpseudouracil
  modified_base 734: mod_base = OTHER; note = N1-methylpseudouracil
  modified_base 742: mod_base = OTHER; note = N1-methylpseudouracil
  modified_base 750: mod_base = OTHER; note = N1-methylpseudouracil
  modified_base 753: mod_base = OTHER; note = N1-methylpseudouracil
  modified_base 755: mod_base = OTHER; note = N1-methylpseudouracil
  modified_base 756: mod_base = OTHER; note = N1-methylpseudouracil
  modified_base 757: mod_base = OTHER; note = N1-methylpseudouracil
  modified_base 761: mod_base = OTHER; note = N1-methylpseudouracil
  modified_base 766: mod_base = OTHER; note = N1-methylpseudouracil
  modified_base 768: mod_base = OTHER; note = N1-methylpseudouracil
  modified_base 772: mod_base = OTHER; note = N1-methylpseudouracil
gggaaataagagagaaaagaagagtaagaagaaatataagaccccggcgccgccaccatggagcgggataccctcctgaaggctctgctggagatcgcaagctgcctggagaaagccctccaggtgttccaccaagtgttcatcttcatgacatactggcatctgctgaacgcctttaccgtgaccgtacccaaggacctgtacgtggtggagagagataccctgctgaaagccctgctggaaatcgcctcgtgcctggagaaggccctgcaggtgtttcaccaggtgttcattttcatgacctattggcacctgttgaacgcttttactgtgaccgtgccaaaggatttatacgtggtggagcgggacacactgctgaaggccctgcttgagatcgccagctgtctcgagaaagccctgcaagtgttccaccaggtattcatctttatgacttactggcacctgctcaacgccttcaccgtgacagtgcccaaggacttgtacgtggtcgagcgggacaccctgttaaaggccctgctggagattgccagctgtctggagaaggcactgcaggtgttccatcaggtgtttatctttatgacctactggcacctcctgaacgccttcacagtgaccgtgcccaaggatctctacgtggtgtgataataggctggagcctcggtggcctagcttcttgccccttgggcctccccccagcccctcctccccttcctgcacccgtacccccgtggtctttgaataaagtctgagtgggcggc

SEQ ID NO: 302 - RNA, 779 nt, synthetic construct
  source 1..779: mol_type = other RNA
  modified_base 7: mod_base = OTHER; note = N1-methylpseudouracil
  modified_base 25: mod_base = OTHER; note = N1-methylpseudouracil
  modified_base 35: mod_base = OTHER; note = N1-methylpseudouracil
  modified_base 37: mod_base = OTHER; note = N1-methylpseudouracil
  modified_base 59: mod_base = OTHER; note = N1-methylpseudouracil
  modified_base 69: mod_base = OTHER; note = N1-methylpseudouracil
  modified_base 74: mod_base = OTHER; note = N1-methylpseudouracil
  modified_base 77: mod_base = OTHER; note = N1-methylpseudouracil
  modified_base 84: mod_base = OTHER; note = N1-methylpseudouracil
  modified_base 86: mod_base = OTHER; note = N1-methylpseudouracil
  modified_base 89: mod_base = OTHER; note = N1-methylpseudouracil
  modified_base 95: mod_base = OTHER; note = N1-methylpseudouracil
  modified_base 103: mod_base = OTHER; note = N1-methylpseudouracil
  modified_base 107: mod_base = OTHER; note = N1-methylpseudouracil
  modified_base 119: mod_base = OTHER; note = N1-methylpseudouracil
  modified_base 125: mod_base = OTHER; note = N1-methylpseudouracil
  modified_base 127: mod_base = OTHER; note = N1-methylpseudouracil
  modified_base 128: mod_base = OTHER; note = N1-methylpseudouracil
  modified_base 137: mod_base = OTHER; note = N1-methylpseudouracil
  modified_base 139: mod_base = OTHER; note = N1-methylpseudouracil
  modified_base 140: mod_base = OTHER; note = N1-methylpseudouracil
  modified_base 143: mod_base = OTHER; note = N1-methylpseudouracil
  modified_base 145: mod_base = OTHER; note = N1-methylpseudouracil
  modified_base 146: mod_base = OTHER; note = N1-methylpseudouracil
  modified_base 149: mod_base = OTHER; note = N1-methylpseudouracil
  modified_base 154: mod_base = OTHER; note = N1-methylpseudouracil
  modified_base 157: mod_base = OTHER; note = N1-methylpseudouracil
  modified_base 162: mod_base = OTHER; note = N1-methylpseudouracil
  modified_base 164: mod_base = OTHER; note = N1-methylpseudouracil
  modified_base 167: mod_base = OTHER; note = N1-methylpseudouracil
  modified_base 175: mod_base = OTHER; note = N1-methylpseudouracil
  modified_base 176: mod_base = OTHER; note = N1-methylpseudouracil
  modified_base 177: mod_base = OTHER; note = N1-methylpseudouracil
  modified_base 182: mod_base = OTHER; note = N1-methylpseudouracil
  modified_base 188: mod_base = OTHER; note = N1-methylpseudouracil
  modified_base 200: mod_base = OTHER; note = N1-methylpseudouracil
  modified_base 202: mod_base = OTHER; note = N1-methylpseudouracil
  modified_base 206: mod_base = OTHER; note = N1-methylpseudouracil
  modified_base 209: mod_base = OTHER; note = N1-methylpseudouracil
  modified_base 219: mod_base = OTHER; note = N1-methylpseudouracil
  modified_base 224: mod_base = OTHER; note = N1-methylpseudouracil
  modified_base 227: mod_base = OTHER; note = N1-methylpseudouracil
  modified_base 236: mod_base = OTHER; note = N1-methylpseudouracil
  modified_base 239: mod_base = OTHER; note = N1-methylpseudouracil
  modified_base 245: mod_base = OTHER; note = N1-methylpseudouracil
  modified_base 250: mod_base = OTHER; note = N1-methylpseudouracil
  modified_base 253: mod_base = OTHER; note = N1-methylpseudouracil
  modified_base 257: mod_base = OTHER; note = N1-methylpseudouracil
  modified_base 269: mod_base = OTHER; note = N1-methylpseudouracil
  modified_base 275: mod_base = OTHER; note = N1-methylpseudouracil
  modified_base 277: mod_base = OTHER; note = N1-methylpseudouracil
  modified_base 278: mod_base = OTHER; note = N1-methylpseudouracil
  modified_base 279: mod_base = OTHER; note = N1-methylpseudouracil
  modified_base 287: mod_base = OTHER; note = N1-methylpseudouracil
  modified_base 289: mod_base = OTHER; note = N1-methylpseudouracil
  modified_base 290: mod_base = OTHER; note = N1-methylpseudouracil
  modified_base 293: mod_base = OTHER; note = N1-methylpseudouracil
  modified_base 294: mod_base = OTHER; note = N1-methylpseudouracil
  modified_base 295: mod_base = OTHER; note = N1-methylpseudouracil
  modified_base 296: mod_base = OTHER; note = N1-methylpseudouracil
  modified_base 299: mod_base = OTHER; note = N1-methylpseudouracil
  modified_base 304: mod_base = OTHER; note = N1-methylpseudouracil
  modified_base 306: mod_base = OTHER; note = N1-methylpseudouracil
  modified_base 307: mod_base = OTHER; note = N1-methylpseudouracil
  modified_base 314: mod_base = OTHER; note = N1-methylpseudouracil
  modified_base 316: mod_base = OTHER; note = N1-methylpseudouracil
  modified_base 317: mod_base = OTHER; note = N1-methylpseudouracil
  modified_base 324: mod_base = OTHER; note = N1-methylpseudouracil
  modified_base 325: mod_base = OTHER; note = N1-methylpseudouracil
  modified_base 326: mod_base = OTHER; note = N1-methylpseudouracil
  modified_base 327: mod_base = OTHER; note = N1-methylpseudouracil
  modified_base 330: mod_base = OTHER; note = N1-methylpseudouracil
  modified_base 332: mod_base = OTHER; note = N1-methylpseudouracil
  modified_base 338: mod_base = OTHER; note = N1-methylpseudouracil
  modified_base 348: mod_base = OTHER; note = N1-methylpseudouracil
  modified_base 349: mod_base = OTHER; note = N1-methylpseudouracil
  modified_base 350: mod_base = OTHER; note = N1-methylpseudouracil
  modified_base 352: mod_base = OTHER; note = N1-methylpseudouracil
  modified_base 356: mod_base = OTHER; note = N1-methylpseudouracil
  modified_base 359: mod_base = OTHER; note = N1-methylpseudouracil
  modified_base 374: mod_base = OTHER; note = N1-methylpseudouracil
  modified_base 377: mod_base = OTHER; note = N1-methylpseudouracil
  modified_base 386: mod_base = OTHER; note = N1-methylpseudouracil
  modified_base 389: mod_base = OTHER; note = N1-methylpseudouracil
  modified_base 390: mod_base = OTHER; note = N1-methylpseudouracil
  modified_base 395: mod_base = OTHER; note = N1-methylpseudouracil
  modified_base 403: mod_base = OTHER; note = N1-methylpseudouracil
  modified_base 405: mod_base = OTHER; note = N1-methylpseudouracil
  modified_base 407: mod_base = OTHER; note = N1-methylpseudouracil
  modified_base 419: mod_base = OTHER; note = N1-methylpseudouracil
  modified_base 425: mod_base = OTHER; note = N1-methylpseudouracil
  modified_base 427: mod_base = OTHER; note = N1-methylpseudouracil
  modified_base 428: mod_base = OTHER; note = N1-methylpseudouracil
  modified_base 437: mod_base = OTHER; note = N1-methylpseudouracil
  modified_base 439: mod_base = OTHER; note = N1-methylpseudouracil
  modified_base 440: mod_base = OTHER; note = N1-methylpseudouracil
  modified_base 443: mod_base = OTHER; note = N1-methylpseudouracil
  modified_base 445: mod_base = OTHER; note = N1-methylpseudouracil
  modified_base 446: mod_base = OTHER; note = N1-methylpseudouracil
  modified_base 447: mod_base = OTHER; note = N1-methylpseudouracil
  modified_base 449: mod_base = OTHER; note = N1-methylpseudouracil
  modified_base 453: mod_base = OTHER; note = N1-methylpseudouracil
  modified_base 454: mod_base = OTHER; note = N1-methylpseudouracil
  modified_base 457: mod_base = OTHER; note = N1-methylpseudouracil
  modified_base 464: mod_base = OTHER; note = N1-methylpseudouracil
  modified_base 467: mod_base = OTHER; note = N1-methylpseudouracil
  modified_base 475: mod_base = OTHER; note = N1-methylpseudouracil
  modified_base 476: mod_base = OTHER; note = N1-methylpseudouracil
  modified_base 482: mod_base = OTHER; note = N1-methylpseudouracil
  modified_base 488: mod_base = OTHER; note = N1-methylpseudouracil
  modified_base 499: mod_base = OTHER; note = N1-methylpseudouracil
  modified_base 500: mod_base = OTHER; note = N1-methylpseudouracil
  modified_base 502: mod_base = OTHER; note = N1-methylpseudouracil
  modified_base 506: mod_base = OTHER; note = N1-methylpseudouracil
  modified_base 509: mod_base = OTHER; note = N1-methylpseudouracil
  modified_base 524: mod_base = OTHER; note = N1-methylpseudouracil
  modified_base 526: mod_base = OTHER; note = N1-methylpseudouracil
  modified_base 527: mod_base = OTHER; note = N1-methylpseudouracil
  modified_base 536: mod_base = OTHER; note = N1-methylpseudouracil
  modified_base 539: mod_base = OTHER; note = N1-methylpseudouracil
  modified_base 545: mod_base = OTHER; note = N1-methylpseudouracil
  modified_base 546: mod_base = OTHER; note = N1-methylpseudouracil
  modified_base 553: mod_base = OTHER; note = N1-methylpseudouracil
  modified_base 555: mod_base = OTHER; note = N1-methylpseudouracil
  modified_base 557: mod_base = OTHER; note = N1-methylpseudouracil
  modified_base 569: mod_base = OTHER; note = N1-methylpseudouracil
  modified_base 575: mod_base = OTHER; note = N1-methylpseudouracil
  modified_base 577: mod_base = OTHER; note = N1-methylpseudouracil
  modified_base 578: mod_base = OTHER; note = N1-methylpseudouracil
  modified_base 582: mod_base = OTHER; note = N1-methylpseudouracil
  modified_base 587: mod_base = OTHER; note = N1-methylpseudouracil
  modified_base 589: mod_base = OTHER; note = N1-methylpseudouracil
  modified_base 590: mod_base = OTHER; note = N1-methylpseudouracil
  modified_base 591: mod_base = OTHER; note = N1-methylpseudouracil
  modified_base 593: mod_base = OTHER; note = N1-methylpseudouracil
  modified_base 595: mod_base = OTHER; note = N1-methylpseudouracil
  modified_base 596: mod_base = OTHER; note = N1-methylpseudouracil
  modified_base 597: mod_base = OTHER; note = N1-methylpseudouracil
  modified_base 599: mod_base = OTHER; note = N1-methylpseudouracil
  modified_base 604: mod_base = OTHER; note = N1-methylpseudouracil
  modified_base 607: mod_base = OTHER; note = N1-methylpseudouracil
  modified_base 614: mod_base = OTHER; note = N1-methylpseudouracil
  modified_base 617: mod_base = OTHER; note = N1-methylpseudouracil
  modified_base 625: mod_base = OTHER; note = N1-methylpseudouracil
  modified_base 626: mod_base = OTHER; note = N1-methylpseudouracil
  modified_base 632: mod_base = OTHER; note = N1-methylpseudouracil
  modified_base 638: mod_base = OTHER; note = N1-methylpseudouracil
  modified_base 648: mod_base = OTHER; note = N1-methylpseudouracil
  modified_base 650: mod_base = OTHER; note = N1-methylpseudouracil
  modified_base 652: mod_base = OTHER; note = N1-methylpseudouracil
  modified_base 656: mod_base = OTHER; note = N1-methylpseudouracil
  modified_base 659: mod_base = OTHER; note = N1-methylpseudouracil
  modified_base 661: mod_base = OTHER; note = N1-methylpseudouracil
  modified_base 664: mod_base = OTHER; note = N1-methylpseudouracil
  modified_base 667: mod_base = OTHER; note = N1-methylpseudouracil
  modified_base 672: mod_base = OTHER; note = N1-methylpseudouracil
  modified_base 679: mod_base = OTHER; note = N1-methylpseudouracil
  modified_base 683: mod_base = OTHER; note = N1-methylpseudouracil
  modified_base 688: mod_base = OTHER; note = N1-methylpseudouracil
  modified_base 692: mod_base = OTHER; note = N1-methylpseudouracil
  modified_base 693: mod_base = OTHER; note = N1-methylpseudouracil
  modified_base 695: mod_base = OTHER; note = N1-methylpseudouracil
  modified_base 696: mod_base = OTHER; note = N1-methylpseudouracil
  modified_base 702: mod_base = OTHER; note = N1-methylpseudouracil
  modified_base 703: mod_base = OTHER; note = N1-methylpseudouracil
  modified_base 709: mod_base = OTHER; note = N1-methylpseudouracil
  modified_base 722: mod_base = OTHER; note = N1-methylpseudouracil
  modified_base 725: mod_base = OTHER; note = N1-methylpseudouracil
  modified_base 730: mod_base = OTHER; note = N1-methylpseudouracil
  modified_base 731: mod_base = OTHER; note = N1-methylpseudouracil
  modified_base 734: mod_base = OTHER; note = N1-methylpseudouracil
  modified_base 742: mod_base = OTHER; note = N1-methylpseudouracil
  modified_base 750: mod_base = OTHER; note = N1-methylpseudouracil
  modified_base 753: mod_base = OTHER; note = N1-methylpseudouracil
  modified_base 755: mod_base = OTHER; note = N1-methylpseudouracil
  modified_base 756: mod_base = OTHER; note = N1-methylpseudouracil
  modified_base 757: mod_base = OTHER; note = N1-methylpseudouracil
  modified_base 761: mod_base = OTHER; note = N1-methylpseudouracil
  modified_base 766: mod_base = OTHER; note = N1-methylpseudouracil
  modified_base 768: mod_base = OTHER; note = N1-methylpseudouracil
  modified_base 772: mod_base = OTHER; note = N1-methylpseudouracil
aggaaataagagagaaaagaagagtaagaagaaatataagaccccggcgccgccaccatggagcgggataccctcctgaaggctctgctggagatcgcaagctgcctggagaaagccctccaggtgttccaccaagtgttcatcttcatgacatactggcatctgctgaacgcctttaccgtgaccgtacccaaggacctgtacgtggtggagagagataccctgctgaaagccctgctggaaatcgcctcgtgcctggagaaggccctgcaggtgtttcaccaggtgttcattttcatgacctattggcacctgttgaacgcttttactgtgaccgtgccaaaggatttatacgtggtggagcgggacacactgctgaaggccctgcttgagatcgccagctgtctcgagaaagccctgcaagtgttccaccaggtattcatctttatgacttactggcacctgctcaacgccttcaccgtgacagtgcccaaggacttgtacgtggtcgagcgggacaccctgttaaaggccctgctggagattgccagctgtctggagaaggcactgcaggtgttccatcaggtgtttatctttatgacctactggcacctcctgaacgccttcacagtgaccgtgcccaaggatctctacgtggtgtgataataggctggagcctcggtggcctagcttcttgccccttgggcctccccccagcccctcctccccttcctgcacccgtacccccgtggtctttgaataaagtctgagtgggcggc

SEQ ID NO: 303-369 - skipped (67 entries)

SEQ ID NO: 370 - RNA, 120 nt, synthetic construct
  source 1..120: mol_type = other RNA
aaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 371-501 - skipped (131 entries)

SEQ ID NO: 502 - RNA, 100 nt, synthetic construct
  source 1..100: mol_type = other RNA
aaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 503 - RNA, 120 nt, synthetic construct; identical to SEQ ID NO: 370
  source 1..120: mol_type = other RNA

SEQ ID NO: 504-707 - skipped (204 entries)

SEQ ID NO: 708 - RNA, 47 nt, unidentified
  source 1..47: mol_type = unassigned RNA
gggaaataagagagaaaagaagagtaagaagaaatataagagccacc

SEQ ID NO: 709 - skipped

SEQ ID NO: 710 - RNA, 21 nt, unidentified
  source 1..21: mol_type = unassigned RNA
cgcgtaccaaaagtaataatg

SEQ ID NO: 711 - RNA, 30 nt, synthetic construct
  source 1..30: mol_type = other RNA
aaaaaaaaaaaaaaaaaaaaaaaaaaaaaa

SEQ ID NO: 712 - RNA, 35 nt, synthetic construct
  source 1..35: mol_type = other RNA
aaaaaaaaaaaaaaaaaaaaaaaaaaaaaaaaaaa

SEQ ID NO: 713 - RNA, 150 nt, synthetic construct
  source 1..150: mol_type = other RNA
  misc_difference 1..150: note = This sequence may encompass 80-150 nucleotides
aaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 714 - protein, 94 aa, synthetic construct
ERDTLLKALLEIASCLEKALQVFHQALLEIASCLERDTLLKALLEIASCLKALLEIASCLEKALEIASCLEKALQVFHQRDTLLKALLEIASCL

SEQ ID NO: 715 - protein, 100 aa, synthetic construct
VFIFMTYWHLLNAFTVTVPKDLYVVLLNAFTVTVVFIFMTYWHLLNAFTTYWHLLNAFTVTVPKLNAFTVTVPKDLYVVFMTYWHLLNAFTVTVAFTVTV

SEQ ID NO: 716 - protein, 201 aa, synthetic construct
MERDTLLKALLEIASCLEKALQVFHQVFIFMTYWHLLNAFTVTVPKDLYVVERDTLLKALLEIASCLEKALQVFHQVFIFMTYWHLLNAFTVTVPKDLYVVERDTLLKALLEIASCLEKALQVFHQVFIFMTYWHLLNAFTVTVPKDLYVVERDTLLKALLEIASCLEKALQVFHQVFIFMTYWHLLNAFTVTVPKDLYVV